(19) United States
(12) Patent Application Publication
(10) Pub. No.: US 2002/0156038 A1
(43) Pub. Date: Oct. 24, 2002

(54) Gene expression profiling of antidepressant action in the brain

(76) Inventors: Pascal Bonaventure, San Diego, CA (US); Hongqing Quo, San Diego, CA (US); Xuejun Liu, San Diego, CA (US); Fredrik Kamme, San Diego, CA (US); Bernhard Meurers, La Jolla, CA (US); Josepha E.M.F. Leysen, Oud-Turnhout (BE); Margot H.M. Bakker, Breda (NL)

Correspondence Address: Philip S. Johnson, Esq., Johnson & Johnson, One Johnson & Johnson Plaza, New Burnswick, NJ, 08933-7003, US

(21) Appl. No.: 09/971,900

(22) Filed: Oct. 4, 2001

Related U.S. Application Data
(60) Provisional application No. 60/238,374, filed on Oct. 6, 2000.
(60) Provisional application No. 60/295,782, filed on Jun. 4, 2001.

(51) Int. Cl. A61K 48/00; C12Q 1/68; C12M 1/34

(52) U.S. Cl. 514/44; 435/6; 435/287.2

(57) ABSTRACT

Implementing gene expression to study drug action in the central nervous system is complicated by functional heterogeneity because of the existence of many different neuronal subtypes within the mammalian brain. The integration of laser capture microdissection (LCM) and RNA amplification with cDNA microarray technology allows for large-scale gene expression analysis at cellular level. Using this approach, we have generated gene expression profiles of imipramine, a reference antidepressant, and a new putative antidepressant, novelR1 in several laser-captured brain nuclei (locus coeruleus, dorsal raphe, hypothalamic paraventricular nucleus and hippocampus) of rats subjected to the chronic mild stress model (CMS) of depression.

### CROSS-REFERENCE TO RELATED APPLICATION

This application claims the benefit of United States Provisional Application Ser. No. 60/238,374, filed on Oct. 6, 2000, and U.S. Provisional Application Ser. No. 60/295,782, filed on Jun. 4, 2001.

### BACKGROUND OF THE INVENTION

Microarray technology represents a potentially powerful approach to identifying genes specifically expressed in different cell of tissue type [P. O. Brown and D. Botstein, “Exploring the new world of the genome with DNA Microarrays” (1999) Nat Genet, 21:33-7; D. Shalon, “Gene expression micro-arrays: a new tool for genomic research”, (1998) Pathol. Biol. (Paris), 46;107-9; S. M. Welford, J. Gregg, E. Chen, D. Garrison, P. H. Sorensen, C. T. Denny and S. F. Nelson, “Detection of differentially expressed genes in primary tumor tissues using representational differences analysis coupled to microarray hybridization” (1998) Nucleic Acids Res, 26:3059-65]. The application of microarray to the study of gene expression patterns in the central nervous system is complicated by functional heterogeneity because of the existence of many different neuronal subtypes within the mammalian brain [D. H. Geschwind, “Mice, microarrays, and the genetic diversity of the brain [In Process Citation]” (2000) Proc. Natl. Acad. Sci. USA, 97:10676-8; S. J. Watson, F. Meng, R. C. Thompson and H. Akil, “The ‘chip’ as a specific genetic tool” (2000) Biol Psychiatry, 48:1147-56]. This heterogeneity consists of literally hundreds of brain nuclei that are in close proximity to each other as well as many different neuronal subtypes in cortical layers of different brain regions. We have demonstrated in a previous study that the integration of laser capture microdissection (LCM), T7 based RNA amplification and cDNA microarrays allows to profile gene expression at cellular level [L. Luo, R. C. Salunga, H. Guo, A. Bittner, K. C. Joy, J. E. Galindo, H. Xiao, K. E. Rogers, J. Wan, M. R. Jackson and M. G. Erlander, “Gene expression profiles of laser-captured adjacent neuronal subtypes” (1999) [published erratum appears in Nat Med 1999 Mar; 5(3):355], Nat Med, 5:117-22.; R. C. Salunga, H. Guo, L. Luo, A. Bittner, K. C. Joy, J. Chambers, J. Wan, M. R. Jackson and M. G. Erlander, “Gene expression analysis via cDNA microarrays of laser capture microdissected cells from fixed tissue” (1999) In M. Schena (Ed.), “DNA microarrays a practical approach”, Oxford University Press, Oxford].

Recently, expression profiling using microarrays has been successfully used to study the action of the topical anesthetic dyclonine in S. cerevisiae [Hughes, 2000 #346]. Implementing gene expression to study drug action in the central nervous system is complicated by functional heterogeneity because of the existence of many different neuronal subtypes within the mammalian brain [Geschwind, 2000 #373] [Watson, 2000 #442]. This heterogeneity consists of literally hundreds of brain nuclei that are in close proximity to each other as well as many different neuronal subtypes in cortical layers of different brain regions. Therefore, we reasoned that to study the in-vivo action of drugs there needed to be from the technical side, the ability to (1) microdissect small numbers of neurons (˜100) from selected nuclei/subnuclei; (2) amplify the extracted mRNA in a reproducible manner; and (3) generate robust cDNA microarray data. The integration of laser capture microdissection (LCM), T7 based aRNA amplification and cDNA microarrays technology allows profiling of gene expression from specific cell type in situ [Luo, 1999 #352] [Salunga, 1999 #347].

Acute tricyclic antidepressants and other classes of antidepressant have a relatively well defined pharmacological action/target in-vitro of presynaptic reuptake inhibition of biogenic amines (norepinephrine, serotonin, dopamine). However, clinical efficacy requires three to six weeks suggesting that in-vivo a more complex mechanism occurs [Nestler, 1998 #409] [Stanford, 1996 #252]. Given this lag in efficacy, it is reasonable to assume that the mechanism of action of chronic antidepressant treatment in vivo may in part involve the modulation of gene expression. Therefore, gene expression profiling of selected brain nuclei may help to elucidate the chronic mechanisms of antidepressant action.

### SUMMARY OF THE INVENTION

The present invention provides gene expression profiles at a cellular level of multiple brain nuclei after CMS±chronic treatment with antidepressant imipramine. Imipramine, a potent inhibitor of norepinephrine (NE) and serotonin (5-HT) uptake was selected as reference compound. In addition, a new putative antidepressant was examined to determine whether different in-vitro pharmacological properties but similar behavioral effects of imipramine and the new compound in the CMS model result in similar gene expression patterns. This new compound displays U2 adrenoceptor and 5-HT₇ receptor antagonism properties [Kennis, 2000 #394]. The present invention also provides potential new targets for drug discovery to identify compounds useful to treat depression and other neurological diseases and/or conditions.

### BRIEF DESCRIPTION OF THE DRAWINGS

FIG. 1: Laser Capture Microdissection from Nissl-Stained Sections (10 μM).

Panel A: hippocampus including CA1, CA3 and DG before capture

Panel B: hippocampus after capture of CA1 cells (c) CA1 captured cells.

Panel C: Scale bar represent 200 μm.

FIG. 2: Body Weight and Sucrose Consumption in Animals Exposed to CMS.

Effect of chronic treatment with vehicle (1 ml/kg 2× per day), imipramine (10 mg/kg/day) and novelR1 (0.16 mg/kg, 2× per day)

Panel A: on the body weight, and

Panel B: the consumption of a 1% sucrose solution in controls (open symbols) and in animals exposed to CMS (closed symbols). Treatment commenced following 2 weeks of stress. Values are means +/−SEM.

*−p<0.05, **−p<0.01, *** −p<0.001; relative to vehicle- or drug-treated control groups.

−p<0.05, −p<0.01, −p<0.001; relative to drug-treated stressed animals at Week 0.

FIG. 3: Experimental Reproducibility of LCM, T7 Based aRNA Amplification and cDNA Microarray Procedure.

Pair-wise similarities of gene expression profiles measured by the linear correlation coefficient.

Panel A: average linear correlation coefficient (R) for each brain area (n =3 to 5 per group)

Panel B: examples of scatter plots showing the individual linear correlation coefficient for two groups of rats (control/vehicle DR and control/vehicle CA3).

FIG. 4: Gene Expression Profiling of Laser Captured Brain Nuclei of Vehicle/Control Rats.

Panel A: table showing the number of genes expressed in each the laser-captured nuclei (100 cells) according our criteria for a call of presence (i.e. 3 fold above background). The number of genes uniquely expressed in one of these nuclei is also given

Panel B: two-dimensional hierarchical cluster analysis of the 871 genes (horizontal axis) detected in at least one of the region investigated (vertical axis). Red, high intensity (log10 scale), black not expressed (below 3 fold the background). The 7 regions on the vertical axis were assembled into a dendrogram, where items are joined by very short branches if they are similar to each other and by increasingly longer branches as their similarity decreases

Panel C: distribution of several genes for specific laser-captured brain region, data represent the average fluorescence intensity. Note that the fluorescence intensity readings for vasopressin and oxytocin in PA and MG correspond to the maximal intensity.

* The anatomical distribution of these genes matched the distribution previously described in the literature: vasopressin [Hallbeck, 1999 #359], oxytocin [Burbach, 1987 #354], somatostatin [Fitzpatrick-McElligott, 1988 #358], dynorphin [Morris, 1986 #364], tyrosine hydroxylase [Tohyama, 1998 #348], serotonin transporter [Swan, 1997 #419], galanin [Tohyama, 1998 #348], RGS14 [Traver, 2000 #416], H₃ histamine receptor [Lovenberg, 1999 #338], mineralocorticoid receptor [Reul, 1985 #424].

FIG. 5: In situ Verification of the cDNA Microarray Data for Tyrosine Hydroxylase mRNA by ISHH.

Autoradiograms of frontal sections (left hemisphere) through the locus coeruleus hybridized with cRNA probes for tyrosine hydroxylase mRNA. (a, c, e) control; (b, d, f) stressed; (a, b) vehicle; (c, d) imipramine treated (10 mg/kg/day); (e, f) novelR1 treated (0.16 mg/kg, 2× per day) (g) Bound radioactivity of tyrosine hydroxylase cRNA probe in locus coeruleus of each group of animals. Values are means ±SEM n =3-5. Differences between control and CMS or drug treated groups were statistically significant. ***p<0.001. Scale bar represents 0.5 cm.

FIGS. 6: Overlapping and Non Overlapping Gene Expression Pattern in Unstressed and CMS Drug Treated Rats in the 7 Different Nuclei.

The diagrams show the number of genes with the indicated patterns.

Panel A: unstressed and CMS imipramine treated animals

Panel B: unstressed and CMS novelR1 treated animals

Panel C: uniquely changed in CMS imipramine and NOVELR1: the subset of genes with an accession number overlapping are listed.

### DETAILED DESCRIPTION OF THE INVENTION

Gene expression profiling of antidepressants should be carried in the context of a behavioral model of depression. The chronic mild stress (CMS) model, a behavioral model of depression for which predictive, face and construct validities have been demonstrated was selected [Willner, 1992 #150] [Willner, 1997 #144] [Yadid, 2000 #422]. The CMS model was developed to mimic anhedonia, a core symptom of depressive disorders. Chronic sequential administration of a variety of mild stressors causes a decrease in responsiveness to rewards in rats, which is reversed by chronic administration of antidepressants drugs [Willner, 1992 #150]. The CMS model of depression also causes a decrease in body weight [Forbes, 1996 #413] but the magnitude of this decrease is minor and the validity, reliability and utility of the CMS is overall well recognized [Yadid, 2000 #422] [Argyropoulos, 1997 #205] #] [Willner, 1996 #226].

The locus coeruleus (LC), dorsal raphe (DR), hypothalamic paraventricular nucleus (PVN) magno and parvocellular division (MG, PA) and hippocampus (CA1, CA3 and DG) were selected for the present study. About one hundred cells were laser-captured in each of these nuclei. These regions were selected based on the location of the sites of action of the drugs and the critical neuroanatomical substrate for the stress response. In an autoradiographic analysis of the in vivo distribution of [³H]imipramine in rat brain, the locus coeruleus (LC) and the hippocampus have been shown to be the primary sites of action of imipramine [Duncan, 1991 #407]. Imipramine has also been shown to interfere with the serotoninergic transmission, therefore the dorsal raphe was selected in the present study. The LC is a critical neuroanatomical substrate for the stress response [Brady, 1994 #37] [Nestler, 1999 #240] [Valentino, 1998 #71], the PVN was added to the list of the investigated nuclei because of its role in the stress response [Weiss, 1994 #36]. Information on the neuroanatomical distribution of genes is critical for the process of understanding their function in the central nervous system. The integration of laser capture microdissection, T7 based RNA amplification and cDNA microarrays allows dissection of neuronal heterogeneity and at the same time to measure the gene expression level of thousands of genes. We have built a catalogue of rat brain-nuclei specific gene expression profiles. The following seven brain nuclei (100 cells) were analyzed: locus coeruleus (LC), dorsal raphe nucleus (DR), hypothalamic paraventricular (PVN, parvocellular division PA and magnocellular division MG) and hippocampus (CA1, CA3 and DG). Of the 2137 genes (unique mRNAs) interrogated, 871 (40.76%) gave a hybridization signal consistent with a call of presence in at least one brain region (i.e. at least three-fold above background level). The analysis was validated by the agreement of our findings with that of previously documented expression patterns of “signature” genes for specific brain nuclei. For example, abundant mRNA expression encoding the serotonin transporter was detected in DR cells but not detected in the other examined nuclei. The different nuclei investigated were found to have about four to seven percent in unique gene expression. The hierarchical clustering analysis shows that the two divisions of the PVN (PA and MG) were closely related and that similarly the three regions of the hippocampus (CA1, CA3 and DG) shared a similar gene expression profile. This study demonstrates the feasibility and utility of cellular brain nuclei profiling.

Using the LCM, T7 based amplification and cDNA microarray approach, we have generated a catalogue of cell specific gene expression profiles that can be used to determine which genes are expressed and where in the adult rat brain. Recently, Sandberg et al. [R. Sandberg, R. Yasuda, D. G. Pankratz, T. A. Carter, J. A. Del Rio, L. Wodicka, M. Mayford, D. J. Lockhart and C. Barlow, “Regional and strain-specific gene expression mapping in the adult mouse brain” (2000) Proc. Natl. Acad. Sci., 97:11038-43] and Zirlinger et al. [M. Zirlinger, G. Kreiman and D. J. Anderson, “Amygdala-enriched genes identified by microarray technology are restricted to specific amygdaloid subnuclei” (2001) Proc. Natl. Acad. Sci. 98:5270-5] have demonstrated the utility of brain region expression profiling. In these studies, gene expression analysis was performed on dissected brain regions. In the present study, a more neuroanatomically refined method (i.e. LCM) was introduced to isolate the cells of interest. The LCM technique allows the procurement of a selected cell population [S. J. Watson, F. Meng, R. C. Thompson and H. Akil, “The ‘chip’ as a specific genetic tool” (2000) Biol Psychiatry, 48:1147-56]. Brain tissues are composed of complex admixtures of different cell types, their biological meaningful analysis necessitate the procurement of a pure sample of the cells of interest. It is likely that general brain region dissection approaches would dilute the concentration of RNAs from neurons with RNAs from millions of potentially unrelated neurons and glia.

The correlation coefficient underlying the pair-wise similarities of gene expression profiles within one nuclei obtained in the present study demonstrate the reproducibility of the LCM, T7 RNA amplification and cDNA microarray approach. The high concordance between our data and published results validates the approach. However, some false negative findings were observed in the present study. Hallbeck et al. [M. Hallbeck, O. Hermanson and A. Blomqvist, “Distribution of preprovasopressin mRNA in the rat central nervous system” (1999) J. Comp. Neurol., 411:181-200] have reported the presence of low level of vasopressin mRNA in the hippocampus by in situ hybridization. In the present study, the level of vasopressin mRNA detected in the hippocampus was found between two- and three-fold different from the background and so, according to our criteria, was classified as not expressed. Low expression of somatostatin mRNA in the hippocampus has also been described in the literature [J. V. Priestley, M. Rethelyi and P. K. Lund, “Semi-quantitative analysis of somatostatin mRNA distribution in the rat central nervous system using in situ hybridization” (1991) J. Chem. Neuroanat., 4:131-53]. As observed for vasopressin, the intensity for somatostatin mRNA in the present study was below the criteria used for a call of presence. The stringent criteria used for a call of presence in the present study could account for these discrepancies. Sandberg et al. [R. Sandberg, R. Yasuda, D. G. Pankratz, T. A. Carter, J. A. Del Rio, L. Wodicka, M. Mayford, D. J. Lockhart and C. Barlow, “Regional and strain-specific gene expression mapping in the adult mouse brain” (2000) Proc. Natl. Acad. Sci., 97:11038-43] reported in their study that approximately 1% of the genes showing hybridization signals on the array were detected as differentially expressed between the four different brain regions investigated. In the present study, we found that the seven different nuclei investigated have about 4% to 7% in unique gene expression. The higher percentage of uniquely expressed found in our study could reflect the higher sensitivity achieved by the LCM approach.

In this study, we were able to classify the different laser-captured brain nuclei by hierarchical clustering analysis. As expected, we found that the two divisions of the PVN (PA and MG) were closely related and, similarly, the three regions of the hippocampus shared similar gene expression profiles. The dendrogram in FIG. 3 represents a new approach to classify brain nuclei. In conclusion, this study demonstrates the feasibility and the utility of cellular brain nuclei profiling. The following examples are intended to illustrate but not limit the present invention.

### EXAMPLE 1

Behavioral Procedure

The stress procedure was conducted on male Wistar rats as previously described [Dziedzicka-Wasylewska, 1997 #224]. On the basis of sucrose intake scores following two weeks of stress, both stressed and control groups were divided further into matched subgroups (n=8), and for the subsequent five weeks received twice-daily intraperitoneal injections of vehicle (isotonic mannitol solution, pH 4.0, 1 ml/kg) or novelR1 (0.16 mg/kg). Once-daily intraperitoneal injections of imipramine (10 mg/kg) were used as the reference treatment. At this dose, imipramine has been shown to cause stable and comparable effects after chronic treatment in all previous studies using this experimental protocol [Willner, 1997 #144]. Sucrose tests were carried out twenty-four hours following the last drug injection (in the case of b.i.d. regime, the second injections on the day preceding the sucrose tests were omitted).

Drugs

The following agents were used: imipramine HCl (RBI, Natick, Mass.) and novelR1.

Statistics

The behavioral results obtained in this study were analyzed by multiple analysis of variance with three between-subjects factors (stress/control, drug treatments and successive sucrose tests). Fisher's LSD test was used for post-hoc comparisons of means.

Tissue Preparation

Twenty-four hours after the final injection of vehicle, imipramine or novelR1, five representative animals from each group were decapitated, and their brains were removed, placed in cryomolds, covered with Tissue-Tek tissue embedding medium (OTC) and snap-frozen in dry ice-cold 2 methyl butane (−60° C.). Ten μm-thick coronal sections in the mid-regions of the PVN (bregma—1.80 mm), hippocampus (bregma—3.30 mm), DR (bregma—1.78 mm) and LC (bregma—9.80 mm) were cut using a cryostat, mounted on non-coated, clear microscope slides and immediately frozen on a block of dry ice. The sections were stored at −70° C.

Laser Capture Microdissection

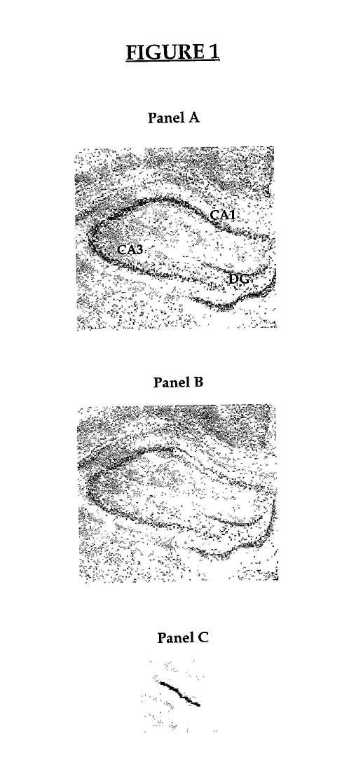

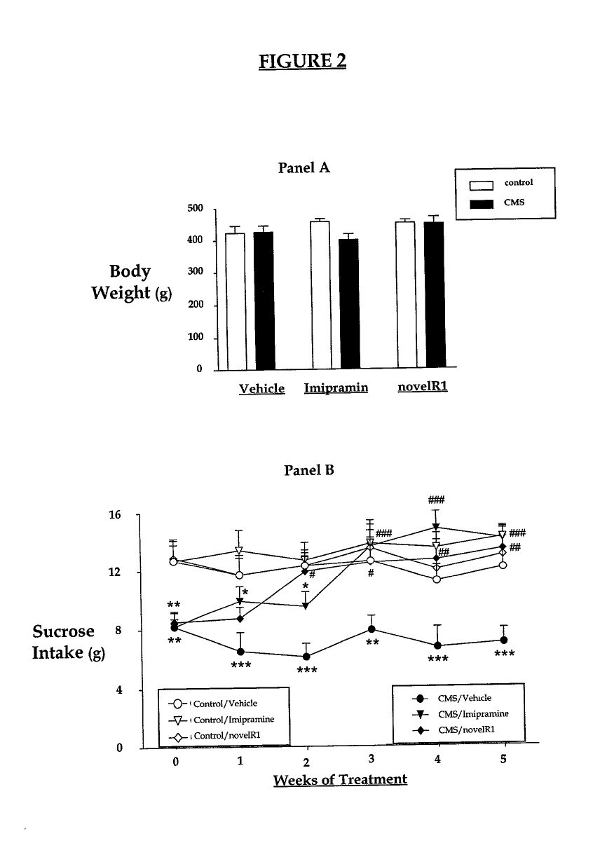

A quick Nissl (cresyl violet acetate) staining was used to identify the neurons as described [Salunga, 1999 #347]. The PixCell II System from Arcturus Engineering, Inc. (Mountain View, Calif.) was used for LCM. According to the manufacturer's protocol, one hundred neurons were captured in each region of interest of each animal (LC, DR, MG, PA, CA1, CA3 and DG). Example of laser-captured cells in CA1 region is shown in FIG. 1.

RNA Extraction and Amplification of LCM Samples

The total RNA was extracted from the individual LCM samples as described [Salunga, 1999 #347] with modifications as specified below. For total RNA extraction, a small volume (20 μl/LCM sample) of denaturing buffer (Micro RNA isolation kit, Stratagene: San Diego, Calif.) containing 2-mercaptoethanol (10 μl/ml, Sigma, St Louis, Mo.) and polyinosine (300 ng/LCM sample, Sigma: St Louis, Mo.) was used. The samples were incubated at 42° C. for ten minutes, and loaded onto a pre-rinsed Microcon-100 column (Millipore: Bedford, Mass.). After two rinses with 500 μl Rnase-free water, the columns were inverted and the samples collected by brief centrifugation. The samples were then processed for two rounds of RNA amplification as described [Salunga, 1999 #347] with modifications for the DNA and RNA purification steps. Quiaquick PCR purification kit (Qiagen: Valencia, Calif.) and RNAeasy Mini Kit (Qiagen: Valencia, Calif.) were used for purification steps. Polyinosine (100 ng) was added to each sample before purification of DNA or aRNA.

cDNA Microarray

A cDNA microarray containing 2147 cDNA clones (corresponds to 2137 unique mRNAs) was used in this study. cDNA clones were obtained commercially from Research Genetics (IMAGE consortium), Incyte Genomics as well as internal sources. All clones were verified by DNA sequencing. Each clone was printed as two independent spots on a given chip. A contact pin microarrayer (Generation III Array Spotter, Molecular Dynamics) was used to spot the clones in duplicate. Microarrays were hybridized and scanned with a confocal laser scanner (Array Scanner, Molecular Dynamics) with excitation at 532nm and collection through a 550 to 600nm band width filter, the PMT set to 630V. Image analysis was performed as previously described [Salunga, 1999 #347].

Statistical Analysis

Hybridization of each sample was performed on two identical microarrays. Each microarray was normalized at the 75 percentile (intensity of the 75 percentile was scaled to 100). Intensity of each clone on an individual microarray was then measured as an average of the intensities of the two corresponding spots, and the intensity of each clone in the sample was further determined as average of the intensities on the two identical chips. The pair-wise similarity of gene expression profiles within a group of same samples (same cell type under identical treatment) was examined by scatter-plot and the correlation coefficient was calculated. Samples having a correlation coefficient of R <0.89 with respect to the majority were treated as out-liers and were excluded for further analysis (at least three rats were included in each group). The intensity of each clone in one particular type of samples was subsequently further defined as the median of intensities of that clone in remaining qualified samples in that group. The resulting intensity matrix (N×M) (N: number of clones; M: number of sample types) was further normalized (spline normalization) so that the distribution of the intensities of all the clones for each individual sample type was fitted to a smooth cubic B-spline. The spline normalization could reduce abnormality in the distribution of the intensities in particular samples caused by saturation in image processing.

In addition to the 2147 clones, fifteen plant cDNA were printed on the same microarray. The intensities of these fifteen plant cDNAs were measured and processed in the same manner as other clones, and used as negative controls. The average intensity of the fifteen plant genes in all M types of samples was defined as background. Each clone was then classified as absent in a cell population if its medium intensity was below the three-fold of the background; or as present if its medium intensity was three-fold above the background.

The ratio (fold change) of gene expression between two samples was calculated after applying *i minimum threshold at the background of plant genes (intensity lower than background was re-set to background). The criteria used to select statistically significant fold changes in gene expression between two samples was at least a 1.8 fold change. Hierarchical clustering was generated by using Cluster software (Stanford).

Quantitative In Situ Hybridization Histochemistry.

In situ hybridization histochemistry was performed according to the procedures described [Bonaventure, 1998 #353] on adjacent tissue sections of the one used for LCM. The plasmid (pBluescript II SK, Stratagene, San Diego, Calif.) containing the cDNA coding for tyrosine hydroxylase (GB:M10244) was linearized and [³⁵S]UTP labeled cRNA probe was generated by in vitro transcription with T7 polymerase. Sections were hybridized at 50° C. with 1.106 c.p.m of [³⁵S]UTP labeled cRNA probe (80 μl per section). After high stringency washes, the sections were exposed to Kodak BiomaxMR film for sixteen hours. [¹⁴C] standard strips (American Radiolabeled chemicals Inc., St Louis, Missouri) were co-exposed to allow densitometry. Autoradiograms were quantified with the aid of an MCID-M4 3.0 Image Analysis system (Imaging Research, St. Catharines, Ontario, Canada). Optical densities in the LC were transformed into levels of bound radioactivity after calibration of the image analyzer with grey-values generated by co-exposed standards. The hybridization signal was expressed as μCi/g. The Student's t-test was used for comparison between the groups.

Body Weight is Unchanged by CMS or Drug Treatment.

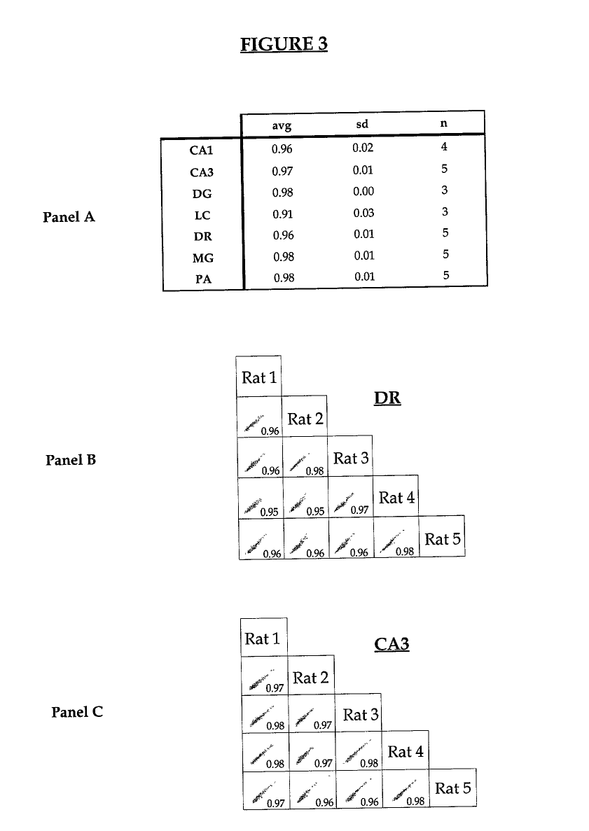

At the start of the study (Baseline) all animals weighed approximately 320 grams and after the initial two weeks of stress (Week 0) the body weights of control and stressed animals were comparable (375 +/−6 and 372 +/−9 g, respectively). At the end of the treatment period (Week 5), body weights of vehicle-treated control and stressed animals were comparable [Group effect: F(1,14)=0.011; p=0.919]. As compared to vehicle-treated groups, neither imipramine nor novelR1 had any significant effect on body weights in controls [IMI: F(1,14)=1.764; NS, novelR1: F(1,14)=1.241; NS] and in stressed [IMI: F(1,14)=1.002; NS, novelR1: F(1,14)=0.518; NS] (FIG. 2a).

Sucrose Intake is Normalized in CMS Rats by Chronic Imipramine/NovelR1 Treatment.

In the final baseline test, all animals drank approximately 13 grams of sucrose solution. Following the initial two weeks of stress (Week 0), the intakes remained at similar level in controls but fell to approximately 8 grams in stressed animals, resulting in a significant Group effect [F(1,14) 11.159; p<0.01]. As shown in FIG. 2b, such a difference between control and stressed animals treated with vehicle persisted for the remainder of the experiment [F(1 1,84)=5.563; p<0.001] and at the end of treatment period, the sucrose consumption in stressed animals receiving vehicle was significantly lower than that measured in control group (p<0.001). As compared to chronic vehicle administration, imipramine did not change the sucrose intake in control animals [Treatment effect: F(1,84)=3.344; NS, Treatment×Weeks interaction: F(5,84)=0.313; NS]. However, in stressed animals, imipramine gradually increased the sucrose consumption, resulting in a significant Treatment effect [F(1,84)=61.277; p<0.001], Treatment×Weeks interaction [F(5,84)=4.117; p<0.01], and Treatment×Group interaction [F(1,168)=13.353; p<0.001] after three weeks of treatment (p<0.001). This effect was maintained thereafter and at the end of treatment period (Week 5), the amount of sucrose solution consumed by these animals was comparable to that of vehicle-treated controls (p=0.168) and significantly higher than that of vehicle-treated stressed animals (p<0.001). The novelR1 like imipramine had no significant effect on the consumption of sucrose solution in control animals [Treatment effect: F(1,84)=0.315; NS, Treatment×Weeks interaction: F(5,84)=0.599; NS]. However, like imipramine, novelR1 fully reversed in stressed animals the CMS-induced deficit in sucrose consumption resulting in a significant Treatment effect [F(1.84)=51.123; p<0.001], Treatment×Weeks interaction [F(5,84)=2.882; p<0.05], and Treatment×Group interaction [F(1,168)=14.238; p<0.001].

As compared to Week 0 scores, the increase of sucrose intake reached statistical significance after two weeks of treatment with novelR1 (p=0.043). This effects was maintained thereafter and at the end of treatment period, the intake in this group of stressed animals did not differ from those measured in vehicle-treated controls (p=0.501) and were significantly higher than those of vehicle-treated stressed animals (p=0.001).

The Greatest Number of Genes Changing Their Expression Level in Response to the CMS Paradigm in Untreated Rats was Found in LC.

A total of 80 genes (3.79 %) were found to change their expression level in response to the CMS paradigm in untreated animals (versus the control/vehicle group). The greatest number of genes changing their expression level was observed in LC (53 genes)>DG (10 genes)>PA (9 genes), CA1 (9 genes)>DR (2 genes), CA3 (2 genes), MG (1 gene) in Table 1 (see below).

TABLE 1

| | | | |
|---|---|---|---|
| | | FOLD | |
| ACC. NO | GENE | (CMS vs ctrl) | NUCLEUS |

Catecholamine synthesis

| | | | |
|---|---|---|---|
| M10144 | Tyrosine hydroxylase | 1.85 | LC |

Neuropeptides

| | | | |
|---|---|---|---|
| A1045437 | Neuropeptide Y** | 2.11 | DG |
| AA955779 | Galanin | 2.06 | PA |
| M10088 | dynorphin** | −2.52 | DG |
| RNU10071 | CART | −1.90 | LC |

Receptors, ion channels

| | | | |
|---|---|---|---|
| AF052746 | Chloride channel CaCC | 3.27 | LC |
| M81830 | Somatostatin R2 (SSR2) | 2.77 | LC |
| AF027955 | GPR27 R | −1.92 | LC |
| Z30425 | Nuclear receptor/orphan | −1.90 | DG |
| | MB67/CAR | | |
| L14618 | Calcitonin R | −1.81 | PA |
| L14618 | Calcitonin R** | −1.80 | LC |
| | GABA signaling | | |
| L08493 | GABA-A R alpha4 subunit | 2.24 | CA1 |
| X54673 | GAT1 | −2.05 | LC |

Glutamate signaling

| | | | |
|---|---|---|---|
| X94552 | mGlu7 R | −2.21 | LC |
| M81886 | glutamate R 1* | −2.03 | CA1 |
| M64752 | glutamate R 1* | −1.89 | CAl |
| HSU08107 | NMDA R subunit | 2.23 | LC |

Calcium

| | | | |
|---|---|---|---|
| AA819346 | Parvalbumin** | 1.93 | DG |
| M31178 | calbindin D28 | 1.87 | CA1 |
| M31178 | calbindin D28 | −2.21 | LC |
| AA819345 | Parvalbumin | −1.99 | LC |
| L10284 | Calnexin | −1.87 | LC |

Signal transduction

| | | | |
|---|---|---|---|
| D83407 | ZAKI-4* | −1.80 | DR |

Heat shock protein

| | | | |
|---|---|---|---|
| M86389 | Hsp27 | −1.85 | PA |

Plasticity

| | | | |
|---|---|---|---|
| X51396 | MAP1B | −2.11 | CA1 |
| X60370 | MAP1B | −1.83 | CA1 |

Proteasome

| | | | |
|---|---|---|---|
| D29956 | ubiquitin isopeptidase | 1.97 | LC |
| D17296 | Polyubiquitin | −2.01 | LC |
| W07641 | ubiquit-like protein SMT3B | −2.00 | LC |

Transcription factor

| | | | |
|---|---|---|---|
| Y10926 | RBP-L | 2.16 | LC |
| Y07688 | NfiX | −2.38 | LC |
| AF057691 | TCF-3 | −1.83 | DR |
| W65013 | similar to X07384 GLI | −1.85 | CA3 |
| | PROTEIN | | |

p53

| | | | |
|---|---|---|---|
| U67885 | p53 binding prot 1 | −2.37 | LC |
| U28789 | PACT | −2.01 | LC |

Others

| | | | |
|---|---|---|---|
| X58526 | L1 element | −2.39 | LC |
| X58526 | L1 element | −2.35 | DG |
| 100256 | Sequence 7 from U.S. Pat. | 2.38 | LC |
| | No. 4900811** | | |
| 100256 | Sequence 7 from U.S. Pat. | −1.93 | PA |
| | No. 4900811 | | |

These genes were classified in functional families (i.e. catecholamine synthesis, neuropeptides, receptors or ion channels, GABA signaling, glutamate signaling, calcium, signal transduction, heat-shock protein, plasticity, proteasome, transcription factors and p53). The genes that could be classified in these functional families and with an accession number are listed in Table 2 (containing 39 genes).

TABLE 2
Number of genes detected in each laser-captured nuclei (100 cells)
according our criteria for a call of presence (i.e. three-fold above
background). The number of genes uniquely expressed in one of these
nuclei is also given.

| | Detected | Unique |
|---|---|---|
| CA1 | 499 | 32 |
| CA3 | 527 | 22 |
| DG | 527 | 24 |
| LC | 513 | 43 |
| DR | 510 | 38 |
| MG | 519 | 21 |
| PA | 520 | 25 |

The increase in expression level of tyrosine hydroxylase mRNA (GB: M10244) in the LC of CMS/vehicle rats was further confirmed by quantitative in situ hybridization (FIG. 5a and b).

For the glutamate receptor 1 (GB: M81886, M64752) and the microtubule associated protein MAPI (GB: X51396, X60370) two different clones (from different species) were spotted on the microarray and consistent changes in gene expression level were observed for both clones as shown in Table 1. MAP1B plays an important role in the extension of axons and dendrites.

The chloride channel CaCC (GB: AF052746) and the somatostatin receptor 2 (GB: M81830) mRNAs showed the biggest amplitudes of changes of their expression levels in response to CMS (i.e 3.37-fold and 2.77-fold, respectively).

Six genes (five with an accession number) were found to change their expression level in the CMS/vehicle group in two different laser-captured nuclei (i.e. calcitonin receptor GB: L14618 in PA and LC, parvalbumin GB: AA819346 in DG and LC, calbindin D28 GB: M31178 in CA1 and LC, LI element GB: X58526 in LC and DG and GB: 100256 in LC and PA) (Table 1). Noteworthy, two calcium-ion binding protein (parvalbumin and calbindin D28) were found among these six genes.

At the end of the behavioral experiment, two different endpoints in sucrose consumption levels were observed: 13 grams of sucrose intake including the following groups vehicle/control, imipramine/control, imipramine/CMS, novelR1/control, novelR1/CMS) and 8 grams of sucrose intake including the vehicle/CMS group (FIG. 2b). The differences in gene expression level between these two groups (i.e. 8 grams versus 13 grams) reflects either changes causal to the decrease in sucrose consumption level or changes in response/adaptation to the CMS paradigm. A total of 30 genes (1.4%) were found to change their expression level in the untreated CMS group uniquely (i.e. no change in any of the other comparisons performed). The largest set of genes changing their expression level after CMS exposure uniquely was observed in LC (26)>PA (2)>DR (1), CA1 (1). These genes are listed in Table 1. Interestingly, in the GABA signaling pathway the expression level of both GABA-A receptor and GAT-1 was changed by the CMS paradigm uniquely.

Overall Both Drugs Changed the Expression Level of a Larger Set of Genes Than the CMS Paradigm and NovelR1 Changed the Expression Level of More Genes Than Imipramine in Unstressed Rats.

Overall imipramine and novelR1 were found to change the expression level of more genes than the CMS paradigm in control animals (CMS: 80 genes, 3.79%; imipramine: 86 genes, 4%; novelR1: 306 genes, 14%). These changes in gene expression levels induced by imipramine or novelR1 in unstressed rats do not reflect a change in sucrose uptake. In all, the nuclei investigated (excepted CA3 and MG), chronic treatment with novelR1 changed the expression level of more genes than imipramine in control animals (FIG. 6).

For both drugs genes that changed their expression levels in more than one of the investigated nuclei were found (imipramine: 12 genes: Table 3, R10747: 42 genes: Table 4). Both drugs were found to increase tyrosine hydroxylase mRNA levels in LC (fold increase versus control/vehicle: imipramine, 2.27; novelR1, 3.37). This up-regulation of tyrosine hydroxylase mRNA level was confirmed by in situ hybridization (FIGS. 5c and 5d). Noteworthy, the pterin-4a-carbinolamine dehydratase (DCoH or PCD, GB: AA901316) which catalyses the recycling of the cofactor biopterin [Depaepe, 2000 #439] shared a similar expression pattern in LC (fold increase versus vehicle/control: imipramine, 1.98; novelR1, 2.70). DcoH is requested in association with tyrosine hydroxylase for dopamine biosynthesis. Co-localization of DCoH and tyrosine hydroxylase in LC neurons has been previously demonstrated [Depaepe, 2000 #439]

TABLE 3

| | | | |
|---|---|---|---|
| | | IMINCMS | |
| ACC. NO. | GENE NAME | vs. IMI | NUCLEI |

Apoptosis

| | | | |
|---|---|---|---|
| U67885 | p53 binding prot 1 | 3.12 | DG |
| U59746 | Bcl-w | 2.00 | CA1 |
| AF022223 | BAG-1 | 1.83 | DR |

Catecholamine synthesis

| | | | |
|---|---|---|---|
| AA142680 | dopamine beta-hydroxylase | 1.95 | LC |
| A1044610 | dopa decarboxylase | 1.81 | LC |

Energy production

| | | | |
|---|---|---|---|
| V01556 | mitochondrial nenome | 2.36 | DG |
| | fragment | | |
| V00680 | mitochondrial genes coding | 1.90 | CA3 |
| | for 16S and 12S | | |
| V00681 | mitochondrial genes for | 1.84 | MG |
| | 16S | | |
| V00681 | mitochondrial genes for | −2.85 | DR |
| | 16S | | |
| X14848 | mitochondrial genome | −2.32 | CA1 |
| V00681 | mitochondrial genes for | −2.07 | CA1 |
| | 16S | | |
| V00681 | mitochondrial genes for | −2.04 | PA |
| | 16S | | |
| V01556 | mitochondrial genome | −1.99 | DR |
| | fragment | | |
| V00680 | mitochondrial genes coding | −1.93 | CA1 |
| | for 16S and 12S | | |

GABA signaling

| | | | |
|---|---|---|---|
| L08495 | GABA receptor alpha6 | 2.56 | DG |
| X54673 | GAT1 | 2.24 | CA1 |
| X54673 | GAT1 | 2.14 | DG |
| X14767 | GABA-A receptor, beta 1 | 1.96 | CA1 |
| | subunit | | |
| X15376 | GABA-A receptor, gamma | 2.00 | DG |
| | 2 subunit | | |

Glutamate signaling

| | | | |
|---|---|---|---|
| AA297071 | Similar to N-methyl-D- | 1.97 | LC |
| | aspartate glutamate receptor | | |
| U16129 | Glutamate receptor (GluR4) | 2.27 | DG |

Growth factors

| | | | |
|---|---|---|---|
| M21571 | platelet-derived growth | 1.96 | CA1 |
| | factor (PDGFA) A chain | | |
| U66198 | FGF13 | 1.91 | CA1 |
| U66198 | FGF13 | 2.27 | DG |

Heat shock protein

| | | | |
|---|---|---|---|
| M19645 | BiP/GRP78/ER resident | 2.18 | LC |
| | HSP70 | | |
| D13627 | TCP-1 Hsp60, theta subunit | 2.06 | DR |
| AA823192 | heat-shock protein 40 | 2.03 | LC |
| X15187 | HSP90 | 1.91 | DR |
| M11942 | 70 kd heat-shock-like | 1.89 | LC |
| | protein | | |
| X63368 | HSJ1 | 1.88 | CA1 |

Neuropeptides

| | | | |
|---|---|---|---|
| AI045437 | neuropeptide Y | 2.56 | LC |
| AA818532 | gamma-preprotachykinin A | 1.99 | DR |
| M10088 | Dynorphin | 1.86 | MG |

Plasticity

| | | | |
|---|---|---|---|
| M16228 | GAP-43 | 2.59 | DG |
| X51396 | MAP1B | −4.65 | DG |

Proteasome

| | | | |
|---|---|---|---|
| U44839 | ubiquitin C-terminal | 4.27 | DG |
| | hydrolase | | |
| D10699 | ubiquitin carboxyl-terminal | 2.17 | LC |
| | hydrolase | | |
| D00761 | proteasome beta 1 subunit | 2.09 | DR |
| U44839 | ubiquitin C-terminal | 1.93 | LC |
| | hydrolase | | |
| AA859300 | proteasomes C2 component | 1.90 | DR |
| W07641 | ubiquit.-like protein | −2.79 | DG |
| | SMT3B (SUMO-1) | | |
| X99585 | ubiquit.-like protein | −2.60 | DG |
| | SMT3B (SUMO-1) | | |
| M29859 | proteasomes C2 component | −2.34 | DG |
| D00763 | proteasome alpha 4 subunit | −2.31 | DG |
| X99585 | ubiquit.-like protein | −1.99 | MG |
| | SMT3B (SUMO-1) | | |
| AF079564 | ubiquitin carboxy-terminal | −1.87 | DR |
| | hydrolase ubp41 | | |
| U75362 | ubiquitin carboxy terminal | −1.84 | DG |
| | hydrolase | | |
| M29859 | proteasomes C2 component | −1.83 | MG |
| AA859229 | ubiquitin conjugating | −1.82 | DG |
| | enzyme | | |

Receptors, ions channels

| | | | |
|---|---|---|---|
| Y07521 | Kv3.1a = NGK2 potassium | 2.94 | DG |
| | channel; V-gated | | |
| U33267 | Human glycine receptor | 2.60 | DG |
| | beta subunit (GLRB) | | |
| | mRNA, complete cds | | |
| | H3 Histamine Rec | 2.09 | DR |
| L14618 | Calcitonin Rec | 2.06 | LC |
| X66842 | Serotonin 2B Rec | 2.05 | MG |
| Z30425 | Nuclear receptor/orphan | 2.03 | MG |
| | MB67/CAR | | |
| AF064876 | BCNG-1 hyperpolarization | 2.03 DG | |
| | NSC channel = HCN1 = | | |
| | HAC2 | | |
| AF027955 | GPR27 Rec | 1.96 | DG |
| Y10148 | NTR2 receptor | 1.94 | DG |
| | H3 Histamine Rec* | 1.93 | LC |
| X97121 | Neurotensin 2 Rec | 1.90 | DG |
| M81253 | Kv3.4 gene | 1.86 | LC |
| AA002993 | Potassium channel Kv3.4a | 1.85 | DR |
| | (Raw3), V-gated | | |
| U07225 | Purinergic (P2U) | 2.88 | DG |
| AI058645 | Kv4.3 potassium channel | 2.60 | DG |
| X66842 | Serotonin 2B Rec | 2.23 | DR |
| X66842 | Setotonin 2B Rec | 1.92 | PA |
| Z30425 | Nuclear receptor/orphan | 1.88 | DR |
| | MB67/CAR | | |
| U07225 | Purinerpic (P2U) | 1.87 | PA |
| U12194 | voltage-gated Na + channel | 1.83 | LC |
| | beta-1 sub (SCN1B) gene | | |
| M21948 | dihydropyridine-sens Ca | 1.83 | CA3 |
| | channel alpha-2 sub | | |
| M24898 | Nuclear receptor alpha1 | 1.81 | DG |
| | for thyroid horm | | |

Signa transduction

| | | | |
|---|---|---|---|
| D83407 | ZAKI-4 | 3.41 | DG |
| M38194 | extracellular signal- | 2.26 | LC |
| | regulated kinase 1 | | |
| M12673 | guanine nucleotide-binding | −4.12 | DR |
| | protein G-s, alpha | | |
| M29871 | ras-related C3 botulinum | −2.69 | LC |
| | toxin substrate (rac) | | |
| D14425 | Calcineurin | −2.24 | DG |
| M60156 | Go alpha | −1.89 | DR |

Transcription factor

| | | | |
|---|---|---|---|
| AF057691 | TCF-3 | 3.35 | DG |
| AF012923 | Wig-1 | 2.22 | LC |
| AF065386 | Jab1 | 2.14 | DR |
| W65013 | Similar to X07384 GLI | −3.24 | DG |
| | PROTEIN | | |
| X17163 | c-jun | −2.50 | DG |
| X12740 | C-Jun of the transcription | −1.85 | DG |
| | factor AP-1 | | |

Others

| | | | |
|---|---|---|---|
| M25888 | Lipophilin | 7.94 | DG |
| I00256 | Sequence 7 from U.S. Pat. | 5.89 | DG |
| | No. 4900811 | | |
| D28875 | Osreonectin | 3.83 | DG |
| U49099 | cis-Golgi p28 (p28) | 2.14 | DG |
| D28875 | Osteonectin | 2.07 | PA |
| P11661 | nadh-ubiquinone ox | 1.89 | DG |
| I00256 | Sequence 7 from U.S. Pat. | 1.89 | LC |
| | No. 4900811 | | |
| Z26634 | ankyrin brain variant, | 2.63 | PA |
| | ANKB | | |
| M89646 | ribosomal protein S24 | 2.32 | DR |
| P11661 | nadh-ubiquinone ox | 2.10 | CA1 |
| M89646 | ribosom protein S24 | 2.10 | DG |
| M25888 | lipophilin | 2.09 | CA1 |
| M89646 | ribosornal protein S24 | −1.98 | LC |
| U49099 | cis-Golgi p28 (p28) | −1.94 | LC |
| Z26634 | ankyrin brain variant, | −1.90 | DG |
| | ANKB | | |

TABLE 4

| | | | |
|---|---|---|---|
| | | R107474NCMS | |
| ACC. No. | GENE NAME | vs. R107474 | NUCLEI |

Apoptosis

| | | | |
|---|---|---|---|
| U59746 | Bcl-w | 2.11 | DR |
| AF022223 | BAG-1 | 2.05 | LC |
| U59746 | Bcl-w | 1.85 | CA1 |
| U67885 | p53 binding prot 1 | 1.84 | PA |

Calcium

| | | | |
|---|---|---|---|
| AA819345 | parvalbumin | 3.18 | CA1 |
| AA819345 | Parvalbumin | 2.23 | DR |
| M31178 | calbindin D28 | 2.20 | CA1 |

catecholamine synthesis

| | | | |
|---|---|---|---|
| AA142680 | dopamine beta- | 2.34 | LC |
| | hydroxylase* | | |

energy production

| | | | |
|---|---|---|---|
| M29341 | glyceraldehyde-3- | 2.24 | LC |
| | phosphate dehydrogenase | | |
| X02231 | glyceraldehyde-3- | 2.09 | LC |
| | phosphate dehydrogenase | | |
| V01556 | mitochondria genome | 2.07 | MG |
| | fragment | | |
| M35826 | mitochondrial NADH- | 1.96 | PA |
| | dehydrogenase (NDI) | | |

glutamate signaling

| | | | |
|---|---|---|---|
| U08107 | N-methyl-D-aspartate | 1.85 | CA3 |
| | receptor subunit | | |
| U08107 | N-methyl-D-aspartate | 1.81 | DG |
| | receptor subunit | | |
| M81886 | glutamate rec type 1 | −2.58 | CA1 |
| | HBGR1) | | |
| U08 107 | N-methyl-D-aspartate | −2.46 | DR |
| | receptor subunit | | |
| M64752 | glutamate rec sub | −2.35 | CA1 |
| | (GluH1) | | |

Plasticity

| | | | |
|---|---|---|---|
| M16228 | GAP-43 | 2.03 | LC |
| X60370 | MAP1B | −2.21 | CA3 |

receptors, ions channels

| | | | |
|---|---|---|---|
| Y07521 | Kv3.1a = NGK2 | 2.41 | PA |
| | potassium channel; V- | | |
| | gated | | |
| AA875142 | 5HT3 receptor mRNA | 2.08 | LC |
| | H3 Histamine Rec* | 2.05 | LC |
| AJ225124 | Hyperpolarization-act | 1.82 | LC |
| | cation channel HAC3 | | |
| AA427267 | SCN9a = PN1 Sodium | 1.81 | LC |
| | Channel alpha, V-gated | | |
| M59980 | K Channel | −2.24 | CA1 |
| AA819622 | thrombin receptor | −2.08 | DG |
| L28175 | Prostaglandin EP2 | −1.99 | LC |
| U12194 | voltage-gated Na+ | −1.83 | LC |
| | channel beta-1 sub- | | |
| | (SCN1B) gene | | |

signal transduction

| | | | |
|---|---|---|---|
| AI071860 | RGS14 | 2.64 | CA1 |
| M23612 | GTPase-activating | 2.16 | CA1 |
| | protein (GAP) | | |
| M29870 | ras-related C3 botulinum | 2.01 | DR |
| | toxin substrate (rac) | | |

transcription factors

| | | | |
|---|---|---|---|
| W65013 | Similar to X07384 GLI | −2.16 | CA1 |
| | PROTEIN. | | |
| J04509 | JUN-D prot | −2.10 | DR |
| AF062566 | Sp1 | −1.94 | LC |

Others

| | | | |
|---|---|---|---|
| M30952 | 28S ribosomal RNA gene | 2.92 | PA |
| | fragment | | |
| U20553 | P57 | 2.18 | PA |
| M30952 | 28S ribosomal RNA gene | 2.14 | MG |
| | fragment | | |
| M93056 | monocyte/neutrophil | 2.12 | PA |
| | elastase inhibitor | | |
| X92106 | Bleomycin hydrolase | 2.08 | PA |
| U20553 | P57 | 1.98 | DG |
| X92106 | Bleomycin hydrolase | 1.87 | CA3 |
| M29548 | elongation factor 1-alpha | 1.83 | DR |
| | (EF1A) | | |
| M93056 | monocyte/neutrophil | 1.80 | DG |
| | elastase inhibitor | | |
| M29548 | elongation factor 1- | −1.98 | CA1 |
| | alpha (EF1A) | | |
| M89646 | ribosomal protein S24 | −1.94 | CA1 |
| M89646 | ribosornal protein S24 | −1.89 | CA1 |
| X95591 | ClD protein | −1.85 | CA1 |
| X95591 | ClD protein | −1.80 | CA3 |

The largest set of genes changing their expression level after chronic treatment with novelR1 was found in DG. Among these genes, the primary site of action of novelR1, the a2c adrenoceptor mRNA (GB: M99376) level was found to be increased (fold difference versus control/vehicle: 3.62).

Overlaps in the gene expression patterns of imipramine and novelR1 in unstressed animals were observed in PA (thirteen genes)>LC (ten genes), DG (ten genes)>DR (five genes)>CA1 (four genes)>MG (two genes). Among these overlapping genes, six (three with an accession number) were found to change their expression levels in two different nuclei (heat-shock protein 27, GB: M86389 in PA and DR; Gs, GB: M12773 in MG and PA; 5-HT₂B receptor, GB: X66842 in DG and CA1).

35% of the Genes Changing Their Expression Level in Response to the CMS Paradigm were Antagonized by Both Drugs Treatment.

Imipramine and novelR1 were found to antagonize the changes in gene expression level induced by the CMS paradigm of a similar number of genes (imipramine: 53 genes, novelR1: 55 genes). This corresponds to 68% to 69% of the changes induced by the CMS paradigm. For twenty-eight of these genes overlap in the gene expression profile of imipramine and novelR1 were observed.

The increase in expression level of tyrosine hydroxylase mRNA induced by the CMS paradigm was not antagonized by chronic imipramine or novelR1 treatment in LC. The level of tyrosine hydroxylase mRNA were found to be up-regulated in the imipramine or novelR1/CMS group (fold increase versus vehicle/control: imipramine, 3.12; novelR1, 4.41). The up-regulation of tyrosine hydroxylase mRNA level was also confirmed by in situ hybridization (FIGS. 5e and 5f). The level of dopamine β hydroxylase (GB: AA142680) mRNA, another enzyme involved in the catecholamine biosynthesis, was also increased by both drugs in CMS rats in LC (fold increase versus vehicle/control: imipramine, 1.95; novelR1, 2.34). Similarly, DcoH mRNA level was increased (fold increase versus vehicle/control: imipramine, 2.30; novelR1, 2.67).

In CMS rats Both Drugs Changed the Expression Level of a Similar Number of Genes in LC, DR, CA1, CA3 and PA but with Minor Overlaps in Their Expression Profiles.

In LC, DR, CA1, CA3 and PA, imipramine changed the expression level of a larger set of genes in CMS rats than in unstressed rats (FIG. 6a), whereas novelR1 changed the expression level of a similar number of genes in CMS and unstressed rats (FIG. 6b). Administration of imipramine or novelR1 to CMS animals normalized the sucrose intake to the control level (FIG. 2b). The genes that change their expression level in the drug treated/CMS group but not in the drug treated/control group could reflect a drug +CMS interaction. These changes in gene expression are either causal to the change in sucrose intake from 8 grams to 13 grams or are responding to the new behavioral state. Both drugs yield a similar number of genes reflecting the drug/CMS interaction in LC, DR, CA1, CA3 and PA. In DG and MG, the imipramine/CMS interaction induced greatest changes in gene expression than the novelR1/CMS interaction.

Minor overlaps in genes expression patterns between imipramine and novelR1 in CMS rats were observed: LC (four genes)>CA1 (two genes)>DR (one gene), PA (one gene). The overlapping genes with an accession numbers are listed in FIG. 6. Imipramine changed the expression level of a total of 184 genes in CMS rats, whereas novelR1 changed the expression level of 91 genes. The genes with an accession number were classified in functional families and are listed in Table 2 (imipramine) and Table 3 (novelR1). Thirty genes were found to change their expression level in more than one nucleus for imipramine and twelve genes for novelR1.

### EXAMPLE 2

2.1. Tissue Preparation

Five adult male Wistar rats (423 g±22) that were used as control animals in a behavioral paradigm were decapitated, their brains were removed, placed in cryomolds, covered with Tissue-Tek tissue embedding medium (OTC) and snap-frozen in dry ice-cold 2 methyl butane (−60° C.). Ten tm-thick coronal sections in the mid-regions of the PVN (bregma—1.80 mm), hippocampus (bregma—3.30 mm), DR (bregma—1.78 mm) and LC (bregma—9.80 mm) were cut using a cryostat, mounted on non-coated, clear microscope slides and immediately frozen on a block of dry ice. The sections were stored at −70° C. All efforts were made to minimize animal suffering and reduce the number of animals used.

2.2. Laser Capture Microdissection

A quick Nissl (cresyl violet acetate) staining was used to identify the neurons as described [R. C. Salunga, H. Guo, L. Luo, A. Bittner, K. C. Joy, J. Chambers, J. Wan, M. R. Jackson and M. G. Erlander, “Gene expression analysis via cDNA microarrays of laser capture microdissected cells from fixed tissue” (1999) In M. Schena (Ed.), “DNA microarrays a practical approach”, Oxford University Press, Oxford]. The PixCell II System from Arcturus Engineering, Inc. (Mountain View, Calif.) was used for LCM. According to the manufacturer's protocol, one hundred neurons were captured in each region of interest of each animal (LC, DR, MG, PA, CA1, CA3 and DG). Example of laser-captured cells in CA1 region is shown in FIG. 1.

2.3. RNA Extraction and Amplification of LCM Samples

The total RNA was extracted from the individual LCM samples as described [R. C. Salunga, H. Guo, L. Luo, A. Bittner, K. C. Joy, J. Chambers, J. Wan, M. R. Jackson and M. G. Erlander, “Gene expression analysis via cDNA microarrays of laser capture microdissected cells from fixed tissue” (1999) In M. Schena (Ed.), “DNA microarrays a practical approach”, Oxford University Press, Oxford] with modifications as specified below. For total RNA extraction, a small volume (20 μl/LCM sample) of denaturing buffer (Micro RNA isolation kit, Stratagene: San Diego, Calif.) containing 2-mercaptoethanol (10 μl/ml, Sigma: St Louis, Mo.) and polyinosine (300 ng/LCM sample, Sigma: St Louis, Mo.) was used. The samples were incubated at 42° C. for ten minutes, and loaded onto a pre-rinsed Microcon-100 column (Millipore: Bedford, Mass.). After two rinses with 500 μl Rnase-free water, the columns were inverted and the samples collected by brief centrifugation. The samples were then processed for two rounds of RNA amplification as described [R. C. Salunga, H. Guo, L. Luo, A. Bittner, K. C. Joy, J. Chambers, J. Wan, M. R. Jackson and M. G. Erlander, “Gene expression analysis via cDNA microarrays of laser capture microdissected cells from fixed tissue” (1999) In M. Schena (Ed.), “DNA microarrays a practical approach”, Oxford University Press, Oxford] with modifications for the DNA and RNA purification steps. Quiaquick PCR purification kit (Qiagen: Valencia, California) and RNAeasy Mini Kit (Qiagen: Valencia, California) were used for purification steps. Polyinosine (100 ng) was added to each sample before purification of DNA or aRNA.

2.4. cDNA Microarray

A cDNA microarray containing 2147 cDNA clones (corresponds to 2137 unique mRNAs) was used in this study. cDNA clones were obtained commercially from Research Genetics (IMAGE consortium), Incyte Genomics, as well as internal sources. All clones were verified by DNA sequencing. Each clone was printed as two independent spots on a given chip. A contact pin microarrayer (Generation III Array Spotter, Molecular Dynamics) was used to spot the clones in duplicate. Microarrays were hybridized and scanned with a confocal laser scanner (Array Scanner, Molecular Dynamics) with excitation at 532 nm and collection through a 550 to 600 nm band width filter, the PMT set to 630V. Image analysis performed as previously described [R. C. Salunga, H. Guo, L. Luo, A. Bittner, K. C. Joy, J. Chambers, J. Wan, M. R. Jackson and M. G. Erlander, “Gene expression analysis via cDNA microarrays of laser capture microdissected cells from fixed tissue” (1999) In M. Schena (Ed.), “DNA microarrays a practical approach”, Oxford University Press, Oxford].

2.5. Statistical Analysis

Hybridization of each sample was performed on two identical microarrays. Each microarray was normalized at the 75 percentile (intensity of the 75 percentile was scaled to 100). Intensity of each clone on an individual microarray was then measured as an average of the intensities of the two corresponding spots, and the intensity of each clone in the sample was further determined as average of the intensities on the two identical chips. The pair-wise similarity of gene expression profiles within a same cell type was examined by scatter-plot and the correlation coefficient was calculated. Samples having a correlation coefficient of R<0.89 with respect to the majority were treated as out-liers and were excluded for further analysis. The intensity of each clone in one particular nucleus was subsequently further defined as the median of intensities of that clone in remaining qualified samples in that nuclei. The resulting intensity matrix (N×M) (N: number of clones; M: number of sample types) was further normalized (spline normalization) so that the distribution of the intensities of all the clones for each individual sample type was fitted to a smooth cubic B-spline. The spline normalization could reduce abnormality in the distribution of the intensities in particular samples caused by saturation in image processing.

In addition to the 2147 clones, fifteen plant cDNA were printed on the same microarray. The intensities of these fifteen plant cDNAs were measured and processed in the same manner as other clones, and used as negative controls. The average intensity of the fifteen plant genes in all M types of samples was defined as background. Each clone was then classified as absent in a cell population if its medium intensity was below the three-fold of the background; or as present if its medium intensity was three-fold above the background. Hierarchical clustering was generated by using Cluster software (Stanford).

Gene Expression Profiling of Brain Nuclei is Reproducible.

Gene expression profiles were measured from seven different laser-captured brain nuclei in six different groups of rats (i.e. control/vehicle, CMS/vehicle, control/imipramine, CMS/imipramine, control/ novelR1, CMS/novelR1). To estimate experimental reproducibility within a group of rats, the pair-wise similarities of gene expression profiles were compared by calculating a linear correlation coefficient (FIG. 3). The average linear correlations coefficients (R) within one region ranged from 0.955 to 0.978 (n =3 to 5 per group, FIG. 3a). For examples, the correlation value for the DR in control/vehicle animals ranged from 0.95 to 0.98 (FIG. 3b). For CA3 the R values ranged from 0.96 to 0.98. These data suggest that the brain nuclei were clearly captured and that these nuclei are relatively homogenous in gene expression.

Different Brain Nuclei Have about 4% to 7% in Unique Gene Expression.

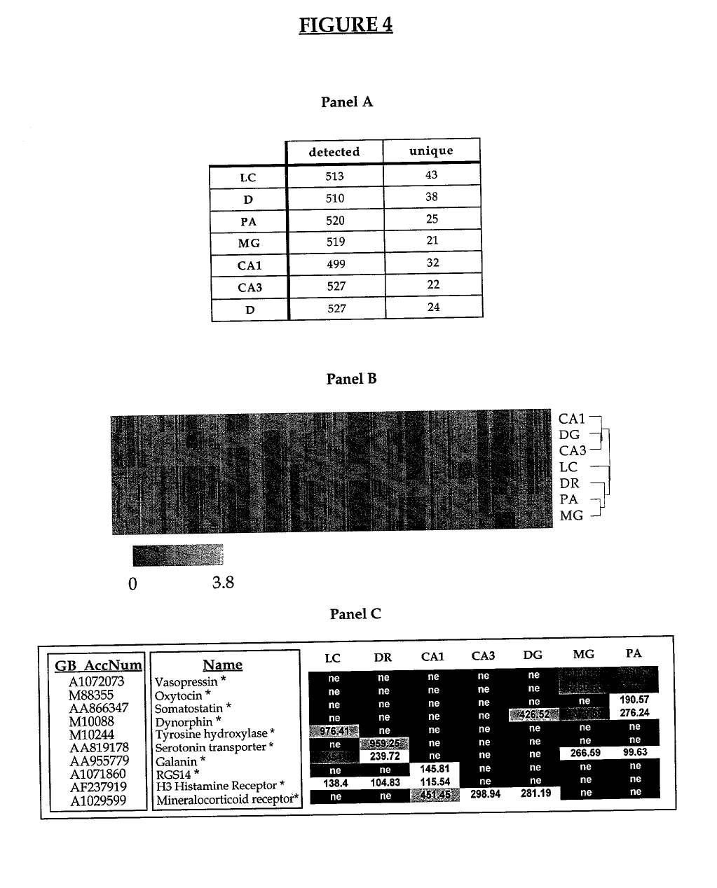

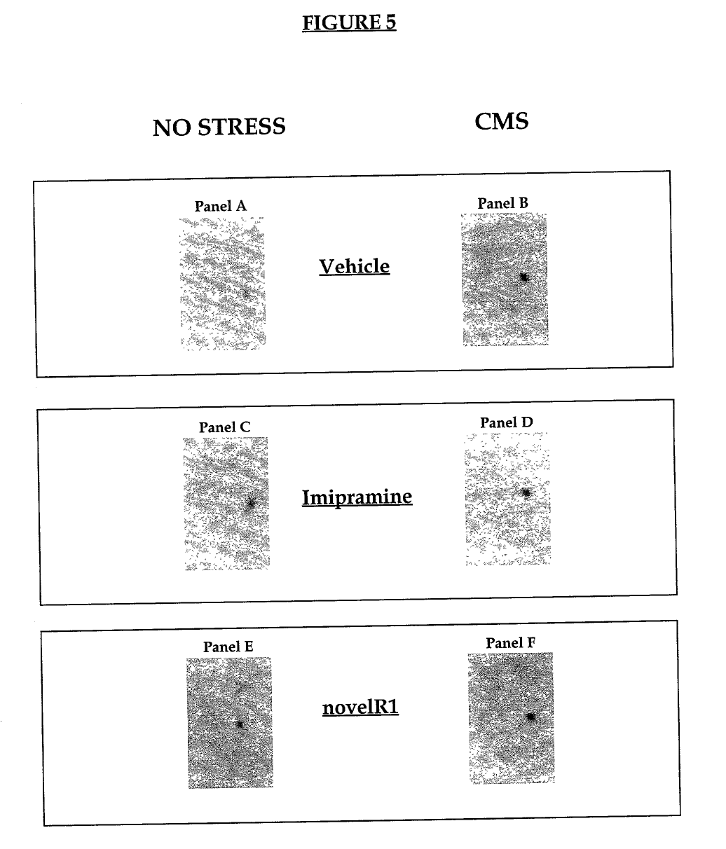

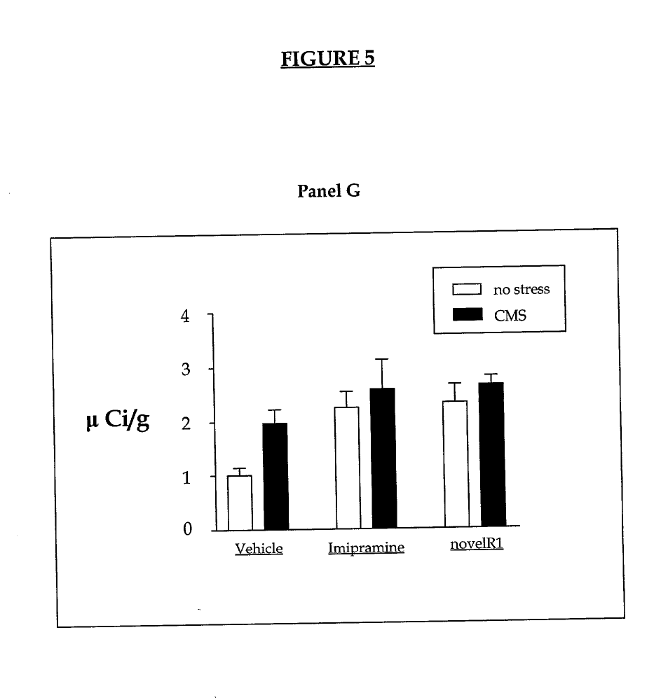

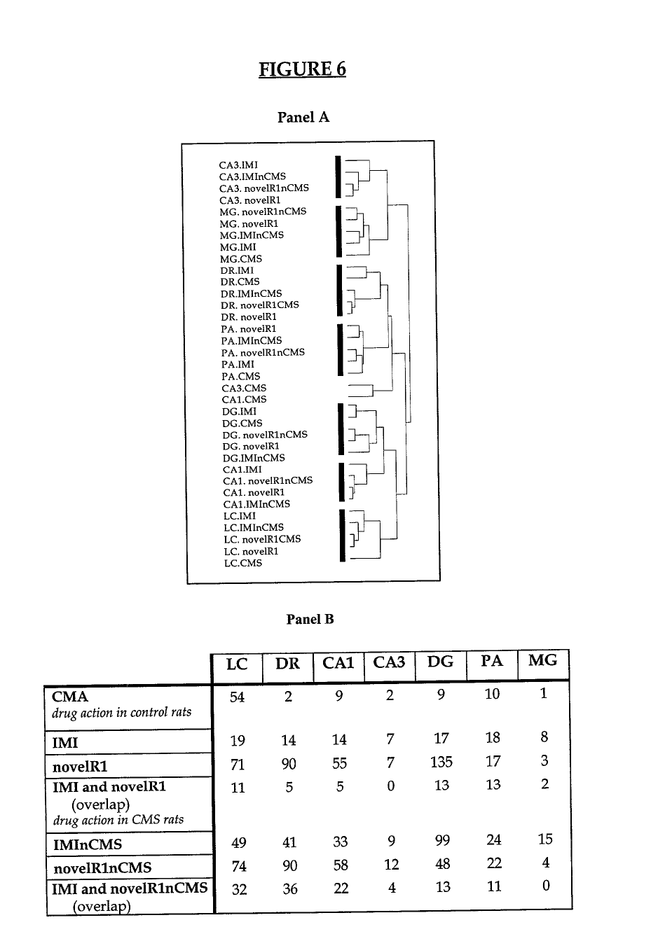

To further validate the LCM/cDNA microarray approach, the gene expression patterns of the seven laser-captured brain nuclei were compared within the control/vehicle groups (FIG. 4). Of the 2137 genes (unique mRNAs) analyzed, 871 (40.76%) gave a hybridization signal consistent with a call of presence in at least one brain region according our criteria (i.e. at least three-fold above background level). The number of genes detected within one region ranged from 499 to 527 (FIG. 4a). The number of genes unique to one of the seven investigated brain nuclei, according our criteria for a call of presence, ranged from 21 to 43 (FIG. 4a). The highest number of unique genes (43) was found in LC. All 419 genes that changed expression levels between the different areas of the brain are listed in Table 5.

To compare the overall gene expression patterns of the different brain areas two-dimensional hierarchical clustering was performed (FIG. 4b). Hierarchical clustering analysis showed similarities in expression patterns between the two divisions of the PVN and between the three regions of the hippocampus (FIG. 4b). For example, the dendrogram on the vertical axis shows that the two divisions of the PVN (PA and MG) were joined by a very short branch reflecting their similarities compared to the other areas (FIG. 4b) and the three regions of the hippocampus (CA1, CA3 and DG) were also located on a common branch. The LC was located on a different branch of the dendrogram, suggesting that this nuclei has the greatest difference in gene expression among the seven nuclei that were examined. This finding is consistent with the LC containing the highest number of genes that are enriched (described above).

The analysis was further validated by the agreement of our findings with that of previously documented expression patterns for “signature” genes for specific brain nuclei (FIG. 4c). For example, in our study, abundant mRNA expression encoding vasopressin (GB:AI072073) and oxytocin (GB:M88355) were detected in PA and MG cells, but not detected in the other examined nuclei. This finding is in agreement with previous studies [Hallbeck, 1999 #359] [Burbach, 1987 #354] [Tohyama, 1998 #348]. Other striking examples included mRNAs encoding tyrosine hydroxylase (GB: M10244) and serotonin transporter (GB: AA819178) to the LC and DR respectively (FIG. 4c).

| | A | B |
|---|---|---|
| 1 | | Name |
| 2 | neuropeptide | 9174\| UI-R-E1-fg-b-04-0-UI\| AA955779\| Rat galanin (a neuropeptide) mRNA, complete cds |
| 3 | neuropeptide | 9168\| UI-R-E1-fg-g-12-0-UI\| AA955767\| Rat mRNA encoding rat pre-prosomatostatin (isolated from a medullary thyroid carcinoma) |
| 4 | | 8723\| UI-R-A1-ev-b-11-0-UI\| AA926266\| Rat VL30 element mRNA |
| 5 | | 8637\| UI-R-E0-br-e-03-0-UI\| AA866450\| Rattus norwegicus interleukin-2 receptor beta chain (p70/75) mRNA, complete cds |
| 6 | | 8615\| UI-R-A0-aj-e-08-0-UI\| AA866381\| Rat mRNA for hnRNP protein, partial |
| 7 | neuropeptide | 8610\| UI-R-A0-bm-a-06-0-UI\| AA866347\| Rat mRNA encoding rat pre-prosomatostatin (isolated from a medullary thyroid carcinoma) |
| 8 | | 8569\| UI-R-A1-el-b-12-0-UI\| AA926045\| Similar to AF020760 H sapiens serine protease (Omi) mRNA |
| 9 | apoptosis | 8540\| UI-R-A1-es-c-03-0-UI\| AA926004\| Rattus norvegicus mRNA for 14-3-3 protein eta-subtype, complete cds |
| 10 | ion channel | 81\| 832845\| AA427267\| SCN9a=PN1 Sodium Channel alpha, V-gated |
| 11 | | 7888\| UI-R-A1-eg-b-04-0-UI\| AA924903\| Similar to HUMKG1C Human mRNA for KIAA0045 gene, mRNA |
| 12 | ion channel | 786\| UI-R-EO-bu-g-03-O-UI\| AA875142\| Rat 5HT3 receptor mRNA, complete cds |
| 13 | | 7732\| UI-R-A1-dz-c-12-0-UI\| AA924656\| Similar to AB005298 H sapiens BAI 2 mRNA |
| 14 | | 735\| UI-R-E0-ck-e-08-0-UI\| AA874900\| Similar to AC004021 kelch protein |
| 15 | | 7233\| UI-R-A1-dp-d-03-0-UI\| AA901316\| Rattus norvegicus DCoH gene |
| 16 | | 6980\| UI-R-E0-dk-d-12-0-UI\| AA900886\| Similar to MMUBF Mouse ubf gene for transcription factor UBFmRNA |
| 17 | | 68\| 775900\| AA276133\| CIC-2 Chloride Channel, V-gated |
| 18 | | 6611\| UI-R-C0-hc-e-09-0-UI\| AF020760\| Omi |
| 19 | hsp | 6565\| UI-R-G0-UI-h-03-0-UI\| L39370\| Human Hsp27 |
| 20 | | 6544\| UI-R-C1-ke-c-01-0-UI\| L28175\| Prostaglandin EP2 |
| 21 | | 6510\| UI-R-E1-fu-h-10-0-UI\| L11667\| Human cyclophilin-40 |
| 22 | | 6505\| 2076313\| L10284\| Human calnexin |
| 23 | | 6457\| 918411\| D42055\| hNEDD-4/KIAA0093, hect domain protein |
| 24 | | 644\| UI-R-E0-cq-c-04-0-UI\| AA875241\| Similar to MMUBCM4GN M. musculus UBcM4 mRNAmRNA |
| 25 | hsp | 6439\| 2064663\| D13627\| TCP-1 Hsp60, theta subunit |
| 26 | | 6407\| 749025\| M11723\| Blood coagulation factor XII |
| 27 | | 6406\| 660902\| M112331 Cathepsin D |
| 28 | | 6351\| UI-R-C0-IG-e-10-0-UI\| M24898\| Nuclear receptor alpha1 for thyroid horm |
| 29 | hsp | 6332\| UI-R-AD0-we-g-01-0-UI\| M196451 Human BiP/GRP78/ER resident HSP70 |
| 30 | GPCR | 6296\| 7006963431 M818301 Somatostatin (55R2) |
| 31 | G protein signaling | 6240\| 2088210\| M60156\| Go alpha (G-protein) |
| 32 | | 6232\| 1152703\| M58051\| FGFR3 |
| 33 | GPCR | 6204\| 700311648\| U07225\| Purinergic (P2U) |
| 34 | | 6150\| UI-R-AE0-xe-f-02-0-UI\| M94335\| Akt1/PKB/RAC |
| 35 | | 6118\| UI-R-CO-gs-d-06-0-UI\| U39738\| PAK3 |
| 36 | | 6068\| UI-R-AA1-aab-c-10-0-UI\| U20553\| P57 |
| 37 | | 60664\| 583865\| U20372\| Calcium channel beta 3 (CCHB3); V-gat,aux |
| 38 | proteasome | 6031\| 478404\| W07641\| ubiquit.-like protein SMT3B (SUMO-1) |
| 39 | GPCR | 5997\| 701899983\| U71092\| GPCR37 (Orphan, public=GPR24) |
| 40 | | 5981\| 1885026\| U59167\| Desmin |
| 41 | ion channel | 5977\| 700254854\| U57352\| ASIC2=MDEG=BNC1 brain Na chan; amil sens |
| 42 | 5958\| 947959\| U47036\| CCR5 | |
| 43 | GPCR | 5957\| UI-R-Y0-acc-e-12-0-UI\| U469231 GPCR8 (Orphan, public) |
| 44 | hsp | 5934\| 464942\| X63368\| H. sapiens HSJ1 mRNA, DnaJ homolog |
| 45 | ion channel | 5897\| UI-R-Y0-aci-c-12-0-UI\| X16476\| Kv2.1=DRK1 potassium channel; V-gated |
| 46 | hsp | 5889\| UI-R-A0-ab-h-12-0-UI\| X15187\| Human gp96/GRP94, ER HSP90 |
| 47 | 5873\| 1211032\| X04434\| IGF1R | |
| 48 | 5855\| 701644254\| Z30425\| Nuclear receptor/orphan MB67/CAR | |
| 49 | ion channel | 5845\| 493098\| Y07521\| Kv3.1a=NGK2 potassium channel; V-gated |
| 50 | transcription factor | 5844\| 944489\| Y00864\| SCFR/c-kit |
| 51 | | 5832\| 677053\| X99585\| ubiquit.-like protein SMT3B (SUMO-1) |
| 52 | GPCR | 5825\| 701293873\| X94552\| Glutamate metabotropic (mGlu7) |
| 53 | | 5824\| 2182578\| X92106\| Bleomycin hydrolase |
| 54 | | 5821\| 700230143\| X89576\| Matrix metalloproteinase MT2-MMP |
| 55 | | 5814\| 368357\| X85237\| splicing factor SF3a120 |
| 56 | | 5809\| 316104\| X82153\| Cathepsin K |
| 57 | hsp | 5789\| UI-R-C0-hc-d-08-0-UI\| X74801\| Human TCP-1 HSP60, gamma subunit |
| 58 | apoptosis | 5763\| 100400\| 100400\| 14-3-3 gamma |
| 59 | GPCR | 5704\| 701035392\| 258\| H3 Histamine Rec (GPCR97) |
| 60 | apoptosis | 5694\| 95332\| 95332\| 14-3-3 eta |
| 61 | | 5681\| 112173\| 112173\| calcineurin A2 |
| 62 | | 4716\| 583498\| AA142680\| S50200 S50200 dopamine beta-hydroxylase [mice, Genomic/mRNA, 2274 nt]. Blastn P. = 4.6E-167 |
| 63 | | 4715\| 580798\| AA139498\| L41351 HUMPROS Homo sapiens prostasin mRNA, complete cds. Blastn P. = 2.5E-141 |
| 64 | | 4699\| 604709\| AA161790\| X05026 HSRHOB Human rho mRNA (clone 12). Blastn P. =1.5E-204 |
| 65 | | 4262\| 577610\| AA426192\| hUBC6 |
| 66 | | 39517\| UI-R-AG0-ww-b-07-0-UI\| U63421\| sulfonylurea rec (SUR1) |
| 67 | transcription factor | 39504\| UI-R-01-kw-b-11-0-UI\| AF012923\| Wig-1 |
| 68 | | 394961UI-R-AD1-zn-f-05-0-UIIU438441cyclinD3gene |
| 69 | | 39495\| UI-R-02p-nz-g-03-0-UI\| U28789\| PACT |
| 70 | polyamine synthesis | 39494\| UI-R-Y0-acv-f-12-0-UI\| J04791\| DCOR |
| 71 | | 39490\| UI-R-C0-jh-b-10-0-UI\| Y14296\| BTEB-1 |
| 72 | transcription factor | 39485\| UI-R-C0-is-f-03-0-UI\| X17163\| c-jun |
| 73 | | 39477\| UI-R-E1-gf-e-12-0-UI\| Y07701\| cytosol alanyl aminopeptidase |
| 74 | | 39475\| UI-R-C3-ti-d-08-0-UI\| Y07836\| helix-loop-helix prot |
| 75 | apoptosis | 39461\| UI-R-C0-jq-f-07-0-UI\| AF022223\| BAG-1 |
| 76 | | 39460\| UI-R-Y0-abk-h-03-0-UI\| U77615\| crx homeodomain prot |
| 77 | | 39456\| UI-R-AE0-xm-h-07-0-UI\| AFO31384\| TWIK-related acid-sens K+ channel (TASK) |
| 78 | GPCR | 39452\| UI-R-C2-nh-f-03-0-UI\| X51585\| Adrenergic alpha1B Rec |
| 79 | | 39451\| UI-R-C2-mq-h-08-0-UI\| U23781\| A1-d prot |
| 80 | | 39428\| UI-R-E0-cd-a-02-0-UI\| D29678\| CDKS |
| 81 | | 39424\| UI-R-C0-jh-c-05-0-UI\| U28068\| neuroD |
| 82 | GPCR | 39418\| UI-R-Y0-mj-a-06-0-UI\| M58316\| Adrenergic alpha2C Rec |
| 83 | | 39416\| UI-R-E0-de-a-06-0-UI\| U1326\| methionyl aminopeptidase II |
| 84 | | 39413\| UI-R-A1-eg-e-06-0-UI\| M62867\| MSY-1 |
| 85 | | 39404\| UI-R-C2p-qw-a-08-0-UI\| M31178\| calbindin D28 |
| 86 | | 39402\| UI-R-AF1-aaq-g-02-0-UI\| D76432\| EF1 delta |
| 87 | | 39401\| UI-R-BT0-qa-h-12-0-UI\| U64828\| SRC-1 |
| 88 | | 39394\| UI-R-C3-ty-c-12-0-UI\| X15052\| NCAM-140 |
| 89 | transcription factor | 39392\| UI-R-G0-uh-h-09-0-UI\| AF062566\| Sp1 |
| 90 | proteasome | 39370\| UI-R-E0-bv-f-08-0-UI\| D26598\| proteasome beta 3 subunit |
| 91 | | 39365\| UI-R-E0-bv-d-02-0-UI\| D29011\| proteasome catalytic subunit 3 |
| 92 | | 39358\| UI-R-C2p-nt-b-07-0-UI\| X65227\| NMZ1 |
| 93 | GPCR | 39356\| UI-R-AA1-aaa-d-04-0-UI\| U15280\| Oxytocin Rec |
| 94 | GPCR | 39351\| UI-R-C0-jn-c-07-0-UI\| X97121\| Neurotensin 2 Rec |
| 95 | | 39334\| UI-R-C2p-ns-f-01-0-UI\| U88716\| Nkx-32 homeodomain prot |
| 96 | proteasome | 39323\| UI-R-E0-cy-e-09-0-UI\| D00761\| proteasome beta 1 subunit |
| 97 | prtoeasome | 39321\| UI-R-AC1-xw-f-06-0-UI\| AF079564\| ubiquitin carboxy-terminal hydrolase ubp41 |
| 98 | transcription factor | 39311\| UI-R-C1-lq-e-10-0-UI\| J04509\| JUN-D prot |
| 99 | | 39296\| UI-R-C2p-rc-d-02-0-UI\| U19597\| Cdk4/Cdk6 inhib prot |
| 100 | | 39290\| UI-R-C1-kc-d-10-0-UI\| U41741\| MMUSF (USF) |
| 101 | | 39281\| UI-R-C1-kb-d-12-0-UI\| AF005885\| cyclin G2 |
| 102 | transcription factor | 39274\| UI-R-C0-hv-a-09-0-UI\| AF065386\| Jab1 |
| 103 | | 39265\| UI-R-Y0-ach-h-07-0-UI\| X91753\| SEF2 |
| 104 | polyamine synthesis | 39260\| UI-R-AC0-yp-f-08-0-UI\| D10706\| OAZ |
| 105 | | 39254\| UI-R-C2p-oh-h-09-0-UI\| D29956\| UBPY ubiquitin isopeptidase (H. sapiens) |
| 106 | | 39253\| UI-R-C2p-ro-d-11-0-UI\| AF017182\| Sox10 |
| 107 | | 39252\| UI-R-C0-il-c-03-0-UI\| U42556\| freac-1 gene |
| 108 | proteasome | 39236\| UI-R-C3-tr-c-02-0-UI\| S71381\| proteasome beta 4 subunit |
| 109 | | 39212\| 101689\| 101689\| Mouse transcriptional regulator RPD3 homolo |
| 110 | | 39209\| 80164\| 80164\| Rat heavy neurofilament polypeptide NF-H |
| 111 | | 39195\| 95418\| 95418\| Rat non-muscle myosin alkali light chain |
| 112 | | 39192\| 476493\| M97200\| krueppel-like factor |
| 113 | | 39191\| 700295357\| M92340\| IL6B |
| 114 | | 39175\| 80186\| 80186 Novel |
| 115 | | 39149\| 102883\| 102883 Novel |
| 116 | | 39140\| 701293538\| M21948 dihydropyridine-sens Ca channel alpha-2 sub |
| 117 | | 39133\| 111979\| 111979\| Novel |
| 118 | | 39132\| 701900803\| M81886\| glutamate rec type I (HBGR1) |
| 119 | | 39112\| 872080\| M26745\| IL6 |
| 120 | | 39111\| 100756\| 100756\| Novel |
| 121 | | 39109\| 109519\| 109519\| Rat PKG binding protein and substrat |
| 122 | ion channel | 39094\| 700771408\| L08494\| GABA-A receptor |
| 123 | | 39092\| 113323\| 113323\| Rat light molecular-weight neurofila |
| 124 | | 39091\| 112184\| 112184\| Novel |
| 125 | | 39083\| 113568\| 113568\| Mouse neurofilament protein (NF-L) gene, 3′ flank |
| 126 | | 39075\| 100749\| 100749\| Mouse transcriptional regulator RPD3 homolo |
| 127 | | 39068\| 112215\| 112215\| retrovirus-related leucine zipper protein p40 - ra |
| 128 | | 39062\| 700773553\| AJ225124\| hyperpolarization-act cation channel HAC3 |
| 129 | | 39058\| 112959\| 112959\| Novel |
| 130 | | 39057\| 571654\| X61506\| E46 for E46 prot |
| 131 | | 39055\| 112214\| 112214\| S-100 beta subunit [rats, Genomic, 423 nt, segment |
| 132 | | 39053\| 112025\| 112025\| Rat ID-A element in the S-100 beta-subunit gene. |
| 133 | | 39049\| 101164\| 101164\| P46783 Human 40s ribosomal protein |
| 134 | | 39025\| 1366563\| X51405\| carboxypeptidase H |
| 135 | | 38988\| 700183273\| M10244\| tyrosine hydroxylase |
| 136 | | 38987\| 1972427\| D14425\| calcineurin |
| 137 | | 38986\| 111692\| 111692\| Novel |
| 138 | | 38973\| 113308\| 113308\| Rat light molecular-weight neurofilament |
| 139 | | 38971\| 109491\| 109491\| Human MUM2 gene |
| 140 | apoptosis | 38967\| 1211267\| U67885\| p53 binding prot 1 |
| 141 | | 38960\| 113236\| 113236\| Rat myelin proteolipid (PLP) |
| 142 | | 38956\| 95821\| 95821\| P09896 Human |
| 143 | | 38955\| 95395\| 95395\| Rat light molecular-weight neurofilament |
| 144 | | 38592\| 80905\| 80905\| Novel |
| 145 | | 38946\| 103913\| 103913\| Novel |
| 146 | | 38945\| 95793\| 95793\| Rat cytoplasmic dynein heavy chain |
| 147 | | 38941\| 111147\| 111147\| Mouse B2 repeat in the 3′ flank of protein 53 (p53 |
| 148 | | 38933\| 1448814\| M25889\| Mylein basic port |
| 149 | GPCR | 38929\| 700148588\| X66842\| Serotonin 2B Rec |
| 150 | | 38922\| 41932\| U12194\| voltage-gated Na+ channel beta-1 sub (SON1B) gene |
| 151 | | 38921\| 700280626\| S76145\| Na/Cl dep neurotransmitter transporter |
| 152 | | 38917\| 1364912\| U36393\| TFEB |
| 153 | | 38916\| 477909\| U68384\| Mrg1b |
| 154 | | 38910\| 112963\| 112963\| Mouse mitochondrial genes coding for three transfe |
| 155 | | 38909\| 101166\| 101166\| Mouse neurofilament protein (NF-L) gene, 3′ flank. |
| 156 | | 38898\| 106216\| 106216\| Horse serpin mRNA, complete cds. |
| 157 | | 38894\| 100750\| 100750\| Sequence 1 from patent U.S. Pat. No. 5457026. |
| 158 | | 38884\| 112218\| 112218\| S-100 beta subunit [rats, Genomic, 423 nt, segment |
| 159 | | 38879\| 112228\| 112228\| Mouse TGF-beta-inducible protein (TSC-36) m |
| 160 | | 38875\| 84488\| 84488\| Novel |
| 161 | | 38865\| 1297351\| M26657\| germinal peptidyl-dipeptidaseA |
| 162 | proteasome | 38863\| 633539\| D00763\| proteasome alpha 4 subunit |
| 163 | | 38856\| 463999\| U63858\| U57939 |
| 164 | | 38845\| 84487\| 84487 Novel |
| 165 | | 38838\| 101073\| 101073\| Rat PAG608 gene |
| 166 | | 38833\| 1210758\| AF057691\| TCF-3 |
| 167 | | 38829\| 108622\| 108622\| ribosomal protein S24 homolog {clone 2-6} [rats, r |
| 168 | ion channel | 38824\| 679093\| AF052746\| chloride channel CaCC |
| 169 | | 38822\| 103933\| 103933\| Rat gene for testicular cell adhesio |
| 170 | | 38816\| 113570\| 113570\| Novel |
| 171 | apoptosis | 38808\| 111644\| 111644\| Rat RNA-binding protein SiahBP mRNA, |
| 172 | GPCR | 38793\| 701472025\| M64236\| Neurokinin 1 Rec |
| 173 | GPCR | 38789\| 402354\| AF0279551 GPR27 Rec |
| 174 | | 38787\| 111636\| 111636\| Novel |
| 175 | | 38783\| 1211735\| Y10926\| RBP-L |
| 176 | | 38779\| 95446\| 95446\| Mouse brain mRNA. |
| 177 | | 38772\| 1162568\| Y076881 NfiX gene |
| 178 | | 38768\| 101714\| 1017141 Novel |
| 179 | | 38764\| 101216\| 101216\| Bos taurus ribosomal protein 52 mRNA, partial cds. |
| 180 | | 38761\| 100721\| 100721\| Novel |
| 181 | proteasome | 38756\| 100763\| 100763\| P11661 Rat nadh-ubiquinone ox |
| 182 | neuropeptide | 38737\| 701905916\| M10088\| dynorphin |
| 183 | G protein signaling | 38729\| 95348\| 95348\| GTP-binding protein rab10 - rat |
| 184 | | 38725\| 102725\| 102725\| Novel |
| 185 | ion channel | 38721\| 700247271\| L08495\| GABA receptor alpha6 |
| 186 | GPCR | 38716\| 701031976\| X63574\| Somatostatin 3 Rec |
| 187 | | 38714\| 112210\| 112210\| Mouse LB9 gene, partial sequence. |
| 188 | | 38712\| 669616\| U89412\| PML isoform 1 |
| 189 | | 38711\| 716499\| U90724\| X-Proaminopeptidase(eukaryote) |
| 190 | GPCR | 38710\| 700369402\| L14618\| Calcitonin Rec |
| 191 | | 38701\| 1331673\| M11597\| CGRP alpha |
| 192 | | 38694\| 112194\| 112194\| Novel |
| 193 | | 38676\| 701896910\| M36418\| GLR1 |
| 194 | GPCR | 38675\| 701901569\| M99376\| Adrenergic alpha2C Rec |
| 195 | | 38667\| 111664\| 111664\| Human (clone hh18) protein tyrosine phospha |
| 196 | | 38664\| 700133895\| 038450\| 954 Rec |
| 197 | | 38663\| 701921173\| M64752\| glutamate rec sub (GluH1) |
| 198 | | 38653\| 106117\| 106117\| Novel |
| 199 | | 38623\| 113304\| 113304\| Rat light molecular-weight neurofilament |
| 200 | | 38621\| 444406\| 444406\| Novel |
| 201 | | 38614\| 111621\| 111621\| Novel |
| 202 | | 38605\| 108620\| 108620\| Rat receptor for hyaluronan-mediated |
| 203 | | 38602\| 101693\| 101693\| Mouse ribosomal protein (ke-3) gene, exons |
| 204 | | 38601\| 100748\| 100748\| Mouse transciptional regulator RPD3 homolo |
| 205 | | 38594\| 113748\| 113748\| Mouse necdin mRNA, complete cds. |
| 206 | ion channel | 38582\| 700773668\| M81253\| Kv3.4 gene |
| 207 | | 38577\| 113515\| 113515\| Novel |
| 208 | | 38558\| 66940\| 66840\| Novel |
| 209 | | 38546\| 96780\| 96780\| Bos taurus clone CSSM015 microsatellite DNA sequen |
| 210 | | 38545\| 101138\| 101138\| Rat ribosomal protein S24 |
| 211 | | 38536\| 111254\| 111254\| Human C14orf3 protein |
| 212 | | 38533\| 475889\| AF020198\| PBX2 |
| 213 | | 38532\| 103937\| 103937\| Rat heavy neurofilament (NF-H) polypeptide, partia |
| 214 | | 38528\| 112216\| 112216\| S-100 beta subunit [rats, Genomic, 423 nt, segment |
| 215 | | 38522\| 463924\| T67463\| other peptidase (C1A) |
| 216 | | 38508\| 66531\| 66531\| Novel |
| 217 | | 38504\| 84486\| 84486\| Novel |
| 218 | | 38491\| 108519\| 108519\| Rat gamma-smooth muscle isoactin pro |
| 219 | | 38487\| 112021\| 112021\| Rat brain ID transcript, clone bBC2- |
| 220 | | 38469\| 95406\| 95406\| Rat light molecular-weight neurofilament |
| 221 | | 38463\| 113514\| 113514\| Novel |
| 222 | | 38417\| 79183\| 79183\| Novel |
| 223 | | 38404\| 1039810\| AA297071\| Similar to N-methyl-D-aspartate glutamate receptor |
| 224 | | 38402\| 701289315\| X99792\| capacitative Ca entry channel 1 |
| 225 | ion channel | 38386\| 700495245\| L08493\| GABA-A receptor |
| 226 | | 37466\| 386514\| W65013\| Similar to X07384 GLI PROTEIN. |
| 227 | | 37464\| 386559\| W65005\| Mouse cDNA clone IMAGE:386559 5′ |
| 228 | | 36393\| 387565\| A18932401 Similar to L14935 Mouse neural retina-specific leucine zipper protein. |
| 229 | ion channel | 33\| 426170\| AA002993\| Potassium channel Kv3.4a (Raw3), V-gated |
| 230 | | 2319\| UI-R-A0-af-e-06-0-UI\| AA817907\| Similar to MUSCDPK mouse cyclin-dependent kinase homologue (p130PITSL) mRNA, complete cdsmRNA |
| 231 | | 21709\| UI-R-AF0-ya-b-04-0-UI\| 988064T6\| LVENNOT03 INCYTE |
| 232 | | 21692\| UI-R-AC1-xn-e-07-0-UI\| 817852H1\| OVARTUT01 INCYTE |
| 233 | | 21664\| 903057\| 580795H1\| NMDA channel-like |
| 234 | DNA repair | 21573\| 1054114\| 3764982H1\| human homologue of yeast RAD23 |
| 235 | DNA repair | 21458\| UI-R-E0-ci-a-06-0-UI\| 2583919H1\| human homologue of yeast RAD23 |
| 236 | | 21418\| 1348327\| 2049923H1\| LIVRFET02 U43892 g1 167981 Mouse ABC transporter-7 mRNA, partial cd gb103rod 83 -55 |
| 237 | DNA repair | 21304\| 420048\| 1676240T6\| cell cycle checkpoint rad9 gene |
| 238 | | 21251\| 975800\| 1275842F6\| Eph-Erk |
| 239 | ion channel | 21230\| 748734\| 2658404T6\| SK2 calcium-activ potassium chann homolo |
| 240 | | 20977\| UI-R-02p-np-g-01-0-UI\| 485639T6\| retinoic acid hydroxylase hP450RAI |
| 241 | | 20971\| 2182407\| 3369279H1\| LON; ATP-Dep Proteas LA |
| 242 | | 20768\| 96782\| 96782\| M58405 R. norvegicus thymosin beta-10 gene, complete cds. 1.0e-72 |
| 243 | | 20765\| 95816\| 95816 X51396 Mouse MAP1B mRNA for MAP1B microtubule-associated 6.5e-99 |
| 244 | axon outgrowth | 20763\| 95791\| 95791\| M16228 Rat neuronal growth protein 43 (GAP-43) mRNA, comp 9.7e-187 |
| 245 | | 20762\| 95494\| 95494\| M93056 Human mononcyte/neutrophil elastase inhibitor mRNA 1.9e-117 |
| 246 | | 20760\| 95467\| 95467\| U43747 Human frataxin (FRDA) mRNA, complete cds. 1.9e-107 |
| 247 | | 20759\| 95416\| 95416\| S77858 non-muscle myosin alkali light chain [rats, Spragu 2.9e-92 |
| 248 | hsp | 20754\| 95386\| 95386\| M11942 Rat 70 kd heat-shock-like protein mRNA, complete c 9.0e-108 |
| 249 | | 20751\| 95321\| 95321\| G27093 human STS SHGC-31976. 1.5e-91 |
| 250 | | 20749\| 95308\| 95308\| S82024 SCG10=neuron-specific growth-associated protein/st 4.4e-102 |
| 251 | | 20748\| 94433\| 94433\| Z92839 Caenorhabditis elegans cosmid T08D2, complete sequ 0.89 |
| 252 | | 20743\| 84483\| 84483\| AC003043 Homo sapiens chromosome 17, clone HRPC1067M6, comp 2.9e-31 |
| 253 | energy production | 20740\| 83925\| 83925\| X02231 Rat mRNA for glyceraldehyde-3-phosphate-dehydrogen 7.9e-210 |
| 254 | | 20739\| 83922\| 83922\| 569874 C-FABP=cutaneous fatty acid-binding protein [rats, 6.0e-194 |
| 255 | | 20738\| 83912183912\| L37086 Homo sapiens FK-506 binding protein (fkbp12.6) gen 1.9e-151 |
| 256 | | 20726\| 82552\| 82552\| D17296 Rat mRNA for polyubiquitin (ten completely repetit 5.4e-90 |
| 257 | | 20724\| 82519\| 82519\| M81225 Rat farnesyltransferase alpha subunit mRNA, comple2.7e-164 |
| 258 | | 20720\| 82475\| 82475\| V00681 R. norvegicus mitochondrial genes for 16S rRNA, tRN 8.6e-82 |
| 259 | | 20716\| 82210\| 82210\| AF014955 Homo sapiens TFAR19 mRNA, complete cds. 1.3e-70 |
| 260 | | 20710\| 82096\| 82096\| X14848 Rat (R. norvegicus) mitochondrial genome. 1.0e-99 |
| 261 | | 2071\| UI-R-AO-at-f-11-0-UI\| AA818381\| Similar to MMCKIT Mouse c-kit mRNAmRNA |
| 262 | axon outgroth | 20709\| 82094\| 82094\| X03369 Rat mRNA for beta-tubulin T beta15. 5.7e-105 |
| 263 | | 20708\| 82093\| 82093\| G15458 human STS SHGC-16876. 6.7e-51 |
| 264 | | 20703\| 82047\| 82047\| M71245 Rat prostatein C3 subunit gene, complete cds. 3.7e-36 |
| 265 | proteasome | 20694\| 81776\| 817761045249 Rat mRNA for proteasome activator rPA28 subunit al 1.4e-178 |
| 266 | | 20693\| 81773\| 81773\| X68273 M. musculus mRNA for macrosialin. 1.0e-134 |
| 267 | | 20690\| 81743\| 81743\| X05608 Human gene for neurofilament subunit NF-L. 5.0e-38 |
| 268 | | 20674\| 80860\| 80860\| X95591 M. musculus mRNA for C1D protein. 1.7e-77 |
| 269 | | 20673\| 80835\| 80835\| AC000083 Home sapiens Chromosome 22q11.2 Cosmid Clone 68a1 0.65 |
| 270 | | 20672\| 80832\| 80832\| U16686 Rattus norvegicus defensin RatNP-1 precursor mRNA, 2.9e-117 |
| 271 | | 20669\| 80637\| 80637\| X70369 R. norvegicus mRNA for pro alpha 1 collagen type II 2.9e-81 |
| 272 | | 20667\| 80178\| 80178\| M58405 R. norvegicus thymosin beta-b gene, complete cds. 6.1e-79 |
| 273 | | 20665\| 80157\| 80157\| D83407 ZAKI-4 mRNA in human skin fibroblast, complete cds 1.2e-59 |
| 274 | | 20661\| 80013\| 80013\| G27219 human STS SHGC-30611. 2.6e-60 |
| 275 | | 20655\| 79192\| 79192\| V00680 Rat mitochondrial genes coding for 16S and 12S rRN 1.7e-109 |
| 276 | | 20654\| 79184\| 79184\| J04517 Rat high molecular weight neurofilament (NF-H) pro 5.3e-141 |
| 277 | | 20591\| 66897\| 66897\| M80899 Human novel protein AHNAK mRNA, partial sequence. 1.3e-38 |
| 278 | | 20590\| 66886\| 66886\| U52828 Human Cri-du-chat region mRNA, clone NIBA2. 2.2e-75 |
| 279 | | 20588\| 66841\| 66841\| X51406 Rat mRNA for carboxypeptidase E (EC 3.4.17.10). 2.4e-167 |
| 280 | | 20587\| 66839\| 66839\| M15563 Human MHC class II HLA-DR-beta-1 pseudogene (DR7), 0.79 |
| 281 | | 20583\| 66706\| 66706\| M30952 Orangutan 28S ribosomal RNA gene fragment. 9.8e-151 |
| 282 | | 20582\| 66692\| 66692\| L81910 Homo sapiens (subclone 3_c10 from PAC H74) DNA seq 0.00048 |
| 283 | | 20581\| 66688\| 66688\| U49099 Rattus nervegicus cis-Golgi p28 (p28) mRNA, comle 6.8e-193 |
| 284 | | 20568\| 115932\| 115932\| M25638 Rat smallest neurofilament protein (NF-L) mRNA, pa 6.7e-157 |
| 285 | | 20566\| 115916\| 115916\| X62952 R. norvegicus mRNA for vimentin. 2.8e-167 |
| 286 | | 20563\| 113752\| 113752\| AL021308 Human DNA sequence from cosmid U246D9 on chromosom 0.93 |
| 287 | | 20562\| 113749\| 113749\| M80840 M. musculus necdin mRNA, complete cds. 1.7e-71 |
| 288 | | 20557\| 113571\| 113571\| D28875 Rat mRNA for osteonectin, complete cds. 2.2e-171 |
| 289 | | 20550\| 113529\| 113529\| M38194 Rat extracellular signal-regulated kinase 1 mRNA, 2.8e-203 |
| 290 | | 20545\| 113318\| 113318\| I00256 Sequence 7 from U.S. Pat. No. 4900811. 1.3e-191 |
| 291 | | 20541\| 113285\| 113285\| X76985 R. norvegicus mRNA for latexin. 4.3e-171 |
| 292 | | 20540\| 113284\| 113284\| M23984 Rat mitochondrial proton/phosphate symporter mRNA, 2.7e-174 |
| 293 | ion channel | 20536\| 113244\| 113244\| X15466 Rat mRNA for GABA(A) receptor beta-1 subunit. 7.3e-172 |
| 294 | | 20532\| 113167\| 113167\| AC002299 Homo sapiens Chromosome 16 BAC clone CIT987-SKA-11 0.84 |
| 295 | | 20530\| 113155\| 113155\| U40933 Caenorhabditis elegans cosmid F20D12. 0.38 |
| 296 | | 20529\| 113063\| 113063\| AC000399 Genomic sequence from Mouse 9, complete sequence. 6.5e-11 |
| 297 | | 20520\| 112951\| 112951\| AC002121 Genomic sequence from Mouse 11, complete sequence. 0.00031 |
| 298 | | 20512\| 112160\| 112160\| M14512 Rat Na+, K+-ATPase alpha(+) isoform catalytic subun 1.5e-259 |
| 299 | | 20505\| 111646\| 111646\| D28875 Rat mRNA for osteonectin, complete cds. 2.5e-206 |
| 300 | | 20501\| 111616\| 111616\| X58526\| Mouse DNA for an L1 element. 1.3e-70 |
| 301 | | 20500\| 111613\| 111613\| M25888 Rat lipophilin mRNA, 3′ end. 4.4e-125 |
| 302 | | 20496\| 111441\| 111441\| U25844 Mus musculus serine proteinase inhibitor (SPI3) mR 1.6e-134 |
| 303 | | 20476\| 109494\| 109494\| M38188 Human unknown protein from clone pHGR74 mRNA, comp 2.8e-84 |
| 304 | | 20475\| 109492\| 109492\| M26232 Rat peripherin gene, complete cds. 1.4e-1 39 |
| 305 | | 20472\| 109006\| 109006\| S80636 injury-inducible gene (ID repetitive element, clon 3.6e-07 |
| 306 | | 20471\| 109001\| 109001\| M89646 Rattus norvegicus ribosomal protein S24 mRNA seque 1.6e-80 |
| 307 | | 20468\| 108618\| 108618\| L35240 Human enigma gene, complete cds. 2.5e-17 |
| 308 | | 20467\| 108615\| 108615\| X55572 R. norvegicus Apo D mRNA. 9.0e-193 |
| 309 | | 20464\| 108534\| 108534\| X77953 R. norvegicus mRNA for ribosomal protein S15a. 1.4e-68 |
| 310 | | 20455\| 106217\| 106217\| M20480 Mouse brain neurofilament-L mRNA, complete cds. 4.7e-122 |
| 311 | | 20448\| 106118\| 106118\| L10426 Mus musculus ets-related protein 81 (2R81) mRNA, c 5.1e-48 |
| 312 | | 20443\| 105946\| 105946\| D83407 ZAKI-4 mRNA in human skin fibroblast, complete cds 5.8e-63 |
| 313 | | 20434\| 105919\| 105919\| X61294 R. norvegicus L1 retroposon, ORF2 mRNA (partial). 1.5e-92 |
| 314 | | 20430\| 105905\| 105905\| D83783 Human mRNA for KIAA0192 gene, partial cds. 3.4e-05 |
| 315 | | 20423\| 105878\| 105878\| U13369 Human ribosomal DNA complete repeating unit. 0.021 |
| 316 | | 20420\| 105801\| 105801\| D17577 Mouse mRNA for kinesin-like protein (Kif1b), compl 2.5e-122 |
| 317 | | 20419\| 105787\| 105787\| X60370 R. norvegicus mRNA for microtubule associated prote 1.2e-97 |
| 318 | | 20416\| 105774\| 105774\| U02983 Rattus norvegicus secretogranin III (SgIII) mRNA, 9.0e-82 |
| 319 | | 20412\| 103932\| 103932\| M35826 Rat mitochondrial NADH-dehydrogenase (NDI) gene, c 2.0e-78 |
| 320 | G protein signaling | 20410\| 103925\| 103925\| M12673 Rat guanine nucleotide-binding protein G-s, alpha 4.1e-66 |
| 321 | | 20406\| 103909\| 103909\| M13100 Rat long interspersed repetitive DNA sequence LINE 4.3e-172 |
| 322 | | 20404\| 103831\| 103831\| D90005 Rat endogenous retroviral sequence, 5′ and 3′ LTR. 4.8e-97 |
| 323 | | 20403\| 103830\| 103830\| M23953 Rat carboxypeptidase B gene, exons 6, 7, and 8. 1.1e-68 |
| 324 | | 20401\| 103821\| 103821\| D10699 Rat mRNA for ubiquitin carboxyl-terminal hydrolase 3.1e-99 |
| 325 | | 20400\| 103820\| 103820\| X60469 R. rattus FE65 mRNA for adaptor protein interacting 3.0e-167 |
| 326 | | 20396\| 103801\| 103801\| AF043698 Caenorhabditis elegans cosmid C54G6. 0.12 |
| 327 | | 20395\| 103795\| 103795\| X70369 R. norvegicus mRNA for pro alpha 1 collagen type II 3.0e-69 |
| 328 | | 20391\| 103703\| 103703\| V01556 Rat mitochondrial genome fragment from Sprague-Daw 1.0e-188 |
| 329 | | 20385\| 102872\| 102872\| L40632 Mus musculus epithelial ankyrin 3(7kb isoform) mR 1.6e-175 |
| 330 | | 20379\| 102748\| 102748\| M16597 Human cytochrome c1 mRNA, partial cds. 2.9e-127 |
| 331 | | 20363\| 101197\| 101197\| G05883 human STS WI-6610. 1.9e-65 |
| 332 | | 20437\| 101132\| 101132\| M89646 Rattus norvegicus ribosomal protein S24 mRNA seque 6.2e-55 |
| 333 | hsp | 20343\| 101112\| 101112\| M86389 Rat heat shock protein (Hsp27) mRNA, complete cds. 1.2e-147 |
| 334 | | 20342\| 101108\| 101108\| X75683 H. spaiens (TL35) mRNA from LNCaP cell line. 1.0e-73 |
| 335 | | 20340\| 101083\| 101083\| X79064 V. unguicutata (Red Caloona) AKCS9 mRNA. 0.041 |
| 336 | | 20334\| 100940\| 100940\| Z12152 R. norvegicus mRNA for neurofilament protein middle 1.5e-206 |
| 337 | | 20328\| 100875\| 100875\| L01693 Rat peptidyiglycine alpha-amidating monooxygenase 3.2e-123 |
| 338 | | 20322\| 100839\| 100839\| X96487 B. taurus mRNA, anonymous. 5.5e-09 |
| 339 | prteasome | 20321\| 100836\| 100836\| M29859 Rat proteasomes C2 component mRNA, complete cds. 1.0e-100 |
| 340 | | 20317\| 100759\| 100759\| M29341 Rat brain glyceraldehyde-3-phosphate dehydrogenase 2.8e-103 |
| 341 | apoptosis | 20308\| 100704\| 100704\| D17614 Rat mRNA for 14-3-3 protein theta-subtype, complet 3.9e-228 |
| 342 | | 20307\| 100574\| 100574\| M22012 Mus musculus synaptosomal associated protein 25 (S 4.3e-72 |
| 343 | | 20306\| 100573\| 100573\| L27153 Mouse kinesin heavy chain (Khcs) mRNA, 5′ end. 1.0e-81 |
| 344 | | 20302\| 100557\| 100557\| U80954 Caenorhabditis elegans cosmid T07F8. 0.84 |
| 345 | axon outgrowth | 20301\| 100465\| 100465\| X03369 Rat mRNA for beta-tubulin T beta15. 7.3e-91 |
| 346 | | 20300\| 100463\| 100463\| X62952 R. norvegicus mRNA for vimentin. 1.2e-114 |
| 347 | | 20298\| 100455\| 100455\| U62800 Human cystatin M (CST6) mRNA, complete cds. 8.9e-91 |
| 348 | | 20296\| 100438\| 100438\| A08267 Nucleotide sequence for P. falciparum RMA-1 gene. 0.87 |
| 349 | | 20295\| 100437\| 100437\| S69874 C-FABP=cutaneous fatty acid-binding protein [rats, 6.7e-160 |
| 350 | | 20272\| 1065569\| Z33905\| nicotinic acethyicholine receptor associated 43 kD protein |
| 351 | | 20264\| 917438\| Z26634\| ankyrin brain variant, ANKB |
| 352 | | 20258\| 1260365\| Z21876\| H. sapiens X-linked ALD gene. |
| 353 | GPCR | 20238\| 700123692\| Y10148\| NTR2 receptor |
| 354 | | 20204\| 613665\| X96427\| Homol. to ScUBC9 |
| 355 | | 20127\| 921632\| X69292\| myosin heavy chain. Cell Type:smooth muscle Tissue Expression: GI wall, Respiration |
| 356 | | 20080\| 1497875\| X61615\| H. sapiens mRNA for leukemia inhibitory factor (LIF) receptor |
| 357 | | 20055\| UI-R-C1-kp-d-10-0-UI\| X57748f properdin |
| 358 | | 20029\| 700247749\| X54673\| H. sapiens GAT1 mRNA for GABA transporter |
| 359 | | 20027\| 1244915\| X54297\| Human mRNA for histidine decarboxylase (EC 4.1.1.22) |
| 360 | | 19976\| 700247354\| X1S376\| Human mRNA for GABA-A receptor, gamma 2 subunit |
| 361 | | 19960\| 700247209\| X14767\| Human mRNA for GABA-A receptor, beta 1 subunit |
| 362 | transcription factor | 19941\| UI-R-BT0-pm-c-04-0-UI\| X12740\| C-Jun of the transcription factor AP-1 |
| 363 | neuroptide | 1986\| UI-R-A0-au-d-10-0-UI\| AA818532\| Rat gamma-preprotachydinin A mRNA, complete cds |
| 364 | neuropeptide | 19136\| 315382\| W12030\| U10071 RNU10071 Rattus norvegicus CART protein mRNA, complete cds. Blastn: P. = 8.5e-117 |
| 365 | proteasome | 18970\| 931025\| U75362\| ubiquitin carboxy terminal hydrolase |
| 366 | growth factor | 18954\| UI-R-Y0-mo-d-05-0-UI\| U66198\| fibroblast growth factor, FGF13 |
| 367 | apoptosis | 18941\| 1050567\| U59746\| Bcl-w |
| 368 | | 18937\| UI-R-AG0-xc-h-06-0-UI\| U58522\| huntingtin interacting protein |
| 369 | proteasome | 18904\| 407454\| U44839\| A ubiquitin C-terminal hydrolase |
| 370 | | 18871\| 700128068\| U33267\| Human glycine receptor beta subunit (GLRB) mRNA, complete cds |
| 371 | | 18862\| 803318\| U30509\| Gamma Glu Tranterase |
| 372 | | 18856\| UI-R-C1-lp-e-07-0-UI\| U28807\| NFATc3 |
| 373 | | 18835\| 789552\| U20240\| CCAAT enhancer binding protein gamma |
| 374 | GPCR | 18815\| 700498633\| U16129\| Glutamate receptor (GluR4) |
| 375 | | 18784\| 1617912\| U08107\| HSU08107 Human N-methyl-D-aspartate receptor subunit splice variant hNR1N (GRIN1) mRNA, partial cds. |
| 376 | | 1824\| UI-R-A0-ar-f-11-0-UI\| AA818840\| Similar to MMU75839 mouse interferon regulatory factor 3 (mirf3) mRNA, alternatively spliced, complete cdsmRNA |
| 377 | | 18104\| 1039340\| M9S106\| CREB-1 |
| 378 | neuropeptide | 18088\| 640127\| M88355\| MUSOXYNEUI Mouse oxytocin-neurophysin I gene, complete cds. |
| 379 | | 18037\| UI-R-C3-ss-b-09-0-UI\| M737461 Homo sapiens lutropin/horiogonadotropin reeptor (LHCGR) mRNA, complete cds. |
| 380 | | 17418\| UI-R-Y0-acb-c-12-0-UI\| R60006\| HUMEAR17A Human trilodothyronine recptor (THRAI, ear1), and ear2 genes, last 2 exons each. BLASTN P. 2.8E-96 |
| 381 | | 1571\| UI-R-A0-bc-d-07-0-UI\| AA819345\| Rat parvalbumin mRNA, complete cds |
| 382 | | 14951\| 700861309\| M59980\| K Channel |
| 383 | | 14925\| UI-R-C0-ie-h-11-0-UI\| M55040\| Human acetylcholinesterase (ACHE) mRNA, complete cds |
| 384 | | 14871\| 492525\| M29871\| Human ras-related C3 botulinum toxin substrate (rac) mRNA, complete cds |
| 385 | G protein signaling | 14870\| UI-R-E0-da-b-11-0-UI\| M29870\| Human ras-related C3 botulinum toxin substrate (rac) mRNA, complete cds |
| 386 | | 14866\| UI-R-AA0-wp-c-07-0-UI\| M29548\| Human elongation factor 1-alpha (EF1A) mRNA, partial cds. |
| 387 | | 14866\| 299720\| M29548\| Human elongation factor 1-alpha (EF1A) mRNA, partial cds. |
| 388 | axon outgrowth | 14839\| 338087\| M25667\| Human neuronal growth protein 43 (GAP-43) mRNA, complete cds |
| 389 | G protein signaling | 14829\| UI-R-C1-lh-b-02-0-UI\| M23612\| Human GTPase-activating protein (GAP) mRNA, complete cds |
| 390 | | 14817\| 349760\| M21571\| Human platelet-derived growth factor (PDGFA) A chain mRNA. |
| 391 | | 14739\| 700124548\| L76224\| Homo sapiens NMDA receptor mRNA, complete cds |
| 392 | | 14727\| 1162580\| L42198\| MUSALOX Mus musculus arachidonate 5-lipoxygenase (Alox5) gene, complete cds. |
| 393 | | 14677\| 1066814\| L16510\| Homo sapiens cathepsin B A cysteine protease associated with tumor invasiveness and neovascurlarisati |
| 394 | GPCR | 1467\| UI-R-A0-an-d-02-0-UI\| AA819622\| Rat G-protein coupled thrombin receptor mRNA, complete cds |
| 395 | neuropeptide | 14620\| 482891\| K01911\| Human neuropeptide Y (NPY) mRNA, complete cds |
| 396 | | 13845\| 963216\| H58254\| monocyte chemoattractant protein 1 receptor (MCP-1RA) |
| 397 | | 13310\| 602816\| D86323\| D86323 Mus musculus mRNA for calmegin, complete cds. |
| 398 | | 13304\| 1246629\| D83004\| similar to Drosophila bendless gene |
| 399 | proteasome | 13275\| UI-R-E0-ct-g-08-0-UI\| D23662\| ubiquitin-like protein |
| 400 | | 13165\| UI-R-BT0-qi-f-04-0-UI\| AI145577\| Rat degenerin channel MDEG mRNA, complete cds |
| 401 | hsp | 1278\| 1246430\| AA823192\| D49547 HUMHSP40 Human mRNA for heat-shock protein 40, complete cds. Blastn P. = 3.5E-167 |
| 402 | | 12712\| UI-R-C2-nf-c-01-0-UI\| AI072073\| Rat vasopressin mRNA, complete cds |
| 403 | G protein signaling | 12702\| UI-R-C2-nh-g-02-0-UI\| A1071860\| Rat regulator of G-protein signalling 14 (RGS14) mRNA, complete cds |
| 404 | | 12681\| UI-R-C2-my-e-04-0-UI\| A1071179\| Rat mRNA for cyclinG-associated kinase, complete cds |
| 405 | | 12678\| UI-R-C2-ms-b-07-0-UI\| AI071095\| Rat vgr mRNA |
| 406 | | 1266\| UI-R-AO-aq-c-10-0-UI\| AA819897\| Rat synuclein 1 mRNA, complete cds |
| 407 | ion channel | 12538\| UI-R-C1-kp-b-07-0-UI\| AI058645\| Rat Kv4.3 potassium channel mRNA, complete cds |
| 408 | neuropeptide | 12490\| UI-R-C1-iz-a-09-0-UI\| AI045437\| Rat neuropeptide Y mRNA, complete cds |
| 409 | | 12463\| UI-R-C1-kd-e-12-0-UI\| AI044870\| R. norvegicus mRNA for elongation factor 1 alpha |
| 410 | | 12448\| UI-R-C1-kc-c-05-0-UI\| AI044610\| Rat dopa decarboxylase (DOC) mRNA, complete cds |
| 411 | | 12427\| UI-R-C1-kb-b-03-0-UI\| AI044262\| Rat mRNA for thyrotropin releasing hormone receptor, complete cds |
| 412 | | 12350\| UI-R-C0-ib-c-02-0-UI\| AI030286\| Rat brain-derived neurotrophic factor (BDNF) mRNA, complete cds |
| 413 | | 12318\| UI-R-CO-iy-b-07-0-UI\| AI029599\| Rat mineralocorticoid receptor (MR) mRNA, complete cds |
| 414 | | 12263\| 659316\| AF075599\| conjugating enzymes |
| 415 | ion channel | 12260\| 700254638\| AF064876\| BCNG-1 hyperpolarization NSC channel=HCN1 = HAC2 |
| 416 | proteasome | 12223\| UI-R-C1-kt-f-03-0-UI\| AF022789\| ubiquitin hydrolyzing enzyme I |
| 417 | | 11788\| UI-R-C0-ho-b-04-0-UI\| AA997314\| Rat cDNA clone UI-R-C0-ho-b-04-0-UI 3′ mRNA |
| 418 | proteasome | 1038\| UI-R-E0-cj-b-04-0-UI\| AA859300\| Rat proteasomes C2 component mRNA, complete cds |
| 419 | proteasome | 1031\| UI-R-E0-cd-c-03-0-UI\| AA859229\| Rattus norvegicus ubiquitin conjugating enzyme mRNA, complete cds |

| | C | D | E | F | G | H | I | J | K | | |
|---|---|---|---|---|---|---|---|---|---|---|---|
| 1 | CA1.CMS | CA1.IMP | CA1.IMPnCMS | CA1.JRF | CA1.JRFnCMS | CA3.CMS | CA3.IMP | CA3.IMPnCMS | CA3.JRF | | |
| 2 | 1 | 1 | 1 | 1 | 1 | 1 | 1 | 1 | 1 | | |
| 3 | 1 | 1 | 1 | 1 | 1.135106383 | 1 | 1 | 1 | 1 | | |
| 4 | 1 | 1.026595745 | 1.069148936 | 1.109148936 | 1.159787234 | 1.053803876 | 1.536399576 | 1.540545753 | 1.389933468 | | |
| 5 | 1.283765785 | 1.117871942 | 1.152476322 | 1.417151736 | 1.500295975 | 1.098971902 | 0.830980194 | 0.88179679 | 0.923074768 | | |
| 6 | 0.985121588 | 1.016195517 | 1.146686505 | 1.191468085 | 1.299080465 | 1.229552015 | 1.146405788 | 0.918101144 | 1.264264585 | | |
| 7 | 1 | 1 | 1 | 1 | 1.154787234 | 1 | 1 | 1 | 1 | | |
| 8 | 0.918237765 | 1.515092312 | 1.8366709 | 2.101396894 | 1.508840481 | 0.833539644 | 0.862206007 | 0.926900775 | 0.864616587 | | |
| 9 | 1.271983351 | 1.490840538 | 1.058110556 | 1.17292499 | 1.134983235 | 1.184266024 | 0.98016667 | 0.86311757 | 1.142719482 | | |
| 10 | 0.786262347 | 0.879611818 | 1.108889705 | 1.083839454 | 1.490824994 | 0.867116391 | 1.20458322 | 1.08546415 | 1.020460802 | | |
| 11 | 1.277605055 | 1.843345703 | 1.294554207 | 1.527329255 | 1.789088356 | 1.071466291 | 0.968051399 | 0.937863052 | 1.243531068 | | |
| 12 | 0.580658633 | 0.857415583 | 0.803006801 | 0.723660661 | 0.930258919 | 0.927113703 | 0.854713314 | 1.140751539 | 1.097910593 | | |
| 13 | 0.760842082 | 0.967954708 | 1.158522509 | 0.800087946 | 0.68949596 | 0.778530809 | 0.868427203 | 0.784918595 | 0.732258316 | | |
| 14 | 1 | 1.955319149 | 1.436808511 | 1.609787234 | 1.482234043 | 1 | 1.249255311 | 1.294361702 | 1.601808511 | | |
| 15 | 1 | 1 | 1 | 1 | 1 | 1 | 1 | 1 | 1 | | |
| 16 | 1 | 1 | 1.083085106 | 1 | 1 | 1 | 1 | 1 | 1 | | |
| 17 | 1.68533171 | 1.843919659 | 1.306025563 | 1.025562995 | 0.761412051 | 1.042526807 | 0.870960683 | 1.147676709 | 0.991100737 | | |
| 18 | 0.918452657 | 1.022825294 | 1.167341251 | 1.187777852 | 1.200384845 | 1.010731303 | 1.033229741 | 0.934244873 | 0.893184172 | | |
| 19 | 1.031509395 | 0.932438571 | 1.124055338 | 0.858145777 | 1.115713401 | 0.971141137 | 0.83235423 | 0.850822441 | 0.850712293 | | |
| 20 | 1 | 1 | 1 | 1 | 1 | 1 | 1 | 1 | 1 | | |
| 21 | 1.330920372 | 1.107606572 | 1.370217166 | 1.141388027 | 1.355883029 | 1.197396918 | 1.041212021 | | | | |
| 22 | 1.001916525 | 1.729840999 | 1.175042589 | 1.122373651 | 1.216283362 | 0.8854339 | 1.01981501 | 1.018987818 | 0.797337946 | | |
| 23 | 1.168235333 | 0.868461614 | 0.829895386 | 0.880070836 | 0.817892631 | 1.282736246 | 1.151929311 | 1.217781459 | 0.648857596 | | |
| 24 | 0.806988969 | 1.21877545 | 1.287776708 | 1.377952173 | 1.213148738 | 0.896036998 | 0.983004963 | 1.312265002 | 0.995262445 | | |
| 25 | 1.548089532 | 1.22034818 | 1.203527018 | 1.708161881 | 1.663124576 | 1.102383239 | 1.001680874 | 1.09205787 | 1.264227398 | | |
| 26 | 1 | 1.348191489 | 1.561276596 | 1.589361702 | 1.994787234 | 0.859468723 | 0.972812963 | 0.773545221 | 0.775103217 | | |
| 27 | 1.128055186 | 0.937950837 | 0.853841305 | 0.756985783 | 0.825232859 | 0.972462265 | 0.848513654 | 0.910617583 | 0.784796636 | | |
| 28 | 1 | 1.147340426 | 1.153297872 | 1.194255319 | 1.194361702 | 1 | 1 | 1 | 1 | | |
| 29 | 0.929867332 | 1.0147409 | 1.260800544 | 1.333484522 | 1.522678308 0.83777447 | 0.778861183 | 0.844324525 | 0.689691105 | | | |
| 30 | 1 | 1 | 1 | 1.025 | 1 | 1 | 1 | 1 | 1 | | |
| 31 | 1.113756425 | 0.948850657 | 1.197387207 | 1.197208738 | 1.221159338 | 1.064588104 | 1.141800247 | 0.889935999 | 0.918677705 | | |
| 32 | 1.001651053 | 0.972291016 | 0.96317433 | 1.059976311 | 0.713398658 | 0.927060922 | 1.004460303 | 0.816130556 | 0.828514457 | | |
| 33 | 0.915764112 | 0.499164687 | 0.602538896 | 0.708686116 | 0.673568888 | 0.884301786 | 1.584374032 | 1.231258816 | 1.503423126 | | |
| 34 | 0.707739249 | 1.05150544 | 1.265663679 | 1.446508415 | 1.305367389 | 0.958500813 | 0.900369795 | 1.187684227 | 0.844704075 | | |
| 35 | 1.045635922 | 1.026555979 | 1.459111131 | 1.488602486 | 1.335229097 | 0.89865223 | 1.152229878 | 1.020670856 | 1.020784432 | | |
| 36 | 0.908402292 | 1.832463399 | 1.196721833 | 1.174633991 | 1.095798854 | 1.068026865 | 1.253434415 | 1.775770835 | 1.577592348 | | |
| 37 | 1.113297872 | 1.338085106 | 1.106489362 | 1.1752127661 | 0.853193351 | 1.20962379 | 1.209623797 | 1.035053952 | 0.86573345 | | |
| 38 | 0.942528893 | 0.933867927 | 0.855850933 | 1.025504768 | 0.825086746 | 1.132523583 | 1.276845591 | 1.315607869 | 0.995005148 | | |
| 39 | 1 | 1.016489362 | 1.344042553 | 1.98 | 1.780212766 | 1 | 1 | 1 | 1 | | |
| 40 | 1 | 1.036595745 | 1 | 1 | 1 | 1 | 1.059574468 | 1 | 1 | | |
| 41 | 1 | 1.036595745 | 1 | 1 | 1 | 1.030212766 | 1.249255319 | 1.164361702 | 1.174574468 | | |
| 42 | 0.928423354 | 0.98207757 | 1.330933625 | 1.296577588 | 1.274000226 | 0.982149165 | 1.018675082 | 0.981466218 | 0.847396557 | | |
| 43 | 0.971175085 | 1.263510529 | 1.663230747 | 2.063944927 | 1.958732145 | 1.007528423 | 0.938664374 | 1.229750645 | 0.800496799 | | |
| 44 | 1.205319149 | 1.418404255 | 1.882234043 | 1.958297872 | 1.946170213 | 1.118771009 | 1.02866073 | 1.150380374 | 1.142772896 | | |
| 45 | 1 | 1 | 1 | 1 | 1 | 1 | 1 | 1 | 1 | | |
| 46 | 1.694640812 | 1.304332372 | 0.958868133 | 1.026420757 | 0.89092058 | 1.539610003 | 0.876365457 | 0.585746033 | 0.952971924 | | |
| 47 | 0.806988969 | 0.966165692 | 0.875101799 | 0.891241578 | 0.886651366 | 1 | 1 | 1 | 1 | | |
| 48 | 0.808675141 | 0.578299043 | 0.655571572 | 0.466998646 | 0.66823921 | 1.209680851 | 1.279148936 | 1.128297872 | | | |
| 49 | 1.00393617 | 1.378297872 | 1.120425532 | 1.146808511 | 1 | 1 | 1.225531915 | 1.056382979 | 1 | | |
| 50 | 0.977017638 | 0.970886912 | 0.804162606 | 0.68296287 | 0.822554784 | 1.20317521 | 1.095218057 | 0.937222647 | 1.197092578 | | |
| 51 | 0.847565517 | 0.91 4465052 | 0.857955956 | 0.990283343 | 0.852459307 | 0.896263683 | 1.098172552 | 1.211076264 | 0.840475257 | | |
| 52 | 1 | 1 | 1 | 1 | 10.841286042 | 0.979841796 | 0.955430807 | 0.852513396 | | | |
| 53 | 1.303913818 | 1.909671325 | 1.164766256 | 1.036645988 | 0.906355098 | 1.062618209 | 1.006415724 | 1.339699611 | 1.61611348 | | |
| 54 | 0.757270603 | 0.967695158 | 1.006767099 | 1.0963506 | 1.91976154 | 0.925404696 | 0.975864081 | 0.871518157 | 0.85773662 | | |
| 55 | 0.992468022 | 1.104051685 | 1.37030063 | 1.268781248 | 1.541263554 | 0.860852567 | 1.014633884 | 1.063825972 | 0.885385843 | | |
| 56 | 1.534014194 | 1.104985286 | 1.897178466 | 1.681841786 | 1.43110611 | 1 | 1.162234043 | 1 | 1.19 | | |
| 57 | 1.112251548 | 1.075051591 | 1.611980015 | 1.387078672 | 1.433040078 | 1.300651235 | 0.838534071 | 1.051217998 | 1.360764155 | | |
| 58 | 1.228716794 | 1.700507317 | 1.103320224 | 1.328279653 | 1.77938274 | 1.21985654 | 1.040759567 | 0.719098387 | 1.170470526 | | |
| 59 | 0.855115112 | 1.433010213 | 1.505452657 | 2.118660204 | 1.800501991 | 1 | 1 | 1 | 1 | | |
| 60 | 1.412610123 | 0.900950078 | 0.74451546 | 0.867852824 | 0.806944205 | 1.325264926 | 1.402358755 | 0.86140869 | 0.801416797 | | |
| 61 | 1.311790816 | 1.256009503 | 1.151183145 | 1.493967053 | 1.302397549 | 1.074979172 | 1.44027581 | 1.548198417 | 0.847455568 | | |
| 62 | 1 | 1 | 1 | 1 | 1 | 1 | 1 | 1 | 1 | | |
| 63 | 0.672108555 | 0.643459133 | 0.567634899 | 0.538044631 | 0.730971059 | 0.812586363 | 0.48107104 | 0.695699919 | 0.606111076 | | |
| 64 | 1.270667179 | 1.072979725 | 0.870984273 | 0.858300503 | 0.724304154 | 1.350444203 | 1.495203492 | 1.335179564 | 1.241996588 | | |
| 65 | 1.116187147 | 1.111616026 | 1.030841624 | 0.808631353 | 0.772653939 | 1.086217209 | 1.216739298 | 1.1548744818 | 1.509356575 | | |
| 66 | 1.206040951 | 1.139644221 | 1.052407361 | 0.902093608 | 0.920496632 | 1.110717409 | 1.041554839 | 0.906094509 | 0.747896626 | | |
| 67 | 1 | 1.026595745 | 1.334361702 | 1.329255319 | 1.518297872 | 0.954445612 | 0.965428326 | 0.941457358 | 0.971158438 | | |
| 68 | 1.306231353 | 1.101972009 | 0.985058336 | 1.119193732 | 0.926674267 | 0.951589282 | 0.714795487 | 0.826509354 | 0.685423155 | | |
| 69 | 1 | 1 | 1 | 1 | 1 | 1 | 1 | 1 | 1 | | |
| 70 | 1.09787234 | 1.297978723 | 1.422021277 | 1.368510638 | 1.214148936 | 0.974358974 | 0.840080972 | 0.959598516 | 0.870023617 | | |
| 71 | 0.73667584 | 0.870033722 | 1.103647428 | 1.388193341 | 1.439610062 | 0.783437291 | 1.018781504 | 1.242893641 | 0.90936083 | | |
| 72 | 1 | 1 | 1 | 1 | 1 | 1.169861203 | 0.988323419 | 1.018873467 | 1.168465888 | | |
| 73 | 0.919550054 | 0.871412423 | 1.397149238 | 1.447961181 | 1.35868876 | 0.711905307 | 0.395935179 | 0.997628508 | 1.119926775 | | |
| 74 | 1.113297872 | 1.468617021 | 2.645851064 | 1.925957447 | 1.828191489 | 1 | 1 | 1 | 1 | | |
| 75 | 1 | 1 | 1 | 1 | 1 | 1.021626754 | 0.995885642 | 1.162253402 | 1.103597426 | | |
| 76 | 0.745266611 | 0.966697299 | 1.095951272 | 1.072977345 | 1.518375345 | 0.938618594 | 0.888933685 | 1.018425461 | 0.96207878 | | |
| 77 | 0.803854277 | 1.124274023 | 0.968585005 | 0.941789863 | 0.875065998 | 0.913779465 | 0.743553235 | 0.945420028 | 0.88482835 | | |
| 78 | 1 | 1 | 1 | 1 | 1 | 1 | 1 | 1 | 1 | | |
| 79 | 1 | 1.348191489 | 1.476276596 | 1.65106383 | 1.913829787 | 1.130332943 | 1.213485026 | 1.029780827 | 1.176008031 | | |
| 80 | 0.815993416 | 1.057197723 | 1.162334545 | 1.213839929 | 1.012207668 | 0.801179065 | 0.979343864 | 0.949282211 | 0.722469736 | | |
| 81 | 1 | 1.227659574 | 1.106489362 | 1.261382979 | 1.100851064 | 0.940328737 | 1.288229337 | 1.036690816 | 0.860091922 | | |
| 82 | 1.205319149 | 1.086914894 | 1.162659574 | 1 | 1 | 1 | 1 | 1 | 1 | | |
| 83 | 1.128723404 | 1.488723404 | 1.683404255 | 1.755425532 | 1.679893617 | 0.97835059 | 1.334226341 | 1.065744696 | 1.177829267 | | |
| 84 | 1.074490777 | 0.903780794 | 0.661228699 | 0.63659377 | 0.587068076 | 0.926811099 | 0.849338879 | 0.970202992 | 1.071450742 | | |
| 85 | 1.874702466 | 2.074645339 | 2.031705227 | 1.313624679 | 2.20384652 | 1 | 1 | 1 | 1 | | |
| 86 | 0.82265375 | 1.091805901 | 1.163259865 | 0.693965517 | 0.591317099 | 0.963163653 | 0.877351118 | 0.955191997 | 0.661595373 | | |
| 87 | 0.872390104 | 0.938709533 | 0.977100265 | 0.97539401 | 0.848816847 | 1.000695998 | 1.006512553 | 0.818493661 | 0.723738504 | | |
| 88 | 1.173070553 | 1.422932538 | 1.421055537 | 1.505299768 | 1.080269405 | 0.951358435 | 1.001411361 | 1.004082867 | 1.182015223 | | |
| 89 | 1 | 1 | 1 | 1 | 1.003829787 | 1 | 1 | 1 | 1 | | |
| 90 | 0.895325849 | 1.01030855 | 0.727458872 | 0.58253901 | 0.667372732 | 1.013615601 | 0.912508784 | 1.014757554 | 0.906711174 | | |
| 91 | 0.872927371 | 0.860852982 | 1.259332143 | 1.22904899 | 1.233920047 | 1.124972175 | 0.755960401 | 0.964278601 | 1.022142815 | | |
| 92 | 0.723958825 | 1.042827462 | 1.095429219 | 0.826234772 | 0.887808103 | 0.86882034 | 0.832733403 | 0.7817925 | 0.731489497 | | |
| 93 | 1.029948644 | 1.117212794 | 1.515504633 | 1.312953556 | 1.580258457 | 1.031693304 | 0.790571212 | 0.786977482 | 0.845410914 | | |
| 94 | 1 | 1 | 1.00893617 | 1 | 1 | 1 | 1 | 1 | 1 | | |
| 95 | 0.761058688 | 1.098734177 | 1.557008055 | 1.873970081 | 1.798895282 | 0.837204421 | 1.041066182 | 1.054288513 | 0.873233525 | | |
| 96 | 0.988922062 | 1.071443537 | 1.171249738 | 1.1345066 | 1.240441022 | 0.97558229 | 0.657987256 | 0.818776093 | 1.146561195 | | |
| 97 | 1.075671927 | 1.16947242 | 0.955673548 | 0.938729404 | 0.976900199 | 0.938490674 | 0.929714686 | 0.869700752 | 0.793779396 | | |
| 98 | 0.657545256 | 0.836369551 | 0.852520188 | 0.664682919 | 0.556048835 | 0.66883818 | 0.987078894 | 1.269471638 | 1.175280737 | | |
| 99 | 1 | 1 | 1 | 1 | 1 | 1 | 1 | 1 | 1 | | |
| 100 | 1 | 1 | 1 | 1 | 1 | 1 | 1 | 1 | 1 | | |
| 101 | 2.534574468 | 1.791914894 | 2.377021277 | 2.100106383 | 1.149893617 | 1.052234043 | 1.272978723 | 1.225638298 | 1 | | |
| 102 | 0.970764883 | 1.113230878 | 0.912029677 | 0.914090561 | 0.991197079 | 0.979423287 | 0.679807998 | 0.790915187 | 1.106977729 | | |
| 103 | 1.069533217 | 1.291206663 | 1.294499322 | 1.38194848 | 1.396281232 | 0.910310631 | 0.747921832 | 0.892445676 | 0.862986729 | | |
| 104 | 0.781413253 | 0.921346577 | 1.193617679 | 1.30963019 | 1.237781826 | 0.960575787 | 0.856820588 | 1.183754585 | 0.886116354 | | |
| 105 | 0.716472848 | 0.73642377 | 0.943828017 | 0.901418969 | 0.943561293 | 0.790140936 | 0.703069696 | 1.014737113 | 1.046849862 | | |
| 106 | 1 | 1 | 1.027446809 | 1.280638298 | 1.169574468 | 0.813003946 | 0.930777149 | 1.104906502 | 1.328272431 | | |
| 107 | 0.909853812 | 0.773681233 | 1.07515677 | 1.18255493 | 1.171334809 | 0.898503448 | 1.336004044 | 0.987781498 | 1.231655555 | | |
| 108 | 1.159574468 | 1.468617021 | 1.62712766 | 1.915106383 | 1.881702128 | 0.802655909 | 1.251190676 | 1.214882053 | 0.925365608 | | |
| 109 | 1.297292191 | 1.072732997 | 1.064168766 | 1.608816121 | 1.174370277 | 0.993515437 | 1.057442901 | 1.514690692 | 1.583725468 | | |
| 110 | 0.898518949 | 1.050248333 | 1.021253747 | 0.869479619 | 1.111190657 | 0.823364076 | 0.884497145 | 0.885404772 | 0.939247548 | | |
| 111 | 1.039198581 | 1.060006909 | 1.023407905 | 1.083754398 | 1.250894337 | 1.107081501 | 0.903774665 | 1.112579953 | 0.989648546 | | |
| 112 | 0.899995961 | 1.61775516 | 1.656326992 | 1.455551517 | 1.54965871 | 1.09641048 | 1.111062694 | 1.841004412 | 1.611253336 | | |
| 113 | 1 | 1 | 2.027340426 | 1.915106383 | 1.764361702 | 1.320956112 | 0.947481974 | 1.244819168 | 1.116220973 | | |
| 114 | 0.924956775 | 0.79126469 | 0.76771619 | 0.784204526 | 0.707557571 | 1.156379727 | 1.501436978 | 1.04664477 | 1.208156562 | | |
| 115 | 0.990010852 | 0.685724152 | 0.39386839 | 0.351963299 | 0.34007498 | 0.914254643 | 1.332942948 | 1.266538012 | 1.277480341 | | |
| 116 | 1.279818121 | 1.08722641 | 1.051354987 | 0.788260516 | 0.73980607 | 0.881632545 | 0.662625744 | 0.547455016 | 0.674119793 | | |
| 117 | 1.530531915 | 1.237765957 | 1.120425532 | 1.060957447 | 1 | 1.472284264 | 0.714314721 | 0.755397631 | 0.636209814 | | |
| 118 | 0.492816046 | 0.687731028 | 0.71479541 | 0.661245219 | 0.387965671 | 0.737833595 | 0.793485086 | 1.083516484 | 0.906357928 | | |
| 119 | 0.842094228 | 1.235295839 | 1.253459659 | 1.480646388 | 1.274383405 | 0.939248014 | 1.183941689 | 1.070729131 | 0.857807773 | | |
| 120 | 0.945841357 | 0.881110206 | 1.308639461 | 1.236835284 | 1.318138054 | 1.037698821 | 0.970487636 | 1.145786218 | 0.898155244 | | |
| 121 | 1 | 1 | 1 | 1 | 1 | 1.008297872 | 1 | 1 | 1 | | |
| 122 | 1.304477612 | 1.477698716 | 1.634276293 | 2.137556404 | 1.692693509 | 1.247583828 | 0.979847105 | 0.97412909 | 1.206998353 | | |
| 123 | 1.072574911 | 0.928593195 | 0.759827324 | 0.68166582 | 0.661249365 | 1.402035969 | 1.12129284 | 0.839616559 | 0.875992535 | | |
| 124 | 1 | 1 | 1 | 1 | 1 | 1 | 1 | 1 | 1 | | |
| 125 | 1.123349743 | 1.131237413 | 0.646229582 | 0.583240098 | 0.847560976 | 1.278923038 | 1.018803098 | 0.877529985 | 0.993065967 | | |
| 126 | 1.088523159 | 1.22423789 | 1.203977308 | 2.016582507 | 1.561373979 | 0.81847162 | 1.003719118 | 1.294474453 | 1.094173382 | | |
| 127 | 0.843937156 | 0.696871223 | 0.851080972 | 0.718235531 | 0.762535249 | 1.033529098 | 1.093059263 | 1.133582488 | 1.098451682 | | |
| 128 | 1 | 1 | 1 | 1 | 1 | 1 | 1 | 1 | 1 | | |
| 129 | 1 | 1 | 1 | 1 | 1 | 1 | 1 | 1 | 1 | | |
| 130 | 0.973284082 | 1.30787733 | 1.588952839 | 1.857056954 | 1.571514475 | 0.824506697 | 0.907166956 | 1.067733361 | 0.867856047 | | |
| 131 | 0.757563023 | 0.705827477 | 0.864667346 | 0.775994444 | 0.872005341 | 1.41784193 | 1.674934903 | 1.860488568 | 1.950646458 | | |
| 132 | 0.946389999 | 0.743617655 | 0.858715877 | 0.884503784 | 0.778287269 | 0.941115776 | 1.454321987 | 1.168853371 | 1.305275403 | | |
| 133 | 1 | 1 | 1 | 1 | 1 | 1.052234043 | 1 | 1 | 1 | | |
| 134 | 1.162653798 | 0.772696104 | 1.009412237 | 1.2088999 | 1.029485206 | 1.033046715 | 0.886459762 | 0.94052476 | 1.035809682 | | |
| 135 | 1 | 1 | 1 | 1 | 1 | 1 | 1 | 1 | 1 | | |
| 136 | 1.218245301 | 1.222310795 | 0.822521244 | 0.643878641 | 0.669435454 | 1.280797706 | 1.279263656 | 0.910891749 | 0.910958447 | | |
| 137 | 1.00393617 | 1 | 1 | 1 | 1 | 1.027405152 | 0.773598881 | 0.773598881 | 0.773598881 | | |
| 138 | 1.059360983 | 0.855003046 | 0.837698654 | 1.094080514 | 1.141259206 | 1.177029353 | 0.983552896 | 0.913019435 | 0.943844377 | | |
| 139 | 0.911497132 | 0.77368317 | 0.791251895 | 0.654130134 | 0.823818314 | 0.932227635 | 0.888154334 | 1.004098044 | 1.015193691 | | |
| 140 | 0.90812401 | 0.891906163 | 0.72520178 | 0.826582183 | 0.749490835 | 1.652701989 | 1.28250308 | 1.033004753 | 0.920700581 | | |
| 141 | 1 | 1 | 1 | 1 | 1 | 0.981415744 | 1.101900188 | 1.609835039 | 1.092190436 | | |
| 142 | 1.192433644 | 0.810324026 | 0.691140986 | 0.602924279 | 0.560611283 | 1.062429696 | 1.039192469 | 1.2064413 | 1.316026286 | | |
| 143 | 1.269110502 | 1.038774234 | 1.062154231 | 1.316880743 | 1.556790302 | 1.110391092 | 1.052514489 | 0.859503491 | 0.947930452 | | |
| 144 | 0.856820615 | 0.904993118 | 0.604985472 | 0.612020187 | 0.6512846 | 1.069870793 | 0.838038022 | 0.906674348 | 1.115710641 | | |
| 145 | 1 | 1 | 1 | 1 | 1 | 1 | 1 | 1 | 1 | | |
| 146 | 0.688615426 | 1.023863752 | 1.057211816 | 1.103086651 | 0.990906619 | 0.782402006 | 0.7001837 | 0.723568255 | 0.599334707 | | |
| 147 | 1 | 1 | 1 | 1 | 1 | 1 | 1 | 1 | 1 | | |
| 148 | 1 | 1 | 1 | 1 | 1 | 1 | 1 | 1 | 1 | | |
| 149 | 0.909661463 | 0.452933344 | 0.323307982 | 0.328480705 | 0.313231415 | 1.054771242 | 1.299912854 | 1.091895425 | 1.282745098 | | |
| 150 | 0.679777789 | 0.826000694 | 1.048013491 | 0.958732206 | 1.179405783 | 1.062556168 | 1.303950299 | 0.943578322 | 1.023248545 | | |
| 151 | 1.066702128 | 1.097021277 | 1.556276596 | 1.861489362 | 1.617021277 | 1 | 1.075425532 | 1 | 1.027340426 | | |
| 152 | 0.872956909 | 1.133079495 | 1.160568351 | 1.263837296 | 1.420690936 | 1.335797509 | 1.105423865 | 1.063640016 | 0.927842507 | | |
| 153 | 1.384787234 | 1.107021277 | 1.125106383 | 1.090319149 | 1.047340426 | 1 | 1 | 1 | 1 | | |
| 154 | 1.045472142 | 0.528660307 | 0.191220794 | 0.129959906 | 0.155219135 | 1.183728226 | 1.222116734 | 0.768100388 | 1.778915215 | | |
| 155 | 1 | 1.006382979 1 | 1.309787234 | 1.224148936 | 0.749362245 | 0.93614477 | 0.906967474 | 0.931999362 | | | |
| 156 | 0.966036465 | 1.172367345 | 1.386251022 | 1.953288113 | 1.911723673 | 1.058765984 | 0.865228545 | 0.868283139 | 0.77888138 | | |
| 157 | 1.359417874 | 1.133930742 | 0.839394255 | 0.820638394 | 0.706401306 | 1.221036004 | 1.049131216 | 0.693883109 | 1.20093687 | | |
| 158 | 0.914359991 | 0.666666667 | 0.65010974 | 0.672292186 | 0.782522534 | 0.915537096 | 0.891617897 | 0.833641684 | 0.889748914 | | |
| 159 | 0.95996732 | 0.95996732 | 0.95996732 | 0.95996732 | 0.95996732 | 1.107340426 | 1 | 1 | 1 | | |
| 160 | 0.812916112 | 0.78985595 | 0.745430335 | 0.668623653 | 0.648650284 | 0.982563579 | 0.859828187 | 0.918601684 | 0.799523688 | | |
| 161 | 1.066906067 | 0.723222723 | 0.484461484 | 0.352323352 | 0.336123336 | 0.980411103 | 1.197909346 | 0.95686929 | 0.776748067 | | |
| 162 | 1.057441462 | 0.992061909 | 0.768319438 | 0.674059596 | 0.697514859 | 1.290162643 | 0.804781984 | 0.775774987 | 1.031370187 | | |
| 163 | 1.049487786 | 1.07537431 | 1.100630418 | 1.300197006 | 1.03715524 | 1.021503538 | 1.228573382 | 1.011726095 | 1.168131004 | | |
| 164 | 1 | 1 | 1 | 1 | 1 | 1 | 1 | 1 | 1 | | |
| 165 | 0.861169898 | 0.920934564 | 1.096503852 | 1.026834843 | 1.241174977 | 0.951898734 | 1.113822785 | 0.990886076 | 1.169316456 | | |
| 166 | 1 | 1 | 1 | 1 | 1 | 1.47 | 1 | 1 | 1 | | |
| 167 | 0.954891801 | 1.116854617 | 1.066900335 | 1.283907345 | 1.072691253 | 0.852879479 | 1.236045933 | 1.093012694 | 0.864060033 | | |
| 168 | 1 | 1 | 1 | 1 | 1 | 1 | 1 | 1 | 1 | | |
| 169 | 1.143297381 | 1.02312565 | 1.026832866 | 0.953669682 | 0.913226134 | 1.288242644 | 1.412503173 | 1.059860599 | 0.948222339 | | |
| 170 | 1.015361976 | 0.889056387 | 0.96317433 | 0.9285381 | 1.017838556 | 1.119188805 | 0.962760911 | 1.222189279 | 1.21163425 | | |
| 171 | 0.949585028 | 1.052400325 | 1.004621644 | 1.112839707 | 1.352318959 | 0.943605981 | 0.909129288 | 0.953421284 | 0.963447669 | | |
| 172 | 1 | 1 | 1 | 1 | 1 | 1 | 1 | 1 | 1 | | |
| 173 | 0.809244733 | 0.67137018 | 0.993643495 | 0.714543323 | 0.628497581 | 0.886214012 | 1.150535521 | 1.273705414 | 1.205143952 | | |
| 174 | 1.038813621 | 0.694870856 | 0.47736811 | 0.60890629 | 0.447567811 | 1.070269366 | 1.282583236 | 1.365651665 | 1.199933077 | | |
| 175 | 1 | 1 | 1 | 1 | 1 | 1 | 1 | 1 | 1 | | |
| 176 | 1 | 1 | 1 | 1 | 1.008723404 | 1.077168367 | 1.043128189 | 0.969945791 | 0.903300383 | | |
| 177 | 1.053421952 | 0.942392695 | 0.849043926 | 0.851982579 | 0.716610268 | 1.01657117 | 0.969544233 | 0.910875475 | 0.949707964 | | |
| 178 | 1.468330951 | 1.329005382 | 1.253899717 | 1.191656278 | 1.296804208 | 1.178741935 | 1.117870968 | 1.263193548 | 1.116225806 | | |
| 179 | 1 | 1 | 1 | 1 | 1 | 1.40664693 | 0.897717505 | 0.897717505 | 0.897717505 | | |
| 180 | 0.835792863 | 0.647303461 | 0.765830427 | 0.717131741 | 0.630533942 | 0.924014548 | 1.337658508 | 1.146662735 | 0.963629215 | | |
| 181 | 0.71089242 | 0.646826338 | 0.476049324 | 0.450794404 | 0.583313572 | 0.90112099 | 1.208581368 | 1.197448782 | 1.092307692 | | |
| 182 | 1 | 1 | 1 | 1 | 1 | 1 | 1 | 1 | 1 | | |
| 183 | 1 | 1 | 1 | 1 | 1 | 1 | 1 | 1 | 1 | | |
| 184 | 1.029001223 | 0.9223112 | 1.025052831 | 1.035285285 | 1.193471249 | 1.048929332 | 1.13165782 | 0.981222692 | 1.223413859 | | |
| 185 | 0.817570476 | 1.040832795 | 0.978803529 | 1.023294599 | 1.116365397 | 0.933552767 | 1.32592745 | 0.936844271 | 0.927244051 | | |
| 186 | 1 | 1 | 1 | 1 | 1.269042553 | 0.864769686 | 0.953162417 | 1.085998028 | 0.980067615 | | |
| 187 | 1 | 1 | 1 | 1 | 1 | 1 | 1 | 1 | 1 | | |
| 188 | 1.063953046 | 1.079342342 | 0.675696575 | 0.47336663 | 0.381360444 | 0.790765791 | 0.834488541 | 0.861307993 | 0.923968698 | | |
| 189 | 1.498051682 | 0.981469003 | 0.692048518 | 0.535875425 | 0.35295324 | 0.800642081 | 0.9049803 | 0.819508433 | 0.677750214 | | |
| 190 | 1 | 1 | 1 | 1 | 1 | 1 | 1 | 1 | 1 | | |
| 191 | 0.926143003 | 0.746786778 | 0.695827523 | 0.574912367 | 0.509159097 | 1.052888246 | 1.005588762 | 0.73724187 | 0.895906609 | | |
| 192 | 1 | 1 | 1 | 1 | 1 | 1 | 1 | 1 | 1 | | |
| 193 | 0.523450911 | 0.511573177 | 0.59374827 | 0.459604629 | 0.308267346 | 0.679455358 | 0.785334373 | 1.003900433 | 0.961421176 | | |
| 194 | 1.310638298 | 1.167446809 | 1.387765957 | 1.137340426 | 1 | 1 | 1 | 1 | 1 | | |
| 195 | 0.832679604 | 0.77595995 | 1.188996697 | 1.235858794 | 1.248916185 | 0.963345258 | 1.160694387 | 0.838484546 | 0.551287315 | | |
| 196 | 1 | 1.086914894 | 1.181489362 | 1.407978723 | 1.559787234 | 1 | 1 | 1 | 1 | | |
| 197 | 0.528817246 | 0.684321627 | 0.701081047 | 0.65460884 | 0.42560609 | 0.640370597 | 0.825328701 | 0.988350705 | 0.869882145 | | |
| 198 | 1.082234043 | 1.066808511 | 1.14393617 | 1.213404255 | 1.169574468 | 0.924014548 | 0.924014548 | 1.329008159 | 1.31858842 | | |
| 199 | 1.042959626 | 0.913580247 | 0.926537649 | 1.246774552 | 1.285035035 | 1.092721745 | 0.868602874 | 0.747171284 | 1.038147028 | | |
| 200 | 1 | 1 | 1.134468085 | 1.24212766 | 1.299148936 | 1 | 1 | 1 | 1 | | |
| 201 | 1 | 1 | 1 | 1 | 1 | 1 | 1 | 1 | 1 | | |
| 202 | 1.005078844 | 1.213863602 | 0.539412028 | 0.527931155 | 0.4428543 | 0.718916177 | 0.897902253 | 1.935078418 | 1.534295428 | | |
| 203 | 0.817386245 | 0.999720703 | 1.292330967 | 1.50315373 | 1.34556034 | 0.77223517 | 0.972264921 | 0.780763954 | 0.631063949 | | |
| 204 | 1.437339986 | 1.061905075 | 1.004070111 | 1.467537583 | 1.062627191 | 1.075659696 | 1.22457024 | 1.421876711 | 1.537994087 | | |
| 205 | 1.028540207 | 0.966445973 | 0.847399884 | 0.721604422 | 0.604229607 | 1.048020943 | 1.212753487 | 1.346461923 | 0.87520025 | | |
| 206 | 1 | 1 | 1 | 1 | 1 | 1.052234043 | 1.233404255 | 1.118191489 | 1 | | |
| 207 | 1.382095907 | 1.459964147 | 0.789102181 | 0.59784882 | 0.668098297 | 1.071212427 | 1.335955887 | 0.813395199 | 1.017281958 | | |
| 208 | 0.624969 | 0.811765289 | 0.688309112 | 0.736223402 | 0.862358018 | 1 | 1 | 1.225638298 | 1.120531915 | | |
| 209 | 1.23092352 | 1.031132816 | 0.910874684 | 0.947850353 | 1.007499782 | 0.924014548 | 1.278875455 | 1.12543006 | 0.992430945 | | |
| 210 | 1.357872713 | 0.740803815 | 0.457376411 | 0.457376411 | 0.457376411 | 1.00115556 | 1.122434381 | 1.225499367 | 1.163044076 | | |
| 211 | 1.032804745 | 1.153371263 | 0.990134366 | 1.016704999 | 1.171952548 | 1.191763611 | 0.741461145 | 0.837040484 | 0.816333178 | | |
| 212 | 1.029394351 | 0.918782433 | 1.430258071 | 1.043603942 | 1.460453453 | 1.149957647 | 1.158810831 | 1.383255458 | 1.264748477 | | |
| 213 | 1.158582709 | 1.270851304 | 1.339592595 | 1.049167275 | 1.359763785 | 0.869298648 | 0.848240889 | 0.719946138 | 0.802830621 | | |
| 214 | 0.885686893 | 0.662813251 | 0.702280458 | 0.696579313 | 0.623706038 | 0.826568059 | 1.368826512 | 1.180771493 | 1.370717427 | | |
| 215 | 1.023648373 | 0.928687923 | 1.567724048 | 1.611188127 | 1.745576123 | 1.301578444 | 1.468989158 | 1.245934311 | 1.33075574 | | |
| 216 | 1.353798614 | 1.428116612 | 1.141862788 | 0.985985392 | 0.991416443 | 1.150247058 | 0.874942693 | 0.900208853 | 0.99689267 | | |
| 217 | 1.061753594 | 0.907326182 | 1.102734471 | 1.222449009 | 1.218859021 | 0.919472477 | 0.979107986 | 0.597810069 | 0.70122554 | | |
| 218 | 0.855538585 | 0.679392549 | 0.689825126 | 0.648380781 | 0.640323373 | 0.939326565 | 1.730682104 | 1.483066253 | 1.764857231 | | |
| 219 | 1.006141557 | 0.869562678 | 0.98287822 | 1.128420088 | 1.118149716 | 1.309995132 | 1.510283141 | 0.997140191 | 1.036082265 | | |
| 220 | 1.180192117 | 1.356409407 | 1.03516617 | 1.18758971 | 1.732527327 | 1.369763165 | 0.838092917 | 0.613000421 | 0.9531205 | | |
| 221 | 1.308049259 | 2.121274317 | 1.003034089 | 0.838836338 | 0.89889345 | 1 | 1 | 1 | 1 | | |
| 222 | 0.870579067 | 0.663469608 | 0.563481597 | 0.563481597 | 0.563481597 | 1 | 1.185957447 | 1.25957447 | 1.04287234 | | |
| 223 | 0.840122544 | 1.192976784 | 0.63222797 | 0.404874069 | 0.564551389 | 0.800168934 | 0.772117009 | 0.734995999 | 0.805059127 | | |
| 224 | 1 | 1.307978723 | 1.845106383 | 1.797659574 1.309148936 | 1 | 1 | 1 | 1 | | | |
| 225 | 2.239912281 | 0.681641604 | 1.105451128 | 1.341353383 | 0.588972431 | 1 | 1 | 1.017659574 | 1.213085106 | | |
| 226 | 1.033286761 | 0.862862112 | 0.566213083 | 0.582815589 | 0.462763269 | 0.540268147 | 0.212409869 | 0.750362828 | 0.784963948 | | |
| 227 | 1.100829611 | 0.828028079 | 0.748283344 | 0.704275686 | 0.520485003 | 0.993322596 | 1.102073686 | 1.082679015 | 1.423360848 | | |
| 228 | 1.163294798 | 1.124587118 | 1.481 523534 | 1.557648637 | 1.502477291 | 0.942812803 | 1.166207565 | 0.955887488 | 1.048806984 | | |
| 229 | 1 | 1 | 1 | 1 | 1 | 1 | 1 | 1 | 1 | | |
| 230 | 1.078369646 | 1.222019717 | 1.383646757 | 1.293761909 | 1.283655041 | 0.883856068 | 0.775236534 | 0.847575058 | 0.945988229 | | |
| 231 | 0.909078328 | 0.986051527 | 1.07844935 | 1.143301965 | 1.120496586 | 0.793122044 | 1.208422909 | 1.245173844 | 0.706805081 | | |
| 232 | 0.780819127 | 0.722633521 | 0.753152664 | 0.641739682 | 0.759763635 | 0.7363115 | 0.633987323 | 0.668035014 | 0.637971627 | | |
| 233 | 1 | 1.448510638 | 1.310212766 | 1.407978723 | 1.410531915 | 0.971375426 | 1.20584892 | 1.227756536 | 1.496951535 | | |
| 234 | 1.052363039 | 1.321874865 | 1.34669434 | 1.253599689 | 1.815583517 | 1.151781583 | 1.071263304 | 0.840898574 | 1.015985865 | | |
| 235 | 1.068432876 | 1.04115998 | 0.813322689 | 0.846573715 | 0.783636262 | 1.042087502 | 0.837145956 | 0.904013499 | 1.252139327 | | |
| 236 | 1.037959585 | 0.862685939 | 0.905697446 | 0.968074656 | 0.94716531 | 0.962774869 | 0.830314136 | 0.916439791 | 0.818167539 | | |
| 237 | 0.912105954 | 1.128064799 | 1.217053415 | 1.281414186 | 1.338879159 | 1.029201937 | 1.158429749 | 0.952241896 | 0.903116105 | | |
| 238 | 0.897803152 | 0.860543536 | 0.491553152 | 0.426382212 | 0.402978098 | 1.152399086 | 0.996572734 | 0.964737243 | 1.114166032 | | |
| 239 | 0.984156363 | 1.410824017 | 1.681868661 | 2.018731028 | 1.678759162 | 1.005047374 | 1.184229169 | 1.115691136 | 1.153280793 | | |
| 240 | 1.09787234 | 1.076914894 | 1.632234043 | 1.568829787 | 1.329361702 | 1 | 1 | 1 | 1 | | |
| 241 | 1 | 1 | 1 | 1 | 1 | 1 | 1 | 1 | 1 | | |
| 242 | 1.085456528 | 0.808631572 | 0.443243779 | 0.385843164 | 0.335910908 | 1 | 1 | 1 | 1 | | |
| 243 | 0.474956212 | 0.244652986 | 0.15105012 | 0.15105012 | 0.15105012 | 0.985961885 | 0.752293938 | 0.859736491 | 0.790212532 | | |
| 244 | 0.749535508 | 0.766205615 | 0.811725846 | 0.816061107 | 0.557855078 | 0.833762193 | 0.701773283 | 0.91372948 | 0.865657351 | | |
| 245 | 0.931560462 | 1.593677873 | 0.875388861 | 0.751756147 | 0.822779729 | 1.170018282 | 1.144777968 | 1.594090936 | 1.870672878 | | |
| 246 | 1.00393617 | 1 | 1 | 1 | 1.219148936 | 1 | 1 | 1.009893617 | 1 | | |
| 247 | 1.09375593 | 1.13125 | 1.04147889 | 1.074881404 | 1.348031309 | 1.08893192 | 0.944019605 | 1.237459932 | 1.159473563 | | |
| 248 | 1 | 1 | 1 | 1 | 1 | 1 | 1 | 1 | 1 | | |
| 249 | 1.154396891 | 0.877249015 | 1.097546045 | 0.910039391 | 1.034334079 | 1.022269591 | 1.018539294 | 1.081365345 | 0.994951478 | | |
| 250 | 1.327738319 | 1.323613721 | 0.842500876 | 0.76758926 | 0.848186132 | 1.410320822 | 1.130753128 | 1.118124726 | 1.481933694 | | |
| 251 | 0.983422447 | 0.677935987 | 0.658416455 | 0.705227797 | 0.650053303 | 0.962922931 | 1.671628836 | 1.18173332 | 1.488732763 | | |
| 252 | 0.93067653 | 0.976375134 | 0.973034244 | 0.931690729 | 1.100465338 | 1.175323686 | 1.116222391 | 0.866488957 | 1.211805027 | | |
| 253 | 0.893510825 | 0.90358757 | 0.990079795 | 0.680674967 | 0.77977667 | 0.98782554 | 0.928453796 | 0.892709361 | 0.943366422 | | |
| 254 | 1.007696887 | 1.260588535 | 1.106225549 | 0.97763225 | 1.585005953 | 1.047746076 | 0.967022346 | 0.847637826 | 1.028802289 | | |
| 255 | 0.95629276 | 0.952256731 | 1.240192942 | 1.432298076 | 1.103214057 | 1.010490694 | 0.845211506 | 0.78463621 | 0.73258832 | | |
| 256 | 0.940914436 | 0.772238056 | 0.836027345 | 0.884220597 | 0.773962091 | 0.917123921 | 0.680502466 | 0.643071825 | 0.688840937 | | |
| 257 | 0.717037778 | 0.718330414 | 0.820323576 | 0.780335251 | 1.05545826 | 0.915473068 | 0.889919978 | 1.158429157 | 1.473135633 | | |
| 258 | 0.827142324 | 1.223205852 | 0.482600004 | 0.365173648 | 0.277121455 | 0.626066413 | 0.631771449 | 1.463429146 | 1.363302271 | | |
| 259 | 1.450548454 | 1.037621249 | 1.270691687 | 1.310851237 | 1.094189103 | 0.995043325 | 1.056689675 | 1.305845205 | 1.172602438 | | |
| 260 | 0.68085961 | 0.822454308 | 0.430541608 | 0.381803575 | 0.566847426 | 0.727926837 | 1.346109878 | 1.41846547 | 1.374016542 | | |
| 261 | 1.082234043 | 1.318085106 | 1.14393617 | 1.339042553 | 1.264042553 | 1.254870024 | 1.299302886 | 0.89009056 | 0.980975959 | | |
| 262 | 1.274563782 | 1.018547098 | 1.261133125 | 1.134223149 | 1.103524505 | 1.157578383 | 0.926551828 | 1.112371306 | 1.081811215 | | |
| 263 | 0.909432403 | 0.848164983 | 0.936290898 | 0.979827955 | 0.952819203 | 0.943419724 | 0.872366207 | 0.719320094 | 0.749175362 | | |
| 264 | 1.037586117 | 0.923955699 | 0.880265109 | 0.863172582 | 0.987180605 | 1 | 1 | 1 | 1 | | |
| 265 | 1 | 1 | 1 | 1 | 1 | 0.991665788 | 0.991665788 | 0.991665788 | 0.991665788 | 0.991665788 | 0.991665788 |
| 266 | 1 | 1 | 1 | 1 | 1 | 1.052234043 | 1 | 1 | 1 | | |
| 267 | 1.097622028 | 0.784313725 | 0.784313725 | 0.784313725 | 0.784313725 | 1.647169473 | 1.847456109 | 0.841992118 | 0.871551415 | | |
| 268 | 0.772090308 | 0.59366485 | 0.603688206 | 0.712874659 | 0.53946088 | 1.132725528 | 0.733829175 | 0.883661228 | 0.807653551 | | |
| 269 | 1.056918029 | 0.864447781 | 0.522800578 | 0.383633634 | 0.348793238 | 1 | 1 | 1 | 1 | | |
| 270 | 1 | 1 | 1 | 1 | 1 | 1 | 1 | 1 | 1 | | |
| 271 | 0.927421184 | 1.250829914 | 1.284830615 | 1.318522031 | 1.037526547 | 0.904151499 | 1.076046265 | 1.148994398 | 0.779787262 | | |
| 272 | 1.450062628 | 1.186927286 | 0.796591733 | 0.501977718 | 0.382358758 | 1 | 1 | 1 | 1 | | |
| 273 | 0.887386355 | 1.184964474 | 1.164871266 | 0.940637176 | 1.565665826 | 1 | 1.027978723 | 1 | 1.159148936 | | |
| 274 | 1.066702128 | 1 | 1 | 1 | 1 | 1 | 1 | 1.009893617 | 1 | | |
| 275 | 0.736158355 | 1.102046521 | 0.518562318 | 0.511823721 | 0.350187709 | 0.539720226 | 0.8203125 | 1.899023036 | 1.805553773 | | |
| 276 | 0.893111978 | 1.055275426 | 1.183093902 | 1.038340786 | 1.282656265 | 0.791386594 | 0.914662983 | 0.822577646 | 0.869497064 | | |
| 277 | 1.195298557 | 1.136250064 | 1.120442609 | 1.199326908 | 1.394013564 | 1.00199636 | 1.001878927 | 0.900504962 | 0.849715225 | | |
| 278 | 0.744456918 | 0.796449621 | 0.934675835 | 0.928641594 | 0.87349144 | 0.977403055 | 0.806563334 | 0.88735593 | 0.733151649 | | |
| 279 | 0.951944381 | 0.780579112 | 1.546413019 | 1.886469555 | 2.055924822 | 0.951061399 | 0.778633864 | 0.737001514 | 0.710752145 | | |
| 280 | 0.891596639 | 1.191848739 | 1.309495798 | 1.495798319 | 1.92302521 | 1.023048011 | 0.912062974 | 0.93872775 | 1.113254379 | | |
| 281 | 1.020361132 | 1.788011062 | 1.234247912 | 1.277437371 | 1.208355927 | 1.241904353 | 1.069568947 | 2.032897276 | 2.051168775 | | |
| 282 | 1.096576873 | 0.878720151 | 0.905246442 | 0.856134572 | 0.99011881 | 1.003133789 | 1.371313781 | 1.545198875 | 1.540136601 | | |
| 283 | 0.91711175 | 0.936010682 | 0.905901514 | 0.777181888 | 1.111940956 | 0.848944297 | 1.274967896 | 0.838378995 | 0.829428526 | | |
| 284 | 1.27883885 | 0.939880629 | 1.500759831 | 2.126424308 | 2.540314704 | 1.060498021 | 0.769522272 | 0.597972935 | 0.630318523 | | |
| 285 | 0.752120339 | 0.752120339 | 0.752120339 | 0.752120339 | 0.752120339 | 1 | 1 | 1 | 1 | | |
| 286 | 1.064377811 | 0.904317841 | 0.694002999 | 0.648215892 | 0.716131934 | 0.898512686 | 0.858899582 | 1.092592593 | 1.098911247 | | |
| 287 | 1.098424169 | 1.135208079 | 0.672079053 | 0.512188752 | 0.614966046 | 0.974699685 | 0.788604792 | 1.148882625 | 1.112609892 | | |
| 288 | 0.916783165 | 0.69163417 | 0.89163417 | 0.69163417 | 0.69163417 | 1 | 1 | 1 | 1 | | |
| 289 | 1 | 1 | 1 | 1 | 1 | 1 | 1 | 1 | 1 | | |
| 290 | 1 | 1.066808511 | 1 | 1 | 1.066808511 | 1.065302247 | 1.482962338 | 1.780778653 | 1.437809896 | | |
| 291 | 1.074103272 | 0.926290895 | 0.926290895 | 0.926290895 | 0.926290895 | 1.070428475 | 0.792847503 | 0.792847503 | 0.802041161 | | |
| 292 | 1.195830005 | 1.091287568 | 1.756911548 | 1.484702891 | 1.715976731 | 1.019646441 | 0.853040293 | 1.058034719 | 0.982876254 | | |
| 293 | 0.822771403 | 0.722241836 | 1.126345984 | 1.85423654 | 2.012842012 | 0.991082134 | 1.120690947 | 1.039418465 | 0.883917866 | | |
| 294 | 0.85695247 | 1.22848385 | 1.174432884 | 1.355557883 | 1.445763406 | 0.870942884 | 1.052566039 | 1.003735783 | 0.933594304 | | |
| 295 | 1 | 1 | 1 | 1 | 1 | 1 | 1 | 1 | 1 | | |
| 296 | 1 | 1 | 1 | 1 | 1 | 1.36455676 | 0.749362245 | 0.749362245 | 0.749362245 | | |
| 297 | 1 | 1 | 1 | 1 | 1 | 1 | 1 | 1 | 1 | | |
| 298 | 1.211243612 | 0.8459682 | 0.929230551 | 0.899985804 | 0.82673197 | 1.130572065 | 1.00083581 | 0.955980684 | 0.944372214 | | |
| 299 | 0.897078384 | 0.633169747 | 0.64289318 | 0.779617484 | 0.925744164 | 1 | 1 | 1 | 1 | | |
| 300 | 1.164877471 | 0.695935441 | 0.695935441 | 0.695935441 | 0.695935441 | 1 | 1 | 1 | 1 | | |
| 301 | 0.751753106 | 1.025949525 | 0.479122913 | 1.514968142 | 0.658420247 | 1.413864344 | 1.681170936 | 1.516655012 | 1.229928167 | | |
| 302 | 1 | 1 | 1 | 1 | 1 | 1 | 1 | 1 | 1 | | |
| 303 | 0.922592958 | 0.752951786 | 0.941509553 | 0.931275297 | 0.872512216 | 1.005249908 | 1.024560973 | 1.220864546 | 1.240359818 | | |
| 304 | 1 | 1 | 1 | 1 | 1 | 1 | 1 | 1 | 1 | | |
| 305 | 0.834813642 | 0.620654811 | 0.670333019 | 0.757182669 | 0.68153658 | 1.074743563 | 1.8983562 | 1.406631048 | 1.683992032 | | |
| 306 | 1.062001532 | 1.156851259 | 0.752579642 | 0.664488803 | 0.529040689 | 0.958388713 | 1.315100705 | 1.008553999 | 0.756159902 | | |
| 307 | 0.804019765 | 0.743956998 | 0.910590278 | 0.718215812 | 0.744825053 | 0.825699074 | 0.778742386 | 0.912417862 | 0.814283659 | | |
| 308 | 0.889202541 | 0.794424841 | 0.663373324 | 0.663373324 | 0.805434016 | 1.285494803 | 1.000813376 | 0.849525531 | 0.849525531 | | |
| 309 | 0.916009251 | 1.140251151 | 0.78580846 | 0.678085893 | 0.618734735 | 0.894159197 | 1.13729128 | 1.160851197 | 0.951561347 | | |
| 310 | 0.945361505 | 1.110817652 | 1.025118301 | 1.233602949 | 1.674947728 | 1.593465288 | 0.937869895 | 0.590813094 | 0.984767605 | | |
| 311 | 0.846784554 | 0.92080405 | 0.947933197 | 0.908788634 | 1.189904028 | 1 | 1 | 1 | 1 | | |
| 312 | 1 | 1.157340426 | 1.167446809 | 1 | 1.269042553 | 1 | 1 | 1 | 1 | | |
| 313 | 0.893744644 | 0.732258316 | 0.732258316 | 0.732258316 | 0.732258316 | 1 | 1 | 1 | 1 | | |
| 314 | 1 | 1 | 1 | 1 | 1 | 1 | 1 | 1 | 1 | | |
| 315 | 0.725557726 | 0.573815154 | 0.828364347 | 0.843117097 | 0.901408451 | 0.938459105 | 0.752649897 | 1.602990033 | 1.599588673 | | |
| 316 | 0.704719559 | 0.608078427 | 0.611614078 | 0.659559651 | 0.586489527 | 0.871253189 | 0.811782526 | 1.947464923 | 1.662547832 | | |
| 317 | 0.547753627 | 0.547753627 | 0.547753627 | 0.547753627 | 0.547753627 | 0.821625372 | 0.464141394 | 0.514143434 | 0.632066615 | | |
| 318 | 0.98698026 | 0.98698026 | 0.98698026 | 0.98698026 | 0.98698026 | 1 | 1 | 1 | 1 | | |
| 319 | 0.842217184 | 0.72088364 | 0.918718476 | 1.052898895 | 1.386724311 | 0.801346986 | 2.147474858 | 1.213437004 | 0.811440253 | | |
| 320 | 0.795805269 | 0.725196806 | 0.58112371 | 0.442720323 | 0.489243186 | 0.931445375 | 1.010020487 | 0.704388667 | 0.54754505 | | |
| 321 | 1.260029718 | 1.185642645 | 0.872956909 | 0.872956909 | 0.872956909 | 1 | 1 | 1 | 1 | | |
| 322 | 1 | 1 | 1 | 1 | 1 | 1.259777645 | 0.933095096 | 0.933095096 | 0.933095096 | | |
| 323 | 1 | 1 | 1 | 1 | 1 | 0.990589198 | 0.961538462 | 0.961538462 | 0.961538462 | | |
| 324 | 0.860478947 | 0.74707163 | 0.987596241 | 0.849944247 | 1.196378697 | 0.79318267 | 0.884489897 | 0.895059709 | 0.682514738 | | |
| 325 | 0.695810831 | 0.809243176 | 0.423277624 | 0.350788206 | 0.347704715 | 0.753281088 | 0.57437919 | 0.651449344 | 0.611533377 | | |
| 326 | 1.111037863 | 0.843761644 | 1.208117172 | 0.803147124 | 0.745306544 | 0.700875458 | 0.72222288 | 0.94451684 | 0.705963536 | | |
| 327 | 1 | 1.237765957 | 1.267021277 | 1.251702128 | 1.528617021 | 0.924825477 | 0.561818497 | 0.542318122 | 0.542318122 | | |
| 328 | 0.753382865 | 0.88546714 | 0.777287247 | 0.734611105 | 1.259323068 | 1.011495164 | 1.233272554 | 0.86629935 | 0.846123355 | | |
| 329 | 0.845399766 | 0.850795935 | 0.845399766 | 0.845399766 | 0.845399766 | 1 | 1 | 1 | 1 | | |
| 330 | 0.807253202 | 0.875614715 | 1.101360627 | 0.987245208 | 1.239821363 | 0.98258426 | 0.839732231 | 0.991945444 | 0.931178292 | | |
| 331 | 1 | 1 | 1 | 1 | 1 | 1 | 1 | 1 | 1 | | |
| 332 | 0.95894405 | 1.071355398 | 0.633018125 | 0.596217494 | 0.51678487 | 0.92859352 | 1.474251914 | 1.525632104 | 1.015193691 | | |
| 333 | 1 | 1 | 1 | 1 | 1 | 1.151595745 | 1 | 1 | 1 | | |
| 334 | 1 | 1.147340426 | 1.200425532 | 1.915106383 | 1.349574468 | 1 | 1 | 1.195 | 1.066276596 | | |
| 335 | 1.144148936 | 1.016489362 | 1 | 1 | 11.053385883 | 0.841992118 | 0.841992118 | 0.841992118 | | | |
| 336 | 0.738508576 | 0.725190444 | 0.577861844 | 0.657045137 | 0.79022646 | 1.153766545 | 0.933656517 | 0.942099243 | 1.453292663 | | |
| 337 | 0.994606244 | 1.223287512 | 1.130370457 | 1.100675676 | 1.101432898 | 0.906604403 | 1.021347565 | 1.093087699 | 1.201390671 | | |
| 338 | 1.407893579 | 1.511469303 | 1.246296677 | 1.131332316 | 1.510948637 | 1.344494932 | 1.005867873 | 1.0126998 | 1.154036493 | | |
| 339 | 0.881003174 | 1.216140278 | 1.126801603 | 1.604193767 | 0.972995473 | 0.777144325 | 1.039856189 | 1.49424756 | 1.006111967 | | |
| 340 | 0.796981774 | 0.955322072 | 0.803578539 | 0.739228691 | 0.611884939 | 0.840370584 | 1.049907548 | 0.982337212 | 0.754849585 | | |
| 341 | 1.105339757 | 0.98067851 | 1.196627522 | 1.25529459 | 1.161648098 | 0.969927956 | 1.024861701 | 0.830792487 | 0.908079249 | | |
| 342 | 1.028626129 | 0.864802843 | 0.739283232 | 0.687276256 | 1.202402339 | 1.212553588 | 0.934395766 | 0.704579908 | 0.836261091 | | |
| 343 | 1 | 1 | 1.139255319 | 1.378404255 | 1.264042553 | 1.104368634 | 0.576757884 | 0.636028961 | 0.673027365 | | |
| 344 | 0.844010727 | 0.922973778 | 1.047675805 | 1.266239571 | 1.276445173 | 0.857194966 | 1.145540762 | 1.096480029 | 0.933977749 | | |
| 345 | 1.210310049 | 0.749700175 | 0.931069736 | 0.789729273 | 0.650138608 | 1.307980883 | 1.396076366 | 1.347191531 | 1.169073436 | | |
| 346 | 0.996290408 | 0.996290408 | 0.996290408 | 0.996290408 | 0.996290408 | 1 | 1 | 1 | 1 | | |
| 347 | 0.902544407 | 0.902544407 | 0.902544407 | 0.902544407 | 0.902544407 | 0.855700245 | 0.717733496 | 0.922914262 | 0.892630193 | | |
| 348 | 1.007508005 | 1.284807331 | 1.227669206 | 1.251628575 | 1.476758308 | 1.050151238 | 1.046603682 | 1.041188991 | 1.062287613 | | |
| 349 | 0.933836019 | 1.005468782 | 1.093632183 | 0.974437585 | 1.194928947 | 0.99657444 | 0.853637828 | 0.877001903 | 0.962377397 | | |
| 350 | 0.725987211 | 0.950029666 | 0.880908432 | 0.863306744 | 1.174500626 | 1.046633711 | 1.390699506 | 1.022879391 | 1.07187541 | | |
| 351 | 0.823974788 | 0.784138514 | 0.573965179 | 0.37737807 | 0.358372728 | 1.013015991 | 1.444987515 | 1.391010997 | 1.1788397174 | | |
| 352 | 1 | 1 | 1 | 1 | 1 | 1 | 1 | 1 | 1 | | |
| 353 | 1 | 1 | 1 | 1 | 1 | 1 | 1 | 1 | 1 | | |
| 354 | 0.837285432 | 0.856885046 | 0.57266966 | 0.441264112 | 0.57061317 | 1.044607654 | 1.063548512 | 0.984847313 | 0.864785466 | | |
| 355 | 0.858909796 | 1.107081902 | 0.782738785 | 0.869276639 | 0.661265513 | 0.924901634 | 1.173463983 | 1.072204146 | 0.884269068 | | |
| 356 | 1 | 1 | 1 | 1 | 1 | 1 | 1 | 1 | 1 | | |
| 357 | 1.209906618 | 1.425091352 | 1.420567252 | 1.618989618 | 1.321269068 | 0.819803643 | 0.800776159 | 0.723681543 | 0.679139274 | | |
| 358 | 1.372111276 | 1.527339322 | 2.244789396 | 2.209296241 | 2.656492544 | 1.486865851 | 1.680770002 | 1.341387608 | 1.04882695 | | |
| 359 | 1.1441 48936 | 1.227659574 | 1.219361702 | 1.146808511 | 1.43606383 | 1.459336571 | 1.460369949 | 1.078743412 | 1.058385863 | | |
| 360 | 1.260572544 | 1.079279728 | 1.208523097 | 1.130123617 | 1.139289678 | 0.976833525 | 0.944053353 | 0.856516059 | 1.056005148 | | |
| 361 | 0.914475635 | 0.960734407 | 1.959251754 | 2.446130276 | 3.190718592 | 0.955225873 | 0.888655823 | 0.876527908 | 0.944375584 | | |
| 362 | 1 | 1 | 1 | 1 | 1 | 1.115842764 | 1.04030176 | 1.214909668 | 1.060055589 | | |
| 363 | 1 | 1 | 1 | 1 | 1 | 1 | 1 | 1 | 1 | | |
| 364 | 1.29470332 | 1.043779055 | 1.052783777 | 1.013836524 | 1.067608624 | 1.092970791 | 1.215625742 | 1.018522916 | 1.133341249 | | |
| 365 | 1 | 1 | 1 | 1 | 1 | 1 | 1 | 1 | 1 | | |
| 366 | 1.455028644 | 1.493984723 | 1.907893062 | 2.259707193 | 2.132972629 | 1.227277286 | 1.252197894 | 1.144041451 | 1.218017717 | | |
| 367 | 1.123284092 | 1.093721692 | 2.001515574 | 1.774719532 | 1.847083827 | 1.072928038 | 1.078022461 | 1.104736809 | 1.287508627 | | |
| 368 | 1 | 1 | 1 | 1 | 1 | 1 | 1 | 1 | 1 | | |
| 369 | 0.946656217 | 0.855067921 | 0.992998955 | 0.977742947 | 1.006321839 | 1.08752992 | 1.046228939 | 1.192518891 | 1.09729197 | | |
| 370 | 0.918603277 | 1.081508961 | 0.869176876 | 0.786752283 | 0.844342576 | 0.835062886 | 0.986209094 | 0.910495653 | 0.860968246 | | |
| 371 | 1 | 1 | 1 | 1 | 1 | 1 | 1.122765957 | 1 | 1.035106383 | | |
| 372 | 0.604395604 | 0.832444255 | 0.943828017 | 0.594100075 | 0.730395818 | 0.971375426 | 1.052288933 | 1.123592022 | 0.971375426 | | |
| 373 | 1 | 1.458617021 | 1.729893617 | 2.166276596 | 1.622234043 | 0.868360277 | 0.88594204 | 0.922446547 | 0.79557476 | | |
| 374 | 1.198828842 | 1.633765645 | 1.134291429 | 1.040640013 | 0.929153178 | 1.227704371 | 1.080964135 | 1.000726006 | 1.366429505 | | |
| 375 | 0.80751082 | 1.649109961 | 0.706287221 | 0.98174363 | 0.75018712 | 0.950673593 | 0.877401917 | 1.168503737 | 1.324549179 | | |
| 376 | 1 | 1.026595745 | 1.101808511 | 1.071595745 | 1.299148936 | 1 | 1 | 1 | 1 | | |
| 377 | 0.987031033 | 1.059918312 | 0.774601036 | 0.787991073 | 0.625584235 | 1.081811942 | 1.0061312 | 0.848318463 | 0.776801647 | | |
| 378 | 1 | 1 | 1 | 1 | 1 | 1 | 1 | 1 | 1 | | |
| 379 | 1.757057732 | 1.524372375 | 1.607599883 | 2.194099834 | 1.796913158 | 1.044602359 | 1.181811309 | 1.022074388 | 1.1005443 | | |
| 380 | 1.085181027 | 1.141664328 | 1.530592198 | 1.747544204 | 1.749929834 | 0.876528202 | 0.724228952 | 0.951132259 | 0.875382051 | | |
| 381 | 1.077035455 | 2.262628533 | 2.349159971 | 1.423498342 | 3.175591392 | 1.138477157 | 1.093739425 | 0.696717428 | 1.340169205 | | |
| 382 | 0.696719182 | 0.592457328 | 0.864962332 | 0.733225348 | 0.445683898 | 0.692066806 | 0.578249439 | 0.879803603 | 0.822546973 | | |
| 383 | 1 | 1 | 1 | 1 | 1 | 1 | 1 | 1 | 1 | | |
| 384 | 1 | 1 | 1 | 1 | 1 | 1 | 1 | 1 | 1 | | |
| 385 | 1.329587063 | 1.207282343 | 1.167743084 | 1.277817331 | 1.179287022 | 0.947535936 | 0.971063635 | 1.130265649 | 0.998044559 | | |
| 386 | 0.713741987 | 1.383847489 | 1.496961806 | 1.580328526 | 1.124031784 | 0.970026047 | 0.996273292 | 1.206050892 | 0.82163895 | | |
| 387 | 1.0696199 | 0.940630567 | 0.646307385 | 0.609649518 | 0.50391 6897 | 0.964072134 | 0.957726463 | 1.017279482 | 0.880093503 | | |
| 388 | 1.057379261 | 1.258566418 | 1.13625326 | 1.107293821 | 0.922475043 | 1.109041969 | 0.759448473 | 0.876235992 | 1.105581191 | | |
| 389 | 0.9527833 | 1.466302187 | 1.472962227 | 1.699502982 | 2.160636183 | 0.684930614 | 0.867092319 | 1.131409212 | 0.878624015 | | |
| 390 | 1.050503077 | 1.107648725 | 1.964051968 | 1.951304093 | 1.820504054 | 1.043136815 | 1.088685701 | 1.066584394 | 1.410865541 | | |
| 391 | 1 | 1 | 1 | 1 | 1 | 1 | 1 | 1 | 1 | | |
| 392 | 0.972771971 | 1.009784009 | 0.89233875 | 0.772218369 | 0.689871693 | 1.107698795 | 1.115169403 | 0.833816162 | 0.883223374 | | |
| 393 | 0.945964957 | 0.828494692 | 0.999552372 | 0.888284947 | 1.202327663 | 0.788267288 | 1.310929811 | 1.066045066 | 03873219373 | | |
| 394 | 0.912976399 | 0.646735051 | 0.607997665 | 0.563547661 | 0.646109582 | 1.044526627 | 1.178550296 | 0.884023669 | 0.827366864 | | |
| 395 | 1 | 1.197553191 | 1.069148936 | 1.203829787 | 1.219148936 | 1 | 1 | 1 | 1 | | |
| 396 | 2.3688923 | 1.674597084 | 2.028651829 | 1.500724823 | 1.34228703 | 1 | | | | | |
| 397 | 0.779081559 | 0.771886619 | 0.636583128 | 0.516248974 | 0.594234391 | 0.869260978 | 0.806640486 | 0.858550518 | 1.064976794 | | |
| 398 | 1.233835317 | 0.965735459 | 0.76583451 | 0.714877928 | 0.549846837 | 1.068628613 | 1.055159003 | 1.114515402 | 0.989921506 | | |
| 399 | 0.749524107 | 1.038099838 | 1.050629315 | 1.128903032 | 0.976674148 | 0.740349876 | 1.031343094 | 1.245832541 | 0.988478798 | | |
| 400 | 1.00393617 | 1 | 1 | 1 | 1 | 0.95106214 | 0.933095096 | 0.942326782 | 0.933095096 | | |
| 401 | 1 | 1 | 1 | 1 | 1 | 0.915109034 | 1.099785826 | 1.135611371 | 1.088979751 | | |
| 402 | 1 | 1 | 1 | 1 | 1 | 1 | 1 | 1 | 1 | | |
| 403 | 1.069885467 | 1.267197037 | 1.32096564 | 1.346821206 | 2.635141623 | 1 | 1 | 1 | 1 | | |
| 404 | 1.265744681 | 1.318085106 | 1.276595745 | 1.137340426 | 1.400319149 | 0.91260732 | 1.162313601 | 1.164121103 | 1.199367375 | | |
| 405 | 0.93105365 | 0.89535554 | 0.667579363 | 0.797854819 | 0.87573599 | 0.80516226 | 1.367121915 | 1.319978699 | 1.481142714 | | |
| 406 | 1.059814742 | 1.444225463 | 1.63618447 | 1.684765471 | 1.663874655 | 0.698933441 | 1.009990549 | 0.939966698 | 0.716034382 | | |
| 407 | 1 | 1 | 1 | 1 | 1 | 1.134733158 | 1.102686789 | 1.202158064 | 0.940507437 | | |
| 408 | 1 | 1.65 | 1.422021277 | 1.630425532 | 1.590957447 | 1 | 1 | 1 | 1 | | |
| 409 | 1.064658712 | 0.940849521 | 0.601602803 | 0.609297357 | 0.493831628 | 1.044193225 | 0.886810354 | 1.012792994 | 0.936267378 | | |
| 410 | 1 | 1 | 1 | 1 | 1 | 1 | 1 | 1 | 1 | | |
| 411 | 1 | 1.700531915 | 1.62712766 | 1.203829787 | 1.244042553 | 0.75011262 | 0.731771671 | 0.856425767 | 0.913894073 | | |
| 412 | 1 | 1 | 1 | 1 | 1 | 0.915473068 | 0.884876605 | 1.069665792 | 1.07477641 | | |
| 413 | 0.908472699 | 1.063661535 | 1.630302359 | 1.807043969 | 1.816303024 | 0.925202382 | 1.018097277 | 0.8358868 | 0.864253696 | | |
| 414 | 1.034842773 | 1.156067448 | 1.021419398 | 0.754236235 | 0.77263123 | 0.894804872 | 0.90857569 | 0.873626647 | 0.783942332 | | |
| 415 | 0.988227503 | 1.029651976 | 1.014200574 | 0.987565301 | 1.025163711 | 0.946519482 | 1.192247593 | 1.241897273 | 1.097232723 | | |
| 416 | 1.412992323 | 1.151487096 | 1.450487984 | 1.26459129 | 1.143849092 | 1.063879148 | 0.86366334 | 0.987510116 | 0.96870785 | | |
| 417 | 1.369501466 | 1.479301644 | 1.4231057761 | 1.502966651 | 1.729864284 | 1.012023414 | 1.040895428 | 1.058297738 | 1.162790698 | | |
| 418 | 0.924456655 | 0.913846635 | 0.973419107 | 1.008867743 | 1.115437021 | 1.084594445 | 0.802897481 | 0.99531697 | 1.081941497 | | |
| 419 | 1.395025189 | 1.39697733 | 1.161649874 | 1.127267003 | 1.570528967 | 1.252402789 | 1.20993781 | 1.042967523 | 1.106476537 | | |
| | L | M | N | O | P | Q | R | S | T | | |
| 1 | CA3.JRFnCMS | DG.CMS | DG.IMP | DG.IMPnCMS | DG.JRF | DG.JRFnCMS | DR.CMS | DR.IMP | DR.IMPnCMS | | |
| 2 | 1 | 1 | 1 | 1 | 1 | 1 | 1.103871183 | 0.617720674 | 1.7585750042 | | |
| 3 | 1.101702128 | 1 | 1 | 1 | 1 | 1 | 1.038510638 | 1 | 1.33893617 | | |
| 4 | 1.257255809 | 0.740795287 | 0.490586315 | 1.034191776 | 0.374159137 | 0.422282371 | 1 | 1.751382979 | 1.157340426 | | |
| 5 | 1.018895145 | 1.124839329 | 1.056286399 | 0.595109256 | 0.434105083 | 0.722662139 | 1 | 1 | 1.275319149 | | |
| 6 | 1.076125307 | 0.947703704 | 0.913407407 | 0.791679012 | 0.499975309 | 0.848493827 | 1.030540614 | 0.979168566 | 1.287218525 | | |
| 7 | 1 | 1 | 1 | 1 | 1 | 1 | 1.795 | 1 | 1.747234043 | | |
| 8 | 0.780506873 | 1.005395324 | 1.02511157 | 1.157130487 | 2.296542996 | 1.414707254 | 0.850643131 | 0.643130816 | 1.264709907 | | |
| 9 | 1.028533628 | 1.245547349 | 1.435157589 | 0.750773905 | 1.005658503 | 1.609455315 | 1.310610406 | 0.983449406 | 1.728029496 | | |
| 10 | 1.296049205 | 0.909671324 | 1.098173385 | 1.005611544 | 0.395613834 | 0.859883188 | 0.93899243 | 1.356720584 | 1.052475042 | | |
| 11 | 1.209910227 | 1.102421181 | 1.073419265 | 0.868663996 | 1.356906462 | 1.175492075 | 1 | 1 | 1.444042553 | | |
| 12 | 1.033041788 | 0.977387705 | 1.114211252 | 1.104783075 | 1.908860954 | 1.592595432 | 0.952665424 | 0.940070774 | 1.245576652 | | |
| 13 | 0.762561346 | 0.930115128 | 0.904009291 | 1.099222379 | 2.517521713 | 1.876792567 | 0.855400855 | 0.855400855 | 1.154336154 | | |
| 14 | 1.734574468 | 1 | 1.118617021 | 1 | 1.101382979 | 1.290638298 | 1 | 1 | 1 | | |
| 15 | 1 | 1 | 1 | 1 | 1 | 1 | 0.908032299 | 1.063790905 | 1.335826604 | | |
| 16 | 1 | 1.117156016 | 1.224620994 | 1.366628286 | 1.864901171 | 1.888025331 | 1 | 1 | 1 | | |
| 17 | 0.848712525 | 1.313788228 | 1.65324801 | 1.495051715 | 0.699456805 | 1.055435672 | 1 | 1 | 1 | | |
| 18 | 0.79125751 | 1.102656637 | 0.99721727 | 1.191921388 | 1.466324623 | 1.098612983 | 1.492798403 | 1.44241099 | 1.883681133 | | |
| 19 | 0.9074754 | 0.994816529 | 0.892904208 | 1.484229947 | 2.387178962 | 1.270682637 | 1.120951499 | 1.007371898 | 1.140327908 | | |
| 20 | 1 | 1 | 1 | 1 | 1 | 1 | 1 | 1 | 1 | | |
| 21 | 1.009797653 | 0.900424854 | 0.836453355 | 0.65582404 | 0.687975748 | 0.960125686 | 1.037447823 | 1.260405486 | 1.661359571 | | |
| 22 | 0.782862085 | 1.014555168 | 0.898386393 | 0.59043829 | 0.664467946 | 0.867913214 | 1 | 1 | 1 | | |
| 23 | 0.648857596 | 0.953076976 | 0.71825695 | 0.498019261 | 0.394132914 | 0.859640735 | 1.326096913 | 1.654850234 | 1.37849226 | | |
| 24 | 0.962024365 | 1.059982914 | 1.002867952 | 1.034720527 | 1.669270198 | 1.217415182 | 0.914840368 | 0.679586039 | 1.028239989 | | |
| 25 | 1.064983792 | 1.03853491 | 1.10754483 | 0.700925219 | 0.448302175 | 0.908098054 | 1.023243802 | 1.596177686 | 2.061363636 | | |
| 26 | 0.839760069 | 1 | 1.064468085 | 1.343191489 | 1.043723404 | 1.101808511 | 1.017888428 | 0.907791609 | 0.866758875 | | |
| 27 | 0.974536237 | 0.981601926 | 0.984871896 | 1.182687125 | 0.736030759 | 0.962485177 | 0.809792361 | 1.265316585 | 1.168332906 | | |
| 28 | 1 | 0.92055089 | 0.932014489 | 0.551247861 | 1.781395534 | 1.306452255 | 1 | 1 | 1 | | |
| 29 | 0.796315594 | 0.983527178 | 1.00517446 | 1.193543793 | 2.059340138 | 1.289627344 | 1.125736016 | 0.947767419 | 0.916192345 | | |
| 30 | 1 | 1 | 1.041914894 | 1 | 1.262234043 | 1.164468085 | 1 | 1 | 1 | | |
| 31 | 0.993247607 | 0.979115939 | 0.991477805 | 0.943669226 | 0.877692452 | 0.895813823 | 0.934879271 | 0.989362301 | 0.509070749 | | |
| 32 | 0.834916304 | 1.101290442 | 0.799147938 | 0.52713633 | 0.650469252 | 0.750864411 | 1 | 1 | 1.036382979 | | |
| 33 | 1.314308322 | 0.978795776 | 0.634663741 | 0.346984867 | 0.267160426 | 0.228436886 | 0.924531516 | 1.539449988 | 0.599075201 | | |
| 34 | 0.997838398 | 1.0127772 | 0.841037547 | 2.02838844 | 2.520246578 | 1.609735009 | 1.045008578 | 0.669884546 | 1.187601428 | | |
| 35 | 0.750170364 | 1.029442818 | 0.910687719 | 0.320223641 | 0.580097903 | 0.617651293 | 1.180385257 | 1.293498796 | 1.52604186 | | |
| 36 | 1.797649333 | 1.005809468 | 1.443017292 | 1.327327191 | 1.616121273 | 1.97816911 | 1.115050505 | 1.098080808 | 1.15 | | |
| 37 | 1.248002333 | 0.865833034 | 0.676574591 | 0.555065854 | 0.730589863 | 0.687285399 | 1.110205584 | 1.056522425 | 0.619801251 | | |
| 38 | 0.933202549 | 0.994965969 | 1.02648747 | 0.358184612 | 0.381018097 | 0.669761315 | 1.313651465 | 1.715795832 | 1.195560254 | | |
| 39 | 1 | 1 | 1 | 1 | 1 | 1 | 1 | 1 | 1.370531915 | | |
| 40 | 1.161808511 | 1 | 1.050957447 | 1.679574468 | 1.331170213 | 1.155531915 | 0.990388874 | 1.193506954 | 1.026175923 | | |
| 41 | 1.154255319 | 1 | 1 | 1 | 1 | 1 | 0.84125852 | 0.929069583 | 1.270486606 | | |
| 42 | 1.130372324 | 1.088880213 | 1.216637387 | 0.761663088 | 0.503253302 | 0.952176459 | 1.140636669 | 0.991971621 | 1.410380881 | | |
| 43 | 0.964555269 | 1.087534527 | 1.094983516 | 1.139623986 | 2.057881137 | 1.287570168 | 0.851992262 | 0.622359447 | 1.026224767 | | |
| 44 | 1.088105207 | 1.001698754 | 1.140713477 | 1.284470555 | 1.102774632 | 1.176741223 | 0.917400475 | 0.898717648 | 1.399872769 | | |
| 45 | 1 | 1 | 1 | 1 | 1 | 1 | 1 | 1 | 1 | | |
| 46 | 0.640959028 | 0.883288006 | 0.805451901 | 1.080661143 | 1.038032593 | 0.978664643 | 0.904647275 | 0.866599799 | 1.907255099 | | |
| 47 | 1 | 1.141220994 | 0.936161737 | 0.936161737 | 0.936161737 | 1.132158152 | 1 | 1 | 1 | | |
| 48 | 1.221914894 | 0.524964809 | 0.308334023 | 0.235385443 | 0.194584748 | 0.194584748 | 0.908479747 | 1.426247105 | 0.531043444 | | |
| 49 | 1 | 1 | 1 | 2.93712766 | 1.147340426 | 1.110744681 | 0.904063366 | 1.29082812 | 0.919411913 | | |
| 50 | 1.26855394 | 0.961353586 | 1.132666795 | 0.795103506 | 0.602448247 | 0.815740563 | 0.9290144241 | 1.304266607 | 0.735784204 | | |
| 51 | 0.921338319 | 0.949799826 | 0.827554395 | 0.384386423 | 0.414482158 | 0.579704091 | 1.335097181 | 1.533123028 | 1.127709372 | | |
| 52 | 0.862805137 | 1 | 1 | 1 | 1 | 1.048297872 | 1 | 1 | 1 | | |
| 53 | 1.873187465 | 0.83237586 | 1.050848805 | 1.829573013 | 2.421699201 | 1.748479307 | 0.965321312 | 0.839864447 | 1.059010902 | | |
| 54 | 0.889018521 | 1.385040716 | 1.452297175 | 1.558158239 | 1.861264703 | 1.60058309 | 1 | 1.141170213 | 1.058723404 | | |
| 55 | 0.902404907 | 1.017979241 | 1.110946712 | 0.822068285 | 0.992108862 | 1.093415832 | 0.93772513 | 1.116395764 | 1.230515582 | | |
| 56 | 1.266914894 | 11.118617021 | 1.238510638 | 1.400425532 | 1.173404255 | 1 | 1 | 1.243297872 | | | |
| 57 | 1.018685111 | 1.06132114 | 0.905813669 | 1.641504042 | 2.376071691 | 1.701804524 | 0.900267514 | 0.8663894 | 1.580902023 | | |
| 58 | 0.957543427 | 1.540170671 | 1.614326517 | 0.984439889 | 1.407366521 | 1.487480068 | 1.225028681 | 1.010436327 | 1.339310166 | | |
| 59 | 1 | 1 | 1 | 1 | 1.043723404 | 1 | 1.012591815 | 0.896689879 | 2.08976438 | | |
| 60 | 0.91881406 | 1.055205521 | 1.353285329 | 0.726372637 | 0.669166917 | 1.401390139 | 0.849832746 | 1.385317783 | 1.001898563 | | |
| 61 | 1.338031102 | 1.46948589 | 1.50828456 | 0.520182453 | 0.449564477 | 0.970791086 | 1.008471074 | 1.108161157 | 1.186570248 | | |
| 62 | 1 | 1 | 1 | 1 | 1 | 1 | 1 | 1 | 1 | | |
| 63 | 0.831556503 | 0.810053109 | 0.741246416 | 1.605278 | 1.305329699 | 1.113878836 | 0.989600682 | 1.066526785 | 1.047223133 | | |
| 64 | 1.442209274 | 0.953370052 | 1.090596014 | 0.651979313 | 0.312010272 | 0.780301667 | 1.280011857 | 1.190470309 | 0.865724731 | | |
| 65 | 1.145988208 | 0.796242658 | 1.063641807 | 0.667988155 | 0.253919966 | 0.595171443 | 1.04457325 | 1.459991306 | 0.902825677 | | |
| 66 | 0.802644673 | 1.168877343 | 0.996664722 | 1.553138794 | 1.990445474 | 1.261967924 | 0.93901366 | 0.788840985 | 0.742966499 | | |
| 67 | 1.171043835 | 1 | 1.141382979 | 1 | 1.020638298 | 1.137553191 | 1.219148936 | 1.276808511 | 1.548723404 | | |
| 68 | 0.764546936 | 0.931890895 | 0.89618242 | 0.803589827 | 0.735019793 | 0.929098205 | 0.953429402 | 0.907848352 | 0.955037417 | | |
| 69 | 1 | 1 | 1.145851064 | 1 | 1 | 1 | 1 | 1 | 1 | | |
| 70 | 0.927125506 | 1.095564339 | 1.021607378 | 1.443039087 | 2.049187527 | 1.514624506 | 1 | 1 | 1.674042553 | | |
| 71 | 1.073745023 | 1.221236586 | 1.231336589 | 0.873384498 | 1.290375096 | 1.206917173 | 0.950592054 | 1.004508024 | 1.410049889 | | |
| 72 | 0.941984284 | 0.859453156 | 0.641083868 | 0.399300334 | 1.189032436 | 1.200079786 | 1 | 1 | 1 | | |
| 73 | 0.848806973 | 0.951680841 | 0.791216162 | 0.837388995 | 1.019343761 | 0.852171813 | 1.395205184 | 1.336846652 | 1.1613131749 | | |
| 74 | 1 | 1 | 1 | 1 | 1 | 1 | 1 | 1 | 1 | | |
| 75 | 1.085030066 | 0.965185044 | 1.135062953 | 0.739173979 | 1.066959176 | 1.017312095 | 1.051844185 | 4.468234851 | 1.831482875 | | |
| 76 | 1.180184591 | 1.002048664 | 1.402644639 | 1.103616385 | 1.604169634 | 1.821919609 | 1.183887745 | 1.184349679 | 1.257509144 | | |
| 77 | 1.070558456 | 0.94876881 | 1.086730506 | 0.954924761 | 2.03255814 | 1.478043776 | 1.139059305 | 1.215713438 | 1.592123491 | | |
| 78 | 1 | 1 | 1 | 1 | 1 | 1 | 0.781681497 | 1.28198834 | 1.365296103 | | |
| 79 | 1.465534549 | 0.976688404 | 1.352372366 | 1.012892587 | 1.209654449 | 1.285960573 | 0.950485195 | 0.853363192 | 1.541262337 | | |
| 80 | 0.852931911 | 1.047319226 | 1.093470077 | 1.284239907 | 2.799558614 | 1.61547449 | 0.813207208 | 0.646440759 | 0.742621086 | | |
| 81 | 1.026797538 | 1.081992557 | 0.778700258 | 0.592499284 | 0.704093902 | 0.59782422 | 1 | 1 | 1 | | |
| 82 | 1 | 1 | 1 | 1 | 2.456382979 | 1.948723404 | 1 | 1 | 1 | | |
| 83 | 1.108391862 | 1.079455253 | 1.096420233 | 0.876420233 | 1.352140078 | 0.891284047 | 0.87255175 | 0.87255175 | 1.469785575 | | |
| 84 | 1.00769756 | 0.930976927 | 0.950967108 | 0.755169367 | 0.839567992 | 0.909631811 | 1.219954091 | 1.328988359 | 2.135923922 | | |
| 85 | 1 | 0.561973169 | 0.722980727 | 0.591653805 | 0.769183585 | 0.992322609 | 1 | 1 | 1 | | |
| 86 | 0.764236961 | 1.015604102 | 1.064199733 | 1.236736514 | 1.892618305 | 1.253232278 | 0.716803654 | 0.666352692 | 0.943523309 | | |
| 87 | 0.824757644 | 1.211308684 | 1.280035751 | 1.737087214 | 2.543371907 | 1.990638818 | 0.875189448 | 0.749969279 | 1.008315242 | | |
| 88 | 1.198447502 | 0.964168451 | 1.139700214 | 0.617958601 | 0.980785153 | 1.272319772 | 1.249825565 | 1.66225928 | 1.417387664 | | |
| 89 | 1 | 1 | 1 | 1 | 1 | 1 | 1 | 1 | 1 | | |
| 90 | 0.822997189 | 1.03375267 | 1.187230002 | 1.111417042 | 1.75005934 | 1.070021362 | 1.016506816 | 1.316930871 | 1.49975809 | | |
| 91 | 0.935820983 | 1.052880688 | 0.96819119 | 1.533202796 | 2.236809004 | 1.727115941 | 0.899897843 | 0.871281954 | 1.127743989 | | |
| 92 | 0.860299813 | 0.913536716 | 1.1244826 | 1.117813889 | 2.230032194 | 1.86432623 | 1.072339382 | 0.700735655 | 1.069641981 | | |
| 93 | 1.022850861 | 1.024844226 | 1.149312362 | 1.571990885 | 2.081177989 | 1.776848178 | 0.986691982 | 0.818235606 | 1.6886241611 | | |
| 94 | 1 | 1 | 1 | 1.898723404 | 1.112872341 | 1 | 1.044723142 | 1.054365831 | 1.029637956 | | |
| 95 | 0.872673849 | 1 | 1.046382979 | 1.232659574 | 1.043723404 | 1 | 0.859719597 | 0.830917874 | 1.128701953 | | |
| 96 | 1.018072951 | 0.777135802 | 0.898419753 | 1.523580247 | 1.971308642 | 1.573530864 | 1.107228693 | 1.335287748 | 2.086760162 | | |
| 97 | 0.775925321 | 1.106164576 | 1.0579377 | 0.947741837 | 0.667436921 | 0.765966545 | 1.000090831 | 0.888072907 | 0.533469467 | | |
| 98 | 0.806399367 | 1.148100616 | 0.831215777 | 0.889609001 | 0.338017625 | 0.840648528 | 0.900495219 | 0.951072974 | 0.714810464 | | |
| 99 | 1 | 1 | 1 | 1 | 1 | 1 | 1 | 1.015531915 | 1.047553191 | | |
| 100 | 1 | 1 | 1 | 1 | 1 | 1 | 1 | 1 | 1 | | |
| 101 | 1.139255319 | 1 | 1 | 1 | 1 | 1 | 1 | 1 | 1 | | |
| 102 | 1.162496029 | 0.915716989 | 1.050295977 | 0.799178512 | 1.449120122 | 1.2171707 | 1.485274239 | 1.095154221 | 2.136238889 | | |
| 103 | 0.88384133 | 0.94960855 | 1.029638375 | 1.221759662 | 2.196905679 | 1.308189387 | 1.133491144 | 1.012089445 | 1.265333728 | | |
| 104 | 1.050580851 | 1.182500839 | 0.827581385 | 1.808633516 | 2.246112541 | 1.465558228 | 1.050341742 | 0.726357334 | 1.150327595 | | |
| 105 | 0.930240041 | 1.133824965 | 1.015996096 | 1.126287821 | 1.219607418 | 0.915844269 | 1.201292961 | 1.267985413 | 1.450160239 | | |
| 106 | 1.039715217 | 1.100472813 | 1.04964539 | 1.098108747 | 1.714766321 | 1.026004728 | 1 | 1 | 1.465 | | |
| 107 | 1.163457044 | 1.22817437 | 1.481764753 | 1.395471689 | 1.184049346 | 1.111554254 | 1.072264963 | 1.23325075 | 1.44547529 | | |
| 108 | 1.041855774 | 1.019343422 | 0.939101782 | 1.203563583 | 2.004271418 | 1.312911887 | 0.728415844 | 0.73236922 | 0.980462672 | | |
| 109 | 1.49334328 | 1.312081208 | 1.238423842 | 0.470047005 | 0.501450145 | 0.649264926 | 1 | 1 | 1 | | |
| 110 | 0.877587469 | 1.273558613 | 1.285490333 | 2.52421181 | 1.743250305 | 1.295593102 | 1.202892674 | 1.275209516 | 1.178201316 | | |
| 111 | 0.989168098 | 1.245199462 | 1.33280884 | 1.105579802 | 1.393797213 | 1.644293821 | 1.24600197 | 1.054703593 | 1.762178192 | | |
| 112 | 1.789966774 | 0.828706571 | 1.055070666 | 1.657863618 | 2.019257841 | 1.545807759 | 0.9780087 | 0.693709867 | 0.746219706 | | |
| 113 | 1.035371601 | 1.000289519 | 0.883323683 | 1.6403011 | 1.959988419 | 1.83358425 | 0.834794063 | 0.834034146 | 0.996048432 | | |
| 114 | 1.272159117 | 0.832568946 | 0.726371103 | 0.772130258 | 0.510539784 | 0.533619362 | 1.032859745 | 1.093603955 | 0.535248504 | | |
| 115 | 1.3997825 | 0.555289221 | 0.402252076 | 0.521526655 | 0.287437055 | 0.363937046 | 0.902819395 | 1.121152275 | 0.640152547 | | |
| 116 | 0.696957353 | 1.109891003 | 1.037052084 | 1.202869447 | 2.309777246 | 1.493621516 | 1.4219039 | 1.114702142 | 1.404390798 | | |
| 117 | 0.636209814 | 1 | 1 | 1 | 2.092340426 | 1.815638298 | 0.750057825 | 0.434842948 | 0.434842948 | | |
| 118 | 0.912637363 | 0.815846306 | 0.731215693 | 0.752362098 | 1.192027355 | 0.92652749 | 1 | 1 | 1 | | |
| 119 | 0.972097408 | 1.017676925 | 1.039965541 | 1.158054878 | 1.438763128 | 1.230720164 | 0.950735234 | 0.754829879 | 0.719232135 | | |
| 120 | 0.876362073 | 1.261593272 | 1.211190282 | 2.08071487 | 1.836993342 | 1.421416891 | 1.025498891 | 1.230413895 | 1.337891106 | | |
| 121 | 1 | 1 | 1 | 1 | 2.117021277 | 1 | 1 | 1 | 1 | | |
| 122 | 1.11523043 | 1.626336249 | 1.41122449 | 0.753109815 | 0.888483965 | 1.296890185 | 1 | 1 | 1 | | |
| 123 | 0.856464201 | 1.25361497 | 1.100120499 | 0.628296002 | 0.401368018 | 0.716827332 | 0.821828418 | 0.863476425 | 0.601369074 | | |
| 124 | 1 | 1 | 1 | 1 | 1 | 1 | 1 | 1 | 1 | | |
| 125 | 1.190873313 | 1.190002863 | 0.971530249 | 0.624575613 | 0.384505256 | 0.734609564 | 1 | 1 | 1 | | |
| 126 | 1.177897813 | 1.458998182 | 1.282468188 | 0.474651586 | 0.871642093 | 0.812765098 | 0.953380911 | 0.651486369 | 0.752477281 | | |
| 127 | 1.107848372 | 0.426077648 | 0.330286687 | 0.272976793 | 0.236581717 | 0.215310085 | 1.001437683 | 1.187918235 | 0.645964032 | | |
| 128 | 1 | 1 | 1 | 1 | 1 | 1 | 1 | 1 | 1.200425532 | | |
| 129 | 1 | 1 | 1 | 1.867021277 | 1 | 1 | 0.963806008 | 0.963806008 | 0.963806008 | | |
| 130 | 0.888252735 | 0.949186028 | 0.777303619 | 0.742848111 | 1.248854117 | 0.818476371 | 0.6917355963 | 0.862683052 | 1.136360291 | | |
| 131 | 2.278902452 | 0.803321904 | 0.569454081 | 0.59568897 | 0.309265271 | 0.488067684 | 1.049837995 | 1.444243631 | 0.646639517 | | |
| 132 | 1.259683375 | 1.073693065 | 1.000740436 | 0.766460455 | 0.558485656 | 0.58222341 | 0.996806204 | 1.275384521 | 0.91710493 | | |
| 133 | 1 | 1 | 1 | 1.129361702 | 1 | 1 | 0.862490632 | 0.594296083 | 0.566525975 | | |
| 134 | 1.048358723 | 1.072739868 | 0.801220991 | 1.875389678 | 0.448259439 | 0.569579148 | 1.010625536 | 1.440659811 | 1.402527849 | | |
| 135 | 1 | 1 | 1 | 1 | 1 | 1 | 1 | 1 | 1 | | |
| 136 | 1.031614754 | 0.963687489 | 0.943097297 | 0.446090585 | 0.584144645 | 1.264739675 | 1 | 1 | 1 | | |
| 137 | 0.77359888 | 1 | 1 | 1 | 1 | 1.262234043 | 1 | 0.762237039 | 0.485863441 | 0.562309402 | |
| 138 | 0.800565481 | 1.153033437 | 1.212030334 | 0.792433644 | 0.822905895 | 0.811892451 | 1.055970783 | 1.123577374 | 1.769492101 | | |
| 139 | 0.9471507 | 0.812765807 | 0.971363765 | 1.066664304 | 1.964240147 | 1.29327332 | 1.114698906 | 1.189583493 | 1.082587293 | | |
| 140 | 1.147597254 | 0.941891468 | 0.844651819 | 3.119667503 | 1.371392723 | 0.998117942 | 0.80349083 | 1.270727675 | 0.997035166 | | |
| 141 | 1.235957402 | 1 | 1 | 3.404042553 | 1 | 1 | 0.991649433 | 0.841470119 | 0.893301222 | | |
| 142 | 1.146646143 | 1.038045261 | 0.795124084 | 0.772439051 | 0.444107358 | 0.708702307 | 1.120515138 | 1.198599187 | 0.746588342 | | |
| 143 | 0.735853147 | 1.109368995 | 1.145748469 | 0.906489381 | 0.905823278 | 1.034278687 | 1.042040963 | 1.212450593 | 1.492993173 | | |
| 144 | 0.96263682 | 1.172281993 | 1.124575311 | 0.665345413 | 0.665345413 | 0.937146093 | 1.096840659 | 1.28483124 | 0.966149922 | | |
| 145 | 1 | 1 | 1 | 1 | 1 | 1 | 1 | 1 | 1 | | |
| 146 | 0.671883921 | 1.055585337 | 1.264916911 | 1.26330873 | 2.066718898 | 1.237907715 | 0.812355407 | 0.638088852 | 0.703956412 | | |
| 147 | 1 | 0.566802261 | 0.49659253 | 0.752443341 | 0.609699403 | 0.49659253 | 1 | 1 | 1 | | |
| 148 | 1 | 1 | 1 | 3.736595745 | 1 | 1 | 1 | 1 | 1 | | |
| 149 | 1.351067538 | 0.557174167 | 0.368658067 | 0.279770148 | 0.260961062 | 0.189006771 | 0.910505933 | 1.271688991 | 0.448703717 | | |
| 150 | 1.148165514 | 1.043416502 | 1.117094862 | 0.985795455 | 1.052124506 | 1.064970356 | 1 | 1 | 1 | | |
| 151 | 1.101702128 | 0.936161737 | 1.055671746 | 0.961557614 | 0.936161737 | 0.936161737 | 1 | 1 | 1 | | |
| 152 | 1.195741262 | 1.006998583 | 1.271881644 | 2.979270021 | 1.350815025 | 1.472802977 | 0.668511397 | 0.848091173 | 0.960512835 | | |
| 153 | 1 | 1 | 1 | 1 | 1 | 1 | 1 | 1 | 1 | | |
| 154 | 2.030710807 | 0.714688137 | 0.506453031 | 0.374595437 | 0.187797179 | 0.187797179 | 1.11203966 | 1.242422096 | 0.348689802 | | |
| 155 | 0.77471301 | 1.189890578 | 1.122010264 | 1.85077951 | 2.108840902 | 1.516219618 | 0.861500371 | 0.758752166 | 0.895865313 | | |
| 156 | 0.675584216 | 1.254257827 | 1.347588517 | 0.843126887 | 0.939255734 | 0.868067949 | 0.856950597 | 0.823136881 | 1.646946672 | | |
| 157 | 0.911433085 | 1.046312498 | 1.120211459 | 0.52812628 | 0.522579204 | 0.856520606 | 1.000505051 | 1.39010101 | 0.949494949 | | |
| 158 | 0.949952803 | 0.961900436 | 0.915338013 | 0.890617381 | 0.664845404 | 0.553465631 | 0.852271611 | 1.067313547 | 0.491072656 | | |
| 159 | 1 | 1 | 1 | 1 | 1 | 1 | 0.979392625 | 0.930919406 | 1.066577674 | | |
| 160 | 0.826571404 | 1.175492075 | 0.956714858 | 2.678366138 | 1.552778262 | 1.242901933 | 0.87596822 | 0.847226719 | 1.014146597 | | |
| 161 | 0.876625088 | 1.207121845 | 1.054714352 | 1.115590649 | 0.924013005 | 0.820560458 | 0.894463464 | 1.047928226 | 0.65785228 | | |
| 162 | 0.828413883 | 0.936973103 | 0.745305321 | 0.433025559 | 0.437619876 | 0.698425443 | 1 | 1.276808511 | 1 | | |
| 163 | 1.15661003 | 0.983357362 | 1.058076851 | 0.737760923 | 0.678869178 | 0.688929982 | 0.993022174 | 0.889491911 | 1.381144363 | | |
| 164 | 1 | 1.170120605 | 1.162646509 | 1.053677595 | 1.930609818 | 1.256072703 | 1.06893617 | 1.156489362 | 1.146489362 | | |
| 165 | 1.227443038 | 1 | 1.186914894 | 1 | 1 | 1.110744681 | 1.167122833 | 1.437505184 | 1.272621714 | | |
| 166 | 1 | 1 | 1 | 3.348297872 | 1 | 1 | 0.545835251 | 1.106366565 | 0.969479901 | | |
| 167 | 0.816310201 | 1.329722501 | 1.142728185 | 0.761584754 | 0.737713139 | 0.794032096 | 0.87111915 | 0.722165509 | 0.502163734 | | |
| 168 | 1 | 0.972682119 | 0.972682119 | 0.972682119 | 0.972682119 | 0.972682119 | 1 | 1 | 1 | | |
| 169 | 1.020588063 | 1.078949093 | 1.095597776 | 1.147230098 | 1.213754931 | 0.919963129 | 0.980588728 | 0.923820121 | 0.445577932 | | |
| 170 | 1.17498814 | 1.009916342 | 1.190069234 | 0.744555027 | 0.819666811 | 1.020878408 | 1.128216938 | 1.353100351 | 1.461454315 | | |
| 171 | 1.127352682 | 1.047322073 | 1.386891872 | 1.025321207 | 1.858495741 | 1.906395265 | 1.070953534 | 1.232812156 | 1.531424796 | | |
| 172 | 1 | 1 | 1 | 1 | 1 | 1 | 1 | 1 | 1 | | |
| 173 | 1.357964037 | 0.992164098 | 0.861015845 | 1.955285435 | 0.737464728 | 1.0459084 | 1.279049023 | 1.227014169 | 1.314277588 | | |
| 174 | 1.375857454 | 0.972884386 | 1.119368296 | 0.560190703 | 0.560190703 | 0.692669845 | 0.998514806 | 1.177852038 | 1.29657477 | | |
| 175 | 1 | 0.877684407 | 0.877684407 | 0.877684407 | 0.877684407 | 0.877684407 | 1 | 1 | 1 | | |
| 176 | 0.938137755 | 1.117156016 | 1.182594512 | 2.209748609 | 1.128094416 | 1.123296872 | 1.036936387 | 1.120902889 | 0.921745882 | | |
| 177 | 0.769113113 | 0.961506644 | 1.069625224 | 0.972501754 | 0.688084442 | 1.002722599 | 1.057485751 | 1.180711674 | 1.375570543 | | |
| 178 | 1.185903226 | 1.123265821 | 1.1868833 | 1.058127458 | 1.218413829 | 1.060835374 | 1.231792217 | 1.310157087 | 1.404498393 | | |
| 179 | 0.897717505 | 1 | 1 | 1 | 1 | 1 | 1.019656631 | 0.77963009 | 0.77963009 | | |
| 180 | 0.924014548 | 0.798191145 | 0.888093952 | 1.261406587 | 1.879859611 | 1.540226782 | 0.688898488 | 0.77511879 | 0.832354212 | | |
| 181 | 1.095632006 | 1.117156016 | 1.216177317 | 1.892535022 | 0.901938208 | 0.901938208 | 1.573738528 | 1.244259925 | 0.729888728 | | |
| 182 | 1 | 0.396112726 | 0.50754947 | 0.220388258 | 1.319656757 | 1.256635093 | 1 | 1 | 1 | | |
| 183 | 1 | 1 | 1 | 1 | 1 | 1 | 0.612891986 | 0.540766551 | 0.732404181 | | |
| 184 | 1.010760842 | 0.983129237 | 1.088032796 | 0.811583539 | 0.901298155 | 1.174804226 | 1.263908702 | 1.344190838 | 1.728483119 | | |
| 185 | 1.088733457 | 0.910154366 | 0.955318379 | 2.560057887 | 0.566811384 | 0.634647853 | 0.748684775 | 1.025447177 | 0.846108881 | | |
| 186 | 0.978165939 | 1 | 1 | 1.095212766 | 1.884574468 | 1.299680851 | 1 | 1 | 1 | | |
| 187 | 1 | 1 | 1.028510638 | 1.981808511 | 1 | 1 | 1.193278466 | 1.206419045 | 1.006501542 | | |
| 188 | 0.957037451 | 1.226900319 | 1.456499122 | 0.497789193 | 0.29353339 | 0.751430522 | 1.084459247 | 1.311380632 | 0.68833658 | | |
| 189 | 0.654130793 | 0.962439299 | 0.694497357 | 0.398894642 | 0.367799748 | 0.527915649 | 1 | 1 | 1 | | |
| 190 | 1 | 1 | 1 | 1 | 1 | 1 | 1 | 1 | 1 | | |
| 191 | 0.80143711 | 0.963343109 | 0.883502611 | 0.809241113 | 0.926990201 | 0.796849295 | 1.237863209 | 1.130307671 | 0.892862115 | | |
| 192 | 1 | 1 | 1 | 1 | 1 | 1 | 1 | 1 | 1 | | |
| 193 | 0.834550741 | 0.776124919 | 0.601774 | 1.634620715 | 1.128014414 | 0.829668299 | 1 | 1 | 1 | | |
| 194 | 1 | 0.942406489 | 0.909418657 | 0.884632717 | 3.616584047 | 1.972780532 | 1 | 1 | 1.30712766 | | |
| 195 | 0.702598088 | 1.013515601 | 1.421995675 | 0.774482546 | 1.609051591 | 1.456518381 | 0.7808011782 | 1.017193764 | 0.700935412 | | |
| 196 | 1 | 1.135433202 | 1.136268694 | 0.785362186 | 1.385746512 | 1.507895396 | 1 | 1 | 1.433617021 | | |
| 197 | 0.89311261 | 0.918840859 | 0.821940888 | 1.008857225 | 1.27534418 | 1.062578223 | 1 | 1 | 1 | | |
| 198 | 1.080507225 | 0.998936924 | 0.886428065 | 0.860028349 | 0.878809355 | 1.195694543 | 1 | 1 | 1.124680851 | | |
| 199 | 0.73260500 | 1.125234365 | 1.13313708 | 0.856489513 | 0.901890694 | 0.956508788 | 0.99801919802 | 1.002271404 | 1.53960396 | | |
| 200 | 1 | 1 | 1 | 1.163723404 | 1 | 1 | 1 | 1 | 1 | | |
| 201 | 1 | 1 | 1 | 1 | 1.124361702 | 1 | 1 | 1 | 1 | | |
| 202 | 1.602981384 | 0.892174556 | 0.69815373 | 0.89988991 | 0.265047194 | 0.450391633 | 1.034285568 | 1.38302405 | 0.34628988 | | |
| 203 | 0.679492901 | 0.949551854 | 1.151947598 | 1.500657442 | 2.511369531 | 2.073006261 | 0.773748968 | 0.506079468 | 0.96198938 | | |
| 204 | 1.376929815 | 1.459472196 | 1.320040057 | 0.553722903 | 0.628946748 | 0.805136664 | 1 | 1 | 1 | | |
| 205 | 1.056577971 | 1.020698772 | 0.91898017 | 0.520944287 | 0.813673277 | 0.57170916 | 0.964910837 | 1.018052126 | 0.412866941 | | |
| 206 | 1.124255319 | 0.95588059 | 1.26927227 | 0.665387843 | 1.529372977 | 1.373516365 | 1 | 1 | 1.080851064 | | |
| 207 | 1.010221236 | 1.170120605 | 1.204178699 | 1.340920673 | 0.798369288 | 0.798369288 | 0.8546567 | 1.339243082 | 0.352328024 | | |
| 208 | 1.304468085 | 1.241973841 | 1.403601155 | 1.495583489 | 1.081195855 | 1.464582979 | 1.268103034 | 0.934062827 | 1.644725309 | | |
| 209 | 1.052688489 | 1.154238152 | 1.19279769 | 1.147698318 | 1.044419908 | 1.023186683 | 0.825490085 | 0.858426078 | 0.994407096 | | |
| 210 | 0.975843284 | 1.001454636 | 1.007190059 | 0.719504592 | 0.390673704 | 0.550268069 | 1.256771919 | 2.014665812 | 0.753325854 | | |
| 211 | 0.985621219 | 0.886284062 | 0.896787943 | 1.207388238 | 2.492109403 | 1.773177044 | 1.140080093 | 0.96637744 | 1.058318038 | | |
| 212 | 1.263901413 | 0.926342561 | 1.004450748 | 0.756847305 | 0.898415338 | 0.968233395 | 1.261029829 | 1.363955994 | 1.275377112 | | |
| 213 | 0.833457483 | 1 | 1.395106383 | 2.874680851 | 1.586808511 | 1.254468085 | 1.049859104 | 0.923870557 | 0.857967458 | | |
| 214 | 1.453651567 | 0.933982304 | 0.815135941 | 0.731390451 | 0.456645908 | 0.569470141 | 0.939829954 | 1.30520506 | 0.6316881794 | | |
| 215 | 1.390943878 | 1.102339787 | 1.378041205 | 1.888290713 | 1.752200021 | 1.009162439 | 0.95323675 | 0.841665138 | 1.139116083 | | |
| 216 | 1.064770007 | 0.747079186 | 0.954601183 | 1.238497043 | 1.112072696 | 0.972739074 | 1.158106039 | 1.044907082 | 0.504449319 | | |
| 217 | 0.653453711 | 0.937971118 | 0.883463849 | 0.410367452 | 0.661429758 | 0.800465624 | 1.085875417 | 1.118943861 | 1.092369478 | | |
| 218 | 1.829529882 | 0.971401542 | 0.826037654 | 0.673377547 | 0.425422724 | 0.669405449 | 1.078100834 | 1.444042684 | 0.797569192 | | |
| 219 | 1.091290767 | 1.041766154 | 0.892441427 | 0.987141223 | 0.649498831 | 0.67376759~ | 1.028442724 | 1.039232746 | 0.738128537 | | |
| 220 | 0.927886213 | 1.214646064 | 1.098391344 | 0.706666244 | 0.92705524 | 1.167020973 | 1.008191489 | 1 | 1.653191489 | | |
| 221 | 1 | 1 | 1 | 1.587765957 | 1 | 1 | 0.962163782 | 1.040008991 | 0.234759372 | | |
| 222 | 1.003617021 | 1.102765957 | 1.041914894 | 1 | 1 | 1 | 1 | 1.187021277 | 1 | | |
| 223 | 0.970436561 | 0.947328002 | 0.836634712 | 0.657274509 | 1.307160638 | 1.62949526 | 1.090332129 | 0.99026667 | 0.997467058 | | |
| 224 | 1 | 1 | 1 | 1 | 1 | 1 | 1 | 1 | 1 | | |
| 225 | 1 | 0.915215067 | 0.324238557 | 0.324238557 | 0.324238557 | 0.324238557 | 1 | 1 | 1 | | |
| 226 | 1.032953996 | 1.057207501 | 0.971135914 | 0.308965948 | 0.535675404 | 0.545056942 | 1.152582307 | 0.990148821 | 0.812833281 | | |
| 227 | 1.090228844 | 0.780110132 | 0.889432383 | 0.600672198 | 0.210617132 | 0.525755098 | 1.262090424 | 1.952521799 | 1.004455883 | | |
| 228 | 1.06614937 | 0.920856463 | 0.915107058 | 0.858207772 | 0.547898493 | 0.7370341 | 0.958659165 | 1.219149342 | 1.067757009 | | |
| 229 | 1 | 1.121125337 | 1.453672507 | 0.791778976 | 1.598214286 | 1.596192722 | 1 | 1 | 1.102765957 | | |
| 230 | 0.782015943 | 1.114766759 | 1.019616929 | 1.383070745 | 2.046184739 | 1.442539388 | 1 | 1.125744681 | 1.872553191 | | |
| 231 | 1.067234607 | 1.355688771 | 1.301875965 | 1.303279642 | 0.523734558 | 0.979169736 | 1.177523718 | 0.930142001 | 1.03812581 | | |
| 232 | 0.600271657 | 0.94049135 | 0.894306923 | 2.298506072 | 2.557722769 | 1.184489238 | 0.962828685 | 0.847205522 | 1.066588398 | | |
| 233 | 1.854810375 | 1 | 1.237340426 | 1 | 1.273723404 | 1.272553191 | 1 | 1 | 1 | | |
| 234 | 1.082495478 | 1.005513614 | 0.802570193 | 0.568368818 | 0.44825685 | 0.788362032 | 1.075666925 | 1.141057225 | 0.892869712 | | |
| 235 | 1.189731228 | 1.024649196 | 0.912182883 | 0.582589908 | 0.667224236 | 0.88825388 | 1.248596112 | 1.168552916 | 1.628941685 | | |
| 236 | 0.93973822 | 0.830813295 | 0.864717245 | 0.626124026 | 0.732764937 | 0.819423167 | 0.979822355 | 0.946192946 | 0.605226678 | | |
| 237 | 1.088899568 | 1.001359291 | 1.113654301 | 0.962772954 | 0.842059107 | 1.049942997 | 1.062567678 | 0.952196075 | 1.08985025 | | |
| 238 | 1.18750952 | 0.969870875 | 1.121347127 | 1.42950993 | 1.807218908 | 1.465151401 | 0.845035284 | 0.716159384 | 0.873904056 | | |
| 239 | 1.262020721 | 1.093718777 | 1.164857124 | 0.837340971 | 0.626124026 | 0.996602944 | 0.7857792 | 0.830128322 | 0.919262787 | | |
| 240 | 1 | 0.883161512 | 0.771773907 | 0.556938026 | 1.158371845 | 1.106647707 | 1 | 1 | 1 | | |
| 241 | 1 | 1 | 1 | 1 | 1 | 1 | 0.896689879 | 1.037012306 | 1.200610512 | | |
| 242 | 1 | 1 | 1 | 1 | 1.158829787 | 1 | .003829787 | 1.39920429 | 1.018197544 | 1.402456322 | |
| 243 | 0.827739001 | 0.946950925 | 0.612262783 | 0.21520307 | 0.172745679 | 0.351867521 | 1.027419773 | 1.738084941 | 0.813078453 | | |
| 244 | 0.725769453 | 1 | 1 | 2.592553191 | 1 | 1 | 0.856167927 | 0.700403229 | 1.069797236 | | |
| 245 | 1.594680663 | 1.080780452 | 1.308123632 | 0.961068563 | 1.091037564 | 1.804932531 | 1.084111572 | 1.527985652 | 0.876059125 | | |
| 246 | 1 | 1.034980566 | 0.996761059 | 1.445030539 | 1.971589857 | 1.457153433 | 0.972459193 | 1.020689192 | 1.07684557 | | |
| 247 | 1.100172853 | 1.142689615 | 1.335819166 | 1.131376969 | 1.398716355 | 1.521105002 | 1.025552608 | 0.979126685 | 1.265488688 | | |
| 248 | 1 | 1 | 1 | 1 | 1.740531915 | 1.326808511 | 0.721080086 | 0.721080086 | 1.124271249 | | |
| 249 | 1.050204746 | 1.066043462 | 1.096734248 | 1.477285419 | 2.405414593 | 1.783707661 | 1.020776567 | 1.277836265 | 1.150606886 | | |
| 250 | 1.110754102 | 0.725352789 | 0.631698186 | 0.803283095 | 0.928616684 | 0.927023135 | 1.255770547 | 1.165743794 | 1.198759825 | | |
| 251 | 1.346664046 | 0.936611816 | 0.83793201 | 0.866820689 | 0.554778632 | 0.549148946 | 0.926231702 | 1.129615802 | 0.74886234 | | |
| 252 | 1.106321401 | 1.112438189 | 1.187980221 | 0.715100799 | 0.779307722 | 1.072499049 | 1.299580543 | 0.842004563 | 0.837809993 | | |
| 253 | 0.891169266 | 0.946127567 | 0.96447321 | 1.395948067 | 1.853102965 | 1.540387436 | 1.032808536 | 1.032880903 | 1.103443077 | | |
| 254 | 1.011675559 | 0.972804566 | 1.142674121 | 1.706848358 | 1.302818542 | 1.350687165 | 1.262669903 | 1.031116505 | 1.256990291 | | |
| 255 | 0.667343486 | 1 | 1 | 1.296489362 | 1.776382979 | 1.200425532 | 0.732801786 | 0.525051826 | 0.766640089 | | |
| 256 | 0.633708385 | 0.9131443 | 0.964366892 | 1.018011709 | 1.141304098 | 1.16227758 | 1.209514622 | 1.313139724 | 1.421222583 | | |
| 257 | 1.496940354 | 0.922831588 | 1.045136241 | 2.331692436 | 1.845385842 | 1.183318311 | 1.150102722 | 0.958739942 | 1.453561034 | | |
| 258 | 1.446104038 | 0.743960035 | 0.698843751 | 0.899291574 | 0.202296504 | 0.382629925 | 1.213028292 | 1.318651764 | 0.3514146 | | |
| 259 | 1.027720664 | 1.030213753 | 0.979188867 | 0.458847866 | 0.436500972 | 0.651194133 | 0.962612164 | 0.95789903 | 0.66418925 | | |
| 260 | 1.443682335 | 1.138355699 | 1.054272125 | 1.410225922 | 0.798369288 | 0.798369288 | 0.963570128 | 1.638737098 | 0.954219794 | | |
| 261 | 1.043716203 | 1.123119015 | 1.215116279 | 0.642954856 | 0.937688098 | 1.01751026 1 | 1 | 1 | | | |
| 262 | 1.223942958 | 0.846782605 | 0.868461105 | 0.928652549 | 1.0352913 | 1.154337631 | 0.943020446 | 1.12131744 | 1.619199544 | | |
| 263 | 0.749276338 | 1.054635108 | 1.238001315 | 1.518343195 | 1.43182117 | 1.263773833 | 1.024516161 | 0.911138268 | 0.811776736 | | |
| 264 | 1 | 1 | 1.141382979 | 1.078085106 | 1.435212766 | 2.093297872 | 1.026990553 | 1.239786529 | 1.13390995 | | |
| 265 | 0.991665788 | 0.962475993 | 0.933003398 | 1.484118777 | 2.121435958 | 1.195449845 | 0.853627479 | 0.994779232 | 1.130360042 | | |
| 266 | 1 | 1 | 1 | 1.117978723 | 2.042978723 | 1.057234043 | 0.95100029 | 0.681356915 | 0.681356915 | | |
| 267 | 1.161680401 | 0.963312154 | 0.963312154 | 0.963312154 | 0.963312154 | 0.963312154 | 1 | 1 | 1 | | |
| 268 | 0.554846449 | 1.041936157 | 0.836386397 | 0.666054663 | 1.203588567 | 0.779386605 | 1.015699201 | 0.6471311125 | 0.703167775 | | |
| 269 | 1 | 0.877684407 | 0.894771242 | 0.877684407 | 1.219047619 | 0.998412698 | 1.16216746 | 1.105656679 | 1.363791655 | | |
| 270 | 1 | 1 | 1 | 1 | 1 | 1 | 0.942544871 | 0.942544871 | 0.942544871 | | |
| 271 | 0.799705677 | 1.103543666 | 1.150517343 | 1.241655498 | 1.693486302 | 1.320291642 | 0.918950106 | 0.659507776 | 1.021364725 | | |
| 272 | 1 | 0.936161737 | 0.936161737 | 0.936161737 | 1.64067324 | 1.031470969 | 0.87832818 | 0.953221968 | 1.180238895 | | |
| 273 | 1 | 1.577220481 | 1.370323929 | 3.405015674 | 1.284952978 | 0.982236155 | 0.754782609 | 0.838015608 | 1.325529543 | | |
| 274 | 1 | 0.951292728 | 0.932022853 | 0.910235306 | 0.910235306 | 0.910235306 | 1.135146981 | 1.350311492 | 0.679710735 | | |
| 275 | 1.788300501 | 0.779022546 | 0.59312888 | 0.848924054 | 0.123670821 | 0.340559212 | 1.219077151 | 1.657638964 | 0.627698592 | | |
| 276 | 0.843486809 | 1.102765957 | 1.575 | 2.613085106 | 1.704893617 | 1.236489362 | 1.085402007 | 1.08383041 | 1.045719184 | | |
| 277 | 0.867007222 | 1.031428955 | 1.029259127 | 1.209108805 | 1.823438618 | 0.885670186 | 1.04416511 | 0.89875998 | 0.798369288 | | |
| 278 | 0.648100835 | 1.164224446 | 0.839885633 | 0.905468192 | 1.342655468 | 0.872944961 | 0.906410088 | 0.955536582 | 0.904439774 | | |
| 279 | 0.780706182 | 1 | 1 | 1.072446809 | 1 | 1 | 1 | 1 | 1.025212766 | | |
| 280 | 1.095666974 | 1.214365119 | 1.287240613 | 1.765686759 | 2.26117836 | 1.760251976 | 1.289359581 | 1.007276352 | 1.617542985 | | |
| 281 | 2.0997641 | 1.983105335 | 2.530300958 | 3.218194254 | 3.086730506 | 3.73372093 | 1.335296283 | 1.600767653 | 1.177401546 | | |
| 282 | 1.402008839 | 1.1285118 | 1.217290095 | 0.754535238 | 0.754535238 | 1.001123776 | 1.036352201 | 1.614779874 | 1.310314465 | | |
| 283 | 0.897984491 | 1.08449425 | 1.301486597 | 2.139577128 | 0.381947134 | 0.978495773 | 1.139385843 | 1.101386075 | 0.72836569 | | |
| 284 | 0.654109627 | 1.020689878 | 1.133837649 | 1.370542786 | 1.926841884 | 0.998868522 | 0.952308362 | 0.744120209 | 1.225783972 | | |
| 285 | 1 | 1 | 1 | 1 | 1 | 1 | 0.993919722 | 0.641217884 | 0.90779007 | | |
| 286 | 1.215417517 | 0.971076339 | 0.864706812 | 0.881460408 | 0.742848111 | 0.742848111 | 1.20869943 | 1.44535097 | 0.849763445 | | |
| 287 | 1.24625864 | 0.886822366 | 0.84065687 | 0.596436431 | 1.047182982 | 1.022124417 | 1.35875364 | 1.053391145 | 0.52728007 | | |
| 288 | 1 | 1 | 1 | 1.5392553191 | 1 | 1.00610977 | 1.367305026 | 1.27370745 | | | |
| 289 | 1 | 0.919810563 | 0.881762723 | 0.890993739 | 1.882324611 | 1.369963076 | 0.769862532 | 0.621766437 | 0.962141508 | | |
| 290 | 1.502584661 | 1 | 1 | 5.887659574 | 1 | 1 | 0.884616237 | 0.820341763 | 1.178040293 | | |
| 291 | 0.792847503 | 1.122021277 | 1 | 1.867021277 | 1.1588297871 | 1.184021348 | 1.016773772 | 0.889064372 | | | |
| 292 | 0.880299411 | 0.892853792 | 0.903434975 | 1.265718009 | 1.572061506 | 1.195162888 | 0.923096201 | 0.831055569 | 1.201473586 | | |
| 293 | 0.861136091 | 1.306007841 | 1.510896055 | 0.580263483 | 0.858121711 | 1.040476106 | 0.995046093 | 1.219178082 | 1.085207203 | | |
| 294 | 0.883870172 | 1.200896283 | 1.294156343 | 0.73998191 | 0.867119833 | 0.891185177 | 0.948332279 | 1.061154289 | 1.184322825 | | |
| 295 | 1 | 1 | 1 | 1.02712766 | 1 | 1 | 1 | 1 | 1 | | |
| 296 | 0.749362245 | 1 | 1 | 2.137446809 | 1.181808511 | 1 | 1 | 1 | 1 | | |
| 297 | 1 | 0.73151751 | 0.73151751 | 0.73151751 | 0.73151751 | 0.73151751 | 1 | 1 | 1 | | |
| 298 | 0.974832838 | 1.083510638 | 1 | 2.436489362 | 1 | 1 | 1.225801728 | 0.997949919 | 1.008273539 | | |
| 299 | 1 | 1 | 1.059893617 | 3.832765957 | 1.158829787 | 1.057234043 | 0.971768325 | 1.105070786 | 1.018126488 | | |
| 300 | 1 | 0.42478196 | 0.42478196 | 0.42478196 | 0.42478196 | 0.42478196 | 1 | 1 | 1 | | |
| 301 | 1.60447524 | 1.621627054 | 0.517712678 | 7.937691573 | 0.472338073 | 0.472338073 | 0.927142135 | 1.119997472 | 0.970427163 | | |
| 302 | 1 | 1.076460142 | 1.160220994 | 1.010359116 | 1.255919495 | 1.416436464 | 1 | 1.125744681 | 1.391595745 | | |
| 303 | 0.976974088 | 0.88589555 | 0.869038862 | 0.776664215 | 1.070215766 | 1.339738875 | 0.861701374 | 1.154023233 | 1.224093356 | | |
| 304 | 1 | 1 | 1 | 1 | 1 | 1 | 1 | 1 | 1 | | |
| 305 | 1.825939647 | 0.997632256 | 1.025247727 | 0.832722117 | 0.444067596 | 0.520908505 | 0.889901543 | 1.218541186 | 0.637514044 | | |
| 306 | 0.926558293 | 1.085760561 | 0.994264928 | 0.818256644 | 0.683880732 | 0.744098983 | 0.850403317 | 1.093196645 | 0.329819713 | | |
| 307 | 0.891841335 | 1.267662399 | 1.209103841 | 1.930930931 | 2.605579263 | 1.468942627 | 1.163033556 | 1.227772301 | 1.168108957 | | |
| 308 | 0.849525531 | 1 | 1 | 7.322340426 | 1.170319149 | 1 | 0.89881055 | 1.022776245 | 1.057231512 | | |
| 309 | 0.870979279 | 1.34261713 | 1.424699056 | 1.108564995 | 1.278704425 | 1.100355071 | 0.862149894 | 0.847112723 | 0.661495004 | | |
| 310 | 0.932356769 | 1.19745708 | 1.109585564 | 1.006553443 | 1.324372346 | 1.229993539 | 1.07556662 | 0.968310772 | 1.420413879 | | |
| 311 | 1 | 1.106922851 | 1.211938985 | 2.115869757 | 0.689351716 | 1.033880903 | 1.263617021 | 1.047340426 | 1 | | |
| 312 | 1 | 1.073829787 | 1.19606383 | 2.349255319 | 1.101382979 | 1 | 0.554299308 | 0.610376072 | 0.577740358 | | |
| 313 | 1 | 0.275098197 | 0.22932982 | 0.22932982 | 0.22932982 | 0.22932982 | 1 | 1 | 1 | | |
| 314 | 1 | 1 | 1 | 1 | 1 | 1 | 1 | 1 | 1 | | |
| 315 | 1.436797975 | 1.02479204 | 0.927418039 | 1.020714402 | 0.932392758 | 1.100717664 | 1.078406072 | 1.417077799 | 1.477419355 | | |
| 316 | 1.385204082 | 1.230957429 | 1.08024792 | 0.766595988 | 0.766595988 | 0.789920078 | 1.468191489 | 1.737978723 | 1.036382979 | | |
| 317 | 0.451528634 | 0.787155323 | 0.820942408 | 1.10617801 | 1.010471204 | 0.960139616 | 1.29847868 | 1.298978644 | 1.264195415 | | |
| 318 | 1 | 1 | 1 | 1.848085106 | 1.610319149 | 1 | 0.797340945 | 0.706076767 | 0.706076767 | | |
| 319 | 1.150796693 | 1.217403904 | 1.319756567 | 1.610032405 | 0.496851701 | 0.640777722 | 1.244050955 | 1.593019108 | 0.760840764 | | |
| 320 | 0.513400137 | 1.099541304 | 0.788592618 | 0.878166931 | 0.953916063 | 0.594547091 | 1.002166303 | 1.166762309 | 0.242669 | | |
| 321 | 1 | 0.308549986 | 0.126905791 | 0.183590306 | 0.108982216 | 0.101068748 | 1.084809875 | 0.935695799 | 0.935695799 | | |
| 322 | 0.933095096 | 0.748646066 | 0.748646066 | 0.760512902 | 0.748646066 | 0.791494106 | 1 | 1 | 1 | | |
| 323 | 0.961538462 | 0.839885633 | 0.839885633 | 0.967744818 | 0.839885633 | 0.839885633 | 0.985683885 | 0.909266783 | 0.909266783 | | |
| 324 | 0.716011072 | 1.045011371 | 1.465470266 | 1.076760262 | 1.372430254 | 1.327306298 | 1.038080433 | 1.059479655 | 1.245318352 | | |
| 325 | 0.702318006 | 0.962404779 | 1.037047186 | 1.413273415 | 1.903271771 | 2.180522826 | 1.210393027 | 1.080019734 | 1.479526394 | | |
| 326 | 0.657369126 | 0.890000632 | 0.784764767 | 0.91562981 | 0.415432023 | 0.649245917 | 0.961985616 | 0.983453199 | 0.508300438 | | |
| 327 | 0.542318122 | 1 | 1 | 1 | 1 | 1 | 1.157338649 | 0.468477448 | 0.468477448 | | |
| 328 | 1.005589028 | 1.323831022 | 1.286681716 | 2.356788133 | 1.069719445 | 1.328152209 | 1.211938403 | 0.950785839 | 0.503458599 | | |
| 329 | 1 | 0.951292728 | 0.910235306 | 0.929795681 | 1.117555921 | 0.910235306 | 1 | 1 | 1 | | |
| 330 | 0.764896454 | 1.107748336 | 0.940524928 | 1.909431119 | 3.084566118 | 1.658369961 | 1.436152271 | 1.130385897 | 1.607075453 | | |
| 331 | 1 | 1 | 1 | 1 | 1 | 1 | 1.129397669 | 0.615401372 | 1.198344407 | | |
| 332 | 0.991997216 | 1.076479716 | 0.936057538 | 0.476785142 | 0.378309811 | 0.650385477 | 1.106804944 | 1.11376616 | 0.430373633 | | |
| 333 | 1 | 1 | 1 | 1 | 1 | 1 | 0.673262216 | 0.547487956 | 0.540605643 | | |
| 334 | 1.094148936 | 1.293625914 | 1.347335423 | 0.982236155 | 0.982236155 | 0.982236155 | 0.884456154 | 0.884456154 | 0.886902522 | | |
| 335 | 0.841992118 | 0.98887379 | 0.936640054 | 0.887973501 | 0.998556141 | 0.979955835 | 1 | 1 | 1 | | |
| 336 | 1.28716161 | 1.148311848 | 1.39907919 | 1.496009822 | 0.956414979 | 0.902516882 | 1.277102804 | 1.420093458 | 1.837476636 | | |
| 337 | 1.239749577 | 1.400180397 | 1.661062029 | 0.608423146 | 0.46292613 | 1.01706832 | 1.346160298 | 1.280135539 | 0.963230114 | | |
| 338 | 1.290655749 | 1.124915505 | 1.11457577 | 0.754813109 | 0.610106902 | 1.005545402 | 0.994226652 | 0.931491945 | 0.720931457 | | |
| 339 | 1.017719569 | 0.966750652 | 0.887069198 | 0.427247368 | 0.422928102 | 0.588005039 | 0.854372543 | 0.914302412 | 0.610243332 | | |
| 340 | 0.853139498 | 0.850509599 | 0.994579365 | 1.165895269 | 1.641380296 | 1.675750698 | 0.767628102 | 0.734987254 | 0.753425014 | | |
| 341 | 0.897915863 | 1.006763681 | 0.963312154 | 2.008710801 | 2.539557286 | 1.43594999 | 0.80949364 | 0.689945904 | 1.166820995 | | |
| 342 | 0.800829154 | 0.887750302 | 0.729766215 | 0.944059234 | 0.702624205 | 1.072058252 | 1.381478085 | 1.240418118 | 1.530166881 | | |
| 343 | 0.576757884 | 1.095707599 | 0.936161737 | 0.936161737 | 0.936161737 | 0.936161737 | 1.134297681 | 0.543938571 | 0.503451485 | | |
| 344 | 0.950848076 | 1.200970055 | 1.346478279 | 1.425347954 | 2.29122733 | 1.822718965 | 0.885188733 | 0.621230121 | 0.95807626 | | |
| 345 | 1.109281052 | 0.901648582 | 1.005451591 | 0.841536929 | 0.627326099 | 1.148359281 | 0.771106445 | 1.163646629 | 1.367644724 | | |
| 346 | 1 | 1 | 1 | 1.157978723 | 1 | 1 | 1.000383757 | 0.621790399 | 0.774455763 | | |
| 347 | 0.783847058 | 0.734138057 | 0.782118161 | 0.624469247 | 1.847992236 | 0.992357152 | 1.065714792 | 1.301791415 | 1.212575381 | | |
| 348 | 0.871690504 | 1.20803583 | 1.368233156 | 1.974879917 | 1.695767883 | 1.119304167 | 0.941589595 | 0.728834893 | 0.974552619 | | |
| 349 | 0.81952862 | 1.169768108 | 1.190724306 | 1.985113083 | 1.594732322 | 1.189636416 | 0.937530374 | 0.886562449 | 1.07077191 | | |
| 350 | 1.163830439 | 0.900792514 | 0.948152392 | 0.966262453 | 1.001527738 | 0.840415035 | 0.853625105 | 1.061753036 | 0.767055429 | | |
| 351 | 1.452159592 | 0.743307778 | 0.867549498 | 0.5266011 | 0.359508506 | 0.686517463 | 0.999440881 | 1.662818615 | 0.56921559 | | |
| 352 | 1 | 1 | 1 | 1.106595745 | 1 | 1 | 0.707296701 | 1.011842969 | 0.822623948 | | |
| 353 | 1 | 1 | 1 | 1.943297872 | 1.147340426 | 1 | 1.11185 | 0.84805 | 0.86045 | | |
| 354 | 1.068264399 | 0.898347626 | 0.821323991 | 1.368803598 | 1.436937879 | 1.30140138 | 1.076373491 | 0.829853412 | 0.445306726 | | |
| 355 | 0.780587924 | 1.144569918 | 0.859303589 | 0.752853893 | 0.393581046 | 0.68845941 | 1.052631579 | 1.201913442 | 0.805315646 | | |
| 356 | 1 | 1 | 1 | 1 | 1 | 1 | 1 | 1 | 1 | | |
| 357 | 0.757855715 | 0.900445376 | 0.763601432 | 0.879966815 | 1.140730067 | 0.77827264 | 0.860733441 | 0.816702008 | 0.753897967 | | |
| 358 | 1.392420293 | 1.350475466 | 1.232940281 | 2.140129327 | 1.513883606 | 1.033320654 | 1 | 1 | 1 | | |
| 359 | 1.099307637 | 1 | 1 | 1 | 1 | 1 | 1 | 1 | 1 | | |
| 360 | 0.908153117 | 1.282813679 | 1.314013913 | 0.499916185 | 0.509093957 | 1.110699019 | 0.875420875 | 1.191646192 | 1.172354172 | | |
| 361 | 0.813409355 | 1.221319556 | 1.201712429 | 0.812983789 | 1.190722214 | 1.377939243 | 1.100789314 | 1.032695811 | 1.1423436551 | | |
| 362 | 1.070081398 | 0.828597737 | 0.947119179 | 0.541568008 | 1.232131895 | 1.233008857 | 1 | 1 | 1 | | |
| 363 | 1 | 1 | 1 | 1.296489362 | 1 | 1 | 1 | 1 | 1.987340426 | | |
| 364 | 1.084540489 | 1.294933562 | 1.1677356 | 1.503544358 | 1.791358517 | 1.322551938 | 0.853420773 | 0.880024814 | 1.332435307 | | |
| 365 | 1 | 0.830232817 | 0.713267238 | 0.543667149 | 0.769633508 | 0.911217556 | 1.468191489 | 1.602765957 | 1.454574468 | | |
| 366 | 1.212853084 | 1.269082359 | 1.337694441 | 0.441336067 | 0.483118293 | 1.09713011 | 1.340722496 | 1.091885605 | 1.338464696 | | |
| 367 | 1.358742707 | 1.051496149 | 1.028610865 | 0.835917797 | 0.489400859 | 1.016341763 | 1.062207684 | 0.84448079 | 1.178818398 | | |
| 368 | 1 | 1 | 1 | 1 | 1 | 1 | 1.323323479 | 2.150044085 | 1.161402417 | | |
| 369 | 1.166799643 | 0.94137418 | 0.983910959 | 4.27175051 | 0.713372638 | 0.838778996 | 0.722301886 | 0.812775628 | 0.865533941 | | |
| 370 | 1.209814452 | 0.991910278 | 0.985263835 | 2.595615003 | 0.60965251 | 0.717990439 | 1.395051125 | 1.154569381 | 0.881961592 | | |
| 371 | 1.071595745 | 1 | 1 | 1 | 1 | 1 | 1.145595855 | 0.347890452 | 0.484122872 | | |
| 372 | 0.971375426 | 0.898197199 | 0.708718328 | 0.745226069 | 0.509424664 | 0.800583138 | 0.832904162 | 0.828690482 | 0.44009551 | | |
| 373 | 0.908291738 | 1.11114937 | 0.979477997 | 0.942083879 | 1.235658701 | 1.163143034 | 1 | 1 | 1.17893617 | | |
| 374 | 1.252272397 | 1.069077926 | 0.998358983 | 0.441477802 | 0.272697033 | 0.644786446 | 1.285152864 | 1.274345489 | 1.068311853 | | |
| 375 | 1.850711601 | 0.799503079 | 0.972669331 | 0.989377408 | 1.426614094 | 1.806416766 | 0.948859935 | 0.676547231 | 0.721009772 | | |
| 376 | 1 | 1.089837528 | 1.111420835 | 1.793883402 | 1.93501115 | 1.261070405 | 0.808115543 | 0.995787483 | 0.908872077 | | |
| 377 | 0.916495081 | 0.944628016 | 0.949467641 | 1.100697574 | 1.9155902671 | 1.337705684 | 0.966339467 | 0.737193498 | 0.87445853 | | |
| 378 | 1 | 1 | 1 | 1 | 1 | 1 | 1 | 1 | 1 | | |
| 379 | 1.416540671 | 1.102765957 | 1.376382979 | 1 | 1 | 1.012659574 | 1 | 1 | 1 | | |
| 380 | 0.896221173 | 1.089229792 | 1.199337358 | 1.611764172 | 1.934825035 | 1.207053057 | 0.822564663 | 0.670482113 | 1.038815048 | | |
| 381 | 0.636209814 | 1.929042553 | 3.302234043 | 6.955212766 | 1.681170213 | 1 | 1 | 1 | 1 | | |
| 382 | 0.688394031 | 0.956948292 | 0.807363804 | 0.839950754 | 1.138504155 | 0.990304709 | 1.008191489 | 1 | 1.508106383 | | |
| 383 | 1 | 1 | 1 | 1 | 1 | 1 | 0.686673165 | 0.506510235 | 0.772838045 | | |
| 384 | 1 | 1 | 1 | 1.198085106 | 1 | 1 | 0.911148649 | 1.433004158 | 0.869984407 | | |
| 385 | 1.258908698 | 1.133128531 | 1.363820049 | 0.820213599 | 0.98696961 | 1.14176735 | 1.005333792 | 0.915290204 | 1.455838495 | | |
| 386 | 1.034021238 | 0.892464956 | 0.872071251 | 0.454632863 | 0.767673701 | 0.745382319 | 0.728719492 | 0.628552324 | 1.425743898 | | |
| 387 | 0.919223206 | 1.001594833 | 1.001914942 | 0.667314508 | 0.433022751 | 0.771064365 | 1.135632345 | 1.279908788 | 1.017042624 | | |
| 388 | 0.851653483 | 1 | 1 | 2.052765957 | 1 | 1 | 0.933880996 | 0.786216928 | 1.142245485 | | |
| 389 | 0.844941039 | 0.969870875 | 1.016914596 | 0.709808956 | 0.838858265 | 0.896851167 | 1.011521441 | 1.019609339 | 1.178238975 | | |
| 390 | 1.284091547 | 1.127131938 | 1.064550582 | 1.159340476 | 0.933713828 | 0.919428591 | 1.175109346 | 1.32934206 | 1.084313056 | | |
| 391 | 1 | 1 | 1 | 1.661170213 | 1 | 1 | 1 | 1 | 1 | | |
| 392 | 0.835988888 | 1.04666923 | 1.17079007 | 0.949177456 | 0.916600436 | 1.130521728 | 1.02395432 | 1.028571125 | 1.007609769 | | |
| 393 | 1.023439523 | 1.056842756 | 0.725980846 | 0.725980846 | 0.824606117 | 0.754556688 | 0.6118221 | 0.949142914 | 0.712467003 | | |
| 394 | 0.818195266 | 0.91490272 | 0.706285016 | 0.896118977 | 1.008169502 | 0.480167057 | 0.688183737 | 0.713837886 | 0.601369074 | | |
| 395 | 1 | 1.535531915 | 1.555851064 | 1.599893617 | 1 | 1 | 1 | 1 | 1 | | |
| 396 | 1 | 1 | 1 | 1 | 1 | 1 | 1 | 1 | 1 | | |
| 397 | 0.986647626 | 1.003502962 | 1.003248201 | 0.825234062 | 1.049232533 | 1.24164066 | 1.109353508 | 1.235534159 | 0.946309033 | | |
| 398 | 0.993194053 | 0.876555781 | 0.905250387 | 0.644940802 | 0.596347368 | 0.767388455 | 0.671020071 | 1.174388232 | 0.676862799 | | |
| 399 | 0.989700196 | 1.229515443 | 1.582518371 | 1.372921156 | 2.046190397 | 1.890546439 | 0.814625629 | 0.65972613 | 0.994776561 | | |
| 400 | 1.119118523 | 1 | 1 | 1 | 1 | 1 | 1 | 1 | 1.17893617 | | |
| 401 | 1.138726636 | 1 | 1 | 1 | 1 | 1 | 1 | 1 | 1 | | |
| 402 | 1 | 1 | 1 | 1 | 1 | 1 | 1 | 1 | 1 | | |
| 403 | 1 | 1 | 1 | 1 | 1 | 1.039361702 | 1 | 1 | 1 | | |
| 404 | 1.261545413 | 0.961923848 | 1.13421128 | 0.697795591 | 0.629888348 | 0.997537933 | 1 | 1.20212766 | 1.30712766 | | |
| 405 | 1.321638892 | 0.76947241 | 0.946005098 | 0.840744651 | 0.33895512 | 0.719133816 | 1.015603217 | 1.203538874 | 0.944530831 | | |
| 406 | 0.775527654 | 0.885811294 | 1.062365976 | 0.886436741 | 1.421908506 | 1.383488206 | 0.66494772 | 0.489437371 | 0.994203002 | | |
| 407 | 1.10125401 | 1.068344334 | 1.238758251 | 0.385313531 | 0.478788504 | 0.762066832 | 1 | 1 | 1 | | |
| 408 | 1 | 2.112234043 | 2.323191489 | 3.055425532 | 1.516595745 | 1 | 1 | 1 | 1 | | |
| 409 | 0.91112949 | 1.111936029 | 1.110659144 | 0.785217845 | 0.937758182 | 0.881069549 | 0.952445479 | 0.791489815 | 0.867141313 | | |
| 410 | 1 | 1 | 1 | 1 | 1 | 1 | 0.695634077 | 0.560583312 | 1.207568393 | | |
| 411 | 0.680095244 | 0.778984006 | 1.138062484 | 1.064556228 | 1.254412862 | 0.90014088 | 1.165278904 | 1.147630978 | 1.236828531 | | |
| 412 | 0.967318943 | 0.810306327 | 0.788834812 | 0.538219296 | 0.538219296 | 0.564214143 | 1 | 1 | 1 | | |
| 413 | 0.855890814 | 1.257939472 | 1.211351755 | 0.998150717 | 1.172552367 | 1.294285003 | 1 | 1 | 1 | | |
| 414 | 0.910166542 | 1.0339565G5 | 1.359023274 | 1.63801984 | 1.841949638 | 2.134586036 | 0.942946248 | 0.887360117 | 1.18651012 | | |
| 415 | 1.122082766 | 1.064908722 | 0.860254083 | 2.032454361 | 2.034803032 | 1.288192591 | 0.957985224 | 0.776772768 | 0.846509701 | | |
| 416 | 0.900566496 | 1.09606502 | 0.982545745 | 1.045434358 | 1.503330965 | 1.202300586 | 1 | 1 | 1.444042553 | | |
| 417 | 0.992327163 | 1.024110032 | 1.078883495 | 0.898058252 | 0.820064725 | 0.933495146 | 1.012591815 | 1.158160832 | 1.903462749 | | |
| 418 | 0.98046046 | 0.921979746 | 0.946560167 | 0.848785242 | 0.903199734 | 0.709432796 | 1.032142028 | 1.29890926 | 1.904618241 | | |
| 419 | 1.22671022 | 0.949252113 | 0.927018844 | 0.550725293 | 0.580143703 | 0.905689367 | 0.993762554 | 0.993762554 | 0.993762554 | | |
| | U | V | W | X | Y | Z | AA | AB | AC | | |
| 1 | DR.JRF | DR.JRFnCMS | LC.CMS | LC.IMP | LC.IMPnCMS | LC.JRF | LC.JRFnCMS | MG.CMS | MG.IMP | | |
| 2 | 1.611129651 | 1.156933089 | 0.928919578 | 1.433143786 | 1.512059671 | 1.589203161 | 1.703020456 | 1.042762294 | 1.038673619 | | |
| 3 | 1.232021277 | 1 | 1 | 1 | 1 | 1 | 1 | 1 | 1 | | |
| 4 | 1.342021277 | 1.421170213 | 1.410920595 | 1.21750798 | 0.934493376 | 0.789073755 | 0.739103415 | 1.026808511 | 1 | | |
| 5 | 1.042978723 | 1.243510638 | 0.965282552 | 0.932283789 | 1.001 993675 | 1.005499794 | 0.717998075 | 1 | 1 | | |
| 6 | 1.372321998 | 1.765429848 | 0.936288089 | 0.906759003 | 0.852022161 | 1.087977839 | 0.939778393 | 0.871344323 | 0.916130903 | | |
| 7 | 1.378297872 | 1 | 1 | 1 | 1 | 1 | 1 | 1 | 1 | | |
| 8 | 1.307197592 | 1.105090312 | 0.827537635 | 0.894885113 | 0.827537635 | 0.827537635 | 1.004225724 | 0.857719418 | 0.754092769 | | |
| 9 | 2.14092585 | 3.001736993 | 1.236828394 | 1.089150874 | 1.270221089 | 1.304873591 | 1.023718612 | 0.990974006 | 0.92388771 | | |
| 10 | 0.815831495 | 0.957264228 | 0.870267347 | 0.940529542 | 1.030726833 | 1.099495184 | 1.808195539 | 1.124058627 | 0.871162503 | | |
| 11 | 1.584361702 | 1.537553191 | 1.229574468 | 1 | 1.464361702 | 1.351914894 | 1.341489362 | 0.938704225 | 0.781521127 | | |
| 12 | 1.253262813 | 1.25276816 | 1.863879575 | 1.351287722 | 1.659984305 | 1.477491617 | 2.075408433 | 0.986408256 | 0.883513718 | | |
| 13 | 0.998088998 | 0.855400855 | 1 | 1 | 1 | 1 | 1 | 1 | 1 | | |
| 14 | 1 | 1 | 1 | 1 | 1 | 1 | 1 | 0.80129047 | 0.91943128 | | |
| 15 | 2.113429664 | 1.655843604 | 1.154433323 | 1.978970782 | 2.296683854 | 2.702102922 | 2.668385401 | 0.88520216 | 0.720711954 | | |
| 16 | 1 | 1 | 1 | 1 | 1 | 1 | 1 | 1 | 1 | | |
| 17 | 1 | 1 | 1 | 1 | 1 | 1 | 1 | 1 | 1 | | |
| 18 | 2.120359367 | 1.521937539 | 0.774219681 | 0.750917322 | 0.732856802 | 0.716511794 | 0.915034548 | 1.126663586 | 0.850600739 | | |
| 19 | 1.119017886 | 0.992487109 | 1.104549758 | 0.913738974 | 0.821248816 | 0.692804106 | 0.97931928 | 1.196504562 | 0.94600479 | | |
| 20 | 1 | 1 | 1.185009336 | 0.57113897 | 0.67607362 | 0.802133902 | 0.501467058 | 1 | 1 | | |
| 21 | 1.97763864 | 2.017531306 | 0.883091149 | 1.273147592 | 1.374564669 | 1.993154798 | 1.795784796 | 0.962793418 | 0.972991531 | | |
| 22 | 1 | 1 | 0.534945144 | 0.77021536 | 0.632629013 | 0.705526209 | 0.617106867 | 0.872813368 | 1.366414902 | | |
| 23 | 1.279747183 | 1.542639919 | 1 | 1 | 1 | 1 | 1 | 1.055759121 | 1.046120758 | | |
| 24 | 1.224330357 | 0.892485119 | 0.466310311 | 0.552445257 | 0.594223285 | 0.749613166 | 0.652318657 | 0.920070679 | 0.744413263 | | |
| 25 | 2.283471074 | 3.206198347 | 1 | 1.253404255 | 1 | 1 | 1.06606383 | 0.968927355 | 0.977617764 | | |
| 26 | 0.866758875 | 0.904564315 | 1 | 1 | 1 | 1.233617021 | 1.050851064 | 1.210088404 | 1.138429537 | | |
| 27 | 1.286849526 | 1.543194053 | 0.883209621 | 1.257540167 | 1.742553791 | 2.083623039 | 1.81574744 | 1.021905762 | 0.940119062 | | |
| 28 | 1 | 1 | 1 | 1 | 1 | 1 | 1 | 1 | 1 | | |
| 29 | 1.025466143 | 0.754048086 | 1.601727147 | 1.373797485 | 2.177726073 | 2.428296568 | 2.257461184 | 0.882703383 | 0.962997286 | | |
| 30 | 1 | 1 | 2.772124469 | 2.532634134 | 3.809395857 | 4.503857155 | 4.162953974 | 1 | 1 | | |
| 31 | 0.529070749 | 0.529070749 | 1.282828283 | 1.227144325 | 1.30671118 | 1.415254237 | 1.509972607 | 0.929256339 | 0.722885148 | | |
| 32 | 1 | 1 | 1.156779152 | 0.824906929 | 1.090068452 | 1.195268404 | 0.980785397 | 1 | 1 | | |
| 33 | 0.464979314 | 0.62404478 | 0.856679614 | 0.931557836 | 1.009714008 | 0.755246481 | 0.562665219 | 1.094315041 | 1.997275957 | | |
| 34 | 0.8406037 | 0.718787963 | 1.001684042 | 0.836701417 | 0.942502005 | 0.699906442 | 1.023509757 | 1.211821725 | 0.83589065 | | |
| 35 | 1.232098537 | 0.835969624 | 0.912567364 | 1.447596465 | 1.757016598 | 1.728950205 | 2.065186463 | 1.00325032 | 0.771899393 | | |
| 36 | 0.949494949 | 0.949494949 | 1 | 1 | 1 | 1 | 1 | 0.894378651 | 1.002920529 | | |
| 37 | 0.608917398 | 0.563436563 | 0.595804019 | 0.609875135 | 0.670342904 | 0.616720543 | 0.790898431 | 0.946547782 | 0.795976223 | | |
| 38 | 1.231274539 | 1.500679553 | 0.500201045 | 0.651655274 | 0.733246214 | 0.675981772 | 0.647533843 | 0.888424522 | 0.738381857 | | |
| 39 | 1.278085106 | 1 | 1 | 1 | 1 | 1 | 1 | 1 | 1 | | |
| 40 | 0.53972894 | 0.615997874 | 1.350202545 | 1.386717636 | 1.190506076 | 0.74861642 | 1.017801107 | 1.153621068 | 0.885241405 | | |
| 41 | 2.152956094 | 1.77746077 | 0.745854162 | 1.298659049 | 1.199079584 | 0.864000635 | 1.355946997 | 0.886341129 | 0.716081359 | | |
| 42 | 1.430358476 | 1.572115385 | 0.569617515 | 0.907255971 | 0.934795777 | 0.798221703 | 0.910263067 | 0.789662265 | 0.66654502 | | |
| 43 | 1.074961405 | 1.084966682 | 0.55864057 | 0.708086785 | 0.702260658 | 0.752935822 | 0.718585951 | 0.98219956 | 0.878525901 | | |
| 44 | 1.465497037 | 1.356379951 | 1.380028055 | 1.598741328 | 1.503279372 | 1.316525761 | 1.422451378 | 0.928481807 | 0.753615532 | | |
| 45 | 1 | 1 | 0.930600931 | 1.765468765 | 1.46955747 | 1.850806851 | 2.082566083 | 1 | 1 | | |
| 46 | 2.283784687 | 2.959946506 | 0.756478352 | 1.012795751 | 0.804764204 | 0.899163045 | 0.907130211 | 0.843546089 | 1.154068852 | | |
| 47 | 1 | 1.320106383 | 1 | 1 | 1 | 1 | 1 | 0.912354135 | 1.058219178 | | |
| 48 | 0.531043444 | 0.628382577 | 1.36704157 | 0.985466317 | 0.918726981 | 1.058943996 | 0.759979521 | 1 | 1.046808511 | | |
| 49 | 0.419417391 | 0.534772511 | 0.617922839 | 0.436102741 | 0.27783982 | 0.292937061 | 0.396521208 | 1.22504075 | 1.351212306 | | |
| 50 | 0.793095718 | 1.033325861 | 1.180851064 | 1.639255319 | 1.63606383 | 2.131914894 | 2.186170213 | 0.949168392 | 1.055974885 | | |
| 51 | 1.098097079 | 1.295105322 | 0.583356471 | 0.604260045 | 0.668002063 | 0.601800801 | 0.667883037 | 0.916693864 | 0.836569843 | | |
| 52 | 1 | 1 | 0.451640801 | 0.970739442 | 1.177917648 | 1.116369577 | 0.904819103 | 1 | 1 | | |
| 53 | 0.553450259 | 0.366885023 | 3.54637032 | 1.360416549 | 0.740186351 | 1.211808509 | 1.22792992 | 1.234755417 | 1.567946045 | | |
| 54 | 1.432446809 | 1.148723404 | 1 | 1 | 1 | 1 | 1 | 1 | 1 | | |
| 55 | 1.460350173 | 1.265138618 | 0.712224067 | 1.077163508 | 1.637819887 | 2.030889822 | 2.205435632 | 0.979894018 | 0.973177487 | | |
| 56 | 1.477340426 | 1.43787234 | 1 | 1.04287234 | 1.017340426 | 1 | 1 | 1.245265503 | 0.872632752 | | |
| 57 | 1.614341247 | 1.670414646 | 0.899364924 | 1.315974597 | 1.246116268 | 1.307718613 | 1.312652662 | 1.128036015 | 0.97430387 | | |
| 58 | 1.7171089151 | .776710706 | 0.988396636 | 1.007054574 | 1.283988938 | 1.922027202 | 1.668480163 | 0.787709938 | 0.777748574 | | |
| 59 | 1.69731947 | 1.21406086 | 1.268063584 | 1.489956647 | 1.932153179 | 1.726445087 | 2.051372832 | 1 | 1 | | |
| 60 | 1.240213362 | 1.946388211 | 0.947580645 | 0.947580645 | 0.947580645 | 0.947580645 | 0.947580645 | 1.142158338 | 1.222757375 | | |
| 61 | 1.160330579 | 1.298347107 | 0.647796045 | 0.746771503 | 0.963831308 | 0.826495116 | 0.748248749 | 0.789441914 | 0.810678812 | | |
| 62 | 1 | 1 | 1.12563 | 1.319722992 | 1.947146814 | 1.475844875 | 2.344487535 | 1 | 1 | | |
| 63 | 1.011841846 | 1.20771097 | 1.404916619 | 0.914558493 | 1.640632557 | 1.584821147 | 1.628898282 | 1.104914617 | 1.114362407 | | |
| 64 | 1.019390376 | 1.010250963 | 0.508779856 | 1.163825526 | 1.211681239 | 1.458139989 | 1.269892053 | 1.161122904 | 0.864404188 | | |
| 65 | 1.167540936 | 1.379394291 | 0.630957175 | 0.951738488 | 0.822862129 | 1.309101893 | 1.155457108 | 1.110648494 | 0.956544509 | | |
| 66 | 0.602417457 | 0.728756517 | 1.254577995 | 1.025847777 | 1.2860211 | 0.860870384 | 1.034118312 | 1.04833124 | 1.154675235 | | |
| 67 | 2.723510638 | 2.034148936 | 1.180851064 | 1.329255319 | 2.219787234 | 2.611808511 | 2.302340426 | 1.095943236 | 0.952182631 | | |
| 68 | 1.262230194 | 1.177871235 | 0.497450765 | 1.100803092 | 0.997067546 | 0.969542471 | 0.812889466 | 1.07197757 | 1.02605501 | | |
| 69 | 1 | 1 | 0.496881277 | 0.754202347 | 0.734749974 | 0.72259224 | 0.617295697 | 1 | 1 | | |
| 70 | 1.575425532 | 1.62 | 1 | 1 | 1 | 1 | 1 | 0.847556122 | 0.748288489 | | |
| 71 | 1.049708481 | 1.185550279 | 1.328915744 | 0.670949983 | 0.498046324 | 0.474843907 | 0.365505203 | 1 | 1 | | |
| 72 | 1 | 1 | 1 | 1 | 1 | 1 | 1 | 0.815684949 | 0.794388574 | | |
| 73 | 1.356760259 | 1.156025918 | 1.296123599 | 0.844804788 | 0.654186095 | 0.654186095 | 0.654186095 | 1.030224435 | 0.817131791 | | |
| 74 | 1 | 1 | 1.703444391 | 1.084178336 | 1.162079511 | 0.756478352 | 0.771849348 | 1 | 1 | | |
| 75 | 2.20031991 | 2.405626647 | 0.96165356 | 1.199248382 | 1.317767416 | 1.708608811 | 2.048924769 | 0.996082699 | 0.762948857 | | |
| 76 | 1.575906613 | 1.524088496 | 0.890982584 | 0.7496882 | 0.999924867 | 1.091481465 | 0.670934199 | 0.831016278 | 0.952419733 | | |
| 77 | 1.537370539 | 1.597928623 | 1 | 1 | 1 | 1 | 1 | 0.914837223 | 0.95303947 | | |
| 78 | 1.957809144 | 1.93840135 | 1 | 1 | 1 | 1 | 1 | 1 | 1 | | |
| 79 | 1.820021564 | 1.504188438 | 1.227530078 | 0.940299262 | 1.11257709 | 1.147811141 | 1.026488727 | 0.981981335 | 0.751830581 | | |
| 80 | 0.73804107 | 0.499550979 | 0.636327024 | 0.886494832 | 0.951201504 | 0.871526379 | 0.985568533 | 0.868985849 | 0.897287736 | | |
| 81 | 1 | 1 | 1.173013608 | 0.552842406 | 0.515803336 | 0.765144864 | 0.515803336 | 1 | 1 | | |
| 82 | 1.241276596 | 1.157340426 | 1 | 1 | 1 | 1 | 1 | 1 | 1 | | |
| 83 | 1.834029518 | 1.865868375 | 1 | 1 | 1 | 1 | 1 | 1.082630945 | 0.707405178 | | |
| 84 | 1.69970487 | 2.292097065 | 0.767379176 | 0.783238262 | 0.724942017 | 0.591299442 | 0.680749702 | 1.217244554 | 0.984657871 | | |
| 85 | 1 | 1 | 0.453024332 | 0.479170576 | 0.293988866 | 0.478763996 | 0.293988866 | 0.946036904 | 0.866194731 | | |
| 86 | 0.958671501 | 0.592572999 | 1.013239287 | 0.944159879 | 1.610882526 | 1.480952781 | 1.997200996 | 0.927551583 | 1.044476389 | | |
| 87 | 1.006635809 | 0.891123582 | 0.99263376 | 1.152486812 | 1.411567445 | 0.895685757 | 0.829559156 | 0.831346969 | 0.891501584 | | |
| 88 | 1.826611778 | 1.820401898 | 0.404818059 | 0.641004043 | 0.668463612 | 0.871226415 | 0.761623989 | 1.031948593 | 0.896596977 | | |
| 89 | 1 | 1 | 1.105901563 | 0.692725516 | 0.733413309 | 0.555366723 | 0.5147789 | 1 | 1 | | |
| 90 | 1.625949853 | 1.253066568 | 1.121393901 | 1.031229966 | 0.855160729 | 0.556552798 | 1.013142229 | 0.911511355 | 0.818444672 | | |
| 91 | 1.731089043 | 1.479687352 | 1.056676273 | 1.193497632 | 1.269849945 | 1.370250754 | 1.60469045 | 0.949679338 | 0.882079895 | | |
| 92 | 1.111623345 | 0.829671408 | 1.25393617 | 1 | 1.420957447 | 1.816276596 | 1.425 | 1.023030104 | 1.065964797 | | |
| 93 | 1.897227128 | 1.77455846 | 1.184923072 | 1.198903748 | 0.949402439 | 1.193144958 | 0.931518968 | 0.983965903 | 1.134486627 | | |
| 94 | 0.881034075 | 0.799574065 | 1 | 1 | 1 | 1 | 1 | 1.294787234 | 1 | | |
| 95 | 1.413463558 | 1.207834489 | 0.693984525 | 0.840502538 | 1.259963391 | 1.0104002 | 0.91039188 | 0.8433033 | 0.989184842 | | |
| 96 | 2.95755751 | 2.621054856 | 1.186911575 | 1.103101348 | 1.033228724 | 1.573149345 | 1.628161344 | 1.030046108 | 0.861105819 | | |
| 97 | 0.382084802 | 0.462816562 | 0.722820247 | 0.842597737 | 1.063114405 | 1.209901393 | 1.067529149 | 1.203566852 | 1.309272418 | | |
| 98 | 0.641895811 | 0.477254138 | 1.083803886 | 1.032127419 | 0.888917014 | 0.430828693 | 0.507554811 | 0.874254758 | 0.86299014 | | |
| 99 | 1.637553191 | 1.286170213 | 1.131808511 | 1.196170213 | 1.478723404 | 1.974574468 | 1.963829787 | 1.045696371 | 0.893026791 | | |
| 100 | 1 | 1 | 1.739965096 | 1.555376561 | 1.728755538 | 1.805544368 | 2.132635253 | 1 | 1 | | |
| 101 | 1 | 1 | 0.638500204 | 0.721097677 | 0.638500204 | 0.638500204 | 0.638500204 | 1 | 1 | | |
| 102 | 2.369311432 | 2.217302257 | 1.495113077 | 1.149104659 | 1.295141614 | 2.158165085 | 2.175215809 | 1.011974181 | 0.871464654 | | |
| 103 | 1.35355286 | 1.200828507 | 1 | 1 | 1 | 1.307765957 | 1.17 | 1.089723368 | 0.967908275 | | |
| 104 | 0.937735085 | 0.855936462 | 1.248323618 | 1.108248812 | 0.890015837 | 0.965191899 | 0.965242444 | 0.997082821 | 0.736820781 | | |
| 105 | 1.270029838 | 1.106531108 | 1.974352083 | 0.951698983 | 0.960376272 | 0.477289307 | 0.598003456 | 1.106402722 | 0.970770182 | | |
| 106 | 1.831595745 | 1.479574468 | 1.033810376 | 1.205008945 | 0.996690519 | 0.84078712 | 0.883542039 | 0.922787376 | 0.907248335 | | |
| 107 | 1.333922285 | 1.052040683 | 1.115858587 | 0.944515691 | 0.670052375 | 0.689938249 | 0.558380572 | 1.179071469 | 1.226367997 | | |
| 108 | 0.799168516 | 0.804091106 | 1 | 1 | 1 | 1 | 1 | 0.794425986 | 0.855682075 | | |
| 109 | 1 | 1.11393617 | 0.983675178 | 0.983675178 | 1.326182503 | 1.109564671 | 0.983675178 | 0.88612368 | 0.88612368 | | |
| 110 | 1.206452194 | 1.449400739 | 0.836294516 | 0.902960311 | 1.001633005 | 0.963917122 | 0.796318554 | 0.871224897 | 1.040165568 | | |
| 111 | 2.339048832 | 2.453352175 | 1.080214356 | 1.199357947 | 1.557072102 | 1.052760025 | 1.173599685 | 0.840948461 | 0.722667997 | | |
| 112 | 0.378650832 | 0.324518401 | 1 | 1 | 1 | 1 | 1 | 1.454978038 | 1.508756617 | | |
| 113 | 0.805106642 | 0.728253711 | 1.476328079 | 1.002656159 | 0.63502627 | 0.305283129 | 0.556859311 | 0.828264006 | 0.953744891 | | |
| 114 | 0.412230029 | 0.447650273 | 1.418761259 | 0.862304079 | 0.720938063 | 0.750826333 | 0.644051247 | 1.118098608 | 1.379272291 | | |
| 115 | 0.640152547 | 0.640152547 | 1.588451792 | 0.970335063 | 0.667772817 | 0.556614778 | 0.552190177 | 1.193320022 | 1.204029769 | | |
| 116 | 1.320476264 | 1.501204146 | 1.378149389 | 1.104858556 | 0.960121405 | 1.096116437 | 1.359657346 | 1.093302316 | 1.005697964 | | |
| 117 | 0.434842948 | 0.434842948 | 1.217395015 | 1.119665415 | 1.163280983 | 0.941532946 | 0.962103107 | 1 | 1 | | |
| 118 | 1 | 1 | 1 | 1 | 1 | 1 | 1 | 1 | 1 | | |
| 119 | 0.693666202 | 1.026692335 | 0.49564269 | 0.555458132 | 0.65225992 | 0.577565467 | 0.624512142 | 0.922121241 | 0.954443559 | | |
| 120 | 1.472345405 | 1.226071693 | 0.895900125 | 0.949750041 | 0.994199315 | 0.767008097 | 0.926615226 | 0.853587371 | 0.904278715 | | |
| 121 | 1 | 1 | 1 | 1 | 1 | 1 | 1 | 1 | 1 | | |
| 122 | 1.119680851 | 1.157340426 | 1 | 1 | 1 | 1 | 1 | 0.908104323 | 0.822685104 | | |
| 123 | 0.601369074 | 0.601369074 | 0.605175139 | 0.878523909 | 0.743411622 | 0.790642114 | 0.732862866 | 1 | 1.197765957 | | |
| 124 | 1 | 1 | 1.820957447 | 1.196170213 | 1 | 1 | 1 | 1 | 1 | | |
| 125 | 1 | 1.096382979 | 0.948575567 | 0.756478352 | 0.781345566 | 1.623531305 | 0.982858522 | 1 | 1 | | |
| 126 | 0.820198182 | 0.942804333 | 0.715699711 | 0.715699711 | 0.943962235 | 0.718212273 | 0.74120603 | 0.807369371 | 0.983554229 | | |
| 127 | 0.607094312 | 0.680024049 | 1.441950976 | 1.080838377 | 0.604353751 | 0.445069299 | 0.514077089 | 1.061354489 | 1.25245389 | | |
| 128 | 1.396382979 | 1.052446809 | 1.196170213 | 1.245106383 | 1.278191489 | 1.817978723 | 0.947479912 | 0.788093499 | | | |
| 129 | 0.963806008 | 0.963806008 | 1 | 1 | 1 | 1.003510638 | 1 | 1 | 1 | | |
| 130 | 1.236588417 | 1.379203768 | 1 | 1 | 1 | 1 | 1 | 0.951117917 | 1.039816233 | | |
| 131 | 0.586528343 | 0.738943551 | 1.130290134 | 0.810065617 | 0.756762924 | 0.876115477 | 0.755930195 | 1.123079597 | 1.418534932 | | |
| 132 | 0.871188133 | 0.762537529 | 1.119334065 | 0.988280278 | 0.888218075 | 0.796084888 | 0.738569338 | 1.040234239 | 1.045128397 | | |
| 133 | 0.370794052 | 0.400023668 | 1.008510638 | 1 | 1.200425532 | 1 | 1 | 1 | 1.063723404 | | |
| 134 | 1.50235647 | 2.087189374 | 0.214881701 | 0.473311236 | 0.792341982 | 0.614904561 | 0.686341296 | 1.095309576 | 1.056322099 | | |
| 135 | 1.157446809 | 1 | 1.854477412 | 2.269099171 | 3.119553595 | 3.369326383 | 4.406789628 | 1.460618146 | 1.413160518 | | |
| 136 | 1 | 1.226382979 | 1 | 1.338829787 | 1.406489362 | 1.439680851 | 1.09606383 | 0.835271558 | 0.858097241 | | |
| 137 | 0.580555125 | 0.489843387 | 0.965290614 | 0.965290614 | 0.965290614 | 0.965290614 | 0.965290614 | 0.969272015 | 1.193235719 | | |
| 138 | 2.087226091 | 2.457363683 | 0.515803336 | 0.883834504 | 0.78500878 | 0.996268657 | 0.819743196 | 0.998329828 | 0.89533589 | | |
| 139 | 1.671742291 | 1.205830591 | 1.769357411 | 1.242167001 | 1.066934198 | 1.383906982 | 1.742707208 | 0.886316324 | 0.971287129 | | |
| 140 | 0.415125535 | 0.521245649 | 0.422180759 | 0.414201632 | 0.29149601 | 0.266284397 | 0.455068111 | 1.311887823 | 1.506495515 | | |
| 141 | 0.508703961 | 0.456820502 | 2.247943284 | 1.155135156 | 2.1235167 | 0.698563142 | 0.4427342 | 1 | 1 | | |
| 142 | 0.874423859 | 1.050655219 | 0.810258757 | 1.374240785 | 0.999167984 | 1.221066644 | 1.108037274 | 1.063616121 | 1.008906702 | | |
| 143 | 1.650107797 | 1.893550126 | 0.602525479 | 0.823729248 | 0.8298827 | 0.955002884 | 0.825139414 | 0.940375736 | 0.896274479 | | |
| 144 | 1.204081633 | 1.524430926 | 1 | 1.100531915 | 2.055744681 | 2.017553191 | 1.77787234 | 0.95710898 | 0.992576554 | | |
| 145 | 1 | 1 | 0.863680789 | 0.750520546 | 0.544341988 | 0.480771682 | 0.461479624 | 1 | 1 | | |
| 146 | 0.57766974 | 0.721005868 | 1.080999188 | 1.109063035 | 0.911750383 | 0.949373253 | 0.891460005 | 0.932191032 | 1.209705513 | | |
| 147 | 1 | 1 | 2.052066815 | 1.360978962 | 0.994079712 | 0.852309969 | 0.499312824 | 1 | 1 | | |
| 148 | 1 | 1 | 1 | 1 | 1 | 1 | 1 | 1 | 1 | | |
| 149 | 0.459187449 | 0.54771948 | 0.782238564 | 0.774487396 | 1.01318361 | 1.022441949 | 0.764202193 | 1.267950324 | 1.444896681 | | |
| 150 | 1 | 1 | 0.728254848 | 0.588144044 | 0.54598338 | 0.71933518 | 0.547257618 | 1 | 1 | | |
| 151 | 1 | 1 | 1 | 1 | 1 | 1 | 1 | 1.007446809 | 1 | | |
| 152 | 0.735484476 | 0.82589188 | 0.916485841 | 0.921232505 | 0.562493219 | 0.694314853 | 0.645546273 | 1.04601725 | 1.22587519 | | |
| 153 | 1 | 1 | 1 | 1.224787234 | 1.847659574 | 1.541489362 | 1.670425532 | 1 | 1 | | |
| 154 | 0.33286119 | 0.33286119 | 0.860537096 | 0.675921711 | 0.67935063 | 0.968472159 | 0.829555454 | 1.832821745 | 1.48836692 | | |
| 155 | 0.872938846 | 0.818767022 | 0.747930186 | 0.738811815 | 0.785186843 | 0.640803312 | 0.660774222 | 0.909454293 | 1.335331943 | | |
| 156 | 1.562608647 | 1.597207424 | 0.609360819 | 0.609360819 | 0.609360819 | 0.895047323 | 0.748282121 | 0.909980172 | 0.836351619 | | |
| 157 | 0.99030303 | 1.293535354 | 0.570422963 | 0.998482918 | 0.981795012 | 1.21609321 | 0.937435524 | 0.847729984 | 0.984001123 | | |
| 158 | 0.362416767 | 0.369625425 | 1.218879436 | 1.058427788 | 1.005890523 | 0.843764726 | 0.736954042 | 1.22902423 | 1.814486369 | | |
| 159 | 0.78424829 | 0.914483564 | 1.36195584 | 1.195526901 | 1.058138871 | 1.265247903 | 1.330689793 | 0.973948945 | 1.053846601 | | |
| 160 | 0.973226072 | 1.327887226 | 1.096179734 | 0.989532563 | 1.47734763 | 0.906883907 | 0.990669823 | 0.944122305 | 0.885279865 | | |
| 161 | 0.618817141 | 0.590091685 | 0.697972338 | 1.062757102 | 0.853480387 | 1.081835261 | 1.331130762 | 1.123546073 | 0.908836858 | | |
| 162 | 1 | 1.157340426 | 1 | 1 | 1 | 1.425106383 | 1 | 0.942900798 | 0.919472582 | | |
| 163 | 1.631364036 | 1.851088024 | 0.562022224 | 0.746355111 | 0.653164158 | 0.592481677 | 0.65852313 | 0.983174731 | 0.877379042 | | |
| 164 | 1.204148936 | 1.148723404 | 1 | 1 | 1 | 1 | 1 | 1 | 1 | | |
| 165 | 1.912996599 | 1.604628017 | 1 | 1.253404255 | 1.805638298 | 2.778617021 | 2.328085106 | 0.930108303 | 1.012490975 | | |
| 166 | 0.812607368 | 0.939595097 | 1 | 1 | 1 | 1 | 1 | 1.099661175 | 1.669996495 | | |
| 167 | 0.532200794 | 0.581689266 | 0.461077623 | 0.429089161 | 0.453348619 | 0.671424873 | 0.627306682 | 0.869839259 | 1.046277029 | | |
| 168 | 1 | 1 | 3.273465067 | 1.616737238 | 1.144024935 | 1.154904728 | 0.863502705 | 1 | 1 | | |
| 169 | 0.390472833 | 0.421572732 | 1.184377709 | 0.876133697 | 0.712521934 | 0.569390711 | 0.524285956 | 1.100338219 | 2.201754816 | | |
| 170 | 1.73331798 | 1.961022511 | 1.406904358 | 1.262900653 | 1.306014303 | 1.242835829 | 1.038431857 | 1.174746855 | 0.984657871 | | |
| 171 | 1.891037216 | 1.852851795 | 1.095762637 | 1.115690977 | 1.224926712 | 1.749637263 | 1.641052974 | 0.897061304 | 1.036978306 | | |
| 172 | 1 | 1 | 0.988002181 | 1.942192329 | 3.726504272 | 2.656153427 | 2.84548264 | 1 | 1 | | |
| 173 | 0.930305616 | 1.180240976 | 0.520525452 | 0.573092797 | 0.888008698 | 0.92710691 | 1.068521724 | 1.552102416 | 0.991185155 | | |
| 174 | 1.482873851 | 1.449271326 | 0.626404656 | 0.801878638 | 0.81663113 | 0.740275457 | 0.875583473 | 0.833777228 | 0.702606297 | | |
| 175 | 1 | 1 | 2.162234043 | 1.36712766 | 1.215425532 | 1 | 1.035638298 | 1 | 1 | | |
| 176 | 1.055071821 | 1.434529422 | 1 | 1 | 1 | 1 | 1 | 0.779672968 | 0.739112917 | | |
| 177 | 1.415241729 | 1.794082249 | 0.419926014 | 0.860410003 | 0.602269662 | 0.57047898 | 0.587453277 | 0.94100039 | 0.876434779 | | |
| 178 | 1.719832203 | 2.015173152 | 0.735483302 | 1.111133139 | 1.119129439 | 1.274121068 | 1.06632743 | 0.824712815 | 0.940955895 | | |
| 179 | 0.77963009 | 0.77963009 | 1 | 1 | 1 | 1 | 1 | 1 | 1 | | |
| 180 | 0.800561555 | 0.494470842 | 0.84078712 | 0.957602862 | 1.231216458 | 0.84078712 | 1.174776386 | 1 | 1 | | |
| 181 | 0.696977847 | 0.995293643 | 0.987179487 | 0.838703182 | 0.859846959 | 1.136931132 | 0.93381662 | 1.177926704 | 1.87767916 | | |
| 182 | 1 | 1 | 1 | 1 | 1 | 1 | 1 | 0.676440102 | 0.918185838 | | |
| 183 | 0.575391986 | 0.643031359 | 1 | 1 | 1 | 1 | 1 | 1 | 1 | | |
| 184 | 2.109684578 | 2.406007291 | 1.565092565 | 1.104346104 | 1.089199089 | 1.175626176 | 1.209088209 | 0.997502498 | 0.949959132 | | |
| 185 | 0.667705857 | 0.644363041 | 1.258993202 | 0.859121808 | 0.62340621 | 0.715542899 | 0.45252618 | 1.507952345 | 1.323669229 | | |
| 186 | 1 | 1 | 1 | 1 | 1 | 1 | 1 | 1 | 1 | | |
| 187 | 1.142346448 | 0.982086386 | 1.049162615 | 0.840163618 | 1.217874508 | 1.670448406 | 1.328548275 | 0.976899827 | 1.117659758 | | |
| 188 | 0.686954063 | 0.876139006 | 0.63211454 | 0.882400683 | 0.789648929 | 0.608141011 | 1.063325911 | 0.958494852 | 0.791308298 | | |
| 189 | 1 | 1 | 1 | 1 | 1 | 1.081914894 | 1 | 1 | 1 | | |
| 190 | 1 | 1 | 0.556658079 | 1.306499816 | 2.059919028 | 1.636555024 | 1.383496503 | 1 | 1 | | |
| 191 | 0.733517631 | 0.606769953 | 0.990935067 | 1.052714553 | 1.009064933 | 1.092521431 | 1.154941374 | 1.157773944 | 0.915577889 | | |
| 192 | 1 | 1 | 2.296382979 | 1.167553191 | 1 | 1.097340426 | 1.020319149 | 1 | 1 | | |
| 193 | 1 | 1 | 1 | 1 | 1 | 1 | 1 | 1 | 1 | | |
| 194 | 1.477340426 | 1.131276596 | 1 | 1 | 1 | 1 | 1 | 1 | 1 | | |
| 195 | 0.663385301 | 0.7365256121 | 1 | 1 | 1.931276596 | 1.497978723 | 1.697340426 | 1.117401906 | 1.129771669 | | |
| 196 | 2.214255319 | 1.832234043 | 1.257325241 | 1.2047928 | 1.789870239 | 2.097111762 | 2.137714525 | 0.903664112 | 0.970442398 | | |
| 197 | 1 | 1 | 1 | 1 | 1 | 1 | 1 | 1 | 1 | | |
| 198 | 1.138617021 | 1 | 1.267663719 | 1.365944549 | 1.036768359 | 0.533936202 | 0.67196661 | 1 | 1 | | |
| 199 | 1.523005242 | 1.904251602 | 0.62401015 | 0.693266833 | 0.754079713 | 0.900205626 | 0.753117207 | 0.947282213 | 1.003397234 | | |
| 200 | 1 | 1 | 1.081228831 | 0.863509418 | 0.628439004 | 0.558559629 | 0.603838612 | 1 | 1 | | |
| 201 | 1 | 1 | 2.08610977 | 0.919362627 | 0.756478352 | 0.756478352 | 0.756478352 | 1 | 1.130957447 | | |
| 202 | 0.195544262 | 0.175626909 | 1.500708737 | 1.081344144 | 0.675738504 | 1.069701952 | 1.225283967 | 1.238422634 | 1.674594122 | | |
| 203 | 0.949813712 | 1.002119448 | 1.014027234 | 1.010242107 | 0.852942555 | 0.744779338 | 0.669803361 | 0.773818859 | 0.801955339 | | |
| 204 | 1 | 1 | 0.767409584 | 0.767409584 | 0.989550167 | 1.205241244 | 0.841129888 | 0.986151909 | 0.986151909 | | |
| 205 | 0.341399177 | 0.336076818 | 0.980243232 | 1.1368063 | 1.307953963 | 1.15423326 | 1.163459298 | 0.799787975 | 1.14011308 | | |
| 206 | 1.278085106 | 1.191914894 | 1.024542152 | 1.441981167 | 1.863794765 | 2.16675317 | 1.940016312 | 0.949314079 | 0.979783394 | | |
| 207 | 0.236429103 | 0.414726103 | 0.680675422 | 1.073889931 | 1.059933292 | 1.276057953 | 1.088805503 | 1.102478464 | 0.943526872 | | |
| 208 | 1.973789125 | 1.653631917 | 1.675467207 | 1.360146333 | 2.794032087 | 2.900652327 | 2.315100494 | 0.832501387 | 0.947981434 | | |
| 209 | 1.014518954 | 0.895147167 | 0.683064298 | 1.214627108 | 1.992785643 | 1.369780864 | 1.638470556 | 0.914200554 | 1.225179192 | | |
| 210 | 0.753325854 | 0.753325854 | 1 | 1 | 1 | 1 | 1 | 1.172264616 | 1.028600613 | | |
| 211 | 1.12339396 | 1.199315869 | 1 | 1 | 1 | 1 | 1 | 0.889892367 | 1.199678337 | | |
| 212 | 1.312521266 | 1.509186798 | 0.557173245 | 0.919309856 | 0.71434649 | 0.633230915 | 0.724225951 | 1.088323554 | 0.786088592 | | |
| 213 | 0.960685392 | 1.134896828 | 0.653917373 | 0.711332115 | 0.712067887 | 0.657167033 | 0.598293009 | 0.873035603 | 0.957915298 | | |
| 214 | 0.632981863 | 0.641667597 | 0.956840931 | 1.011044931 | 0.972814161 | 0.782691901 | 0.732300772 | 1.19964347 | 1.693517274 | | |
| 215 | 0.933174399 | 0.797909408 | 0.877598152 | 0.957138976 | 0.747316941 | 0.638500204 | 0.651473985 | 0.92553917 | 1.196658426 | | |
| 216 | 0.62675386 | 0.788253797 | 0.877346809 | 0.977732366 | 0.915036693 | 0.978608172 | 0.799037619 | 1.191302572 | 1.211132095 | | |
| 217 | 1.265402034 | 1.458600359 | 0.905880211 | 0.982644578 | 1.021967352 | 1.49778506 | 1.27653377 | 1 | 1 | | |
| 218 | 0.868403025 | 0.772731446 | 0.924675837 | 0.889693684 | 0.740201633 | 0.735240091 | 0.655812892 | 1.144349119 | 1.339430195 | | |
| 219 | 0.373219281 | 0.41047616 | 1.439359787 | 0.950733899 | 0.81592424 | 0.919176952 | 0.699682886 | 1.313626731 | 1.28709192 | | |
| 220 | 2.049893617 | 2.378085106 | 0.84078712 | 0.84078712 | 0.84078712 | 1.515026834 | 0.84078712 | 0.869263923 | 0.951494591 | | |
| 221 | 0.234759372 | 0.265577783 | 1.339801051 | 1.22425394 | 1.647904325 | 1.794322119 | 1.301732424 | 0.904570736 | 1.125580228 | | |
| 222 | 1 | 1 | 0.930600931 | 1.13978814 | 1.089199089 | 1.326205326 | 1.129294129 | 1.107135492 | 0.998247061 | | |
| 223 | 1.204684521 | 1.291259925 | 1.228749355 | 1.540518028 | 1.970251081 | 2.722779739 | 2.171988418 | 1.16250575 | 1.07656923 | | |
| 224 | 1 | 1 | 1 | 1 | 1 | 1 | 1 | 1 | 1 | | |
| 225 | 1 | 1 | 1 | 1 | 1 | 1 | 1 | 1 | 1 | | |
| 226 | 0.789568335 | 0.956907492 | 0.856285912 | 0.95530243 | 1.062479991 | 0.844344842 | 0.829353913 | 0.87124332 | 0.777659853 | | |
| 227 | 1.250528176 | 1.381554181 | 0.487956811 | 0.790801495 | 0.784468439 | 1.012406561 | 1.136004983 | 1.360228855 | 0.667381837 | | |
| 228 | 0.961520122 | 1.005102041 | 0.738350768 | 0.945184078 | 1.170764842 | 1.067518 | 1.203233256 | 0.958322468 | 0.879182964 | | |
| 229 | 1.185531915 | 1.061276596 | 1 | 1.291382979 | 1.847659574 | 2.160531915 | 2.417533191 | 0.952240408 | 0.840711922 | | |
| 230 | 1.866595745 | 1.832234043 | 1 | 1 | 1 | 1 | 1 | 0.923570319 | 0.728445075 | | |
| 231 | 0.791844157 | 0.863255498 | 0.821112684 | 0.378419422 | 0.535512734 | 0.915231861 | 1.017682831 | 1.018825055 | 0.95631404 | | |
| 232 | 1.024228595 | 1.003474754 | 1.276380814 | 1.096899225 | 0.990213178 | 1.288299419 | 1.262936047 | 1.169955525 | 0.927388012 | | |
| 233 | 1 | 1 | 1 | 1 | 1 | 1 | 1 | 0.884189508 | 0.950535487 | | |
| 234 | 0.899978884 | 1.077285845 | 1.253127744 | 1.126975417 | 1.331595698 | 1.5470259 | 1.545050483 | 0.938209954 | 0.60928497 | | |
| 235 | 1.900561555 | 1.922332613 | 0.957902973 | 1.219405321 | 1.532316119 | 2.043740219 | 2.102503912 | 1.022700038 | 0.799842504 | | |
| 236 | 0.607947836 | 0.550187401 | 0.852128126 | 0.81671159 | 1.039052216 | 0.718046762 | 0.944399173 | 1.067294423 | 0.837324458 | | |
| 237 | 0.920080289 | 0.772073 | 0.834029906 | 1.128138448 | 1.5338844 | 1.281477354 | 1.899817343 | 0.936843723 | 0.758157046 | | |
| 238 | 0.847601397 | 0.804690284 | 0.93067768 | 1.032684963 | 0.753752554 | 0.758193445 | 0.825295319 | 1.002511688 | 1.038495971 | | |
| 239 | 0.961976081 | 1.089934203 | 0.870610633 | 1.024608659 | 1.119235651 | 0.902482019 | 1.005076858 | 0.691436777 | 0.82793472 | | |
| 240 | 1 | 1 | 1 | 1 | 1 | 1 | 1 | 1 | 1 | | |
| 241 | 1.970046742 | 1.274348946 | 1 | 1 | 1 | 1.642978723 | 2.06893617 | 1.230242622 | 1.319274972 | | |
| 242 | 1.781871649 | 1.186265352 | 0.902636917 | 0.978814514 | 0.982946435 | 1.272481406 | 1.141461949 | 1.28873842 | 1.15740913 | | |
| 243 | 0.813078453 | 0.813078453 | 1.045567215 | 0.896430637 | 0.668388003 | 0.576055523 | 0.622407668 | 1 | 1.097340426 | | |
| 244 | 1.176008278 | 0.934998809 | 0.896438953 | 1.110125969 | 1.218507752 | 1.229360465 | 2.027252907 | 0.90438173 | 0.872632752 | | |
| 245 | 0.926216835 | 0.727936174 | 1.446170213 | 1.376595745 | 1.03287234 | 1.248510638 | 1.035638298 | 0.964633464 | 1.004141018 | | |
| 246 | 1.10096057 | 0.907906227 | 1 | 1 | 1 | 1 | 1.004893617 | 1.084680851 | 1 | | |
| 247 | 1.787719151 | 2.056549098 | 1.038625963 | 1.284487825 | 1.594555492 | 1.129612233 | 1.385124307 | 1.004798038 | 0.856250479 | | |
| 248 | 1.333384474 | 1.114682418 | 1.033297872 | 1.243829787 | 1.889574468 | 1.468829787 | 1.548404255 | 0.852924188 | 0.79032491 | | |
| 249 | 1.558861779 | 1.334097102 | 0.814464609 | 1.138884653 | 1.063121685 | 1.098435938 | 1.139071052 | 1.02845267 | 0.899574447 | | |
| 250 | 1.975155022 | 1.594454468 | 1.271580504 | 1.298454788 | 0.995183136 | 1.66060187 | 1.324709081 | 0.963759768 | 1.212740123 | | |
| 251 | 0.803195311 | 0.729476324 | 1.088252716 | 0.777517782 | 0.843217788 | 0.690082584 | 0.548784249 | 1.136889061 | 1.599732078 | | |
| 252 | 0.871366546 | 1.017661344 | 1.460820551 | 0.989281094 | 1.271946036 | 2.098133432 | 1.602199224 | 0.907248335 | 1.236174114 | | |
| 253 | 1.146200112 | 1.091389895 | 1.153640996 | 1.077855273 | 1.490564808 | 1.542270964 | 2.08862671 | 1.001475331 | 0.826265468 | | |
| 254 | 1.94684466 | 1.85973301 | 1.152952134 | 1.384992306 | 1.021037499 | 1.77533004 | 1.887341055 | 0.805760897 | 1.114750616 | | |
| 255 | 0.89118163 | 0.882155956 | 1 | 1.071808511 | 1.185425532 | 1.173510638 | 1.299255319 | 0.799244903 | 1.101283665 | | |
| 256 | 1.667059301 | 1.403432169 | 0.497034721 | 0.572927161 | 0.581755487 | 0.580727246 | 0.617484239 | 0.97975823 | 0.776012396 | | |
| 257 | 1.463897449 | 1.732901044 | 1.717886892 | 1.144454187 | 1.18643139 | 1.291977203 | 1.357737834 | 1.17912892 | 0.996062718 | | |
| 258 | 0.223332169 | 0.312579213 | 1.358159756 | 0.921119684 | 0.57429218 | 1.062701566 | 0.842239367 | 1.071370618 | 1.589720367 | | |
| 259 | 0.754010695 | 0.700625397 | 0.646565227 | 0.915585142 | 0.770026852 | 0.909543522 | 0.864175431 | 0.990314608 | 0.627880569 | | |
| 260 | 0.490012143 | 0.612537948 | 1.137029741 | 0.970739442 | 0.853408927 | 0.728967472 | 0.790851871 | 0.950043215 | 1.407490637 | | |
| 261 | 1 | 1 | 0.340460584 | 0.515545585 | 0.400469698 | 0.371656911 | 0.370955869 | 1 | 1 | | |
| 262 | 2.889815091 | 2.631486057 | 0.935771194 | 1.32141876 | 1.514893217 | 2.24528261 | 1.826841047 | 0.938081662 | 0.894495819 | | |
| 263 | 0.785265363 | 0.591081405 | 1.029144212 | 0.921233024 | 1.205733994 | 1.131666307 | 1.430135805 | 1.022490086 | 1.246921311 | | |
| 264 | 1.370690713 | 1.595816464 | 1.705106383 | 1.215319149 | 1.259893617 | 1.381276596 | 1.534787234 | 1.05628228 | 1.082154752 | | |
| 265 | 1.041224971 | 1.004806927 | 0.687687688 | 0.865700752 | 0.789696913 | 0.73972942 | 1.005293954 | 0.932566269 | 0.870084474 | | |
| 266 | 0.681356915 | 0.681356915 | 1 | 1 | 1 | 1 | 1 | 1 | 1.080531915 | | |
| 267 | 1 | 1 | 0.834580509 | 0.839070341 | 1.042609385 | 1.395457347 | 0.931684127 | 1 | 1 | | |
| 268 | 0.738085927 | 0.811833063 | 1 | 1 | 1 | 1 | 1 | 1 | 1.378723404 | | |
| 269 | 1.874264912 | 1.156230748 | 0.658060763 | 1.029576267 | 1.063492774 | 1.777126493 | 1.694706698 | 1.102332127 | 1.149348038 | | |
| 270 | 0.942544871 | 0.942544871 | 1.84393617 | 1 | 1 | 1 | 1 | 1 | 1 | | |
| 271 | 1.036003996 | 0.080946558 | 0.577963793 | 0.725960877 | 0.805100834 | 0.825467026 | 0.807755798 | 0.786921695 | 0.83901315 | | |
| 272 | 1.384526103 | 0.902818249 | 0.399616049 | 0.823612978 | 0.859589173 | 1.072528316 | 1.125897485 | 0.904483793 | 0.993681807 | | |
| 273 | 1.679063545 | 1.610345596 | 0.711770632 | 1.165172009 | 0.658920874 | 0.82926225 | 0.941766173 | 0.900181984 | 0.840650591 | | |
| 274 | 0.656669693 | 0.603324672 | 0.541104912 | 0.993275553 | 1.283926634 | 1.771291315 | 1.536561068 | 1.025125628 | 0.816766761 | | |
| 275 | 0.267973826 | 0.36703655 | 1.608805619 | 0.988357228 | 0.665562413 | 1.289777179 | 1.401792465 | 1.290240811 | 1.273034409 | | |
| 276 | 1.039539951 | 1.295567382 | 0.791784239 | 0.95864058 | 0.959763037 | 0.893101567 | 0.782523969 | 0.887794433 | 1.065024982 | | |
| 277 | 0.798369288 | 0.798369288 | 1.088357702 | 1.052992831 | 0.898699148 | 0.829080688 | 0.763624082 | 1.056153101 | 1.428149225 | | |
| 278 | 0.928608958 | 0.892814922 | 0.527003795 | 0.835155211 | 0.81134646 | 0.710694476 | 0.802815531 | 1 | 1.197765957 | | |
| 279 | 1.119680851 | 1.387553191 | 1 | 1 | 1 | 1 | 1 | 0.927141614 | 1.317543205 | | |
| 280 | 2.253426364 | 2.580363818 | 1.56980809 | 1.500820432 | 1.99522009 | 3.269387173 | 2.241706499 | 0.884051204 | 1.141978911 | | |
| 281 | 0.983016983 | 0.681634155 | 1 | 1 | 1 | 1 | 1 | 1.209195978 | 0.927208945 | | |
| 282 | 1.840125786 | 1.711383648 | 1 | 1.433191489 | 2.055744681 | 1.686276596 | 1.548404255 | 0.873075453 | 0.802592708 | | |
| 283 | 0.583487434 | 0.654502398 | 0.57013144 | 0.690176711 | 0.516269259 | 0.958175785 | 0.68393909 | 0.975506815 | 1.101109095 | | |
| 284 | 1.097865854 | 1.175391986 | 0.735524257 | 0.767057903 | 0.736697966 | 0.795774648 | 0.828090767 | 0.989006302 | 0.997820215 | | |
| 285 | 0.452363537 | 0.905184237 | 1 | 1 | 1 | 1 | 1 | 1 | 1.280531915 | | |
| 286 | 0.898667568 | 1.028483151 | 0.766758001 | 1.022795966 | 1.499392673 | 1.862969973 | 1.531877631 | 1.031686047 | 0.801356589 | | |
| 287 | 0.442389849 | 0.530723288 | 1.617511057 | 1.361044096 | 1.731905911 | 1.868884868 | 1.529386141 | 0.864257882 | 0.95940037 | | |
| 288 | 0.786Q55347 | 1.228320583 | 1 | 1 | 1 | 1 | 1 | 1.112351492 | 1.29498893 | | |
| 289 | 1.054987044 | 1.060564803 | 1.490766823 | 1.684976526 | 2.26486698 | 1.951799687 | 2.047339593 | 0.892422305 | 1.031244788 | | |
| 290 | 0.738425054 | 0.834504311 | 2.378682105 | 1.181309949 | 1.888415915 | 0.759662864 | 0.576252909 | 1 | 1 | | |
| 291 | 0.751661039 | 0.78286679 | 0.646225767 | 0.772996013 | 0.646225767 | 0.646225767 | 0.646225767 | 1.115762798 | 1.061632032 | | |
| 292 | 1.833996057 | 2.334948374 | 0.814410677 | 0.973541347 | 0.693155379 | 0.821787357 | 0.680963117 | 0.808554122 | 0.935129297 | | |
| 293 | 1.038166624 | 1.149651073 | 0.71748591 | 0.955232931 | 1.132410644 | 1.361810658 | 1.212456303 | 0.917858487 | 0.845878811 | | |
| 294 | 1.030693428 | 0.947937456 | 0.777345491 | 1.261049589 | 1.719225795 | 1.657106839 | 2.051979526 | 0.786103971 | 0.738023298 | | |
| 295 | 1 | 1 | 1.564574468 | 1 | 1 | 1 | 1.184574468 | 1 | 1.50787234 | | |
| 296 | 1 | 1 | 0.659745257 | 0.775640175 | 0.831564283 | 1.07861218 | 1.257463182 | 1 | 1.297021277 | | |
| 297 | 1 | 1 | 1.705106383 | 1 | 1 | 1 | 1 | 1 | 1.836595745 | | |
| 298 | 0.847927954 | 1.773539318 | 1 | 1 | 1 | 1 | 1 | 1.261653201 | 1.150670337 | | |
| 299 | 0.594965599 | 0.89122453 | 1.288324302 | 1.290100974 | 1.235794024 | 0.74638003 | 0.857984661 | 0.929071794 | 0.965068005 | | |
| 300 | | 1 | 1 | 0.419221373 | 0.796122626 | 0.689534242 | 0.624596107 | 0.588659469 | 1 | 1 | |
| 301 | 0.850134805 | 1.159111972 | 1.416666667 | 1.185324869 | 1.563106796 | 0.849103809 | 0.684889221 | 1.054783444 | 0.723909638 | | |
| 302 | 1.980319149 | 2.290319149 | 1 | 2.705106383 | 2.739893617 | 1.931489362 | 2.199148936 | 0.772203412 | 0.821037896 | | |
| 303 | 2.266663125 | 1.817803513 | 0.874272847 | 1.074341653 | 0.828642022 | 1.086069932 | 1.08137862 | 0.931518345 | 0.881553425 | | |
| 304 | 1 | 1 | 1 | 1 | 1 | 1.097340426 | 1.227978723 | 1 | 1 | | |
| 305 | 0.774141092 | 0.657752351 | 0.868643303 | 0.880948632 | 0.793397984 | 0.673720595 | 0.584791167 | 1.220011355 | 1.112243685 | | |
| 306 | 0.315068933 | 0.391104541 | 0.506165527 | 0.581336492 | 0.506165527 | 0.506165527 | 0.584782726 | 0.9943101 | 1.497403983 | | |
| 307 | 1.082411363 | 0.887172746 | 1.112366533 | 1.15558443 | 1.48985933 | 1.409185922 | 1.444890119 | 1.175604679 | 1.164502379 | | |
| 308 | 0.78333477 | 0.673633856 | 0.947111728 | 0.888988322 | 0.712022909 | 0.623920533 | 0.593494116 | 1 | 1.411276596 | | |
| 309 | 0.629003471 | 0.655452029 | 0.515366793 | 0.653000476 | 0.640357203 | 0.509321345 | 0.582971476 | 0.87410894 | 0.83583529 | | |
| 310 | 1.453295991 | 1.960790415 | 0.999150434 | 0.796043449 | 0.760665089 | 1.313429213 | 1.08252928 | 0.913580247 | 1.01449515 | | |
| 311 | 1 | 1 | 1.313496388 | 0.64156274 | 0.596440247 | 1.254247891 | 0.587056544 | 1 | 1 | | |
| 312 | 0.482185337 | 0.474594392 | 0.746039948 | 0.971633733 | 0.849402206 | 0.728346667 | 0.824367906 | 0.863247296 | 0.871408666 | | |
| 313 | 1 | 1 | 1.399354578 | 1.159453697 | 0.824410765 | 0.594421714 | 0.560998098 | 1 | 1 | | |
| 314 | 1 | 1 | 1 | 1 | 1 | 1 | 1 | 1 | 1 | | |
| 315 | 0.792106262 | 0.713472486 | 1.324242844 | 0.582160926 | 0.926640793 | 0.416349019 | 0.58347718 | 1.158498162 | 1.200857693 | | |
| 316 | 1 | 1 | 1 | 1 | 1 | 1 | 1 | 1 | 1 | | |
| 317 | 0.882651239 | 0.671380616 | 1 | 1 | 1 | 1 | 1 | 1 | 1.181170213 | | |
| 318 | 0.706076767 | 0.706076767 | 1 | 1 | 1 | 1 | 1 | 1 | 1 | | |
| 319 | 0.604165605 | 0.646178344 | 0.880580921 | 0.646527614 | 0.64133841 | 0.671327964 | 0.752266065 | 0.940996992 | 1.12875761 | | |
| 320 | 0.189530013 | 0.163060943 | 1.006789806 | 1.023034838 | 0.795407996 | 0.920196399 | 1.167566051 | 1.268970098 | 2.011456777 | | |
| 321 | 0.935695799 | 0.935695799 | 0.899729891 | 0.911949088 | 0.589362309 | 0.272574755 | 0.360989168 | 1.406355004 | 2.327027027 | | |
| 322 | 1 | 1 | 1.846919302 | 1.238367866 | 0.878044348 | 0.475236278 | 0.487186478 | 1 | 1 | | |
| 323 | 0.909266783 | 0.909266783 | 2.316515945 | 1.43944442 | 0.774176799 | 0.525896759 | 0.629786682 | 0.986151909 | 1.246537977 | | |
| 324 | 1.683611505 | 1.617004836 | 1.520592021 | 1.76137221 | 2.165424045 | 2.13753162 | 2.252585097 | 1.030649335 | 1.052181901 | | |
| 325 | 1.44206545 | 1.32468344 | 1.185825498 | 1.084369629 | 1.144474775 | 1.320341725 | 1.239156809 | 0.994642947 | 1.261232122 | | |
| 326 | 0.520250906 | 0.508300438 | 1.573032344 | 1.040475757 | 1.069846471 | 1.036045877 | 0.700467261 | 0.969422992 | 1.065800213 | | |
| 327 | 0.468477448 | 0.468477448 | 1 | 1 | 1 | 1 | 1 | 0.963454888 | 1.45779629 | | |
| 328 | 0.484884222 | 0.605266142 | 1.350114109 | 1.299704658 | 1.536582092 | 2.262384213 | 1.653309169 | 1.108662004 | 1.491515707 | | |
| 329 | 1 | 1 | 2.125213383 | 1.232843974 | 0.878200751 | 0.905001707 | 0.802321611 | 1 | 1 | | |
| 330 | 1.4264948 | 1.475966751 | 1.080462957 | 1.080116432 | 0.930903042 | 0.7546261 | 0.799916834 | 0.903714535 | 0.855155466 | | |
| 331 | 0.511926805 | 0.87964274 | 0.868600998 | 0.868600998 | 0.868600998 | 0.868600998 | 0.868600998 | 1 | 1 | | |
| 332 | 0.423156698 | 0.623909646 | 0.670614254 | 0.670614254 | 0.670614254 | 0.670614254 | 0.704715702 | 0.96129269 | 1.209251842 | | |
| 333 | 0.539114476 | 0.539114476 | 0.715699711 | 0.760164459 | 0.805238313 | 0.715699711 | 0.715699711 | 1 | 1.164468085 | | |
| 334 | 1.031990967 | 1.130033873 | 0.467057538 | 0.873298221 | 0.985789526 | 1.154476796 | 0.780184836 | 0.859546452 | 1.086503292 | | |
| 335 | 1 | 1 | 0.854390111 | 1.095255408 | 1.151881476 | 1.527176877 | 1.711688784 | 1 | 1.130957447 | | |
| 336 | 1.975420561 | 4.114859813 | 0.709495612 | 0.821359777 | 0.73403724 | 0.945922808 | 0.823500036 | 0.887794433 | 1.000927909 | | |
| 337 | 1.102425605 | 1.107122488 | 1.178784388 | 1.229367123 | 2.002303835 | 2.239192302 | 1.841814609 | 0.975056958 | 0.921531497 | | |
| 338 | 1.048432948 | 1.584898045 | 0.688343487 | 1.054886758 | 1.233611216 | 1.969918675 | 1.554172618 | 0.741739026 | 0.827655332 | | |
| 339 | 0.81340984 | 1.25119541 | 0.814769871 | 0.814769871 | 1.550836439 | 1.597122302 | 1.470312906 | 0.664783937 | 0.803976943 | | |
| 340 | 0.633331062 | 0.624669323 | 1.305114654 | 1.002119357 | 1.171058496 | 1.239159898 | 2.242061507 | 0.815678188 | 0.857057137 | | |
| 341 | 1.209220722 | 1.293337882 | 1 | 1 | 1 | 1 | 1 | 0.903752964 | 0.999793793 | | |
| 342 | 1.725508894 | 2.224170182 | 1.057862546 | 1.105585064 | 0.851118622 | 1.655882826 | 1.171334299 | 0.910475447 | 1.133798692 | | |
| 343 | 0.489218956 | 0.44078182 | 0.938520939 | 0.930600931 | 0.930600931 | 0.930600931 | 0.930600931 | 0.690719377 | 1.557351753 | | |
| 344 | 0.920521173 | 0.738532286 | 1.025565784 | 0.989950557 | 1.285886562 | 0.981629618 | 0.966274068 | 0.941332547 | 0.932134434 | | |
| 345 | 1.801206839 | 1.781075216 | 0.820273322 | 1.305840958 | 1.479963364 | 2.115540522 | 1.671941454 | 0.717002407 | 0.734058666 | | |
| 346 | 0.327937483 | 0.542038794 | 0.749633403 | 0.95060585 | 0.725476576 | 1.097244733 | 0.725476576 | 1 | 1.247553191 | | |
| 347 | 1.216566158 | 0.877350124 | 1.156382979 | 1.17712766 | 1.094574468 | 1 | 1.155319149 | 1.147409105 | 0.983191767 | | |
| 348 | 1.320326894 | 1.191758332 | 0.815347894 | 0.971716229 | 0.80586314 | 1.009556541 | 1.181251234 | 0.972111947 | 1.015435828 | | |
| 349 | 1.521504941 | 1.422606512 | 1.018662191 | 1.152794806 | 0.910749101 | 1.496824312 | 1.46798183 | 0.973177442 | 0.999541495 | | |
| 350 | 0.725814597 | 0.645352892 | 1.181685544 | 1.405068151 | 1.763409484 | 1.8 | 1.71906316 | 1.011935875 | 1.190111815 | | |
| 351 | 0.522677191 | 0.794211478 | 0.930600931 | 0.97950698 | 0.930600931 | 2.261063261 | 2.058608059 | 0.921703808 | 1.040106416 | | |
| 352 | 0.635373784 | 0.62209892 | 0.69340556 | 0.845470174 | 0.319666548 | 0.304907717 | 0.347789419 | 1 | 1 | | |
| 353 | 0.83605 | 0.75365 | 1 | 1 | 1 | 1 | 1 | 1.19 | 1 | | |
| 354 | 0.359324957 | 0.40166913 | 1.242113827 | 1.013719907 | 1.088458267 | 1.104470988 | 0.993676269 | 1.059146833 | 1.047019968 | | |
| 355 | 0.618289832 | 1.086248032 | 0.506617503 | 0.622875562 | 0.61078824 | 0.44334831 | 0.546127173 | 0.865266994 | 1.132655548 | | |
| 356 | 1 | 1 | 1.867021277 | 1.13893617 | 1 | 1 | 1 | 1 | 1 | | |
| 357 | 0.71142572 | 0.632468504 | 0.785095797 | 0.740482146 | 0.714753017 | 0.528711619 | 0.561532752 | 1.100013962 | 1.276725462 | | |
| 358 | 1.278085106 | 1.669255319 | 0.487956811 | 0.983232973 | 0.650695598 | 0.865137043 | 0.735568937 | 1 | 1 | | |
| 359 | 1 | 1 | 1 | 1 | 1.094574468 | 1.859574468 | 1.299255319 | 1 | 1 | | |
| 360 | 1.248339248 | 1.357630358 | 0.767379176 | 0.866670846 | 0.905158904 | 1.095718674 | 0.757224347 | 0.934937475 | 0.916684502 | | |
| 361 | 1.889101396 | 1.720431087 | 0.828889719 | 1.051306945 | 1.333980583 | 1.445170525 | 1.186880757 | 0.9731415 | 0.935971495 | | |
| 362 | 1 | 1 | 1 | 1 | 1 | 1 | 1 | 0.784940283 | 0.803369453 | | |
| 363 | 3.235425532 | 2.640425532 | 1 | 1 | 1 | 1 | 1 | 1 | 1 | | |
| 364 | 1.620046083 | 1.14751861 | 0.52660431 | 0.816151026 | 0.558844691 | 0.57100764 | 0.71244047 | 1.013540254 | 0.738888765 | | |
| 365 | 1.530957447 | 1.65287234 | 0.955645161 | 1.205745968 | 1.277520161 | 1.748387097 | 1.570060484 | 1.075 | 1.181170213 | | |
| 366 | 1.654282978 | 1.611247947 | 0.507054598 | 1.04488221 | 0.732470392 | 1.170657147 | 0.92819274 | 1 | 1 | | |
| 367 | 1.54474764 | 2.114286534 | 1 | 1 | 1 | 1 | 1 | 0.904574578 | 0.806360588 | | |
| 368 | 1.425081687 | 2.076915098 | 1 | 1 | 1 | 1 | 1 | 1 | 1 | | |
| 369 | 0.874624604 | 1.016612105 | 1.281010444 | 1.643669291 | 1.927149251 | 1.215774039 | 1.406492772 | 0.992527534 | 0.764896138 | | |
| 370 | 0.582719279 | 0.754433394 | 1.13425386 | 0.848810607 | 1.044696416 | 1.314406643 | 1.234552952 | 1.18336969 | 1.258520353 | | |
| 371 | 0.356069578 | 0.356920799 | 0.65172556 | 0.944452083 | 1.001993675 | 0.646225767 | 0.929740135 | 1.251101675 | 1.278343081 | | |
| 372 | 0.44009551 | 0.44009551 | 1.33202235 | 1.179613757 | 1.012666811 | 0.5471954 | 0.60662363 | 0.985749572 | 1.260307809 | | |
| 373 | 1.26893617 | 1.235 | 0.388333471 | 0.538254978 | 0.706601669 | 0.67731141 | 0.752375444 | 0.735278434 | 0.725375181 | | |
| 374 | 1.160723574 | 1.689475813 | 0.561686919 | 0.954777222 | 1.066857279 | 1.929473433 | 1.82749583 | 1.107846801 | 0.525875759 | | |
| 375 | 0.806290717 | 0.406168567 | 2.234498475 | 1.313816297 | 1.412577645 | 1.342044327 | 1.322600801 | 0.951581983 | 1.243091017 | | |
| 376 | 1.003094911 | 0.821870702 | 1.353154164 | 1.051608955 | 0.865571931 | 1.035697894 | 1.054209802 | 1.057813444 | 1.300371042 | | |
| 377 | 0.811071812 | 0.68139729 | 1.048555785 | 0.957968591 | 1.160289827 | 1.042195338 | 1.233959542 | 1.021617489 | 1.102874222 | | |
| 378 | 1 | 1 | 1 | 1 | 1 | 1 | 1 | 1 | 1 | | |
| 379 | 1 | 1 | 1 | 1 | 1 | 1 | 1 | 1 | 1 | | |
| 380 | 1.018143301 | 0.91440872 | 0.714153636 | 0.852313382 | 0.761144833 | 0.587932629 | 0.595369217 | 0.984542947 | 0.955531247 | | |
| 381 | 1.708297872 | 2.234468085 | 0.502406761 | 0.535384898 | 0.450413375 | 0.392761345 | 0.172698879 | 1 | 1 | | |
| 382 | 1.342021277 | 1 | 1 | 1 | 1 | 1 | 1 | 1.043621943 | 0.836351619 | | |
| 383 | 0.970129508 | 0.681451051 | 1.372391936 | 1.271590809 | 2.505238137 | 2.149837134 | 3.240602166 | 0.877707995 | 0.892950392 | | |
| 384 | 0.360888773 | 0.459459459 | 0.694944756 | 0.659527774 | 0.371071086 | 0.344836285 | 0.399298724 | 1 | 1 | | |
| 385 | 1.526726313 | 2.005849966 | 0.718467988 | 1.022027997 | 0.626518866 | 0.712410289 | 0764780075 | 0.889165668 | 0.836444472 | | |
| 386 | 1.568973587 | 1.829488465 | 0.440755849 | 0.446898298 | 0.683406011 | 0.576405495 | 0.572654382 | 0.901565747 | 1.060663402 | | |
| 387 | 1.391678653 | 1.565592001 | 0.977197267 | 1.138591001 | 1.232129909 | 0.864423921 | 0.909227951 | 1.034619901 | 0.796563121 | | |
| 388 | 1.198756492 | 1.372837432 | 1.139182211 | 1.234683909 | 1.420198167 | 2.348234637 | 3.199516212 | 0.88612368 | 0.88612368 | | |
| 389 | 0.761941096 | 0.71722875 | 0.821721036 | 0.630957175 | 0.700295342 | 0.630957175 | 0.634044838 | 1.042676128 | 0.952863659 | | |
| 390 | 1.277848435 | 1.153674349 | 1.255764179 | 1.154416772 | 1.035947077 | 1.276948147 | 0.98217239 | 1.036976822 | 0.917667167 | | |
| 391 | 1 | 1 | 1.709526136 | 0.962432829 | 0.592134831 | 1.044357596 | 0.459208598 | 1 | 1 | | |
| 392 | 0.860349604 | 1.301790471 | 0.664880549 | 0.902737209 | 0.855459909 | 0.544781894 | 0.633823387 | 0.914661765 | 0.90344307 | | |
| 393 | 0.698198951 | 0.734420423 | 0.45538223 | 0.70400155 | 1.041711075 | 1.490359461 | 1.601346769 | 0.981990521 | 1.206635071 | | |
| 394 | 0.601369074 | 0.601369074 | 1.581543265 | 1.071268813 | 0.93523135 | 0.850682634 | 0.713084199 | 1.130848694 | 2.234082776 | | |
| 395 | 1 | 1 | 1 | 1 | 1.362765957 | 1.845106383 | 2.042765957 | 1 | 1 | | |
| 396 | 1 | 1 | 1 | 1 | 1 | 1 | 1 | 1 | 1 | | |
| 397 | 0.987666208 | 0.817606602 | 0.942013669 | 0.961671246 | 0.931515536 | 1.581131544 | 1.490734869 | 0.895127694 | 1.016120268 | | |
| 398 | 0.646136926 | 0.697071762 | 0.908102518 | 0.847005701 | 0.640356188 | 0.959815347 | 0.618359316 | 1.230757659 | 1.00742115 | | |
| 399 | 1.219066996 | 0.843888432 | 0.97140624 | 1.33852233 | 1.510823075 | 1.187172974 | 1.549186912 | 0.830499064 | 0.725422056 | | |
| 400 | 1.954148936 | 1.815957447 | 0.666051979 | 0.913156227 | 0.947617127 | 0.724370426 | 1.020803319 | 0.937188435 | 0.937188435 | | |
| 401 | 1 | 1 | 1.358990944 | 1.504435409 | 2.033727592 | 1.275827019 | 1.474311588 | 1 | 1 | | |
| 402 | 1 | 1 | 1 | 1 | 1 | 1 | 1 | 1 | 1 | | |
| 403 | 1 | 1 | 1 | 1 | 1 | 1 | 1 | 1 | 1 | | |
| 404 | 1.849148936 | 1.767234043 | 0.9303044 | 1.103714587 | 1.141011151 | 1.279347498 | 1.346820374 | 1.021234799 | 0.879326473 | | |
| 405 | 0.690563003 | 0.755576408 | 0.7744892 | 1.190134267 | 1.311675423 | 1.345300642 | 1.438937536 | 1.211726861 | 1.167495248 | | |
| 406 | 0.993776229 | 0.856177538 | 1 | 1.081382979 | 1.805638298 | 1.292978723 | 1.493723404 | 1.038132807 | 0.750558843 | | |
| 407 | 1 | 1 | 0.839550496 | 0.813922942 | 0.985113835 | 0.773759486 | 0.628166959 | 1 | 1 | | |
| 408 | 1 | 1 | 1.135531379 | 1.702733183 | 2.557648932 | 3.180443147 | 2.278227411 | 1 | 1 | | |
| 409 | 0.936504134 | 1.013912492 | 0.719315219 | 0.904104673 | 0.861031463 | 0.737053937 | 0.698955448 | 0.878072761 | 0.656750474 | | |
| 410 | 1.136636391 | 1.462651068 | 0.91419624 | 1.101920011 | 1.807387888 | 1.307955967 | 1.246269298 | 1 | 1 | | |
| 411 | 1.350526859 | 1.294156658 | 1.057361983 | 1.097673829 | 0.957850035 | 1.868606199 | 1.866070863 | 1.137602086 | 1.164985245 | | |
| 412 | 1 | 1 | 1 | 1 | 1 | 1 | 1 | 1 | 1 | | |
| 413 | 1.00393617 | 1 | 1 | 1 | 1 | 1 | 1 | 1 | 1 | | |
| 414 | 1.596098575 | 1.44306855 | 0.882254389 | 1.135471116 | 1.693874816 | 1.566479024 | 1.409864629 | 0.904023739 | 0.933341246 | | |
| 415 | 0.715770213 | 0.667921011 | 1.437536384 | 1.272790779 | 0.949528467 | 0.867330306 | 1.174991268 | 1.3219676 | 1.059596295 | | |
| 416 | 2.041276596 | 1.864680851 | 1.187664338 | 1.190443994 | 0.941702351 | 0.933889265 | 0.927503568 | 0.966855069 | 0.807480287 | | |
| 417 | 2.185061528 | 2.103787084 | 1 | 1 | 1 | 1 | 1 | 1.044953747 | 0.601392787 | | |
| 418 | 2.517463449 | 2.999187747 | 0.644690782 | 1.238817581 | 1.125891352 | 1.56599248 | 1.426422922 | 0.95957385 | 0.854510531 | | |
| 419 | 1.103287874 | 1.184480389 | 0.979133065 | 1.042842742 | 1.630947581 | 1.680040323 | 1.582862903 | 0.911687259 | 0.864762676 | | |
| | AD | AE | AF | AG | AH | AI | AJ | AK | AL | | |
| 1 | MG.IMPnCMS | MG.JRF | MG.JRFnCMS | PA.CMS | PA.IMP | PA.IMPnCMS | PA.JRF | PA.JRFnCMS | max | | |
| 2 | 1.042724783 | 1.061480176 | 0.934393638 | 2.061728395 | 2.220716652 | 1.612165011 | 1.918598816 | 1.485596708 | 2.220716652 | | |
| 3 | 1 | 1 | 1 | 2.774439063 | 2.988138776 | 1.408520312 | 0.975882179 | 2.397746368 | 2.988138776 | | |
| 4 | 1 | 1.121808511 | 1.052021277 | 1.266808511 | 1.183617021 | 1.006595745 | 1.366276596 | 1.421914894 | 1.751382979 | | |
| 5 | 1 | 1 | 1 | 1.104787234 | 1.082659574 | 1.242021277 | 1.145531915 | 1.248085106 | 1.500295975 | | |
| 6 | 0.966428192 | 0.863150771 | 0.873471262 | 1.240099212 | 1.1604023 | 1.21717681 | 1.268282046 | 1.123906348 | 1.765429848 | | |
| 7 | 1 | 1 | 1 | 1.210001574 | 1.324867503 | 0.501757884 | 0.493257071 | 0.785433174 | 1.795 | | |
| 8 | 0.673885857 | 0.800875398 | 0.831798545 | 1.017539221 | 1.153181738 | 1.060197426 | 1.053851578 | 0.972677596 | 2.296542996 | | |
| 9 | 0.933641128 | 0.841495821 | 0.998708451 | 1.086904413 | 0.831903202 | 1.169639556 | 1.136323648 | 1.370006758 | 3.001736993 | | |
| 10 | 1.167537815 | 1.009579075 | 1.438226698 | 0.818387935 | 1.001350181 | 1.381846436 | 1.1150456 | 1.334004178 | 1.808195539 | | |
| 11 | 0.770591549 | 0.786760563 | 0.939267606 | 1.549893617 | 1.153404255 | 1.301170213 | 1.449680851 | 1.201914894 | 1.843345703 | | |
| 12 | 1.135766423 | 0.806443494 | 0.891970803 | 1.21049036 | 0.98618491 | 1.1734477 | 1.211325338 | 1.066039168 | 2.075408433 | | |
| 13 | 1 | 1 | 1 | 1 | 1 | 1 | 1 | 1 | 2.517521713 | | |
| 14 | 1.032547251 | 0.935533604 | 1.089076686 | 1 | 1.143297872 | 1.075957447 | 1 | 1.34 | 1.955319149 | | |
| 15 | 0.723129081 | 0.778566047 | 0.853748117 | 0.947388342 | 1.044158466 | 1.15640508 | 0.969341408 | 1.036882833 | 2.702102922 | | |
| 16 | 1 | 1 | 1 | 1 | 1 | 1 | 1 | 1 | 1.888025331 | | |
| 17 | 1 | 1 | 1 | 1.228191489 | 1 | 1 | 1 | 1 | 1.843919659 | | |
| 18 | 0.986876155 | 0.99103512 | 0.928650647 | 0.956442556 | 0.993777508 | 0.901148379 | 0.807305104 | 0.861739262 | 2.120359367 | | |
| 19 | 1.469944382 | 1.112740732 | 1.056884983 | 1.127041831 | 1.110166785 | 1.19478765 | 1.079388863 | 0.95792292 | 2.387178962 | | |
| 20 | 1 | 1 | 1 | 1 | 1 | 1 | 1 | 1 | 1.185009336 | | |
| 21 | 0.790824241 | 1.004247094 | 0.929503319 | 1.464515247 | 1.050458088 | 1.182606318 | 1.149842746 | 1.01422125 | 2.017531306 | | |
| 22 | 0.82620436 | 1.346925997 | 1.129573827 | 0.669932691 | 0.93550311 | 0.678509557 | 0.971542984 | 0.855130499 | 1.729840999 | | |
| 23 | 0.903297754 | 1.09160427 | 0.837661303 | 1.394516191 | 1.049778335 | 1.334329221 | 1.356929453 | 1.329124904 | 1.654850234 | | |
| 24 | 0.695457853 | 0.916224925 | 0.900010394 | 0.822128702 | 0.851054762 | 0.956903899 | 0.705092691 | 0.801832517 | 1.669270198 | | |
| 25 | 0.79604143 | 1.1928916 | 1.051787741 | 1.294877092 | 0.952558011 | 1.164428837 | 1.235144254 | 1.265819734 | 3.206198347 | | |
| 26 | 1.166926677 | 1.374310972 | 1.366614665 | 1 | 1 | 1.113404255 | 1 | 1.015531915 | 1.994787234 | | |
| 27 | 0.950819034 | 1.062215478 | 1.145363994 | 1.311621461 | 1.118511186 | 1.224747575 | 1.125678084 | 1.248584439 | 2.083623039 | | |
| 28 | 1 | 1 | 1 | 1.104787234 | 1.072553191 | 1 | 1.00787234 | 1 | 1.781395534 | | |
| 29 | 0.927252267 | 0.922453167 | 0.948169723 | 0.823264104 | 0.801658604 | 0.929400211 | 0.804604808 | 0.810097116 | 2.428296568 | | |
| 30 | 1 | 1 | 1 | 1 | 1 | 1 | 1 | 1 | 4.503857155 | | |
| 31 | 1.227146814 | 1.079160452 | 1.066375453 | 0.971569005 | 1.07845027 | 0.939909406 | 0.907382421 | 1.100086739 | 1.509972607 | | |
| 32 | 1 | 1 | 1 | 1 | 1 | 1 | 1 | 1 | 1.195268404 | | |
| 33 | 1.514607185 | 1.346624556 | 1.062179234 | 0.646814692 | 0.75982569 | 0.53529778 | 1.074891056 | 0.959037145 | 1.997275957 | | |
| 34 | 0.866779457 | 1.017862128 | 0.84455649 | 0.757427743 | 0.928413731 | 0.904359244 | 0.859886582 | 0.767961501 | 2.520246578 | | |
| 35 | 0.719737331 | 0.866807087 | 1.044149837 | 0.924415464 | 0.925495794 | 1.102052628 | 0.917817733 | 0.850065591 | 2.065186463 | | |
| 36 | 1.364932212 | 1.044531376 | 1.598628155 | 1.202901317 | 1.665012407 | 1.021950754 | 0.916714386 | 2.177642044 | 2.177642044 | | |
| 37 | 0.937174211 | 1.120667581 | 0.940329218 | 0.595014251 | 0.787850428 | 0.625515804 | 0.893350917 | 0.800740205 | 1.338085106 | | |
| 38 | 0.570032669 | 1.010948583 | 0.855843659 | 0.866031294 | 1.174891307 | 1.129399916 | 1.187467761 | 1.292230601 | 1.715795832 | | |
| 39 | 1 | 1 | 1 | 1 | 1 | 1 | 1 | 1 | 1.98 | | |
| 40 | 0.859546452 | 1.01892831 | 1.163222385 | 1.09192667 | 1.161290323 | 0.909130971 | 1.114577825 | 1.297109113 | 1.679574468 | | |
| 41 | 0.716081359 | 0.813133237 | 0.716081359 | 1.051601988 | 1.138257375 | 1.404991012 | 1.00058158 | 1.008036375 | 2.152956094 | | |
| 42 | 0.612832592 | 0.88544224 | 0.863346825 | 0.902549722 | 0.97826087 | 1.073803191 | 1.095137604 | 1.065188483 | 1.572115385 | | |
| 43 | 0.68911809 | 0.905898714 | 0.920825125 | 0.900278229 | 0.973823325 | 1.094551356 | 0.754300951 | 0.856178994 | 2.063944927 | | |
| 44 | 0.709172555 | 0.866968236 | 0.866538995 | 0.892290483 | 0.89231992 | 1.050866916 | 0.871213682 | 0.863089105 | 1.958297872 | | |
| 45 | 1 | 1 | 1 | 1 | 1 | 1 | 1 | 1 | 2.082566083 | | |
| 46 | 0.747873697 | 1.052621316 | 0.961554039 | 1.280618973 | 0.939772301 | 1.051601273 | 1.080541541 | 0.93297279 | 2.959946506 | | |
| 47 | 1.260273973 | 1.100329782 | 0.838597159 | 1.592978723 | 1.183617021 | 1.327978723 | 1.820531915 | 1.358297872 | 1.820531915 | | |
| 48 | 2.033297872 | 1.048723404 | 1.02393617 | 1.570623703 | 1.30236236 | 0.929129188 | 1.682020362 | 1.454680241 | 2.033297872 | | |
| 49 | 0.986552567 | 0.984413203 | 1.325539935 | 1.571228606 | 1.718012343 | 1.146083858 | 1.252274607 | 2.41025641 | 2.93712766 | | |
| 50 | 1.302117427 | 1.088972013 | 0.971945762 | 1.036868268 | 0.916045291 | 0.808386265 | 1 .048099052 | 0.962625426 | 2.186170213 | | |
| 51 | 0.502692559 | 1.090323107 | 0.936398499 | 0.865859103 | 1.106788893 | 1.096907646 | 0.939224382 | 1.184440013 | 1.533123028 | | |
| 52 | 1 | 1 | 1 | 1 | 1 | 1 | 1 | 1 | 1.177917648 | | |
| 53 | 1.015685107 | 1.372211054 | 1.134018316 | 1.610238028 | 1.636434176 | 0.978022338 | 0.831555594 | 2.078402658 | 3.54637032 | | |
| 54 | 1 | 1 | 1 | 1 | 1 | 1 | 1 | 1 | 1.91976154 | | |
| 55 | 0.832413808 | 1.03626491 | 0.979981246 | 0.838172428 | 0.886653982 | 1.126467172 | 0.959697546 | 1.21982258 | 2.205435632 | | |
| 56 | 0.888135908 | 0.943093205 | 0.9506127 | 1 | 1 | 1.065319149 | 1 | 1 | 1.897178466 | | |
| 57 | 0.817033181 | 0.90423699 | 0.876910536 | 1.147759623 | 0.875239393 | 1.17717697 | 0.895201781 | 0.79459618 | 2.376071691 | | |
| 58 | 0.73686493 | 0.6006124 | 0.912410965 | 0.987289139 | 0.733456644 | 0.989064812 | 1.171663214 | 1.219754365 | 1.922027202 | | |
| 59 | 1 | 1 | 1 | 1 | 1 | 1 | 1 | 1 | 2.118660204 | | |
| 60 | 0.892158338 | 1.1410295 | 1.119807345 | 0.912001571 | 1.063445296 | 1.205526092 | 1.220454397 | 1.468670202 | 1.946388211 | | |
| 61 | 0.667727597 | 0.932283378 | 0.901607858 | 0.971050455 | 1.200419927 | 1.193612012 | 1.07660495 | 1.228478717 | 1.548198417 | | |
| 62 | 1 | 1 | 1 | 1 | 1 | 1 | 1 | 1 | 2.344487535 | | |
| 63 | 1.166210465 | 1.119942783 | 1.082451834 | 0.859078647 | 0.761527288 | 0.983731065 | 0.917244242 | 0.867337622 | 1.640632557 | | |
| 64 | 0.835638987 | 1.086601425 | 0.977729006 | 0.870923559 | 0.878318223 | 1.015009457 | 1.130813005 | 1.027312158 | 1.495203492 | | |
| 65 | 1.303154091 | 1.102452309 | 0.943818165 | 1.175614195 | 1.110267219 | 0.90969527 | 1.290969527 | 1.309390726 | 1.509356575 | | |
| 66 | 1.432184275 | 1.14683286 | 1.044986085 | 1.141747967 | 0.978857146 | 1.030791714 | 0.923486229 | 0.755454992 | 1.990445474 | | |
| 67 | 0.776800864 | 0.977093938 | 1.141215487 | 1 | 1.001382979 | 1.06 | 1 | 1.285 | 2.723510638 | | |
| 68 | 0.788877073 | 1.122202349 | 1.070068159 | 1.021036755 | 1.068164378 | 1.126220529 | 1.098806594 | 1.029821915 | 1.306231353 | | |
| 69 | 1 | 1 | 1 | 1 | 1 | 1 | 1 | 1 | 1.145851064 | | |
| 70 | 0.748288489 | 0.798439739 | 0.748288489 | 1.164525994 | 0.958205912 | 1.324057085 | 1.006422018 | 0.958205912 | 2.049187527 | | |
| 71 | 1 | 1 | 1 | 0.873358713 | 1.227488152 | 1.036593893 | 1.066350711 | 0.850672053 | 1.439610062 | | |
| 72 | 0.794388574 | 0.902053579 | 0.902138088 | 1.400319149 | 1.052234043 | 1.156276596 | 1.240425532 | 1.118297872 | 1.400319149 | | |
| 73 | 0.725836013 | 0.776913234 | 0.825154753 | 0.904878576 | 0.80024501 | 1.020429488 | 0.715968869 | 0.765078908 | 1.613131749 | | |
| 74 | 1 | 1 | 1 | 1 | 1 | 1 | 1 | 1 | 2.645851064 | | |
| 75 | 0.670402612 | 0.886996736 | 0.89390642 | 1.140970133 | 0.871578484 | 1.14918911 | 1.028693036 | 1.049167095 | 2.405626647 | | |
| 76 | 1.123702944 | 0.833277443 | 0.968880245 | 1.146331069 | 1.121773871 | 1.181861766 | 1.031428952 | 1.055400186 | 1.821919609 | | |
| 77 | 0.894958225 | 0.960357246 | 1.066954768 | 0.994198781 | 0.950985615 | 1.078079678 | 1.069614633 | 0.900846504 | 2.03255814 | | |
| 78 | 1 | 1 | 1 | 1 | 1 | 1 | 1 | 1 | 1.957809144 | | |
| 79 | 0.83977028 | 0.827494616 | 0.870782484 | 1.07212766 | 1.082659574 | 1.172340426 | 1.219361702 | 1.062340426 | 1.913829787 | | |
| 80 | 0.857724057 | 0.944575472 | 1.040035377 | 0.75 | 1.089388283 | 0.841901206 | 0.787406663 | 0.873420448 | 2.799558614 | | |
| 81 | 1 | 1 | 1 | 1 | 1 | 1 | 1 | 1 | 1.288229337 | | |
| 82 | 1 | 1 | 1 | 1 | 1 | 1 | 1 | 1 | 2.456382979 | | |
| 83 | 0.707405178 | 1.013320289 | 1.088801927 | 1.350898324 | 1.432500251 | 1.561678209 | 1.35792432 | 1.35862692 | 1.865868375 | | |
| 84 | 0.951263169 | 1.16231973 | 1.099314718 | 1.540947162 | 1.396029366 | 1.591665805 | 1.427825458 | 1.229603971 | 2.292097065 | | |
| 85 | 0.850266914 | 0.695021469 | 0.845740977 | 1.767919698 | 1.282807868 | 1.379682026 | 1.674885476 | 1.534997305 | 2.20384652 | | |
| 86 | 0.869662023 | 0.914722063 | 0.856348127 | 1.039223205 | 1.159170877 | 1.252007398 | 1.10206153 | 0.987955612 | 1.997200996 | | |
| 87 | 0.943522843 | 0.811617544 | 0.849224244 | 0.900411076 | 0.979135965 | 0.987163577 | 0.843403397 | 0.817265183 | 2.543371907 | | |
| 88 | 0.982939633 | 0.968775455 | 1.197619694 | 1.283341154 | 0.990239324 | 1.16015955 | 1.098967621 | 1.19042703 | 1.826611778 | | |
| 89 | 1 | 1 | 1 | 1.124361702 | 1 | 1 | 1.018510638 | 1 | 1.124361702 | | |
| 90 | 0.979971086 | 0.958918137 | 0.918830191 | 1.034680315 | 1.13261019 | 1.148155221 | 1.113245741 | 0.935604614 | 1.75005934 | | |
| 91 | 0.760484261 | 0.834468516 | 0.838494548 | 1.142975682 | 1.059636772 | 1.360443796 | 1.013490545 | 0.962847469 | 2.236809004 | | |
| 92 | 1.243132094 | 0.916515874 | 1.080769863 | 1 | 1 | 1 | 1 | 1 | 2.230032194 | | |
| 93 | 1.065417066 | 0.898421692 | 1.041765492 | 1.261537121 | 0.919325937 | 1.191407548 | 1.019694667 | 0.92233787 | 2.081177989 | | |
| 94 | 1 | 1.085319149 | 1.344361702 | 1.095371042 | 1.096878664 | 1.380479089 | 1.415321905 | 0.882237981 | 1.898723404 | | |
| 95 | 0.753975678 | 0.988347857 | 0.964715343 | 1.022453648 | 1.229743559 | 1.151366482 | 1.04550927 | 1.258036359 | 1.873970081 | | |
| 96 | 0.892771406 | 0.872670807 | 0.88913234 | 1.070765512 | 0.870168942 | 1.186093754 | 1.018797089 | 0.967960092 | 2.95755751 | | |
| 97 | 1.248675535 | 1.209789623 | 1.298972579 | 1 | 0.884422364 | 1 | 0.986143877 | 0.902838752 | 1.309272418 | | |
| 98 | 1.208123137 | 1.1231 94222 | 1.004442788 | 0.644906687 | 1.057503888 | 1.053479782 | 0.877741058 | 0.700835925 | 1.269471638 | | |
| 99 | 0.893026791 | 0.895591868 | 1.030590918 | 1 | 1 | 1 | 1 | 1 | 1.974574468 | | |
| 100 | 1 | 1 | 1 | 1 | 1 | 1 | 1 | 1 | 2.132635253 | | |
| 101 | 1 | 1 | 1 | 1 | 1 | 1 | 1 | 1 | 2.534574468 | | |
| 102 | 0.917178584 | 0.826623842 | 0.827528142 | 1.364487174 | 0.998893946 | 1.233589994 | 1.236142426 | 0.984685413 | 2.369311432 | | |
| 103 | 1.016804174 | 0.952074739 | 0.977008008 | 1.256778797 | 1.153312946 | 1.354943935 | 1.218858308 | 1.195922528 | 2.196905679 | | |
| 104 | 0.865546389 | 0.893391272 | 0.795301113 | 0.92116702 | 0.820162504 | 1.056500169 | 0.927257088 | 0.685190305 | 2.246112541 | | |
| 105 | 1.05015981 | 0.925250026 | 0.901020724 | 1.13492823 | 0.919279482 | 1.064396285 | 0.957894737 | 0.903349282 | 1.974352083 | | |
| 106 | 0.907248335 | 0.918154618 | 0.928964386 | 1 | 1 | 1 | 1 | 1 | 1.831595745 | | |
| 107 | 1.042344906 | 0.836124888 | 0.985080088 | 1.018801171 | 0.826134305 | 0.969885399 | 1.051646795 | 0.921804732 | 1.48176753 | | |
| 108 | 0.677042676 | 0.910519016 | 0.86548821 | 1.104715721 | 1.197273175 | 1.412608054 | 1.1l476562 | 1.294469042 | 2.004271418 | | |
| 109 | 0.88612368 | 0.904977376 | 0.907334087 | 1.075754752 | 1.278978755 | 1.092620201 | 1.132500932 | 1.085165859 | 1.608816121 | | |
| 110 | 1.201211099 | 0.860723593 | 0.936302315 | 0.815528634 | 0.785242291 | 0.682406388 | 0.647026432 | 0.735613987 | 2.52421181 | | |
| 111 | 0.685770313 | 0.82996577 | 0.868256212 | 1.271474506 | 1.422028524 | 1.432313432 | 1.008918068 | 1.16489463 | 2.453352175 | | |
| 112 | 1.601531704 | 1.113695236 | 1.16015317 | 1.564978541 | 1.786437768 | 1.481716738 | 0.604806867 | 1.100257511 | 2.019257841 | | |
| 113 | 0.782279394 | 0.874969945 | 0.759858139 | 1.402247913 | 1.41554271 | 1.386191394 | 1.074438022 | 0.998779705 | 2.027340426 | | |
| 114 | 1.682495375 | 1.155668798 | 0.979307994 | 0.993958814 | 0.782782619 | 0.78501745 | 0.925617895 | 0.79997755 | 1.682495375 | | |
| 115 | 1.484479942 | 1.244781267 | 1.253040479 | 1.10419914 | 1.400689623 | 0.935383307 | 1.075575079 | 1.085824949 | 1.588451792 | | |
| 116 | 1.101459091 | 0.934245204 | 0.968123795 | 1.337530769 | 0.958964727 | 1.179507975 | 1.164340291 | 0.939058921 | 2.309777246 | | |
| 117 | 1 | 1 | 1 | 0.542412002 | 0.542412002 | 0.542412002 | 0.542412002 | 0.542412002 | 2.092340426 | | |
| 118 | 1 | 1 | 1 | 1 | 1 | 1 | 1 | 1 | 1.92027355 | | |
| 119 | 0.96205318 | 0.924612767 | 0.935598439 | 0.826833369 | 1.453581349 | 0.932974557 | 1.054991856 | 1.229217949 | 1.480646388 | | |
| 120 | 0.820685901 | 0.863473054 | 0.826891671 | 1.009434548 | 1.057848675 | 1.095959282 | 1.015113897 | 0.949165167 | 2.08071487 | | |
| 121 | 1 | 1 | 1 | 1 | 1 | 1 | 1 | 1 | 2.117021277 | | |
| 122 | 0.822685104 | 0.952914406 | 0.896201645 | 1 | 1 | 1.177659574 | 1 | 1.043617021 | 2.137556404 | | |
| 123 | 1.053617021 | 1.007446809 | 1 | 0.898018167 | 0.949793559 | 0.864244426 | 0.938315442 | 0.904128819 | 1.402035969 | | |
| 124 | 1 | 1 | 1 | 1 | 1 | 1 | 1 | 1 | 1.820957447 | | |
| 125 | 1.210638298 | 1 | 1 | 1 | 1 | 1 | 1 | 1 | 1.623531305 | | |
| 126 | 0.844355007 | 0.790992991 | 0.929151343 | 1.077208918 | 1.370437655 | 0.984971098 | 1.125268373 | 1.138728324 | 2.016582507 | | |
| 127 | 1.542896025 | 1.433274459 | 1.214349828 | 1.160124906 | 0.945314044 | 0.708407447 | 0.91450255 | 0.918158821 | 1.542896025 | | |
| 128 | 0.723447772 | 0.894594595 | 0.929218408 | 1 | 1 | 1 | 1 | 1 | 1.817978723 | | |
| 129 | 1 | 1 | 1 | 1 | 1 | 1 | 1 | 1 | 1.867021277 | | |
| 130 | 0.677427259 | 1.018499234 | 0.916508423 | 0.881824464 | 1.088919604 | 0.954695539 | 1.046379482 | 1.117254089 | 1.857056954 | | |
| 131 | 2.146350365 | 1.38408064 | 1.070481404 | 1.206647618 | 1.31580634 | 0.594502452 | 1.761743264 | 1.508345128 | 2.278902452 | | |
| 132 | 1.175083201 | 1.014988892 | 1.047273315 | 0.932202346 | 0.897365385 | 0.840058024 | 1.331219966 | 1.2057565 | 1.455321987 | | |
| 133 | 1.210638298 | 1 | 1 | 0.802463639 | 0.991392104 | 0.836449985 | 0.799050163 | 0.721430692 | 1.210638298 | | |
| 134 | 0.753079312 | 1.217022064 | 1.036232033 | 1.184661819 | 1.297571683 | 1.44022957 | 1.974299172 | 2.255270499 | 2.255270499 | | |
| 135 | 1.492821535 | 1.03000997 | 1.133000997 | 0.957290673 | 0.850957658 | 0.930872532 | 0.826594261 | 0.892712996 | 4.406789628 | | |
| 136 | 0.703946554 | 0.988432513 | 0.812817023 | 1.301004923 | 1.16672287 | 1.083293991 | 1.244351521 | 1.209415256 | 1.439680851 | | |
| 137 | 1.123530625 | 0.969272015 | 1.082800577 | 0.757148611 | 0.757148611 | 0.757148611 | 0.757148611 | 0.775996778 | 1.262234043 | | |
| 138 | 0.855128047 | 0.973339107 | 0.915316096 | 0.814132175 | 0.734735879 | 0.878435856 | 0.791401784 | 0.831237137 | 2.457363683 | | |
| 139 | 1.036709825 | 0.950444275 | 0.941685707 | 1.270879685 | 1.155314051 | 1.244865651 | 1.096226254 | 0.993753209 | 1.964240147 | | |
| 140 | 1.181462006 | 1.107536859 | 1.506495515 | 1.216565526 | 1.208366146 | 0.822856349 | 0.994487423 | 1.836151387 | 3.119667503 | | |
| 141 | 1 | 1 | 1 | 0.721799892 | 0.825232281 | 0.927512862 | 1.151117254 | 0.834062812 | 3.404042553 | | |
| 142 | 1.128301047 | 1.055177021 | 0.950233801 | 1.307042323 | 1.110166785 | 0.879270959 | 1.130259635 | 0.936380831 | 1.374240785 | | |
| 143 | 0.801783156 | 0.988377647 | 0.979063843 | 1.023038811 | 0.926507019 | 1.101734104 | 1.011808423 | 1.138728324 | 1.893550126 | | |
| 144 | 1.043767399 | 1.026394474 | 0.991390865 | 0.996678406 | 0.844485353 | 0.832119417 | 0.944573395 | 0.95493837 | 2.055744681 | | |
| 145 | 1 | 1 | 1 | 1 | 1 | 1 | 1 | 1 | | | |
| 146 | 1.002450683 | 0.995868271 | 1.107283185 | 0.954236965 | 1.151462291 | 0.879827741 | 0.779770503 | 0.893973671 | 2.066718898 | | |
| 147 | 1 | 1 | 1 | 1 | 1 | 1 | 1 | 1 | 2.052066815 | | |
| 148 | 1 | 1 | 1 | 1 | 1 | 1 | 1 | 1 | 3.736595745 | | |
| 149 | 2.046806512 | 1.150699228 | 1.117668545 | 1.104712874 | 1.103981949 | 0.520005085 | 1.092700289 | 1.262147647 | 2.046806512 | | |
| 150 | 1 | 1 | 1 | 1 | 1 | 1 | 1 | 1 | 1.303950299 | | |
| 151 | 1 | 1 | 1 | 1.059042553 | 1.082659574 | 1.118723404 | 1.15606383 | 1.211170213 | 1.861489362 | | |
| 152 | 1.014561136 | 0.954693049 | 1.187620497 | 1.317848882 | 1.200510794 | 1.207835389 1.186150732 | 1.3595798 | 2.979270021 | | | |
| 153 | 1 | 1 | 1 | 1 | 1 | 1 | 1 | 1 | 1.847659574 | | |
| 154 | 1.799857645 | 2.259353174 | 2.289247742 | 0.550818999 | 1.161042922 | 0.38256126 | 0.606381887 | 0.601176074 | 2.289247742 | | |
| 155 | 1.149895725 | 0.975755996 | 0.994873132 | 0.82848581 | 0.980609907 | 0.82848581 | 0.82848581 | 0.82848581 | 2.108840902 | | |
| 156 | 0.81440846 | 0.993985459 | 0.896034369 | 0.885783327 | 1.031574708 | 1.107209021 | 1.137011679 | 1.21240435 | 1.953288113 | | |
| 157 | 1.194933689 | 0.979931233 | 1.021051154 | 1.269401104 | 1.220344757 | 0.994958098 | 1.205082783 | 1.193976971 | 1.39010101 | | |
| 158 | 2.075178076 | 1.341419458 | 1.361615699 | 0.911821355 | 0.851128843 | 0.603695712 | 0.929604796 | 0.888621783 | 2.075178076 | | |
| 159 | 1.051520614 | 0.905448625 | 1.095249171 | 1.089247685 | 0.480412065 | 0.483532267 | 0.46555396 | 0.46555396 | 1.36195584 | | |
| 160 | 0.871277698 | 0.8938169 | 0.901091836 | 1.089558991 | 1.154412054 | 1.246174122 | 1.035918014 | 0.877414532 | 1.678366138 | | |
| 161 | 1.019750755 | 1.125755287 | 1.122715257 | 0.94847098 | 1.184044102 | 0.907655502 | 1.100977741 | 1.07077179 | 1.331130762 | | |
| 162 | 1.263199593 | 0.98409824 | 1.052402241 | 1.180607352 | 0.844846978 | 0.977505476 | 1.092168354 | 0.87118925 | 1.425106383 | | |
| 163 | 0.66366659 | 0.989451961 | 0.862187572 | 0.84862608 | 1.023521701 | 0.912200214 | 0.974366443 | 1.162952714 | 1.851088024 | | |
| 164 | 1 | 1 | 1 | 1 | 1 | 1 | 1 | 1 | 1.930609818 | | |
| 165 | 1.091263538 | 0.942021661 | 1.086209386 | 1 | 1 | 1 | 1.00787234 | 1.127553191 | 2.778617021 | | |
| 166 | 0.882930249 | 0.759726604 | 1.354130155 | 1.479853879 | 1.481325991 | 1.136415681 | 1.167929775 | 2.353197754 | 3.348297872 | | |
| 167 | 1.002578485 | 0.957296662 | 0.92736512 | 0.736106162 | 1.153931002 | 0.802796586 | 0.968809924 | 0.796568752 | 1.329722501 | | |
| 168 | 1 | 1 | 1 | 1 | 1 | 1 | 1 | 1 | 3.273465067 | | |
| 169 | 1.413705211 | 1.429709328 | 1.477305034 | 0.694962399 | 0.640429266 | 0.663531125 | 0.78467934 | 0.61208296 | 2.201754816 | | |
| 170 | 1.141556715 | 1.053697453 | 1.130817224 | 1.358615093 | 1.215309711 | 1.534703814 | 1.498945091 | 1.594644306 | 1.961022511 | | |
| 171 | 1.114949897 | 0.91140052 | 1.005263335 | 1.281909227 | 1.18767313 | 1.175953548 | 1.13365651 | 1.266940124 | 1.906395265 | | |
| 172 | 1 | 1 | 1 | 1 | 1 | 1 | 1 | 1 | 3.726504272 | | |
| 173 | 1.499105584 | 1.295071014 | 1.214487381 | 1.05031298 | 1.127685262 | 1.453171135 | 1.491516082 | 1.162749498 | 1.955285435 | | |
| 174 | 0.71211833 | 0.872443641 | 0.838913726 | 0.901401274 | 0.948127389 | 1.011617834 | 1.127184713 | 1.29666242 | 1.482873851 | | |
| 175 | 1 | 1 | 1 | 1 | 1 | 1 | 1 | 1.192659574 | 2.162234043 | | |
| 176 | 0.749487963 | 0.7302488 | 0.669475875 | 0.864461223 | 0.94092324 | 1.448076032 | 0.974104245 | 1.230710643 | 2.209748609 | | |
| 177 | 0.726639231 | 1.020164701 | 0.960415653 | 1.252649365 | 1.165814945 | 1.300496903 | 1.374991169 | 1.172061559 | 1.794082249 | | |
| 178 | 0.940955895 | 0.931670734 | 0.934349146 | 1.154078693 | 1.008832837 | 1.126163148 | 0.934155212 | 1.03693732 | 2.015173152 | | |
| 179 | 1 | 1 | 1 | 0.43510099 | 0.378208739 | 0.378208739 | 0.378208739 | 0.378208739 | 1.40664693 | | |
| 180 | 1 | 1 | 1 | 1 | 1 | 1 | 1 | 1 | 1.879859611 | | |
| 181 | 1.333272958 | 1.347340458 | 1.481434523 | 1.017318125 | 1.166504382 | 1.45646126 | 1.230143274 | 1.230143274 | 1.892535022 | | |
| 182 | 0.536340507 | 0.851448619 | 0.721757914 | 1.133832899 | 1.219121054 | 1.250543006 | 1.126303215 | 1.164422242 | 1.319656757 | | |
| 183 | 1 | 1 | 1 | 1 | 1 | 1 | 1 | 1 | | | |
| 184 | 0.953092362 | 0.850921805 | 0.851693761 | 1.070617263 | 0.798910963 | 0.93699749 | 0.91325988 | 0.97792147 | 2.406007291 | | |
| 185 | 1.413815365 | 1.294207407 | 1.206937027 | 0.819414185 | 0.829512052 | 1.067953107 | 0.98623647 | 0.934675105 | 2.560057887 | | |
| 186 | 1 | 1 | 1 | 1 | 1 | 1 | 1 | 1 | 1.884574468 | | |
| 187 | 1.612879349 | 1.066740686 | 1.258419689 | 0.973113965 | 0.695699924 | 0.529378221 | 0.638295176 | 0.605875644 | 1.981808511 | | |
| 188 | 0.674788007 | 1.061023622 | 1.107495457 | 1.151817575 | 1.467092835 | 0.925331552 | 1.182902395 | 1.324720924 | 1.467092835 | | |
| 189 | 1 | 1 | 1 | 1.019680851 | 1.052234043 | 1.225957447 | 1.177234043 | 1.23893617 | 1.498051682 | | |
| 190 | 1 | 1 | 1 | 0.552892751 | 0.953350103 | 0.674777041 | 0.77 | | | | |
| 191 | 1.126273157 | 1.082712068 | 0.911302783 | 0.887236563 | 0.809656725 | 0.744848535 | 0.92 | | | | |
| 192 | 1 | 1 | 1 | 1 | 1 | 1 | 1 | 1 | 2.296382979 | | |
| 193 | 1 | 1 | 1 | 1 | 1 | 1 | 1 | 1 | 1.634620715 | | |
| 194 | 1 | 1 | 1 | 1 | 1 | 1 | 1 | 1 | 3.616584047 | | |
| 195 | 1.232454001 | 1.109377078 | 1.254134338 | 0.766189971 | 0.992131393 | 0.959095735 | 0.857116093 | 0.929557235 | 1.931276596 | | |
| 196 | 0.842488248 | 0.783179857 | 0.726254105 | 1.163404255 | 1 | 1.017234043 | 1 | 1 | 2.214255319 | | |
| 197 | 1 | 1 | 1 | 1 | 1 | 1 | 1 | 1 | 1.27534418 | | |
| 198 | 1 | 1 | 1.154042553 | 1 | 1 | 1 | 1 | 1 | 1.365944549 | | |
| 199 | 0.933875273 | 0.984166464 | 0.977008008 | 0.905458897 | 0.727193225 | 0.900073644 | 0.879821412 | 0.858970818 | 1.904251602 | | |
| 200 | 1 | 1 | 1 | 1 | 1.183617021 | 1.06 | 1.145531915 | 1 | 1.229148936 | | |
| 201 | 1.210638298 | 1 | 1 | 1 | 1 | 1 | 1 | 1 | 2.08610977 | | |
| 202 | 1.293357501 | 1.479579772 | 1.397039445 | 0.775781179 | 1.130994832 | 0.564247076 | 0.691721314 | 0.613565622 | 1.935078418 | | |
| 203 | 0.803766982 | 0.842151413 | 0.892483419 | 0.930527494 | 1.334435377 | 1.10590993 | 0.755993458 | 0.94989649 | 2.511369531 | | |
| 204 | 0.986151909 | 0.986151909 | 0.986151909 | 1.082562555 | 1.291792425 | 1.141660987 | 1.173692922 | 1.276409308 | 1.537994087 | | |
| 205 | 0.902935534 | 1.091233783 | 0.767593013 | 0.665300285 | 0.774355438 | 0.447279368 | 0.701311471 | 0.640718181 | 1.346461923 | | |
| 206 | 0.786714801 | 1.050180505 | 1.002815884 | 1.182959375 | 1.13689829 | 1.288820797 | 1.220816448 | 1.304240387 | 2.16675317 | | |
| 207 | 1.674072867 | 1.229884121 | 1.283098792 | 1.158412205 | 1.133477979 | 0.728875525 | 0.858227697 | 0.917851163 | 1.674072867 | | |
| 208 | 1.145021455 | 0.846717459 | 1.026943398 | 1.443313253 | 1.348192771 | 1.453975904 | 1.355 | 1.504337349 | 2.900652327 | | |
| 209 | 1.198992265 | 1.017528919 | 1.14292811 | 0.918295705 | 1.358067181 | 1.015556167 | 1.045223018 | 1.16127478 | 1.992785643 | | |
| 210 | 0.899657514 | 1.210238539 | 1.074265457 | 0.83900347 | 1.196414803 | 0.853556284 | 1.05247687 | 0.942029684 | 2.014665812 | | |
| 211 | 1.110540641 | 0.922677224 | 1.076209328 | 1.50258008 | 1.282835167 | 1.235030669 | 1.096679973 | 0.980722422 | 2.492109403 | | |
| 212 | 1.170779773 | 1.119048685 | 0.941998477 | 1.228270021 | 1.10841464 | 1.192957258 | 1.100193155 | 1.067505324 | 1.509186798 | | |
| 213 | 1.121282074 | 0.917251176 | 0.9323448461 | 1.062446809 | 1.145531915 | 1 | 1.090319149 | 2.874680851 | | | |
| 214 | 2.167156674 | 1.367066211 | 1.156327741 | 0.922464714 | 0.93847268 | 0.58700654 | 1.260321819 | 1.31828846 | 2.167156674 | | |
| 215 | 0.894595516 | 0.749722956 | 0.893103742 | 0.784047042 | 0.607909148 | 0.939985636 | 0.733638567 | 0.634213125 | 1.888290713 | | |
| 216 | 1.430966721 | 1.135106166 | 1.374719273 | 0.91911847 | 0.634313122 | 0.726143864 | 0.656700973 | 0.74134411 | 1.430966721 | | |
| 217 | 1.244468085 | 1.025851064 | 1 | 1.112292149 | 0.989265418 | 0.989265418 | 0.989265418 | 0.989265418 | 1.49778506 | | |
| 218 | 1.651391217 | 1.269752833 | 1.05854541 | 1.054727552 | 1.111038199 | 0.888239974 | 1.492556167 | 1.397718045 | 1.829529882 | | |
| 219 | 1.637050459 | 1.114994421 | 1.269617027 | 1.062721255 | 1.011929672 | 1.045757827 | 1.117710924 | 1.070163317 | 1.637050459 | | |
| 220 | 1.092960365 | 0.912870785 | 0.967548382 | 1.006595745 | 1 | 1 | 1.00787234 | 1 | 2.378085106 | | |
| 221 | 2.509890018 | 1.383389788 | 1.672604859 | 1.020651943 | 0.717261273 | 0.470269696 | 0.578838395 | 0.649253203 | 2.509890018 | | |
| 222 | 1.072798515 | 1.20169107 | 0.992472675 | 1.343652398 | 1.426694837 | 0.919897116 | 1.686110601 | 2.009277972 | 2.009277972 | | |
| 223 | 1.107138377 | 0.917450759 | 1.035461883 | 0.86774263 | 0.841747364 | 0.979728857 | 0.895717667 | 0.844846137 | 2.722779739 | | |
| 224 | 1 | 1 | 1 | 1 | 1 | 1 | 1 | 1 | 1.845106383 | | |
| 225 | 1 | 1 | 1 | 1 | 1 | 1 | 1 | 1 | 2.239912281 | | |
| 226 | 0.951369633 | 0.945977054 | 0.990306065 | 0.932288099 | 0.959843697 | 0.967700645 | 1.317953913 | 1.244256838 | 1.317953913 | | |
| 227 | 0.973985671 | 1.046575482 | 0.991839393 | 1.21715227 | 1.355495657 | 1.060689685 | 1.587066273 | 1.640504 | 1.952521799 | | |
| 228 | 0.774532812 | 0.994263364 | 0.900521512 | 0.963913656 | 1.125621774 | 1.203660253 | 1.329845143 | 1.309103707 | 1.557648637 | | |
| 229 | 0.840711922 | 0.973794831 | 0.9932921921 | 1 | 1 | 1 | 1 | 1.015531915 | 2.417553191 | | |
| 230 | 0.634663426 | 0.821146445 | 0.841806765 | 1.206191843 | 1.024784764 | 1.195408296 | 1.022610662 | 1.027828507 | 2.046184739 | | |
| 231 | 1.145672314 | 1.202345737 | 1.253084011 | 0.841045809 | 1.24887873 | 1.250432537 1.113577684 | 1.186207378 | 1.355688771 | | | |
| 232 | 1.027115554 | 1.004828111 | 1.042631621 | 0.956337067 | 0.690651203 | 1.06561544 | 0.945025199 | 0.687008957 | 2.557722769 | | |
| 233 | 1.018333636 | 1.138319114 | 1.380286803 | 1 | 1 | 1 | 1 | 1.155531915 | 1.854810375 | | |
| 234 | 1.205180267 | 0.967458706 | 1.011798277 | 1.198192889 | 1.086623453 | 1.286846068 | 1.245334905 | 1.147384273 | 1.815583517 | | |
| 235 | 0.856301572 | 0.894066491 | 1.0054439 | 1.175247841 | 0.915005508 | 1.075045304 | 1.181075223 | 1.053014959 | 2.102503912 | | |
| 236 | 0.817598831 | 1.099764591 | 0.97783911 | 0.881539097 | 0.856071035 | 1.026018722 | 0.917813877 | 0.989881608 | 1.099764591 | | |
| 237 | 0.817574143 | 0.961404497 | 0.853224402 | 0.981244801 | 1.158662936 | 1.162292974 | 1.173107464 | 1.261173712 | 1.899817343 | | |
| 238 | 0.954068437 | 0.865239232 | 0.785014424 | 0.712737127 | 0.827812908 | 0.429087624 | 0.588427181 | 0.471979658 | 1.807218908 | | |
| 239 | 0.703200508 | 0.769640737 | 0.606356833 | 0.88003569 | 1.542538479 | 1.037474905 | 1.545750613 | 1.743386125 | 2.018731028 | | |
| 240 | 1 | 1 | 1 | 1 | 1.011595745 | 1 | 1 | 1 | 1.632234043 | | |
| 241 | 1.455164586 | 1.153873439 | 1.267345346 | 0.90375666 | 0.838807581 | 0.916840898 | 1.043704053 | 0.986915762 | 2.06893617 | | |
| 242 | 1.03824547 | 0.894006473 | 1.041593809 | 0.852879883 | 0.770939565 | 0.911009622 | 1.075156298 | 1.00869064 | 1.781871649 | | |
| 243 | 1.244468085 | 1.121808511 | 1.172340426 | 0.857335835 | 1.145440901 | 0.705440901 | 0.956472795 | 0.821688555 | 1.738084941 | | |
| 244 | 1.242294838 | 0.872632752 | 0.909858893 | 0.636542997 | 0.693033382 | 1.145886248 | 0.650830007 | 0.614749637 | 2.592553191 | | |
| 245 | 1.251669465 | 0.987614251 | 1.219473979 | 1.294803991 | 1.522054414 | 0.965759773 | 0.974756011 | 2.121912655 | 2.121912655 | | |
| 246 | 1 | 1 | 1 | 1 | 1.001382979 | 1.049255319 | 1 | 1 | 1.971589857 | | |
| 247 | 0.876155438 | 0.804729056 | 0.891109067 | 1.439560139 | 1.567647783 | 1.598380611 | 1.22659135 | 1.306165714 | 2.056549098 | | |
| 248 | 0.798483755 | 0.932924188 | 0.960794224 | 1 | 1.072553191 | 1.177659574 | 1 | 1.099680851 | 1.889574468 | | |
| 249 | 1.065886486 | 0.896976595 | 0.822529566 | 1.078098162 | 1.134853634 | 1.19906443 | 1.022475606 | 1.016628294 | 2.405414593 | | |
| 250 | 0.962249759 | 1.027351412 | 1.128693297 | 1.252803984 | 1.092804794 | 1.058387658 | 1.045167429 | 1.144268535 | 1.975155022 | | |
| 251 | 1.866066373 | 1.220552694 | 1.179411399 | 0.839070905 | 0.785469787 | 0.717513098 | 1.168529514 | 1.14195599 | 1.866066373 | | |
| 252 | 1.376315028 | 0.926551491 | 0.96293794 | 1.156914894 | 1 | 1 | 1 | 1 | 2.098133432 | | |
| 253 | 0.982539056 | 0.993686955 | 0.862482559 | 1.031549865 | 0.803303526 | 1.057306675 | 0.893470518 | 0.782762115 | 2.08862671 | | |
| 254 | 1.160570142 | 0.879960065 | 1.098776447 | 1.136534879 | 0.735996688 | 0.989443179 | 0.776940592 | 0.888408197 | 1.94684466 | | |
| 255 | 0.987666751 | 0.828844702 | 0.843342562 | 0.739096386 | 0.886987952 | 0.761144578 | 0.867951807 | 0.851144578 | 1.776382979 | | |
| 256 | 0.795516313 | 0.941672713 | 0.866657648 | 1.049613754 | 0.948525176 | 1.061873181 | 1.081163316 | 1.006838693 | 1.667059301 | | |
| 257 | 1.119442509 | 0.926202091 | 1.087595819 | 1.08330562 | 0.752710343 | 1.252909877 | 1.008014633 | 0.885700033 | 2.331692436 | | |
| 258 | 1.84064493 | 1.557723876 | 1.458479565 | 0.957369127 | 0.96507268 | 0.49043305 | 0.737514866 | 0.596320928 | 1.84064493 | | |
| 259 | 0.627880569 | 0.899405517 | 0.96640171 | 0.853819664 | 0.874060018 | 0.841801954 | 1.012579942 | 0.969147516 | 1.450548454 | | |
| 260 | 1.401382887 | 1.407951599 | 1.318121579 | 1.088138059 | 1.53577096 | 1.387926474 | 1.338799259 | 1.217865644 | 1.638737098 | | |
| 261 | 1 | 1 | 1 | 1 | 1 | 1 | 1 | 1 | 1.339042553 | | |
| 262 | 0.796473838 | 0.964671906 | 1.117196502 | 1.449473451 | 1.147811145 | 1.471121178 | 1.286939769 | 1.63522995 | 2.889815091 | | |
| 263 | 1.334690044 | 0.975787936 | 0.991129201 | 0.586637012 | 0.505353908 | 0.485818449 | 0.4361085 | 0.457478275 | 1.518343195 | | |
| 264 | 1.207221123 | 1.142057248 | 1.096876961 | 1.263687322 | 0.976349535 | 1.074867542 | 1.098825155 | 0.920909161 | 2.093297872 | | |
| 265 | 0.943635304 | 0.913741625 | 0.909736382 | 0.903934035 | 0.96830916 | 1.083316776 | 0.991919995 | 0.766507716 | 2.121435958 | | |
| 266 | 1.159148936 | 1 | 1 | 1 | 1 | 1 | 1 | 1 | 2.042978723 | | |
| 267 | 1 | 1 | 1 | 1 | 1 | 1 | 1 | 1 | 1.847456109 | | |
| 268 | 1.017765957 | 1 | 1 | 0.950356189 | 0.697536361 | 0.697536361 | 0.697536361 | 0.697536361 | 1.378723404 | | |
| 269 | 1.148400253 | 1.006146246 | 1.098426101 | 1.072358253 | 1.013300252 | 1.037338323 | 1.097535183 | 0.976978768 | 1.874264912 | | |
| 270 | 1.017765957 | 1 | 1 | 1 | 1 | 1 | 1 | 1 | 1.84393617 | | |
| 271 | 0.699567445 | 0.89468517 | 0.927066188 | 0.777698637 | 1.081814202 | 1.072191184 | 0.90016397 | 0.947789377 | 1.693486302 | | |
| 272 | 0.901351244 | 0.951657862 | 0.869627545 | 0.941124955 | 1.196847259 | 0.984156682 | 1.095776442 | 1.107360376 | 1.64067324 | | |
| 273 | 0.754026388 | 0.919563239 | 0.836055505 | 1.034670616 | 0.865488337 | 1.006290163 | 0.957830098 | 0.859456302 | 3.405015674 | | |
| 274 | 1.317195735 | 1.190433877 | 0.855650202 | 1.181199852 | 1.28707461 | 1.142073227 | 1.148436779 | 1.065572259 | 1.771291315 | | |
| 275 | 1.143878484 | 1.535289801 | 1.443344438 | 0.984009633 | 1.210015451 | 0.671815522 | 0.79438244 | 0.874019214 | 1.899023036 | | |
| 276 | 1.068736617 | 0.862098501 | 0.925910064 | 0.888636793 | 1.06957837 | 1.027509926 | 0.914539611 | 1.026848175 | 2.613085106 | | |
| 277 | 1.228246124 | 1.422432171 | 1.335319767 | 0.646052277 | 0.647130154 | 0.625202102 | 0.656628941 | 0.755557801 | 1.823438618 | | |
| 278 | 1.053617021 | 1 | 1 | 0.868208092 | 0.863904945 | 0.723956326 | 0.755234425 | 0.731213873 | 1.342655468 | | |
| 279 | 0.58435907 | 1.128061669 | 1.111463384 | 1.18287234 | 1.133191489 | 1.473085106 | 1.082021277 | 1.006170213 | 2.055924822 | | |
| 280 | 0.981985137 | 0.862417805 | 0.922532197 | 1.173635437 | 0.969715781 | 1.055025939 | 0.964306301 | 1.068593979 | 3.269387173 | | |
| 281 | 1.082250133 | 1.004792182 | 2.144078679 | 1.584627667 | 1.820365801 | 1.118304543 | 1.003266076 | 2.923210916 | 3.73372093 | | |
| 282 | 0.900705523 | 0.946303827 | 0.960414277 | 1.210673317 | 1.225735661 | 1.274413965 | 1.245985037 | 1.126683292 | 2.055744681 | | |
| 283 | 0.874783814 | 1.300143331 | 1.561063716 | 1.080958583 | 1.698515238 | 1.549494949 | 1.217359845 | 1.453717693 | 2.139577128 | | |
| 284 | 0.736009098 | 0.980002843 | 0.900061603 | 0.803849001 | 0.74495083 | 1.039124458 | 0.766892249 | 0.813418632 | 2.540314704 | | |
| 285 | 1.66712766 | 1.126382979 | 1 | 0.918255964 | 0.576439566 | 0.576439566 | 0.66032992 | 0.576439566 | 1.66712766 | | |
| 286 | 1.271899225 | 1.101162791 | 1.166957364 | 0.94809531 | 0.923761825 | 1.186371225 | 1.109627156 | 1.231243993 | 1.862969973 | | |
| 287 | 1.004245661 | 1.075723548 | 1.005197959 | 1.153857565 | 0.831477223 | 0.751433143 | 0.809140326 | 0.625814587 | 1.868884868 | | |
| 288 | 0.910536655 | 1.104056877 | 1.213664286 | 1.359450663 | 1.109258818 | 1.805403403 | 1.446323199 | 0.966579344 | 1.805403403 | | |
| 289 | 1.048423862 | 0.935564574 | 0.980652694 | 0.850258626 | 0.898710142 | 0.982518169 | 0.866627382 | 0.863942906 | 2.26486698 | | |
| 290 | 1 | 1 | 1 | 0.517398509 | 0.743533094 | 0.692994569 | 1.031022738 | 0.776535027 | 5.887659574 | | |
| 291 | 1.201723264 | 1.077648251 | 1.143233654 | 0.973741442 | 1.00177205 | 1.184373741 | 0.859363673 | 0.790173178 | 1.867021277 | | |
| 292 | 0.772963128 | 0.971652159 | 0.87460025 | 1.265677813 | 1.169073337 | 1.521081193 | 1.211824901 | 1.089813427 | 2.334948374 | | |
| 293 | 0.724219044 | 0.909202108 | 0.897299586 | 1.125433526 | 1.33879255 | 1.354527938 | 1.086769428 | 1.28657675 | 2.012842012 | | |
| 294 | 0.796014266 | 0.844771603 | 0.873797631 | 0.742983745 | 1.124862593 | 0.915522827 | 0.769570124 | 1.010486687 | 2.051979526 | | |
| 295 | 1 | 1 | 1 | 0.660833333 | 0.489583333 | 0.489583333 | 0.489583333 | 0.489583333 | 1.564574468 | | |
| 296 | 1 | 1 | 1 | 0.656141792 | 0.551675568 | 0.551675568 | 0.551675568 | 0.551675568 | 2.137446809 | | |
| 297 | 1.547553191 | 1 | 1 | 1 | 1 | 1 | 1 | 1 | 1.836595745 | | |
| 298 | 0.967415431 | 1.117286691 | 1.069519666 | 1.206153634 | 1.148403084 | 1.570278084 | 1.283039648 | 1.329019824 | 2.436489362 | | |
| 299 | 0.866376639 | 0.978423324 | 1.30878621 | 1.360718434 | 1.406919149 | 2.072139303 | 1.615731913 | 1.313761589 | 3.832765957 | | |
| 300 | 1 | 1.266914894 | 1.10787234 | 0.78608463 | 1.163070748 | 0.78608463 | 1.582622512 | 1.146178291 | 1.582622512 | | |
| 301 | 0.847071309 | 0.640268863 | 0.879264163 | 0.561557142 | 0.735398729 | 0.673976501 | 1.004783281 | 0.916586945 | 7.937691573 | | |
| 302 | 0.738377393 | 0.859552025 | 0.932673525 | 1.694452738 | 1.38191915 | 1.5752075 | 1.387352959 | 1.424852212 | 2.739893617 | | |
| 303 | 0.648374627 | 0.877563935 | 0.899822536 | 1.054658326 | 1.028185429 | 1.332373998 | 1.094450919 | 1.005589515 | 2.266663125 | | |
| 304 | 1 | 1 | 1 | 0.583196469 | 0.546704989 | 0.783104085 | 0.532282899 | 0.65089304 | 1.227978723 | | |
| 305 | 1.516920396 | 1.226428298 | 1.100954619 | 0.874163451 | 0.801830382 | 0.749271501 | 1.216437795 | 1.157418216 | 1.8983562 | | |
| 306 | 1.09470128 | 1.406614509 | 1.251066856 | 0.767113863 | 1.287502315 | 0.696753195 | 1.078193221 | 0.821835887 | 1.497403983 | | |
| 307 | 1.463124504 | 1.026962728 | 1.141356067 | 0.94924916 | 0.952056834 | 0.905560046 | 0.736121155 | 0.759348279 | 2.605579263 | | |
| 308 | 1.622765957 | 1 | 1 | 0.946885035 | 0.939884393 | 0.882851638 | 1.018754014 | 0.803468208 | 7.322340426 | | |
| 309 | 0.834870849 | 0.915883932 | 0.911942301 | 0.950795084 | 1.417248802 | 0.96466912 | 1.063009513 | 1.165224637 | 1.424699056 | | |
| 310 | 1.115906085 | 0.878417108 | 0.887621252 | 0.97611523 | 0.834204449 | 0.887504558 | 0.981585025 | 0.868907257 | 1.960790415 | | |
| 311 | 1 | 1 | 1 | 1 | 1 | 1 | 1 | 1 | 2.115869757 | | |
| 312 | 0.772078827 | 1.053977838 | 0.928836839 | 0.793874602 | 0.924534944 | 0.856444082 | 0.809401171 | 0.841362188 | 2.349255319 | | |
| 313 | 1.517021277 | 1.30287234 | 1.281489362 | 1.013085106 | 1.193617021 | 1 | 1 | 1 | 1.517021277 | | |
| 314 | 1 | 1 | 1 | 0.508520422 | 0.508520422 | 0.508520422 | 0.508520422 | 0.508520422 | 1 | | |
| 315 | 1.502012953 | 1.182128479 | 1.430859443 | 0.967213115 | 0.900940098 | 0.864035428 | 0.797995494 | 0.7850439 | 1.602990033 | | |
| 316 | 1 | 1 | 1.061382979 | 1 | 1 | 1 | 1 | 1 | 1.947464923 | | |
| 317 | 1.089255319 | 1 | 1 | 1 | 1 | 1 | 1 | 1 | 1.298978644 | | |
| 318 | 1 | 1 | 1 | 0.62767094 | 0.62767094 | 0.62767094 | 0.62767094 | 0.62767094 | 1.848085106 | | |
| 319 | 1.073750127 | 1.365496099 | 1.721955047 | 0.87741338 | 1.90035469 | 1.468121538 | 1.553928678 | 1.961560638 | 2.147474858 | | |
| 320 | 1.800827182 | 1.857899742 | 1.590197588 | 0.6011213 | 0.412334299 | 0.209768613 | 0.437249964 | 0.341065235 | 2.011456777 | | |
| 321 | 2.802410519 | 3.130314098 | 2.002629657 | 1.104713506 | 0.991794489 | 1.02918551 | 0.886754627 | 0.933936314 | 3.130314098 | | |
| 322 | 1.017765957 | 1 | 1 | 0.62767094 | 0.62767094 | 0.62767094 0.62767094 | 0.62767094 | 1.846919302 | | | |
| 323 | 1.193873269 | 1.002622744 | 0.986151909 | 1 | 1.123191489 | 1 | 1 | 1.034255319 | 2.316515945 | | |
| 324 | 1.028673871 | 1.034857075 | 1.244394619 | 0.872382213 | 0.942248704 | 1.288149619 | 0.864603332 | 1.188657178 | 2.252585097 | | |
| 325 | 1.263961187 | 0.866730682 | 0.999747309 | 0.822128702 | 0.765075645 | 0.876997656 | 0.855263158 | 0.783986789 | 2.180522826 | | |
| 326 | 1.018396434 | 1.233000565 | 1.147610975 | 0.885882745 | 1.31558176 | 0.858874994 | 1.051828254 | 0.879653844 | 1.573032344 | | |
| 327 | 1.441606413 | 0.909226658 | 0.879597611 | 0.915198131 | 0.915198131 | 0.915198131 | 0.915198131 | 1.066425859 | 1.528617021 | | |
| 328 | 1.397901857 | 1.534395781 | 2.070196056 | 1.226155733 | 1.50040181 | 1.379604957 | 1.247980375 | 1.283551157 | 2.356788133 | | |
| 329 | 1 | 1 | 1 | 1 | 1 | 1 | 1 | 1 | 2.1252123383 | | |
| 330 | 0.91811369 | 0.906009464 | 0.91267085 | 0.953153368 | 0.887496652 | 1.252299176 | 1.070359237 | 0.919670228 | 3.084566118 | | |
| 331 | 1 | 1 | 1 | 1 | 1.04212766 | 1 | 1 | 1 | 1.198344407 | | |
| 332 | 1.02271685 | 1.166264102 | 1.056685113 | 0.772114839 | 1.345323096 | 0.757535924 | 1.087443611 | 1.031966062 | 1.525632104 | | |
| 333 | 1.141808511 | 1 | 1 | 0.54184352 | 0.332484437 | 0.332484437 | 0.332484437 | 0.332484437 | 1.164468085 | | |
| 334 | 0.890270666 | 0.952542063 | 1.054773226 | 1 | 1.163510638 | 1 | 1 | 1 | 1.915106383 | | |
| 335 | 1 | 1.035 | 1.033297872 | 0.552071122 | 0.501807347 | 0.459163736 | 0.459163736 | 0.459163736 | 1.711688784 | | |
| 336 | 0.868522484 | 0.946181299 | 0.919985724 | 1.160187842 | 0.817462388 | 1.024089051 | 1.202104531 | 0.944603879 | 4.114859813 | | |
| 337 | 1.228994684 | 1.023059246 | 1.014892841 | 1.161584251 | 1.063516345 | 1.070017625 | 0.872767783 | 1.125070666 | 2.239192302 | | |
| 338 | 0.779467681 | 0.854195039 | 0.765522351 | 1.407078741 | 1.267912737 | 1.369742075 | 1.279377343 | 1.309601182 | 1.969918675 | | |
| 339 | 0.54605594 | 0.902394677 | 0.879105575 | 1.121718065 | 1.795194133 | 1.205526092 | 1.338047535 | 1.588293066 | 1.795194133 | | |
| 340 | 0.91445144 | 0.987551551 | 0.86244321 | 0.740083314 | 0.99710694 | 0.828665715 | 0.813208929 | 0.944476918 | 2.242061507 | | |
| 341 | 0.80786679 | 0.953809671 | 0.880296938 | 1.032677978 | 0.974055122 | 1.360220846 | 0.972035192 | 0.996139689 | 2.539557286 | | |
| 342 | 1.198477009 | 0.846919848 | 0.758322757 | 1.246484086 | 1.034830167 | 1.184513529 | 1.224401678 | 1.06591825 | 2.224170182 | | |
| 343 | 1.460210155 | 0.806304651 | 0.713718863 1 | 1 | 1 | 1 | 1 | 1.557351753 | | | |
| 344 | 0.964386792 | 0.898820755 | 0.915978774 | 0.959186702 | 1.283689522 | 1.012540813 | 1.105224102 | 1.166295637 | 2.29122733 | | |
| 345 | 0.7148567 | 0.844718197 | 0.910898504 | 1.463194311 | 1.488966156 | 1.477993465 | 1.362780214 | 1.561931054 | 2.115540522 | | |
| 346 | 1.909893617 | 1.057872341 | 1.085212766 | 1.102978723 | 1 | 1.198297872 | 1 | 1.909893617 | | | |
| 347 | 1.003190868 | 0.880814345 | 1.072356299 | 0.897957921 | 0.803960396 | 0.825618812 | 0.703155941 | 0.639665842 | 1.847992236 | | |
| 348 | 1.066872149 | 0.97531747 | 1.190062878 | 0.987956013 | 1.133317818 | 0.977075697 | 0.858943775 | 0.980954597 | 1.974879917 | | |
| 349 | 1.118065108 | 0.924438331 | 1.097294819 | 0.985637657 | 1.022469554 | 0.957472088 | 0.817535338 | 1.041933849 | 1.985113083 | | |
| 350 | 1.387174997 | 1.092738785 | 1.083093089 | 0.91083237 | 0.979242653 | 1.084304481 | 0.998754559 | 1.201405569 | 1.8 | | |
| 351 | 1.709242784 | 1.193580685 | 1.002460165 | 0.932237085 | 0.816364204 | 0.380335691 | 1.011416887 | 0.949504416 | 2.261063261 | | |
| 352 | 1 | 1 | 1 | 1 | 1.28606383 | 1.143297872 | 1 | 1 | 1.503085106 | 1.503085106 | |
| 353 | 1 | 1 | 1.199787234 | 1.197776806 | 0.947542622 | 1.208767876 | 1.128395679 | 0.808030975 | 1.943297872 | | |
| 354 | 1.135135135 | 0.98540238 | 0.905960065 | 1.015951779 | 0.87506469 | 0.607385309 | 0.788304058 | 0.663429633 | 1.436937879 | | |
| 355 | 0.686164595 | 1.03285633 | 0.903833432 | 0.648813898 | 1.16167316 | 0.875455793 | 0.981213693 | 1.072663496 | 1.201913442 | | |
| 356 | 1 | 1 | 1 | 1 | 1 | 1 | 1 | 1 | 1.867021277 | | |
| 357 | 0.88360404 | 1.201796435 | 1.075254805 | 0.634219197 | 0.743879531 | 0.66615342 | 0.729713916 | 0.769008099 | 1.618989618 | | |
| 358 | 1 | 1 | 1 | 0.984644592 | 1.282818695 | 1.355910436 | 1.389804009 | 1.30135686 | 2.656492544 | | |
| 359 | 1 | 1 | 1 | 1 | 1 | 1 | 1 | 1 | 1.859574468 | | |
| 360 | 0.833154982 | 1.110526799 | 0.960834072 | 1.080841669 | 1.038022296 | 1.142849355 | 1.086974297 | 1.115211644 | 1.357630358 | | |
| 361 | 0.864465799 | 0.944717378 | 0.951492739 | 1.46970117 | 1.221017908 | 1.399318068 | 1.17026113 | 1.216443976 | 3.190718592 | | |
| 362 | 0.743494424 | 1.008700467 | 0.858024203 | 1.127217125 | 1.267787971 | 1.164424057 | 1.117940877 | 1.142813456 | 1.267787971 | | |
| 363 | 1 | 1 | 1 | 1 | 1 | 1 | 1 | 1 | 3.235425532 | | |
| 364 | 0.827611891 | 0.863374614 | 0.802143318 | 0.903736627 | 0.74858649 | 0.863259083 | 1.044384723 | 0.665874205 | 1.791358517 | | |
| 365 | 1.071489362 | 1.099042553 | 1.042659574 | 1.18287234 | 1.031914894 | 1.06 | 1.198297872 | 1.015531915 | 1.7483877097 | | |
| 366 | 1 | 1 | 1 | 1.020552344 | 1.25613359 | 1.433590238 | 1.15433526 | 1.573281952 | 2.259707193 | | |
| 367 | 0.711997477 | 0.78493178 | 0.768001195 | 1.040069409 | 0.90072593 | 1.357472618 | 1.191129648 | 1.173522669 | 2.114286534 | | |
| 368 | 1 | 1 | 1 | 1 | 1 | 1 | 1 | 1 | 2.150044085 | | |
| 369 | 0.759793671 | 0.938630977 | 0.862456434 | 0.972080028 | 0.994004038 | 1.205127159 | 1.191034609 | 1.072910081 | 4.27175051 | | |
| 370 | 1.57453322 | 1.356098643 | 1.302717492 | 1.062281282 | 1.093525553 | 1.24456942 | 1 .047538004 | 0.932242302 | 2.595615003 | | |
| 371 | 1.072189728 | 1.227946479 | 1.205352135 | 0.766533475 | 0.766533475 | 0.816602789 | 0.910462366 | 0.892848406 | 1.278343081 | | |
| 372 | 1.779403382 | 1.459908797 | 1.645259358 | 0.682563714 | 0.89822525 | 0.588695433 | 0.671124569 | 0.558541509 | 1.779403382 | | |
| 373 | 0.716081359 | 0.942637312 | 0.846042508 | 1.069335239 | 1.57692995 | 1.196568978 | 1.443084346 | 1.587651894 | 2.166276596 | | |
| 374 | 1.199953293 | 1.059831854 | 0.978701541 | 1.103592248 | 0.861430597 | 0.96608634 | 1.16925319 | 1.033480384 | 1.929473433 | | |
| 375 | 1.169293409 | 0.896377979 | 1.199151752 | 1.007031598 | 1.171384068 | 0.774032043 | 0.612527815 | 0.983622608 | 2.234498475 | | |
| 376 | 1.04996117 | 1.056777979 | 1.212787989 | 0.943159923 | 0.986062942 | 0.801734104 | 0.767951188 | 0.831021195 | 1.93501115 | | |
| 377 | 1.080735412 | 1.119765058 | 0.957877177 | 0.743297566 | 0.867850473 | 0.675339474 | 0.688380337 | 0.70999905 | 1.915590267 | | |
| 378 | 0.94493137 | 0.777034803 | 0.912809884 | 0.641521117 | 0.4714333 | 0.355468583 | 0.252481648 | 0.566878906 | 1 | | |
| 379 | 1 | 1 | 1 | 1 | 1 | 1 | 1 | 1 | 2.194099834 | | |
| 380 | 0.82255303 | 0.891229906 | 0.925853705 | 0.976414891 | 1.006929202 | 1.028233269 | 0.867398321 | 0.778738971 | 1.934825035 | | |
| 381 | 1 | 1 | 1 | 1 | | 1 | 1 | 1 | 6.955212766 | | |
| 382 | 0.773165896 | 0.848446794 | 0.835228024 | 0.936721728 | 0.862092334 | 1.237018213 | 0.827869547 | 0.796272766 | 1.580106383 | | |
| 383 | 0.687036906 | 0.976642439 | 0.844047703 | 0.875885203 | 1.176108833 | 1.069139023 | 0.984811778 | 1.173686172 | 3.240602166 | | |
| 384 | 1 | 1 | 1 | 1 | 1.123191489 | 1 | 1 | 1.303404255 | 1.433004158 | | |
| 385 | 0.737497635 | 0.877719619 | 0.887368355 | 1.15385234 | 1.036244705 | 1.34389577 | 1.147954533 | 1.191491073 | 2.005849966 | | |
| 386 | 0.646593945 | 1.055209261 | 0.968239834 | 0.957858255 | 1.306042493 | 1.041890602 | 1.05952082 | 1.098648852 | 1.829488465 | | |
| 387 | 1.021246344 | 1.102353013 | 0.99348459 | 1.078375967 | 1.103036612 | 0.918838895 | 1.565018117 | 1.341280893 | 1.565592001 | | |
| 388 | 0.88612368 | 0.88612368 | 0.88612368 | 0.834412436 | 0.630571455 | 1.146757999 | 0.897657252 | 0.763723374 | 3.199516212 | | |
| 389 | 0.952863659 | 0.952863659 | 0.952863659 | 1 | 1.193617021 | 1 | 1 | 1.052978723 | 2.160636183 | | |
| 390 | 1.061572453 | 0.969276305 | 0.937760547 | 0.942207265 | 0.884240455 | 1.023151909 | 0.938725775 | 0.912556574 | 1.964051968 | | |
| 391 | 1 | 1 | 1 | 1 | 1 | 1 | 1 | 1 | 1.709526136 | | |
| 392 | 0.627583943 | 0.962766272 | 1.011369679 | 1.103367342 | 1.21882135 | 1.142357618 | 1.18998579 | 1.209433028 | 1.301790471 | | |
| 393 | 1.427132701 | 1.188684834 | 1.250592417 | 0.748587363 | 0.966903941 | 1.237543113 | 1.192338739 | 1.681147721 | 1.681147721 | | |
| 394 | 2.359800149 | 1.718992641 | 1.529066235 | 0.929832768 | 0.905430164 | 0.608505828 | 0.756207851 | 0.777764784 | 2.359800149 | | |
| 395 | 1 | 1 | 1 | 1 | 1 | 1 | 1 | 1 | 2.042765957 | | |
| 396 | 1 | 1 | 1 | 1 | 1 | 1 | 1 | 1 | 2.3688923 | | |
| 397 | 1.354947775 | 0.922840065 | 0.853589326 | 1.250662589 | 1.191641182 | 1.092558614 | 1.208766565 | 1.124974516 | 1.581131544 | | |
| 398 | 1.040114326 | 1.35270521 | 1.124103696 | 1.391611118 | 1.637274393 | 1.378513234 | 1.591743656 | 1.673059681 | 1.673059681 | | |
| 399 | 0.820279246 | 0.748987271 | 0.761345644 | 0.936766312 | 1.288493038 | 1.043987169 | 1.00996451 | 1.12465875 | 2.046190397 | | |
| 400 | 0.937188435 | 0.937188435 | 0.937188435 | 1.014633836 | 1.03906598 | 1.291467837 | 1.082712986 | 1.120188331 | 1.954148936 | | |
| 410 | 1 | 1 | 1 | 1 | 1 | 1 | 1 | 1.090319149 | 2.033727592 | | |
| 402 | 1 | 0.926659063 | 0.973127939 | 0.872309105 | 0.697975742 | 0.686815505 | 0.464098545 | 0.717216693 | 1 | | |
| 403 | 1 | 1 | 1 | 1 | 1 | 1 | 1 | 1 | 2.635141623 | | |
| 404 | 1.079420019 | 0.994480823 | 1.134705332 | 1 | 1.011595745 | 1.129468085 | 1 | 1.275851064 | 1.849148936 | | |
| 405 | 1.261271141 | 1.096042306 | 1.294779939 | 1.106045922 | 1.001480202 | 1.201325133 | 1.221889372 | 1.006061781 | 1.481142714 | | |
| 406 | 0.769099277 | 0.838461538 | 0.841880342 | 0.958205912 | 1.162895005 | 0.958205912 | 0.965749235 | 1.089398573 | 1.805638298 | | |
| 407 | 1 | 1 | 1 | 1 | 1.011595745 | 1 | 1 | 1.043617021 | 1.238758251 | | |
| 408 | 1 | 1 | 1 | 1 | 1 | 1 | 1 | 1 | 3.180443147 | | |
| 409 | 0.866921683 | 0.859707152 | 0.85909311 | 1.11047861 | 0.951699463 | 1.11047861 | 1.11047861 | 1.01601536 | 1.111936029 | | |
| 410 | 1 | 1 | 1.052446809 | 2.281276596 | 1.868404255 | 1.759148936 | 1.583510638 | 2.281276596 | | | |
| 411 | 1.320499623 | 1.093816485 | 1.292636058 | 1.098297872 | 1 | 1.011914894 | 1 | 1.006170213 | 1.868606199 | | |
| 412 | 1 | 1 | 1 | 1 | 1 | 1 | 1 | 1 | 1.07477641 | | |
| 413 | 1 | 1 | 1 | 1 | 1 | 1 | 1 | 1 | 1.816303024 | | |
| 414 | 0.843774481 | 0.887145401 | 0.880545994 | 1.18847045 | 1.107964173 | 1.324549573 | 1.310059826 | 1.22198015 | 2.134586036 | | |
| 415 | 1.162773534 | 1.09509576 | 1.006310196 | 0.736155759 | 0.765694183 | 0.742342452 | 0.8100928 | 0.626978832 | 2.034803032 | | |
| 416 | 0.679366408 | 0.971530249 | 0.97494941 | 1.076604118 | 0.938927542 | 1.08995713 | 1.074003795 | 1.022559561 | 2.041276596 | | |
| 417 | 0.591414614 | 0.878754807 | 0.853861345 | 1.151216609 | 1.097328749 | 1.400952129 | 1.163118223 | 1.104601957 | 2.185061528 | | |
| 418 | 0.769526277 | 0.848212092 | 0.979920934 | 1.211142695 | 0.867879477 | 1.227743036 | 1.042455941 | 1.074792496 | 2.999187747 | | |
| 419 | 0.842346904 | 1.10073594 | 1.092836405 | 1.282111488 | 1.065310027 | 1.094356031 | 1.18505957 | 1.323419428 | 1.680040323 | | |

| | AM | | AM | | AM | | AM |
|---|---|---|---|---|---|---|---|
| 1 | min | 1 | min | 1 | min | 1 | min |
| 2 | 0.617720674 | 49 | 0.27783982 | 96 | 0.657987256 | 143 | 0.602525479 |
| 3 | 0.975882179 | 50 | 0.602448247 | 97 | 0.382084802 | 144 | 0.604985472 |
| 4 | 0.374159137 | 51 | 0.384386423 | 98 | 0.338017625 | 145 | 0.461479624 |
| 5 | 0.434105083 | 52 | 0.451640801 | 99 | 0.893026791 | 146 | 0.57766974 |
| 6 | 0.499975309 | 53 | 0.366885023 | 100 | 1 | 147 | 0.49659253 |
| 7 | 0.493257071 | 54 | 0.757270603 | 101 | 0.638500204 | 148 | 1 |
| 8 | 0.643130816 | 55 | 0.712224067 | 102 | 0.679807998 | 149 | 0.189006771 |
| 9 | 0.750773905 | 56 | 0.872632752 | 103 | 0.747921832 | 150 | 0.54598338 |
| 10 | 0.395613834 | 57 | 0.79459618 | 104 | 0.685190305 | 151 | 0.936161737 |
| 11 | 0.770591549 | 58 | 0.6006124 | 105 | 0.477289307 | 152 | 0.562493219 |
| 12 | 0.580658633 | 59 | 0.855115112 | 106 | 0.813003946 | 153 | 1 |
| 13 | 0.68949596 | 60 | 0.669166917 | 107 | 0.558380572 | 154 | 0.129959906 |
| 14 | 0.80129047 | 61 | 0.449564477 | 108 | 0.677042676 | 155 | 0.640803312 |
| 15 | 0.720711954 | 62 | 0.576620499 | 109 | 0.470047005 | 156 | 0.609360819 |
| 16 | 1 | 63 | 0.48107104 | 110 | 0.647026432 | 157 | 0.522579204 |
| 17 | 0.699456805 | 64 | 0.312010272 | 111 | 0.685770313 | 158 | 0.362416767 |
| 18 | 0.716511794 | 65 | 0.253919966 | 112 | 0.324518401 | 159 | 0.46555396 |
| 19 | 0.692804106 | 66 | 0.602417457 | 113 | 0.305283129 | 160 | 0.648650284 |
| 20 | 0.501467058 | 67 | 0.776800864 | 114 | 0.412230029 | 161 | 0.336123336 |
| 21 | 0.65582404 | 68 | 0.497450765 | 115 | 0.287437055 | 162 | 0.433025559 |
| 22 | 0.534945144 | 69 | 0.496881277 | 116 | 0.547455016 | 163 | 0.562022224 |
| 23 | 0.394132914 | 70 | 0.748288489 | 117 | 0.434842948 | 164 | 1 |
| 24 | 0.46631031171 | 71 | 0.365505203 | 118 | 0.387965671 | 165 | 0.861169898 |
| 25 | 0.448302175 | 72 | 0.399300334 | 119 | 0.49564269 | 166 | 0.545835251 |
| 26 | 0.773545221 | 73 | 0.395935179 | 120 | 0.767008097 | 167 | 0.429089161 |
| 27 | 0.736030759 | 74 | 0.756478352 | 121 | 1 | 168 | 0.863502705 |
| 28 | 0.551247861 | 75 | 0.670402612 | 122 | 0.753109815 | 169 | 0.390472833 |
| 29 | 0.689691105 | 76 | 0.670934199 | 123 | 0.401368018 | 170 | 0.744555027 |
| 30 | 1 | 77 | 0.743553235 | 124 | 1 | 171 | 0.897061304 |
| 31 | 0.529070749 | 78 | 0.781681497 | 125 | 0.384505256 | 172 | 0.988002181 |
| 32 | 0.52713633 | 79 | 0.751830581 | 126 | 0.474651586 | 173 | 0.520525452 |
| 33 | 0.228436886 | 80 | 0.499550979 | 127 | 0.215310085 | 174 | 0.447567811 |
| 34 | 0.669884546 | 81 | 0.515803336 | 128 | 0.723447772 | 175 | 0.877684407 |
| 35 | 0.320223641 | 82 | 1 | 129 | 0.963806008 | 176 | 0.669475875 |
| 36 | 0.894378651 | 83 | 0.707405178 | 130 | 0.677427259 | 177 | 0.419926014 |
| 37 | 0.555065854 | 84 | 0.587066076 | 131 | 0.309265271 | 178 | 0.735483302 |
| 38 | 0.358184612 | 85 | 0.293988866 | 132 | 0.558485656 | 179 | 0.378208739 |
| 39 | 1 | 86 | 0.591317099 | 133 | 0.370794052 | 180 | 0.494470842 |
| 40 | 0.53972894 | 87 | 0.723738504 | 134 | 0.214881701 | 181 | 0.450794404 |
| 41 | 0.716081359 | 88 | 0.404818059 | 135 | 0.826594261 | 182 | 0.220388258 |
| 42 | 0.503253302 | 89 | 0.5147789 | 136 | 0.446090585 | 183 | 0.540766551 |
| 43 | 0.55864057 | 90 | 0.556552798 | 137 | 0.485863441 | 184 | 0.798910963 |
| 44 | 0.709172555 | 91 | 0.755960401 | 138 | 0.515803336 | 185 | 0.425252618 |
| 45 | 0.930600931 | 92 | 0.700735655 | 139 | 0.654130134 | 186 | 0.864769686 |
| 46 | 0.585746033 | 93 | 0.786977492 | 140 | 0.266284397 | 187 | 0.529378221 |
| 47 | 0.806988969 | 94 | 0.799574065 | 141 | 0.4427342 | 188 | 0.29353339 |
| 48 | 0.194584748 | 95 | 0.693984525 | 142 | 0.444107358 | 189 | 0.35295324 |

| | AM | | AM | | AM | | AM | | AM | |
|---|---|---|---|---|---|---|---|---|---|---|
| 1 | min | 1 | min | 1 | min | 1 | min | 1 | min | |
| 190 | 0.552892751 | 237 | 0.758157046 | 284 | 0.597972935 | 331 | 0.511926805 | 378 | 0.252481648 | |
| 191 | 0.509159097 | 238 | 0.402978098 | 285 | 0.452363537 | 332 | 0.378309811 | 379 | 1 | |
| 192 | 1 | 239 | 0.606356833 | 286 | 0.648215892 | 333 | 0.332484437 | 380 | 0.587932629 | |
| 193 | 0.308267346 | 240 | 0.556938026 | 287 | 0.442389849 | 334 | 0.467057538 | 381 | 0.172698879 | |
| 194 | 0.884632717 | 241 | 0.838807581 | 288 | 0.69163417 | 335 | 0.459163736 | 382 | 0.445683898 | |
| 195 | 0.551287315 | 242 | 0.335910908 | 289 | 0.621766437 | 336 | 0.577861844 | 383 | 0.506510235 | |
| 196 | 0.726254105 | 243 | 0.15105012 | 290 | 0.517398509 | 337 | 0.46292613 | 384 | 0.344836285 | |
| 197 | 0.42560609 | 244 | 0.557855078 | 291 | 0.546225767 | 338 | 0.610106902 | 385 | 0.626518866 | |
| 198 | 0.533936202 | 245 | 0.727936174 | 292 | 0.680963117 | 339 | 0.422928102 | 386 | 0.440755849 | |
| 199 | 0.62401015 | 246 | 0.907906227 | 293 | 0.580263483 | 340 | 0.611884939 | 387 | 0.433022751 | |
| 200 | 0.558559629 | 247 | 0.804729056 | 294 | 0.738023298 | 341 | 0.689945904 | 388 | 0.630571455 | |
| 201 | 0.756478352 | 248 | 0.721080086 | 295 | 0.489583333 | 342 | 0.687276256 | 389 | 0.630957175 | |
| 202 | 0.175626909 | 249 | 0.814464609 | 296 | 0.551675568 | 343 | 0.44078182 | 390 | 0.884240455 | |
| 203 | 0.506079468 | 250 | 0.631698186 | 297 | 0.73151751 | 344 | 0.621230121 | 391 | 0.459208598 | |
| 204 | 0.553722903 | 251 | 0.548784249 | 298 | 0.82673197 | 345 | 0.627326099 | 392 | 0.544781894 | |
| 205 | 0.336076818 | 252 | 0.715100799 | 299 | 0.594965599 | 346 | 0.327937483 | 393 | 0.45538223 | |
| 206 | 0.665387843 | 253 | 0.680674967 | 300 | 0.419221373 | 347 | 0.624469247 | 394 | 0.480167057 | |
| 207 | 0.236429103 | 254 | 0.735996688 | 301 | 0.472338073 | 348 | 0.728834893 | 395 | 1 | |
| 208 | 0.624969 | 255 | 0.525051826 | 302 | 0.738377393 | 349 | 0.817535338 | 396 | 1 | |
| 209 | 0.683054298 | 256 | 0.497034721 | 303 | 0.648374627 | 350 | 0.645352892 | 397 | 0.516248974 | |
| 210 | 0.390673704 | 257 | 0.717037778 | 304 | 0.532282899 | 351 | 0.358372728 | 398 | 0.549846837 | |
| 211 | 0.741461145 | 258 | 0.202296504 | 305 | 0.444067596 | 352 | 0.304907717 | 399 | 0.65972613 | |
| 212 | 0.557173245 | 259 | 0.436500972 | 306 | 0.315068933 | 353 | 0.75365 | 400 | 0.666051979 | |
| 213 | 0.598293009 | 260 | 0.381803575 | 307 | 0.718215812 | 354 | 0.359324957 | 401 | 0.915109034 | |
| 214 | 0.456645908 | 261 | 0.340460584 | 308 | 0.593494116 | 355 | 0.393581046 | 402 | 0.464098545 | |
| 215 | 0.607909148 | 262 | 0.796473838 | 309 | 0.509321345 | 356 | 1 | 403 | 1 | |
| 216 | 0.504449319 | 263 | 0.4361085 | 310 | 0.590813094 | 357 | 0.528711619 | 404 | 0.629888348 | |
| 217 | 0.410367452 | 264 | 0.863172582 | 311 | 0.587056544 | 358 | 0.487956811 | 405 | 0.33895512 | |
| 218 | 0.425422724 | 265 | 0.687687688 | 312 | 0.474594392 | 359 | 1 | 406 | 0.489437371 | |
| 219 | 0.373219281 | 266 | 0.681356915 | 313 | 0.22932982 | 360 | 0.499916185 | 407 | 0.385313531 | |
| 220 | 0.613000421 | 267 | 0.784313725 | 314 | 0.508520422 | 361 | 0.812983789 | 408 | 1 | |
| 221 | 0.234759372 | 268 | 0.53946088 | 315 | 0.416349019 | 362 | 0.541568008 | 409 | 0.493831628 | |
| 222 | 0.563481 597 | 269 | 0.348793238 | 316 | 0.586489527 | 363 | 1 | 0.52660431 | 410 | 0.560583312 |
| 223 | 0.404874069 | 270 | 0.942544871 | 317 | 0.451528634 | 364 | 0.52660431 | 411 | 0.680095244 | |
| 224 | 1 | 271 | 0.080946558 | 318 | 0.62767094 | 365 | 0.543667149 | 412 | 0.538219296 | |
| 225 | 0.324238557 | 272 | 0.382358758 | 319 | 0.496851701 | 366 | 0.441336067 | 413 | 0.8358868 | |
| 226 | 0.212409869 | 273 | 0.658920874 | 320 | 0.163060943 | 367 | 0.489400859 | 414 | 0.754236235 | |
| 227 | 0.210617132 | 274 | 0.541104912 | 321 | 0.101068748 | 368 | 1 | 415 | 0.626978832 | |
| 228 | 0.547898493 | 275 | 0.123670821 | 322 | 0.475236278 | 369 | 0.713372638 | 416 | 0.679366408 | |
| 229 | 0.791778976 | 276 | 0.782523969 | 323 | 0.525896759 | 370 | 0.582719279 | 417 | 0.591414614 | |
| 230 | 0.634663426 | 277 | 0.625202102 | 324 | 0.682514738 | 371 | 0.347890452 | 418 | 0.644690782 | |
| 231 | 0.378419422 | 278 | 0.527003795 | 325 | 0.347704715 | 372 | 0.44009551 | 419 | 0.550725293 | |
| 232 | 0.600271657 | 279 | 0.58435907 | 326 | 0.415432023 | 373 | 0.388333471 | | | |
| 233 | 0.884189508 | 280 | 0.862417805 | 327 | 0.468477448 | 374 | 0.272697033 | | | |
| 234 | 0.44825685 | 281 | 0.681634155 | 328 | 0.484884222 | 375 | 0.406168567 | | | |
| 235 | 0.582589908 | 282 | 0.754535238 | 329 | 0.802321611 | 376 | 0.767951188 | | | |
| 236 | 0.550187401 | 283 | 0.381947134 | 330 | 0.7546261 | 377 | 0.625584235 | | | |

What is claimed is:

1. A polynucleotide microarray comprising at least one polynucleotide set forth in Table 1, Table 2, Table 3 or Table 4, wherein expression of said polynucleotide is either increased or decreased in brain cells in response to stress compared to normal brain cells.

2. A method for screening a compound for effectiveness in altering expression of a target polynucleotide wherein said target polynucleotide comprises a polynucleotide selected from Table 1, Table 2, Table 3 or Table 4, wherein said polynucleotide expression is either increased or decreased in brain cells in response to stress compared to normal brain cells.

3. A method of treating depression in a mammal comprising administering a compound identified in the method of claim 2.
